(19) United States
(12) Patent Application Publication
(10) Pub. No.: US 2012/0190089 A1
(43) Pub. Date: Jul. 26, 2012

(54) ENGINEERED MICROOGRANISMS CAPABLE OF PRODUCING TARGET COMPOUNDS UNDER ANAEROBIC CONDITIONS

(75) Inventors: Thomas Buelter, Denver, CO (US); Peter Meinhold, Denver, CO (US); Reid M. Renny Feldman, San Francisco, CA (US); Andrew C. Hawkins, Parker, CO (US); Sabine Bastian, Pasadena, CA (US); Frances Arnold, La Canada, CA (US); Jun Urano, Aurora, CA (US)

(73) Assignees: California Institute of Technology, Pasadena, CA (US); Gevo, Inc., Englewood, CO (US)

(21) Appl. No.: 13/303,884

(22) Filed: Nov. 23, 2011

Related U.S. Application Data
(63) Continuation-in-part of application No. 12/610,784, filed on Nov. 2, 2009.
(60) Provisional application No. 61/110,543, filed on Oct. 31, 2008.
(60) Provisional application No. 61/121,830, filed on Dec. 11, 2008.
(60) Provisional application No. 61/184,580, filed on Jun. 5, 2009.
(60) Provisional application No. 61/184,605, filed on Jun. 5, 2009.
(60) Provisional application No. 61/239,618, filed on Sep. 3, 2009.

(51) Int. Cl. C12P 7/16; C12N 1/19

(52) U.S. Cl. 435/160; 435/254.21; 435/254.22; 435/254.23; 435/254.2

(57) ABSTRACT

The present invention is generally provides recombinant microorganisms comprising engineered metabolic pathways capable of producing C3-C5 alcohols under aerobic and anaerobic conditions. The invention further provides ketol-acid reductoisomerase enzymes which have been mutated or modified to increase their NADH-dependent activity or to switch the cofactor preference from NADPH to NADH and are expressed in the modified microorganisms. In addition, the invention provides isobutyraldehyde dehydrogenase enzymes expressed in modified microorganisms. Also provided are methods of producing beneficial metabolites under aerobic and anaerobic conditions by contacting a suitable substrate with the modified microorganisms of the present invention.

### CROSS-REFERENCE TO RELATED APPLICATIONS

This application is a continuation-in-part appliation of U.S. application Ser. No. 12/610,784, filed Nov. 2, 2009, which claims priority to U.S. Provisional Application Ser. No. 61/110,543, filed Oct. 31, 2008; U.S. Provisional Application Ser. No. 61/121,830, filed Dec. 11, 2008; U.S. Provisional Application Ser. No. 61/184,580, filed Jun. 5, 2009; U.S. Provisional Application Ser. No. 61/184,605, filed Jun. 5, 2009; and U.S. Provisional Application Ser. No. 61/239,618, filed Sep. 3, 2009, all of which are herein incorporated by reference in their entireties for all purposes.

### STATEMENT REGARDING FEDERALLY FUNDED RESEARCH

This invention was made with government support under contract DE-FG02-07ER84893, awarded by the Department of Energy, and contract W911NF-09-2-0022, awarded by the United States Army Research Laboratory. The government has certain rights in the invention.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: GEVO_{—}018_{—}06US_SeqList.txt, date recorded: Nov. 3, 2011, file size: 1.98 megabytes).

### FIELD OF THE INVENTION

The present invention is generally related to genetically engineered microorganisms, methods of producing such organisms, and methods of using such organisms for the production of beneficial metabolites, including C3-C5 alcohols such as isobutanol.

### BACKGROUND

Biofuels have a long history ranging back to the beginning of the 20th century. As early as 1900, Rudolf Diesel demonstrated at the World Exhibition in Paris, France, an engine running on peanut oil. Soon thereafter, Henry Ford demonstrated his Model T running on ethanol derived from corn. Petroleum-derived fuels displaced biofuels in the 1930s and 1940s due to increased supply, and efficiency at a lower cost.

Market fluctuations in the 1970s coupled to the decrease in US oil production led to an increase in crude oil prices and a renewed interest in biofuels. Today, many interest groups, including policy makers, industry planners, aware citizens, and the financial community, are interested in substituting petroleum-derived fuels with biomass-derived biofuels. The leading motivations for developing biofuels are of economical, political, and environmental nature.

One is the threat of ‘peak oil’, the point at which the consumption rate of crude oil exceeds the supply rate, thus leading to significantly increased fuel cost results in an increased demand for alternative fuels. In addition, instability in the Middle East and other oil-rich regions has increased the demand for domestically produced biofuels. Also, environmental concerns relating to the possibility of carbon dioxide related climate change is an important social and ethical driving force which is starting to result in government regulations and policies such as caps on carbon dioxide emissions from automobiles, taxes on carbon dioxide emissions, and tax incentives for the use of biofuels.

Ethanol is the most abundant biofuel today but has several drawbacks when compared to gasoline. Butanol, in comparison, has several advantages over ethanol as a fuel: it can be made from the same feedstocks as ethanol but, unlike ethanol, it is compatible with gasoline at any ratio and can also be used as a pure fuel in existing combustion engines without modifications. Unlike ethanol, butanol does not absorb water and can thus be stored and distributed in the existing petrochemical infrastructure. Due to its higher energy content which is close to that of gasoline, the fuel economy (miles per gallon) is better than that of ethanol. Also, butanol-gasoline blends have lower vapor pressure than ethanol-gasoline blends, which is important in reducing evaporative hydrocarbon emissions.

Isobutanol has the same advantages as butanol with the additional advantage of having a higher octane number due to its branched carbon chain. Isobutanol is also useful as a commodity chemical. For example, it is used as the starting material in the manufacture of isobutyl acetate, which is primarily used for the production of lacquer and similar coatings. In addition, isobutanol finds utility in the industrial synthesis of derivative esters. Isobutyl esters such as diisobutyl phthalate (DIBP) are used as plasticizer agents in plastics, rubbers, and other dispersions.

A number of recent publications have described methods for the production of industrial chemicals such as isobutanol using engineered microorganisms. See, e.g., WO/2007/050671 to Donaldson et al., and WO/2008/098227 to Liao et al., which are herein incorporated by reference in their entireties. These publications disclose recombinant microorganisms that utilize a series of heterologously expressed enzymes to convert sugars into isobutanol. However, the production of isobutanol using these microorganisms is feasible only under aerobic conditions and the maximum yield that can be achieved is limited.

There is a need, therefore, to provide modified microorganisms capable of producing isobutanol under anaerobic conditions and at close to theoretical yield. The present invention addresses this need by providing modified microorganisms capable of producing isobutanol under anaerobic conditions and at high yields.

### SUMMARY OF THE INVENTION

The present invention provides recombinant microorganisms comprising an engineered metabolic pathway capable of producing one or more C3-C5 alcohols under aerobic and anaerobic conditions. In a preferred embodiment, the recombinant microorganism produces the C3-C5 alcohol under anaerobic conditions at a rate higher than a parental microorganism comprising a native or unmodified metabolic pathway. In another preferred embodiment, the recombinant microorganism produces the C3-C5 alcohol under anaerobic conditions at a rate of at least about 2-fold higher than a parental microorganism comprising a native or unmodified metabolic pathway. In another preferred embodiment, the recombinant microorganism produces the C3-C5 alcohol under anaerobic conditions at a rate of at least about 10-fold, of at least about 50-fold, or of at least about 100-fold higher than a parental microorganism comprising a native or unmodified metabolic pathway.

In various embodiments described herein, the C3-C5 alcohol may be selected from 1-propanol, 2-propanol, 1-butanol, 2-butanol, isobutanol, 2-methyl-1-butanol, 3-methyl-1-butanol, and 1-pentanol. In a preferred embodiment, the C3-C5 alcohol is isobutanol. In another preferred embodiment, isobutanol is produced at a specific productivity of at least about 0.025 g l⁻¹ h⁻¹ OD⁻¹.

In one aspect, there are provided recombinant microorganisms comprising an engineered metabolic pathway for producing one or more C3-C5 alcohols under anaerobic and aerobic conditions that comprises an overexpressed transhydrogenase that converts NADH to NADPH. In one embodiment, the transhydrogenase is a membrane-bound transhydrogenase. In a specific embodiment, the membrane-bound transhydrogenase is encoded by the E. coli pntAB genes or homologues thereof.

In another aspect, there are provided recombinant microorganisms comprising an engineered metabolic pathway for producing one or more C3-C5 alcohols under anaerobic and aerobic conditions that comprises an NADPH-dependent glyceraldehyde-3-phosphate dehydrogenase. In one embodiment, the NADPH-dependent glyceraldehyde-3-phosphate dehydrogenase is encoded by the Clostridium acetobutylicum gapC gene. In another embodiment, the NADPH-dependent glyceraldehyde-3-phosphate dehydrogenase is encoded by the Kluyveromyces lactis GDP1 gene.

In yet another aspect, there are provided recombinant microorganisms comprising an engineered metabolic pathway for producing one or more C3-C5 alcohols under anaerobic and aerobic conditions that comprises one or more enzymes catalyzing conversions in said engineered metabolic pathway that are not catalyzed by glyceraldehyde-3-phosphate dehydrogenase, and wherein said one or more enzymes have increased activity using NADH as a cofactor. In one embodiment, said one or more enzymes are selected from an NADH-dependent ketol-acid reductoisomerase (a ketol-acid reductoisomerase may hereinafter be abbreviated as “KARI”) and an NADH-dependent alcohol dehydrogenase (an alcohol dehydrogenase may hereinafter be abbreviated as “ADH”). In various embodiments described herein, the KARI and/or ADH enzymes may be engineered to have increased activity with NADH as the cofactor as compared to the wild-type E. coli KARI IlvC and a native E. coli ADH YqhD, respectively. In some embodiments, the KARI and/or the ADH are modified or mutated to be NADH-dependent. In other embodiments, the KARI and/or ADH enzymes are identified in nature with increased activity with NADH as the cofactor as compared to the wild-type E. coli KARI IlvC and a native E. coli ADH YqhD, respectively.

In various embodiments described herein, the KARI and/or ADH may show at least a 10-fold higher catalytic efficiency using NADH as a cofactor as compared to the wild-type E. coli KARI IlvC and the native ADH YqhD, respectively. In a preferred embodiment, the KARI enhances the recombinant microorganism's ability to convert acetolactate to 2,3-dihydroxyisovalerate under anaerobic conditions. In another embodiment, the KARI enhances the recombinant microorganism's ability to utilize NADH from the conversion of acetolactate to 2,3-dihydroxyisovalerate.

The present invention also provides modified or mutated KARI enzymes that preferentially utilize NADH rather than NADPH, and recombinant microorganisms comprising said modified or mutated KARI enzymes. In general, these modified or mutated KARI enzymes may enhance the cell's ability to produce beneficial metabolites such as isobutanol and enable the production of beneficial metabolites such as isobutanol under anaerobic conditions.

In certain aspects, the invention includes KARIs which have been modified or mutated to increase the ability to utilize NADH. Examples of such KARIs include enzymes having one or more modifications or mutations at positions corresponding to amino acids selected from the group consisting of: (a) alanine 71 of the wild-type E. coli IlvC (SEQ ID NO: 13); (b) arginine 76 of the wild-type E. coli IlvC; (c) serine 78 of the wild-type E. coli IlvC; and (d) glutamine 110 of the wild-type E. coli IlvC, wherein IlvC (SEQ ID NO: 13) is encoded by codon optimized E. coli ketol-acid reductoisomerase (KARI) genes Ec_ilvC_coEc (SEQ ID NO: 11) or Ec_ilvC_coSc (SEQ ID NO: 12).

In one embodiment, the KARI enzyme contains a modification or mutation at the amino acid corresponding to position 71 of the wild-type E. coli IlvC (SEQ ID NO: 13). In another embodiment, the KARI enzyme contains a modification or mutation at the amino acid corresponding to position 76 of the wild-type E. coli IlvC (SEQ ID NO: 13). In yet another embodiment, the KARI enzyme contains a modification or mutation at the amino acid corresponding to position 78 of the wild-type E. coli IlvC (SEQ ID NO: 13). In yet another embodiment, the KARI enzyme contains a modification or mutation at the amino acid corresponding to position 110 of the wild-type E. coli IlvC (SEQ ID NO: 13).

In one embodiment, the KARI enzyme contains two or more modifications or mutations at the amino acids corresponding to the positions described above. In another embodiment, the KARI enzyme contains three or more modifications or mutations at the amino acids corresponding to the positions described above. In yet another embodiment, the KARI enzyme contains four modifications or mutations at the amino acids corresponding to the positions described above.

In one specific embodiment, the invention is directed to KARI enzymes wherein the alanine at position 71 is replaced with serine. In another specific embodiment, the invention is directed to KARI enzymes wherein the arginine at position 76 is replaced with aspartic acid. In yet another specific embodiment, the invention is directed to KARI enzymes wherein the serine at position 78 is replaced with aspartic acid. In yet another specific embodiment, the invention is directed to KARI enzymes wherein the glutamine at position 110 is replaced with valine. In yet another specific embodiment, the invention is directed to KARI enzymes wherein the glutamine at position 110 is replaced with alanine. In certain embodiments, the KARI enzyme contains two or more modifications or mutations at the amino acids corresponding to the positions described in these specific embodiments. In certain other embodiments, the KARI enzyme contains three or more modifications or mutations at the amino acids corresponding to the positions described in these specific embodiments. In an exemplary embodiment, the KARI enzyme contains four modifications or mutations at the amino acids corresponding to the positions described in these specific embodiments. In additional embodiments described herein, the KARI may further comprise an amino acid substitution at position 68 of the wild-type E. coli IlvC (SEQ ID NO: 13).

In one embodiment, the modified or mutated KARI is selected from group consisting of SEQ ID NO: 17, SEQ ID NO: 19, SEQ ID NO: 23, SEQ ID NO: 25, SEQ ID NO: 34, SEQ ID NO: 36, SEQ ID NO: 38, SEQ ID NO: 40, SEQ ID NO: 42 and SEQ ID NO: 44.

Further included within the scope of the invention are KARI enzymes, other than the E. coli IlvC (SEQ ID NO: 13), which contain alterations corresponding to those set out above. Such KARI enzymes may include, but are not limited to, the KARI enzymes encoded by the S. cerevisiae ILV5 gene, the KARI enzyme encoded by the E. coli ilvC gene and the KARI enzymes from Piromyces sp., Buchnera aphidicola, Spinacia oleracea, Oryza sativa, Chlamydomonas reinhardtii, Neurospora crassa, Schizosaccharomyces pombe, Laccaria bicolor, Ignicoccus hospitalis, Picrophilus torridus, Acidiphilium cryptum, Cyanobacteria/Synechococcus sp., Zymomonas mobilis, Bacteroides thetaiotaomicron, Methanococcus maripaludis, Vibrio fischeri, Shewanella sp, Gramella forsetti, Psychromonas ingrhamaii, and Cytophaga hutchinsonii.

In certain exemplary embodiments, the KARI to be modified or mutated is a KARI selected from the group consisting of Escherichia coli (GenBank No: NP_{—}418222, SEQ ID NO 13), Saccharomyces cerevisiae (GenBank No: NP_{—}013459, SEQ ID NO: 70), Methanococcus maripaludis (GenBank No: YP_{—}001097443, SEQ ID NO: 71), Bacillus subtilis (GenBank Nos: CAB14789, SEQ ID NO: 72), Piromyces sp (GenBank No: CAA76356, SEQ ID NO: 73), Buchnera aphidicola (GenBank No: AAF13807, SEQ ID NO: 74), Spinacia oleracea (GenBank Nos: Q01292 and CAA40356, SEQ ID NO: 75), Oryza sativa (GenBank No: NP_{—}001056384, SEQ ID NO: 76) Chlamydomonas reinhardtii (GenBank No: XP_{—}001702649, SEQ ID NO: 77), Neurospora crassa (GenBank No: XP_{—}961335, SEQ ID NO: 78), Schizosaccharomyces pombe (GenBank No: NP_{—}001018845, SEQ ID NO: 79), Laccaria bicolor (GenBank No: XP_{—}001880867, SEQ ID NO: 80), Ignicoccus hospitalis (GenBank No: YP_{—}001435197, SEQ ID NO: 81), Picrophilus torridus (GenBank No: YP_{—}023851, SEQ ID NO: 82), Acidiphilium cryptum (GenBank No: YP_{—}001235669, SEQ ID NO: 83), Cyanobacteria/Synechococcus sp. (GenBank No: YP_{—}473733, SEQ ID NO: 84), Zymomonas mobilis (GenBank No: YP_{—}162876, SEQ ID NO: 85), Bacteroides thetaiotaomicron (GenBank No: NP_{—}810987, SEQ ID NO: 86), Vibrio fischeri (GenBank No: YP_{—}205911, SEQ ID NO: 87), Shewanella sp (GenBank No: YP_{—}732498, SEQ ID NO: 88), Gramella forsetti (GenBank No: YP_{—}862142, SEQ ID NO: 89), Psychromonas ingrhamaii (GenBank No: YP_{—}942294, SEQ ID NO: 90), and Cytophaga hutchinsonii (GenBank No: YP_{—}677763, SEQ ID NO: 91).

In additional aspects, the present application provides modified class II KARI enzymes, wherein said modified class II KARI enzymes are derived from unmodified class II KARI enzymes that have been engineered to comprise an amino acid substitution corresponding to amino acid residue Serine 78 of the E. coli KARI (SEQ ID NO: 13), and wherein said modified class II KARI exhibits an increased ability to use NADH as a cofactor to catalyze the conversion of acetolactate to 2,3-dihydroxyisovalerate as compared to a corresponding unmodified class II KARI. In one embodiment, the amino acid residue corresponding to the Serine 78 of E. coli KARI (SEQ ID NO: 13) is replaced with an amino acid residue selected from the group consisting of aspartic acid and glutamic acid. In further embodiments, the modified class II KARI enzymes may independently or additionally comprise an amino acid substitution corresponding to one or more amino acid residues selected from the group consisting of alanine 71, arginine 76, and glutamine 110 of the E. coli KARI (SEQ ID NO: 13). In some embodiments, the unmodified class II KARI is derived from a genus selected from the group consisting of Escherichia, Shigella, Yersinia, Klebsiella, Vibrio, Providencia, Haemophilus, Shewanella, and Salmonella. In further embodiments, the unmodified class II KARI comprises SEQ ID NO: 13. In yet further embodiments, the unmodified class II KARI comprises an amino acid sequence selected from the group consisting of SEQ ID NOs: 331-676.

In various embodiments described herein, the modified or mutated KARI may exhibit an increased catalytic efficiency with NADH as compared to the wild-type KARI. In one embodiment, the KARI has at least about a 5% increased catalytic efficiency with NADH as compared to the wild-type KARI. In another embodiment, the KARI has at least about a 25%, at least about a 50%, at least about a 75%, at least about a 100%, at least about a 500%, at least about 1000%, or at least about a 10000% increased catalytic efficiency with NADH as compared to the wild-type KARI.

In various embodiments described herein, the modified or mutated KARI may exhibit a decreased Michaelis Menten constant (K_{M}) for NADH as compared to the wild-type KARI. In one embodiment, the KARI has at least about a 5% decreased K_{M} for NADH as compared to the wild-type KARI. In another embodiment, the KARI has at least about a 25%, at least about a 50%, at least about a 75%, at least about a 90%, at least about a 95%, or at least about a 97.5% decreased K_{M} for NADH as compared to the wild-type KARI.

In various embodiments described herein, the modified or mutated KARI may exhibit an increased catalytic constant (k_{cat}) with NADH as compared to the wild-type KARI. In one embodiment, the KARI has at least about a 5% increased k_{cat} with NADH as compared to the wild-type KARI. In another embodiment, the KARI has at least about a 25%, at least about a 50%, at least about a 75%, at least about 100%, at least about 200%, or at least about a 500% increased k_{cat} with NADH as compared to the wild-type KARI.

In some embodiments described herein, the catalytic efficiency of the modified or mutated KARI with NADH is increased with respect to the catalytic efficiency with NADPH of the wild-type KARI. In one embodiment, the catalytic efficiency of said KARI with NADH is at least about 10% of the catalytic efficiency with NADPH of the wild-type KARI. In another embodiment, the catalytic efficiency of said KARI with NADH is at least about 25%, at least about 50%, or at least about 75% of the catalytic efficiency with NADPH of the wild-type KARI. In some embodiments, the modified or mutated KARI preferentially utilizes NADH rather than NADPH.

In various embodiments described herein, the modified or mutated KARI may exhibit an increased Michaelis Menten constant (K_{M}) for NADPH as compared to the wild-type KARI. In one embodiment, the KARI has at least about a 5% increased K_{M} for NADPH as compared to the wild-type KARI. In another embodiment, the KARI has at least about a 25%, at least about a 50%, at least about a 100%, at least about a 500%, at least about a 1000%, or at least about a 5000% increased K_{M} for NADPH as compared to the wild-type KARI.

In various embodiments described herein, the modified or mutated KARI may exhibit an decreased catalytic constant (k_{cat}) with NADPH as compared to the wild-type KARI. In one embodiment, the KARI has at least about a 5% decreased k_{cat} with NADPH as compared to the wild-type KARI. In another embodiment, the KARI has at least about a 25%, at least about a 50%, or at least about a 75%, at least about 90% decreased k_{cat} with NADPH as compared to the wild-type KARI.

In one embodiment, the application is directed to NADH-dependent KARI enzymes having a catalytic efficiency with NADH that is greater than the catalytic efficiency with NADPH. In one embodiment, the catalytic efficiency of the NADH-dependent KARI is at least about 2-fold greater with NADH than with NADPH. In another embodiment, the catalytic efficiency of the NADH-dependent KARI is at least about 4-fold, at least about 5-fold, at least about 6-fold, at least about 7-fold, at least about 8-fold, at least about 9-fold, at least about 10-fold, at least about 25-fold, at least about 50-fold, at least about 100-fold, or at least about 500-fold greater with NADH than with NADPH.

In one embodiment, the application is directed to modified or mutated KARI enzymes that demonstrate a switch in cofactor specificity from NADPH to NADH. In one embodiment, the modified or mutated KARI has at least about a 2:1 ratio of catalytic efficiency (k_{cat}/K_{M}) with NADH over k_{cat} with NADPH. In an exemplary embodiment, the modified or mutated KARI has at least about a 10:1 ratio of catalytic efficiency (k_{cat}/K_{M}) with NADH over catalytic efficiency (k_{cat}/K_{M}) with NADPH.

In some embodiments, the modified or mutated KARI has been modified to be NADH-dependent. In one embodiment, the KARI exhibits at least about a 1:10 ratio of K_{M} for NADH over K_{M} for NADPH.

In additional embodiments, the invention is directed to modified or mutated KARI enzymes that have been codon optimized for expression in certain desirable host organisms, such as yeast and E. coli. In other aspects, the present invention is directed to recombinant host cells (e.g. recombinant microorganisms) comprising a modified or mutated KARI enzyme of the invention. According to this aspect, the present invention is also directed to methods of using the modified or mutated KARI enzymes in any fermentation process where the conversion of acetolactate to 2,3-dihydroxyisovalerate is desired. In one embodiment according to this aspect, the modified or mutated KARI enzymes may be suitable for enhancing a host cell's ability to produce isobutanol and enable the production of isobutanol under anaerobic conditions. In another embodiment according to this aspect, the modified or mutated KARI enzymes may be suitable for enhancing a host cell's ability to produce 3-methyl-1-butanol.

According to this aspect, the present invention is also directed to methods of using the modified or mutated KARI enzymes in any fermentation process where the conversion of 2-aceto-2-hydroxy-butyrate to 2,3-dihydroxy-3-methylvalerate is desired. In one embodiment according to this aspect, the modified or mutated KARI enzymes may be suitable for enhancing a host cell's ability to produce 2-methyl-1-butanol and enable the production of 2-methyl-1-butanol under anaerobic conditions.

In another aspect, there are provided recombinant microorganisms comprising an engineered metabolic pathway for producing one or more C3-C5 alcohols under anaerobic conditions, wherein said engineered metabolic pathway comprises a first dehydrogenase and a second dehydrogenase that catalyze the same reaction, and wherein the first dehydrogenase is NADH-dependent and wherein the second dehydrogenase is NADPH dependent. In an exemplary embodiment, the first dehydrogenase is encoded by the E. coli gene maeA and the second dehydrogenase is encoded by the E. coli gene maeB.

In another aspect, there are provided recombinant microorganisms comprising an engineered metabolic pathway for producing one or more C3-C5 alcohols under anaerobic conditions, wherein said engineered metabolic pathway comprises a replacement of a gene encoding for pyk or homologs thereof with a gene encoding for ppc or pck or homologs thereof. In another embodiment, the engineered metabolic pathway may further comprise the overexpression of the genes mdh and maeB.

In various embodiments described herein, the recombinant microorganisms may further be engineered to express an isobutanol producing metabolic pathway comprising at least one exogenous gene that catalyzes a step in the conversion of pyruvate to isobutanol. In one embodiment, the recombinant microorganism may be engineered to express an isobutanol producing metabolic pathway comprising at least two exogenous genes. In another embodiment, the recombinant microorganism may be engineered to express an isobutanol producing metabolic pathway comprising at least three exogenous genes. In another embodiment, the recombinant microorganism may be engineered to express an isobutanol producing metabolic pathway comprising at least four exogenous genes. In another embodiment, the recombinant microorganism may be engineered to express an isobutanol producing metabolic pathway comprising five exogenous genes.

In various embodiments described herein, the isobutanol pathway enzyme(s) may be selected from the group consisting of acetolactate synthase (ALS), ketol-acid reductoisomerase (KARI), dihydroxyacid dehydratase (DHAD), 2-keto-acid decarboxylase (KIVD), and alcohol dehydrogenase (ADH). In an exemplary embodiment, the ketol-acid reductoisomerase is an NADH-dependent ketol-acid reductoisomerase (an NADH-dependent ketol-acid reductoisomerase may hereinafter be abbreviated as “NKR”).

In another embodiment, the recombinant microorganism further comprises a pathway for the fermentation of isobutanol from a pentose sugar. In one embodiment, the pentose sugar is xylose. In one embodiment, the recombinant microorganism is engineered to express a functional xylose isomerase (XI). In another embodiment, the recombinant microorganism further comprises a deletion or disruption of a native gene encoding for an enzyme that catalyzes the conversion of xylose to xylitol. In one embodiment, the native gene is xylose reductase (XR). In another embodiment, the native gene is xylitol dehydrogenase (XDH). In yet another embodiment, both native genes are deleted or disrupted. In yet another embodiment, the recombinant microorganism is engineered to express a xylulose kinase enzyme.

In another embodiment, the recombinant microorganisms of the present invention may further be engineered to include reduced pyruvate decarboxylase (PDC) activity as compared to a parental microorganism. In one embodiment, PDC activity is eliminated. PDC catalyzes the decarboxylation of pyruvate to acetaldehyde, which is reduced to ethanol by alcohol dehydrogenases via the oxidation of NADH to NAD+. In one embodiment, the recombinant microorganism includes a mutation in at least one PDC gene resulting in a reduction of PDC activity of a polypeptide encoded by said gene. In another embodiment, the recombinant microorganism includes a partial deletion of a PDC gene resulting in a reduction of PDC activity of a polypeptide encoded by said gene. In another embodiment, the recombinant microorganism comprises a complete deletion of a PDC gene resulting in a reduction of PDC activity of a polypeptide encoded by said gene. In yet another embodiment, the recombinant microorganism includes a modification of the regulatory region associated with at least one PDC gene resulting in a reduction of PDC activity of a polypeptide encoded by said gene. In yet another embodiment, the recombinant microorganism comprises a modification of the transcriptional regulator resulting in a reduction of PDC gene transcription. In yet another embodiment, the recombinant microorganism comprises mutations in all PDC genes resulting in a reduction of PDC activity of the polypeptides encoded by said genes.

In another embodiment, the recombinant microorganisms of the present invention may further be engineered to include reduced glycerol-3-phosphate dehydrogenase (GPD) activity as compared to a parental microorganism. In one embodiment, GPD activity is eliminated. GPD catalyzes the reduction of dihydroxyacetone phosphate (DHAP) to glycerol-3-phosphate (G3P) via the oxidation of NADH to NAD⁺. Glycerol is produced from G3P by Glycerol-3-phosphatase (GPP). In one embodiment, the recombinant microorganism includes a mutation in at least one GPD gene resulting in a reduction of GPD activity of a polypeptide encoded by said gene. In another embodiment, the recombinant microorganism includes a partial deletion of a GPD gene resulting in a reduction of GPD activity of a polypeptide encoded by the gene. In another embodiment, the recombinant microorganism comprises a complete deletion of a GPD gene resulting in a reduction of GPD activity of a polypeptide encoded by the gene. In yet another embodiment, the recombinant microorganism includes a modification of the regulatory region associated with at least one GPD gene resulting in a reduction of GPD activity of a polypeptide encoded by said gene. In yet another embodiment, the recombinant microorganism comprises a modification of the transcriptional regulator resulting in a reduction of GPD gene transcription. In yet another embodiment, the recombinant microorganism comprises mutations in all GPD genes resulting in a reduction of GPD activity of a polypeptide encoded by the gene.

In various embodiments described herein, the recombinant microorganisms of the invention may produce one or more C3-C5 alcohols under anaerobic conditions at a yield which is at least about the same yield as under aerobic conditions. In additional embodiments described herein, the recombinant microorganisms of the invention may produce one or more C3-C5 alcohols at substantially the same rate under anaerobic conditions as the parental microorganism produces under aerobic conditions. In the various embodiments described herein, the engineered metabolic pathway may be balanced with respect to NADH and NADPH as compared to a native or unmodified metabolic pathway from a corresponding parental microorganism, wherein the native or unmodified metabolic pathway is not balanced with respect to NADH and NADPH.

In another aspect, the present invention provides a method of producing a C3-C5 alcohol, comprising (a) providing a recombinant microorganism comprising an engineered metabolic pathway capable of producing one or more C3-C5 alcohols under aerobic and anaerobic conditions; (b) cultivating the recombinant microorganism in a culture medium containing a feedstock providing the carbon source, until a recoverable quantity of the C3-C5 alcohol is produced; and (c) recovering the C3-C5 alcohol. In one embodiment, the recombinant microorganism is cultured under anaerobic conditions. In a preferred embodiment, the C3-C5 alcohol is produced under anaerobic conditions at a yield which is at least about the same yield as under aerobic conditions.

In various embodiments described herein, a preferred C3-C5 alcohol is isobutanol. In one embodiment, the microorganism produces isobutanol from a carbon source at a yield of at least about 5 percent theoretical. In another embodiment, the microorganism is selected to produce isobutanol at a yield of at least about 10 percent, at least about 15 percent, about least about 20 percent, at least about 25 percent, at least about 30 percent, at least about 35 percent, at least about 40 percent, at least about 45 percent, at least about 50 percent, at least about 55 percent, at least about 60 percent, at least about 65 percent, at least about 70 percent, at least about 75 percent, at least about 80 percent theoretical, at least about 85 percent theoretical, at least about 90 percent theoretical, or at least about 95 percent theoretical. In one embodiment, the C3-C5 alcohol, such as isobutanol, is produced under anaerobic conditions at about the same yield as under aerobic conditions.

In another aspect, the present invention provides a recombinant microorganism comprising a metabolic pathway for producing a C3-C5 alcohol from a carbon source, wherein said recombinant microorganism comprises a modification that leads to the regeneration of redox co-factors within said recombinant microorganism. In one embodiment according to this aspect, the modification increases the production of a C3-C5 alcohol under anaerobic conditions as compared to the parental or wild-type microorganism. In a preferred embodiment, the fermentation product is isobutanol. In one embodiment, the re-oxidation or re-reduction of said redox co-factors does not require the pentose phosphate pathway, the TCA cycle, or the generation of additional fermentation products. In another embodiment, the re-oxidation or re-reduction of said redox co-factors does not require the production of byproducts or co-products. In yet another embodiment, additional fermentation products are not required for the regeneration of said redox co-factors.

In another aspect, the present invention provides a method of producing a C3-C5 alcohol, comprising providing a recombinant microorganism comprising a metabolic pathway for producing a C3-C5 alcohol, wherein said recombinant microorganism comprises a modification that leads to the regeneration of redox co-factors within said recombinant microorganism; cultivating the microorganism in a culture medium containing a feedstock providing the carbon source, until a recoverable quantity of said C3-C5 alcohol is produced; and optionally, recovering the C3-C5 alcohol. In one embodiment, said microorganism is cultivated under anaerobic conditions. In another embodiment, the C3-C5 alcohol is produced under anaerobic conditions at about the same yield as under aerobic conditions. In a preferred embodiment, the C3-C5 alcohol is isobutanol.

In various embodiments described herein, the recombinant microorganisms may be microorganisms of the Saccharomyces Glade, Saccharomyces sensu stricto microorganisms, Crabtree-negative yeast microorganisms, Crabtree-positive yeast microorganisms, post-WGD (whole genome duplication) yeast microorganisms, pre-WGD (whole genome duplication) yeast microorganisms, and non-fermenting yeast microorganisms.

In some embodiments, the recombinant microorganisms may be yeast recombinant microorganisms of the Saccharomyces clade.

In some embodiments, the recombinant microorganisms may be Saccharomyces sensu stricto microorganisms. In one embodiment, the Saccharomyces sensu stricto is selected from the group consisting of S. cerevisiae, S. kudriavzevii, S. mikatae, S. bayanus, S. uvarum. S. carocanis and hybrids thereof.

In some embodiments, the recombinant microorganisms may be Crabtree-negative recombinant yeast microorganisms. In one embodiment, the Crabtree-negative yeast microorganism is classified into a genera selected from the group consisting of Kluyveromyces, Pichia, Hansenula, or Candida. In additional embodiments, the Crabtree-negative yeast microorganism is selected from Kluyveromyces lactis, Kluyveromyces marxianus, Pichia anomala, Pichia stipitis, Hansenula anomala, Candida utilis, Issatchenkia orientalis and Kluyveromyces waltii.

In some embodiments, the recombinant microorganisms may be Crabtree-positive recombinant yeast microorganisms. In one embodiment, the Crabtree-positive yeast microorganism is classified into a genera selected from the group consisting of Saccharomyces, Kluyveromyces, Zygosaccharomyces, Debaryomyces, Candida, Pichia and Schizosaccharomyces. In additional embodiments, the Crabtree-positive yeast microorganism is selected from the group consisting of Saccharomyces cerevisiae, Saccharomyces uvarum, Saccharomyces bayanus, Saccharomyces paradoxus, Saccharomyces castelli, Saccharomyces kluyveri, Kluyveromyces thermotolerans, Candida glabrata, Z. bailli, Z. rouxii, Debaryomyces hansenii, Pichia pastorius, Schizosaccharomyces pombe, and Saccharomyces uvarum.

In some embodiments, the recombinant microorganisms may be post-WGD (whole genome duplication) yeast recombinant microorganisms. In one embodiment, the post-WGD yeast recombinant microorganism is classified into a genera selected from the group consisting of Saccharomyces or Candida. In additional embodiments, the post-WGD yeast is selected from the group consisting of Saccharomyces cerevisiae, Saccharomyces uvarum, Saccharomyces bayanus, Saccharomyces paradoxus, Saccharomyces castelli, and Candida glabrata.

In some embodiments, the recombinant microorganisms may be pre-WGD (whole genome duplication) yeast recombinant microorganisms. In one embodiment, the pre-WGD yeast recombinant microorganism is classified into a genera selected from the group consisting of Saccharomyces, Kluyveromyces, Candida, Pichia, Debaryomyces, Hansenula, Pachysolen, Issatchenkia, Yarrowia and Schizosaccharomyces. In additional embodiments, the pre-WGD yeast is selected from the group consisting of Saccharomyces kluyveri, Kluyveromyces thermotolerans, Kluyveromyces marxianus, Kluyveromyces waltii, Kluyveromyces lactis, Candida tropicalis, Pichia pastoris, Pichia anomala, Pichia stipitis, Debaryomyces hansenii, Hansenula anomala, Pachysolen tannophilis, Yarrowia lipolytica, Issatchenkia orientalis, and Schizosaccharomyces pombe.

In some embodiments, the recombinant microorganisms may be microorganisms that are non-fermenting yeast microorganisms, including, but not limited to those, classified into a genera selected from the group consisting of Tricosporon, Rhodotorula, or Myxozyma.

In certain specific embodiments, there are provided recombinant microorganisms comprising an engineered metabolic pathway for producing one or more C3-C5 alcohols under anaerobic conditions, wherein the recombinant microorganism is selected from GEVO1846, GEVO1886, GEVO1993, GEVO2158, GEVO2302, GEVO1803, GEVO2107, GEVO2710, GEVO2711, GEVO2712, GEVO2799, GEVO2847, GEVO2848, GEVO2849, GEVO2851, GEVO2852, GEVO2854, GEVO2855 and GEVO2856. In another specific embodiment, the present invention provides a plasmid is selected from the group consisting of pGV1698 (SEQ ID NO: 112), pGV1720 (SEQ ID NO: 115), pGV1745 (SEQ ID NO: 117), pGV1655 (SEQ ID NO: 109), pGV1609 (SEQ ID NO: 108), pGV1685 (SEQ ID NO: 111), and pGV1490 (SEQ ID NO: 104).

In yet another aspect, the present invention provides methods for the conversion of an aldehyde with three to five carbon atoms to the corresponding alcohol is provided. The method includes providing a microorganism comprising a heterologous polynucleotide encoding a polypeptide having NADH-dependent aldehyde reduction activity that is greater than its NADPH-dependent aldehyde reduction activity and having NADH-dependent aldehyde reduction activity that is greater than the endogenous NADPH-dependent aldehyde reduction activity of the microorganism; and contacting the microorganism with the aldehyde.

In another embodiment, a method for the conversion of an aldehyde derived from the conversion of a 2-ketoacid by a 2-ketoacid decarboxylase is provided. The method includes providing a microorganism comprising a heterologous polynucleotide encoding a polypeptide having NADH-dependent aldehyde reduction activity that is greater than its NADPH-dependent aldehyde reduction activity and having NADH-dependent aldehyde reduction activity that is greater than the endogenous NADPH-dependent aldehyde reduction activity of the microorganism; and contacting the microorganism with the aldehyde. In various embodiments described herein, the aldehyde may be selected from 1-propanal, 1-butanal, isobutyraldehyde, 2-methyl-1-butanal, or 3-methyl-1-butanal. In a preferred embodiment, the aldehyde is isobutyraldehyde.

In another embodiment, an microorganism include a heterologous polynucleotide encoding a polypeptide having NADH-dependent aldehyde reduction activity that is greater than its NADPH-dependent aldehyde reduction activity and having NADH-dependent aldehyde reduction activity that is greater than the endogenous NADPH-dependent aldehyde reduction activity of the microorganism is provided. The microorganism converts an aldehyde comprising three to five carbon atoms to the corresponding alcohol.

In another embodiment, an isolated microorganism is provided. The microorganism includes a heterologous polynucleotide encoding a polypeptide having NADH-dependent aldehyde reduction activity that is greater than its NADPH-dependent aldehyde reduction activity and having NADH-dependent aldehyde reduction activity that is greater than the endogenous NADPH-dependent aldehyde reduction activity of the microorganism. The microorganism converts an aldehyde derived from a 2-ketoacid by a 2-ketoacid decarboxylase. In one embodiment, the polypeptide is encoded by the Drosophila melanogaster ADH gene or homologs thereof. In a preferred embodiment, the Drosophila melanogaster ADH gene is set forth in SEQ ID NO: 60. In an alternative embodiment, the Drosophila melanogaster alcohol dehydrogenase is set forth in SEQ ID NO: 61. In another embodiment, the polypeptide possesses 1,2 propanediol dehydrogenase activity and is encoded by a 1,2 propanediol dehydrogenase gene. In a preferred embodiment, the 1,2-propanediol dehydrogenase gene is the Klebsiella pneumoniae dhaT gene as set forth in SEQ ID NO: 62. In an alternative embodiment, the 1,2-propanediol dehydrogenase is set forth in SEQ ID NO: 63. In another embodiment, the polypeptide possesses is encoded by a 1,3-propanediol dehydrogenase gene. In a preferred embodiment, the 1,3-propanediol dehydrogenase gene is the Escherichia coli fucO gene as set forth in SEQ ID NO: 64. In an alternative embodiment, the 1,3-propanediol dehydrogenase is set forth in SEQ ID NO: 65.

In yet another aspect, the present invention provides a recombinant microorganism producing isobutanol, wherein said recombinant microorganism i) does not overexpress an alcohol dehydrogenase; and ii) produces isobutanol at a higher rate, titer, and productivity as compared to recombinant microorganism expressing the S. cerevisiae alcohol dehydrogenase ADH2.

### BRIEF DESCRIPTION OF THE DRAWINGS

Illustrative embodiments of the invention are illustrated in the drawings, in which:

FIG. 1 illustrates an exemplary metabolic pathway for the conversion of glucose to isobutanol via pyruvate.

FIG. 2 illustrates a metabolic pathway for the conversion of glucose to isobutanol via pyruvate in which a transhydrogenase converts NADH from glycolysis to NADPH

FIG. 3 illustrates a metabolic pathway for the conversion of glucose to isobutanol via pyruvate in which an NADPH-dependent glyceraldehyde-3-phosphate dehydrogenase converts generates NADPH during glycolysis.

FIG. 4 illustrates a Transhydrogenase cycle converting NADH to NADPH

FIG. 5 illustrates an exemplary isobutanol pathway; on the left native conversion of PEP to pyruvate; on the right bypass of pyruvate kinase.

FIG. 6 illustrates an amino acid sequence alignment among various members of the KARI enzyme family.

FIG. 7 illustrates the structure alignment of E. coli KARI with rice KARI.

FIG. 8 illustrates growth of GEVO1859 under anaerobic shift conditions over the course of the fermentation.

FIG. 9 illustrates isobutanol production of GEVO1859 under anaerobic shift conditions over the course of the fermentation.

FIG. 10 illustrates that microorganisms featuring an overexpressed E. coli pntAB operon (pGV1745) increased in OD₆₀₀ from 6 h to 24 h by 0.2-1.1 under anaerobic conditions, while microorganisms lacking E. coli pntAB (pGV1720) decreased in OD₆₀₀ by 0.5-1.2.

FIG. 11 illustrates that microorganisms featuring an overexpressed E. coli pntAB operon (pGV1745) continued isobutanol production under anaerobic conditions until the fermentation was stopped at 48 hours while microorganisms lacking E. coli pntAB (pGV1720) did not produce isobutanol between 24 and 48 hours

FIG. 12 illustrates that for strains GEVO1886, GEVO1859 and GEVO1846 stable OD values can be observed under anaerobic shift conditions over the course of the fermentation

FIG. 13 illustrates that over-expression of E. coli pntAB in either strain GEVO1846 or GEVO1886 leads to an improvement in isobutanol production over the course of the fermentation compared to the control strain GEVO1859 which does not over-express E. coli pntAB.

FIG. 14 illustrates that a strain lacking zwf without E. coli pntAB (Δzwf) grew to an OD of about 3, whereas the samples featuring E. coli pntAB (Δzwf+pntAB) reached OD values of about 5-6.

FIG. 15 illustrates an SDS PAGE of crude extracts of E. coli BL21(DE3) and GEVO1777 containing overexpressed KARI from plasmids pGV1777 and pET22[ilvC_co], respectively. The arrow highlights the KARI band. The protein marker (M) was an unstained 200 kDa ladder from Fermentas.

FIG. 16 illustrates an SDS PAGE of crude extract (C), purified KARI over a linear gradient (1), purified KARI over a step gradient (2), and PageRuler™ unstained protein ladder (M, Fermentas). KARI was enriched to high purity with just one purification step.

FIG. 17 illustrates the structure alignment of E. coli KARI with spinach KARI.

FIG. 18 illustrates the characterization of E. coli IlvC and three variants resulting from the site saturation libraries: from top to bottom: Specific activities in U/mg, k_{cat} in 1/s, and catalytic efficiencies in M⁻¹*s⁻¹. All proteins were purified over a nickel sepharose histrap column.

FIG. 19 illustrates the characterization of Ec_IlvC^{B8-his6} and Ec_IlvC^{B8A71S-his6} compared to Ec_IlvC^{his6}, Ec_IlvC^{Q110V-his6}, Ec_IlvC^{Q110A-his6}, and Ec_IlvC^{S78D-his6}.

FIG. 20 illustrates a protein gel of cell lysates from the production strain GEVO1780 harboring the plasmids pGV1490, or pGV1661.

FIG. 21 illustrates plasmid pGV1102 (SEQ ID NO: 101).

FIG. 22 illustrates plasmid pGV1485 (SEQ ID NO: 103).

FIG. 23 illustrates plasmid pGV1490 (SEQ ID NO: 104).

FIG. 24 illustrates plasmid pGV1527.

FIG. 25 illustrates plasmid pGV1572 (SEQ ID NO: 105).

FIG. 26 illustrates plasmid pGV1573 (SEQ ID NO: 106).

FIG. 27 illustrates plasmid pGV1575 (SEQ ID NO: 107).

FIG. 28 illustrates plasmid pGV1609 (SEQ ID NO: 108).

FIG. 29 illustrates plasmid pGV1631.

FIG. 30 illustrates plasmid pGV1655 (SEQ ID NO: 109).

FIG. 31 illustrates plasmid pGV1661 (SEQ ID NO: 110).

FIG. 32 illustrates plasmid pGV1685 (SEQ ID NO: 111).

FIG. 33 illustrates plasmid pGV1698 (SEQ ID NO: 112).

FIG. 34 illustrates plasmid pGV1711 (SEQ ID NO: 113).

FIG. 35 illustrates plasmids pGV1705-A, pGV1748-A, pGV1749-A, and pGV1778-A carrying the ADH genes Ec_yqhD, Ec_fucO, Dm_ADH, and Kp_dhaT, respectively.

FIG. 36 illustrates plasmids pGV1748, pGV1749, and pGV1778 carrying the ADH genes Ec_fucO, Dm_ADH, and Kp_dhaT, respectively.

FIG. 37 illustrates plasmid pGV1716 (SEQ ID NO: 114).

FIG. 38 illustrates plasmid pGV1720 (SEQ ID NO: 115).

FIG. 39 illustrates plasmid pGV1730 (SEQ ID NO: 116) and linearization for integration by NruI digest (SEQ ID NO: 116).

FIG. 40 illustrates plasmid pGV1745 (SEQ ID NO: 117).

FIG. 41 illustrates plasmid pGV1772.

FIG. 42 illustrates plasmid pGV1777 (SEQ ID NO: 118).

FIG. 43 illustrates plasmids pGV1824, pGV1994, pGV2193, pGV2238, and pGV2241 carrying the KARI genes Ec_ilvC_coSc, Ec_ilvC_coSc^{6E6}, Ec_ilvC_coSc^{P2D1-his6}, Ec_ilvC_coSc^{P2D1-A1-his6}, and Ec_ilvC_coSc^{6E6-his6}, respectively.

FIG. 44 illustrates plasmid pGV1914 (SEQ ID NO: 119).

FIG. 45 illustrates plasmids pGV1925, pGV1927, pGV1975 and pGV1776 carrying the Ec_fucO in combination with KARI genes Ec_ilvC_coEc, Ec_ilvC_coEc^{S78D}, Ec_ilvC_coEc^{6E6} and Ec_ilvC_coEc^{2H10}, respectively.

FIG. 46 illustrates plasmid pGV1936 (SEQ ID NO: 120).

FIG. 47 illustrates plasmid pGV1938.

FIG. 48 illustrates plasmid pGV2020 (SEQ ID NO: 121).

FIG. 49 illustrates plasmid pGV2082 (SEQ ID NO: 122).

FIG. 50 illustrates plasmids pGV2227 (SEQ ID NO: 123), pGV2242 (SEQ ID NO: 125) carrying the KARI genes Ec_ilvC_coScQ110V and Ec_ilvC_coSc^{P2D1}, respectively.

FIG. 51 illustrates the alignment of available KARI sequences greater than 415 residues in length as depicted in Table 44. The conserved residue corresponding to the Serine 78 of SEQ ID NO: 13 is boxed.

FIG. 52 illustrates plasmid pGV3195.

### DETAILED DESCRIPTION

### Definitions

As used herein and in the appended claims, the singular forms “a,” “an,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

The term “microorganism” includes prokaryotic and eukaryotic microbial species from the Domains Archaea, Bacteria and Eukarya, the latter including yeast and filamentous fungi, protozoa, algae, or higher Protista. The terms “microbial cells” and “microbes” are used interchangeably with the term microorganism.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophiles (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consist mainly of hyperthermophilic sulfur-dependent prokaryotes and the Euryarchaeota contain the methanogens and extreme halophiles.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The term “genus” is defined as a taxonomic group of related species according to the Taxonomic Outline of Bacteria and Archaea (Garrity, G. M., Lilburn, T. G., Cole, J. R., Harrison, S. H., Euzeby, J., and Tindall, B. J. (2007) The Taxonomic Outline of Bacteria and Archaea. TOBA Release 7.7, March 2007. Michigan State University Board of Trustees.

The term “species” is defined as a collection of closely related organisms with greater than 97% 16S ribosomal RNA sequence homology and greater than 70% genomic hybridization and sufficiently different from all other organisms so as to be recognized as a distinct unit.

The terms “modified microorganism,” “recombinant microorganism” and “recombinant host cell” are used by inserting, expressing or overexpressing endogenous polynucleotides, by expressing or overexpressing heterologous polynucleotides, such as those included in a vector, by introducing a mutations into the microorganism or by altering the expression of an endogenous gene. The polynucleotide generally encodes a target enzyme involved in a metabolic pathway for producing a desired metabolite. It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “wild-type microorganism” describes a cell that occurs in nature, i.e. a cell that has not been genetically modified. A wild-type microorganism can be genetically modified to express or overexpress a first target enzyme. This microorganism can act as a parental microorganism in the generation of a microorganism modified to express or overexpress a second target enzyme. In turn, the microorganism modified to express or overexpress a first and a second target enzyme can be modified to express or overexpress a third target enzyme.

Accordingly, a “parental microorganism” functions as a reference cell for successive genetic modification events. Each modification event can be accomplished by introducing a nucleic acid molecule into the reference cell. The introduction facilitates the expression or overexpression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of heterologous polynucleotides encoding a target enzyme in to a parental microorganism.

The term “mutation” as used herein indicates any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring. In other embodiments, the mutations are the results of artificial mutation pressure. In still other embodiments, the mutations in the microorganism genome are the result of genetic engineering.

The term “biosynthetic pathway”, also referred to as “metabolic pathway”, refers to a set of anabolic or catabolic biochemical reactions for converting one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

The term “heterologous” as used herein with reference to molecules and in particular enzymes and polynucleotides, indicates molecules that are expressed in an organism other than the organism from which they originated or are found in nature, independently on the level of expression that can be lower, equal or higher than the level of expression of the molecule in the native microorganism.

On the other hand, the term “native” or “endogenous” as used herein with reference to molecules, and in particular enzymes and polynucleotides, indicates molecules that are expressed in the organism in which they originated or are found in nature, independently on the level of expression that can be lower equal or higher than the level of expression of the molecule in the native microorganism. It is understood that expression of native enzymes or polynucleotides may be modified in recombinant microorganisms.

The term “carbon source” generally refers to a substance suitable to be used as a source of carbon for prokaryotic or eukaryotic cell growth. Carbon sources include, but are not limited to, biomass hydrolysates, starch, sucrose, cellulose, hemicellulose, xylose, and lignin, as well as monomeric components of these substrates. Carbon sources can comprise various organic compounds in various forms, including, but not limited to polymers, carbohydrates, acids, alcohols, aldehydes, ketones, amino acids, peptides, etc. These include, for example, various monosaccharides such as glucose, dextrose (D-glucose), maltose, oligosaccharides, polysaccharides, saturated or unsaturated fatty acids, succinate, lactate, acetate, ethanol, etc., or mixtures thereof. Photosynthetic organisms can additionally produce a carbon source as a product of photosynthesis. In some embodiments, carbon sources may be selected from biomass hydrolysates and glucose. The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a modified microorganism as described herein.

The term “volumetric productivity” or “production rate” is defined as the amount of product formed per volume of medium per unit of time. Volumetric productivity is reported in gram per liter per hour (g/L/h).

The term “specific productivity” is defined as the rate of formation of the product. To describe productivity as an inherent parameter of the microorganism or microorganism and not of the fermentation process, productivity is herein further defined as the specific productivity in gram product per unit of cells, typically measured spectroscopically as absorbance units at 600 nm (OD₆₀₀ or OD) per hour (g/L/h/OD).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product. For example, the theoretical yield for one typical conversion of glucose to isobutanol is 0.41 g/g. As such, a yield of butanol from glucose of 0.39 g/g would be expressed as 95% of theoretical or 95% theoretical yield.

The term “titre” or “titer” is defined as the strength of a solution or the concentration of a substance in solution. For example, the titre of a biofuel in a fermentation broth is described as g of biofuel in solution per liter of fermentation broth (g/L).

The term “total titer” is defined as the sum of all biofuel produced in a process, including but not limited to the biofuel in solution, the biofuel in gas phase, and any biofuel removed from the process and recovered relative to the initial volume in the process or the operating volume in the process.

A “facultative anaerobic organism” or a “facultative anaerobic microorganism” is defined as an organism that can grow in either the presence or in the absence of oxygen.

A “strictly anaerobic organism” or a “strictly anaerobic microorganism” is defined as an organism that cannot grow in the presence of oxygen and which does not survive exposure to any concentration of oxygen.

An “anaerobic organism” or an “anaerobic microorganism” is defined as an organism that cannot grow in the presence of oxygen.

“Aerobic conditions” are defined as conditions under which the oxygen concentration in the fermentation medium is sufficiently high for an aerobic or facultative anaerobic microorganism to use as a terminal electron acceptor.

In contrast, “Anaerobic conditions” are defined as conditions under which the oxygen concentration in the fermentation medium is too low for the microorganism to use as a terminal electron acceptor. Anaerobic conditions may be achieved by sparging a fermentation medium with an inert gas such as nitrogen until oxygen is no longer available to the microorganism as a terminal electron acceptor. Alternatively, anaerobic conditions may be achieved by the microorganism consuming the available oxygen of the fermentation until oxygen is unavailable to the microorganism as a terminal electron acceptor. “Anaerobic conditions” are further defined as conditions under which no or small amounts of oxygen are added to the medium at rates of <3 mmol/L/h, preferably <2.5 mmol/L/h, more preferably <2 mmol/L/h and most preferably <1.5 mmol/L/h. “Anaerobic conditions” means in particular completely oxygen-free (=0 mmol/L/h oxygen) or with small amounts of oxygen added to the medium at rates of e.g. <0.5 to <1 mmol/L/h.

“Dissolved oxygen,” abbreviated as “DO” is expressed throughout as the percentage of saturating concentration of oxygen in water.

“Aerobic metabolism” refers to a biochemical process in which oxygen is used as a terminal electron acceptor to make energy, typically in the form of ATP, from carbohydrates. Aerobic metabolism occurs e.g. via glycolysis and the TCA cycle, wherein a single glucose molecule is metabolized completely into carbon dioxide in the presence of oxygen.

In contrast, “anaerobic metabolism” refers to a biochemical process in which oxygen is not the final acceptor of electrons contained in NADH. Anaerobic metabolism can be divided into anaerobic respiration, in which compounds other than oxygen serve as the terminal electron acceptor, and substrate level phosphorylation, in which the electrons from NADH are utilized to generate a reduced product via a “fermentative pathway.”

In “fermentative pathways,” NAD(P)H donates its electrons to a molecule produced by the same metabolic pathway that produced the electrons carried in NAD(P)H. For example, in one of the fermentative pathways of certain yeast strains, NAD(P)H generated through glycolysis transfers its electrons to pyruvate, yielding lactate. Fermentative pathways are usually active under anaerobic conditions but may also occur under aerobic conditions, under conditions where NADH is not fully oxidized via the respiratory chain. For example, above certain glucose concentrations, crabtree positive yeasts produce large amounts of ethanol under aerobic conditions.

The term “fermentation product” means any main product plus its coupled product. A “coupled product” is produced as part of the stoichiometric conversion of the carbon source to the main fermentation product. An example for a coupled product is the two molecules of CO₂ that are produced with every molecule of isobutanol during production of isobutanol from glucose according the biosynthetic pathway described herein.

The term “byproduct” means an undesired product related to the production of a biofuel. Byproducts are generally disposed as waste, adding cost to a biofuel process.

The term “co-product” means a secondary or incidental product related to the production of biofuel. Co-products have potential commercial value that increases the overall value of biofuel production, and may be the deciding factor as to the viability of a particular biofuel production process.

The term “non-fermenting yeast” is a yeast species that fails to demonstrate an anaerobic metabolism in which the electrons from NADH are utilized to generate a reduced product via a fermentative pathway such as the production of ethanol and CO₂ from glucose. Non-fermentative yeast can be identified by the “Durham Tube Test” (J. A. Barnett, R. W. Payne, and D. Yarrow. 2000. Yeasts Characteristics and Identification. 3^{rd} edition. p. 28-29. Cambridge University Press, Cambridge, UK.) or by monitoring the production of fermentation productions such as ethanol and CO₂.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs and fragments thereof. A polynucleotide of three or more nucleotides is also called nucleotidic oligomer or oligonucleotide.

It is understood that the polynucleotides described herein include “genes” and that the nucleic acid molecules described herein include “vectors” or “plasmids.” Accordingly, the term “gene”, also called a “structural gene” refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence.

The term “operon” refers two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “protein” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

The term “analog” or “analogous” refers to nucleic acid or protein sequences or protein structures that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

### The Microorganism in General

Microorganism Characterized by Producing C3-C5 Alcohols from Pyruvate Via an Overexpressed Metabolic Pathway

Native producers of butanol, and more specifically 1-butaanol, such as Clostridium acetobutylicum, are known, but these organisms generate byproducts such as acetone, ethanol, and butyrate during fermentations. Furthermore, these microorganisms are relatively difficult to manipulate, with significantly fewer tools available than in more commonly used production hosts such as E. coli. Additionally, the physiology and metabolic regulation of these native producers are much less well understood, impeding rapid progress towards high-efficiency production. Furthermore, no native microorganisms have been identified that can metabolize glucose into isobutanol in industrially relevant quantities or yields.

The production of isobutanol and other fusel alcohols by various yeast species, including Saccharomyces cerevisiae is of special interest to the distillers of alcoholic beverages, for whom fusel alcohols constitute often undesirable off-notes. Production of isobutanol in wild-type yeasts has been documented on various growth media, ranging from grape must from winemaking (Romano, et al., Metabolic diversity of Saccharomyces cerevisiae strains from spontaneously fermented grape musts, 19:311-315, 2003), in which 12-219 mg/L isobutanol were produced, supplemented to minimal media (Oliviera, et al. (2005) World Journal of Microbiology and Biotechnology 21:1569-1576), producing 16-34 mg/L isobutanol. Work from Dickinson, et al. (J Biol. Chem. 272(43):26871-8, 1997) has identified the enzymatic steps utilized in an endogenous S. cerevisiae pathway converting branch-chain amino acids (e.g., valine or leucine) to isobutanol.

A number of recent publications have described methods for the production of industrial chemicals such as C3-C5 alcohols such as isobutanol using engineered microorganisms. See, e.g., WO/2007/050671 to Donaldson et al., and WO/2008/098227 to Liao et al., which are herein incorporated by reference in their entireties. These publications disclose recombinant microorganisms that utilize a series of heterologously expressed enzymes to convert sugars into isobutanol. However, the production of isobutanol using these microorganisms is feasible only under aerobic conditions and the maximum yield that can be achieved is limited.

Recombinant microorganisms provided herein can express a plurality of target enzymes involved in pathways for the production isobutanol from a suitable carbon source under anaerobic conditions.

Accordingly, “engineered” or “modified” microorganisms are produced via the introduction of genetic material into a host or parental microorganism of choice thereby modifying or altering the cellular physiology and biochemistry of the microorganism. Through the introduction of genetic material the parental microorganism acquires new properties, e.g. the ability to produce a new, or greater quantities of, an intracellular metabolite under anaerobic conditions. As described herein, the introduction of genetic material into a parental microorganism results in a new or modified ability to produce isobutanol under anaerobic conditions. The genetic material introduced into the parental microorganism contains gene(s), or parts of genes, coding for one or more of the enzymes involved in a biosynthetic pathway for the production of isobutanol under anaerobic conditions and may also include additional elements for the expression and/or regulation of expression of these genes, e.g. promoter sequences.

An engineered or modified microorganism can also include in the alternative or in addition to the introduction of a genetic material into a host or parental microorganism, the disruption, deletion or knocking out of a gene or polynucleotide to alter the cellular physiology and biochemistry of the microorganism. Through the reduction, disruption or knocking out of a gene or polynucleotide the microorganism acquires new or improved properties (e.g., the ability to produce a new metabolite or greater quantities of an intracellular metabolite, improve the flux of a metabolite down a desired pathway, and/or reduce the production of undesirable by-products).

Microorganisms provided herein are modified to produce under anaerobic conditions metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate), an intermediate (e.g., 2-ketoisovalerate), or an end product (e.g., isobutanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

Exemplary metabolites include glucose, pyruvate, and C3-C5 alcohols, including isobutanol. The metabolite isobutanol can be produced by a recombinant microorganism engineered to express or over-express metabolic pathway that converts pyruvate to isobutanol. An exemplary metabolic pathway that converts pyruvate to isobutanol may be comprised of a acetohydroxy acid synthase (ALS) enzyme encoded by, for example, alsS from B. subtilis, a ketol-acid reductoisomerase (KARI) encoded by, for example ilvC from E. coli, a dihyroxy-acid dehydratase (DHAD), encoded by, for example ilvD from E. coli, a 2-keto-acid decarboxylase (KIVD) encoded by, for example kivd from L. lactis, and an alcohol dehydrogenase (ADH), encoded by, for example, by a native E. coli alcohol dehydrogenase gene, like Ec_yqhD.

Accordingly, provided herein are recombinant microorganisms that produce isobutanol and in some aspects may include the elevated expression of target enzymes such as ALS (encoded e.g. by the ilvIH operon from E. coli or by alsS from Bacillus subtilis), KARI (encoded e.g. by ilvC from E. coli), DHAD (encoded, e.g. by ilvD from E. coli, or by ILV3 from S. cerevisiae, and KIVD (encoded, e.g. by, ARO10 from S. cerevisiae, THI3 from S. cerevisiae, kivd from L. lactis).

The recombinant microorganism may further include the deletion or reduction of the activity of enzymes that (a) directly consume a precursor of the product, e.g. an isobutanol precursor, (b) indirectly consume a precursor of the product, e.g. of isobutanol, or (c) repress the expression or function of a pathway that supplies a precursor of the product, e.g. of isobutanol. These enzymes include pyruvate decarboxylase (encoded, e.g. by PDC1, PDC2, PDC3, PDC5, or PDC6 of S. cerevisiae), glycerol-3-phosphate dehydrogenase (encoded, e.g. by GPD1 or GPD2 of S. cerevisiae) an alcohol dehydrogenase (encoded, e.g., by adhE of E. coli or ADH1, ADH2, ADH3, ADH4, ADH5, ADH6, or ADH7 of S. cerevisiae), lactate dehydrogenase (encoded, e.g., by IdhA of E. coli), fumarate reductase (encoded, e.g., by frdB, frdC or frdBC of E. coli), FNR (encoded, e.g. by fnr of E. coli), 2-isopropylmalate synthase (encoded, e.g. by leuA of E. coli or by LEU4 or LEU9 of S. cerevisiae), valine transaminase (encoded, e.g. by ilvE of E. coli or by BAT1 or BAT2 of S. cerevisiae), pyruvate oxidase (e.g. encoded by poxB of E. coli), Threonine deaminase (encoded, e.g. by ilvA of E. coli or CHA1 or ILV1 of S. cerevisiae), pyruvate-formate-lyase (encoded, e.g. by pflB of E. coli), or phosphate acetyltransferase (encoded, e.g. by pta of E. coli), or any combination thereof, to increase the availability of pyruvate or reduce enzymes that compete for a metabolite in a desired biosynthetic pathway.

In yeast microorganisms, pyruvate decarboxylase (PDC) is a major competitor for pyruvate. During anaerobic fermentation, the main pathway to oxidize the NADH from glycolysis is through the production of ethanol. Ethanol is produced by alcohol dehydrogenase (ADH) via the reduction of acetaldehyde, which is generated from pyruvate by pyruvate decarboxylase (PDC). Thus, most of the pyruvate produced by glycolysis is consumed by PDC and is not available for the isobutanol pathway. Another pathway for NADH oxidation is through the production of glycerol. Dihydroxyacetone-phosphate, an intermediate of glycolysis is reduced to glycerol 3-phosphate by glycerol 3-phosphate dehydrogenase (GPD). Glycerol 3-phosphatase (GPP) converts glycerol 3-phosphate to glycerol. This pathway consumes carbon from glucose as well as reducing equivalents (NADH) resulting in less pyruvate and reducing equivalents available for the isobutanol pathway. These pathways contribute to low yield and low productivity of C3-C5 alcohols, including isobutanol. Accordingly, deletion or reduction of the activity of PDC and GPD may increase yield and productivity of C3-C5 alcohols, including isobutanol.

Reduction of PDC activity can be accomplished by 1) mutation or deletion of a positive transcriptional regulator for the structural genes encoding for PDC or 2) mutation or deletion of all PDC genes in a given organism. The term “transcriptional regulator” can specify a protein or nucleic acid that works in trans to increase or to decrease the transcription of a different locus in the genome. For example, in S. cerevisiae, the PDC2 gene, which encodes for a positive transcriptional regulator of PDC1,5,6 genes can be deleted; a S. cerevisiae in which the PDC2 gene is deleted is reported to have only ˜10% of wildtype PDC activity (Hohmann, Mol Gen Genet, 241:657-666 (1993)). Alternatively, for example, all structural genes for PDC (e.g. in S. cerevisiae, PDC1, PDC5, and PDC6, or in K. lactis, PDC1) are deleted.

Crabtree-positive yeast strains such as Saccharomyces cerevisiae strain that contains disruptions in all three of the PDC alleles no longer produce ethanol by fermentation. However, a downstream product of the reaction catalyzed by PDC, acetyl-CoA, is needed for anabolic production of necessary molecules. Therefore, the Pdc-mutant is unable to grow solely on glucose, and requires a two-carbon carbon source, either ethanol or acetate, to synthesize acetyl-CoA. (Flikweert M T, de Swaaf M, van Dijken J P, Pronk J T. FEMS Microbiol Lett. 1999 May 1; 174(1):73-9. PMID:10234824 and van Maris A J, Geertman J M, Vermeulen A, Groothuizen M K, Winkler A A, Piper M D, van Dijken J P, Pronk J T. Appl Environ Microbiol. 2004 January; 70(1):159-66. PMID: 14711638).

Thus, in an embodiment, such a Crabtree-positive yeast strain may be evolved to generate variants of the PDC mutant yeast that do not have the requirement for a two-carbon molecule and has a growth rate similar to wild type on glucose. Any method, including chemostat evolution or serial dilution may be utilized to generate variants of strains with deletion of three PDC alleles that can grow on glucose as the sole carbon source at a rate similar to wild type (van Maris et al., Directed Evolution of Pyruvate Decarboxylase-Negative Saccharomyces cerevisiae, Yielding a C2-Independent, Glucose-Tolerant, and Pyruvate-Hyperproducing Yeast, Applied and Environmental Microbiology, 2004, 70(1), 159-166).

Another byproduct that would decrease yield of isobutanol is glycerol. Glycerol is produced by 1) the reduction of the glycolysis intermediate, dihydroxyacetone phosphate (DHAP), to glycerol-3-phosphate (G3P) via the oxidation of NADH to NAD⁺ by Glycerol-3-phosphate dehydrogenase (GPD) followed by 2) the dephosphorylation of glycerol-3-phophate to glycerol by glycerol-3-phosphatase (GPP). Production of glycerol results in loss of carbons as well as reducing equivalents. Reduction of GPD activity would increase yield of isobutanol. Reduction of GPD activity in addition to PDC activity would further increase yield of isobutanol. Reduction of glycerol production has been reported to increase yield of ethanol production (Nissen et al., Anaerobic and aerobic batch cultivation of Saccharomyces cerevisiae mutants impaired in glycerol synthesis, Yeast, 2000, 16, 463-474; Nevoigt et al., Method of modifying a yeast cell for the production of ethanol, WO 2009/056984). Disruption of this pathway has also been reported to increase yield of lactate in a yeast engineered to produce lactate instead of ethanol (Dundon et al., Yeast cells having disrupted pathway from dihydroxyacetone phosphate to glycerol, US 2009/0053782).

In one embodiment, the microorganism is a crab-tree positive yeast with reduced or no GPD activity. In another embodiment, the microorganism is a crab-tree positive yeast with reduced or no GPD activity, and expresses an isobutanol biosynthetic pathway and produces isobutanol. In yet another embodiment, the microorganism is a crab-tree positive yeast with reduced or no GPD activity and with reduced or no PDC activity. In another embodiment, the microorganism is a crab-tree positive yeast with reduced or no GPD activity, with reduced or no PDC activity, and expresses an isobutanol biosynthetic pathway and produces isobutanol.

In another embodiment, the microorganism is a crab-tree negative yeast with reduced or no GPD activity. In another embodiment, the microorganism is a crab-tree negative yeast with reduced or no GPD activity, expresses the isobutanol biosynthetic pathway and produces isobutanol. In yet another embodiment, the microorganism is a crab-tree negative yeast with reduced or no GPD activity and with reduced or no PDC activity. In another embodiment, the microorganism is a crab-tree negative yeast with reduced or no GPD activity, with reduced or no PDC activity, expresses an an isobutanol biosynthetic pathway and produces isobutanol.

Any method can be used to identify genes that encode for enzymes with pyruvate decarboxylase (PDC) activity. PDC catalyzes the decarboxylation of pyruvate to form acetaldehyde. Generally, homologous or similar PDC genes and/or homologous or similar PDC enzymes can be identified by functional, structural, and/or genetic analysis. In most cases, homologous or similar PDC genes and/or homologous or similar PDC enzymes will have functional, structural, or genetic similarities. Techniques known to those skilled in the art may be suitable to identify homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous genes, proteins, or enzymes, techniques may include, but not limited to, cloning a PDC gene by PCR using primers based on a published sequence of a gene/enzyme or by degenerate PCR using degenerate primers designed to amplify a conserved region among PDC genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity, then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above mentioned databases in accordance with the teachings herein. Furthermore, PDC activity can be determined phenotypically. For example, ethanol production under fermentative conditions can be assessed. A lack of ethanol production may be indicative of a yeast microorganism with no PDC activity.

Any method can be used to identify genes that encode for enzymes with glycerol-3-phosphate dehydrogenase (GPD) activity. GPD catalyzes the reduction of dihydroxyacetone phosphate (DHAP) to glycerol-3-phosphate (G3P) with the corresponding oxidation of NADH to NAD+. Generally, homologous or similar GPD genes and/or homologous or similar GPD enzymes can be identified by functional, structural, and/or genetic analysis. In most cases, homologous or similar GPD genes and/or homologous or similar GPD enzymes will have functional, structural, or genetic similarities. Techniques known to those skilled in the art may be suitable to identify homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous genes, proteins, or enzymes, techniques may include, but not limited to, cloning a GPD gene by PCR using primers based on a published sequence of a gene/enzyme or by degenerate PCR using degenerate primers designed to amplify a conserved region among GPD genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity, then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above mentioned databases in accordance with the teachings herein. Furthermore, GPD activity can be determined phenotypically. For example, glycerol production under fermentative conditions can be assessed. A lack of glycerol production may be indicative of a yeast microorganism with no GPD activity.

The recombinant microorganism may further include metabolic pathways for the fermentation of a C3-C5 alcohols from five-carbon (pentose) sugars including xylose. Most yeast species metabolize xylose via a complex route, in which xylose is first reduced to xylitol via a xylose reductase (XR) enzyme. The xylitol is then oxidized to xylulose via a xylitol dehydrogenase (XDH) enzyme. The xylulose is then phosphorylated via an xylulokinase (XK) enzyme. This pathway operates inefficiently in yeast species because it introduces a redox imbalance in the cell. The xylose-to-xylitol step uses NADH as a cofactor, whereas the xylitol-to-xylulose step uses NADPH as a cofactor. Other processes must operate to restore the redox imbalance within the cell. This often means that the organism cannot grow anaerobically on xylose or other pentose sugar. Accordingly, a yeast species that can efficiently ferment xylose and other pentose sugars into a desired fermentation product is therefore very desirable.

Thus, in one aspect, the recombinant microorganism is engineered to express a functional exogenous xylose isomerase. Exogenous xylose isomerases functional in yeast are known in the art. See, e.g., Rajgarhia et al, US20060234364, which is herein incorporated by reference in its entirety. In an embodiment according to this aspect, the exogenous xylose isomerase gene is operatively linked to promoter and terminator sequences that are functional in the yeast cell. In a preferred embodiment, the recombinant microorganism further has a deletion or disruption of a native gene that encodes for an enzyme (e.g. XR and/or XDH) that catalyzes the conversion of xylose to xylitol. In a further preferred embodiment, the recombinant microorganism also contains a functional, exogenous xylulokinase (XK) gene operatively linked to promoter and terminator sequences that are functional in the yeast cell. In one embodiment, the xylulokinase (XK) gene is overexpressed.

The disclosure identifies specific genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutation and silent mutations. Such modified or mutated polynucleotides and polypeptides can be screened for expression of a functional enzyme using methods known in the art.

Due to the inherent degeneracy of the genetic code, other polynucleotides which encode substantially the same or a functionally equivalent polypeptide can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as they modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating metabolites are encompassed by the microorganisms and methods provided herein.

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used comparing a molecule sequence to a database containing a large number of sequences from different organisms is the computer program BLAST (Altschul, S. F., et al. (1990) “Basic local alignment search tool.” J. Mol. Biol. 215:403-410; Gish, W. and States, D. J. (1993) “Identification of protein coding regions by database similarity search.” Nature Genet. 3:266-272; Madden, T. L., et al. (1996) “Applications of network BLAST server” Meth. Enzymol. 266:131-141; Altschul, S. F., et al. (1997) “Gapped BLAST and PSI-BLAST: a new generation of protein database search programs.” Nucleic Acids Res. 25:3389-3402; Zhang, J. and Madden, T. L. (1997) “PowerBLAST: A new network BLAST application for interactive or automated sequence analysis and annotation.” Genome Res. 7:649-656), especially blastp or tblastn (Altschul, S. F., et al. (1997) “Gapped BLAST and PSI-BLAST: a new generation of protein database search programs.” Nucleic Acids Res. 25:3389-3402). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, W. R. (1990) “Rapid and Sensitive Sequence Comparison with FASTP and FASTA” Meth. Enzymol. 183:63-98). For example, percent sequence identity between amino acid sequences can be determined using FASTA with its default parameters (a word size of 2 and the PAM250 scoring matrix), as provided in GCG Version 6.1, hereby incorporated herein by reference.

It is understood that a range of microorganisms can be modified to include recombinant metabolic pathways suitable for the production of C3-C5 alcohols, including isobutanol. In various embodiments, microorganisms may be selected from bacterial or yeast microorganisms. Microorganisms for the production of C3-C5 alcohols, including isobutanol may be selected based on certain characteristics:

One characteristic may include the ability to metabolize a carbon source in the presence of a C3-C5 alcohol, including isobutanol. A microorganism capable of metabolizing a carbon source at a high isobutanol concentration is more suitable as a production microorganism compared to a microorganism capable of metabolizing a carbon source at a low isobutanol concentration. Another characteristic may include the property that the microorganism is selected to convert various carbon sources into C3-C5 alcohols, including isobutanol. Accordingly, in one embodiment, the recombinant microorganism herein disclosed can convert a variety of carbon sources to products, including but not limited to glucose, galactose, mannose, xylose, arabinose, lactose, sucrose, and mixtures thereof.

Another characteristic specific to a yeast microorganism may include the property that the microorganism is able to metabolize a carbon source in the absence of pyruvate decarboxylase (PDC). In an embodiment, it is preferable that the yeast microorganism is able to metabolize 5- and 6-carbon sugar in the absence of PDC. In one embodiment, it is even more preferred that a yeast microorganism is able to grow on 5- and 6-carbon sugars in the absence of PDC.

Another characteristic may include the property that the wild-type or parental microorganism is non-fermenting. In other words, it cannot metabolize a carbon source anaerobically while the yeast is able to metabolize a carbon source in the presence of oxygen. Non-fermenting yeast refers to both naturally occurring yeasts as well as genetically modified yeast. During anaerobic fermentation with fermentative yeast, the main pathway to oxidize the NADH from glycolysis is through the production of ethanol. Ethanol is produced by alcohol dehydrogenase (ADH) via the reduction of acetaldehyde, which is generated from pyruvate by pyruvate decarboxylase (PDC).

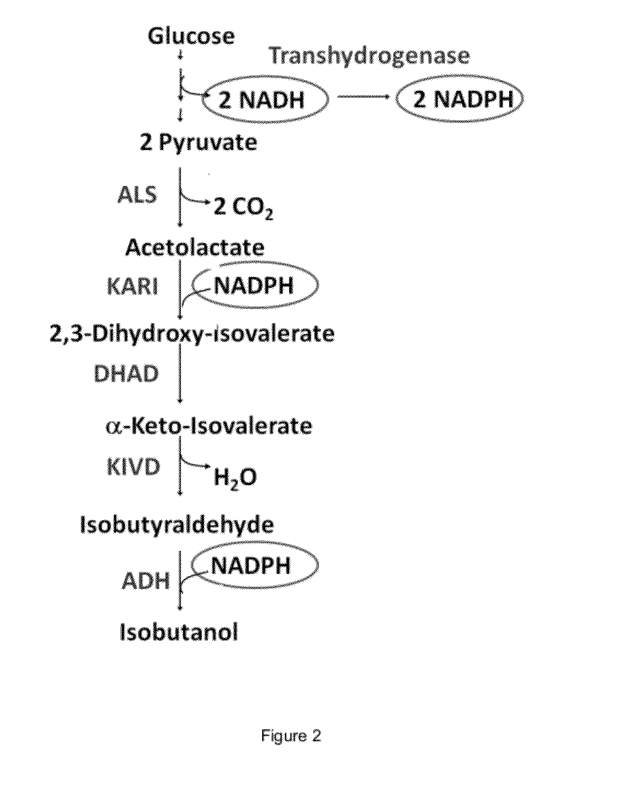

Thus, in one embodiment, a fermentative yeast can be engineered to be non-fermentative by the reduction or elimination of the native PDC activity. Thus, most of the pyruvate produced by glycolysis is not consumed by PDC and is available for the isobutanol pathway. Deletion of this pathway increases the pyruvate and the reducing equivalents available for the isobutanol pathway. Fermentative pathways contribute to low yield and low productivity of isobutanol. Accordingly, deletion of PDC may increase yield and productivity of isobutanol. In one embodiment, the yeast microorganisms may be selected from the “Saccharomyces Yeast Clade”, defined as an ascomycetous yeast taxonomic class by Kurtzman and Robnett in 1998 (“Identification and phylogeny of ascomycetous yeast from analysis of nuclear large subunit (26S) ribosomal DNA partial sequences.” Antonie van Leeuwenhoek 73: 331-371, see FIG. 2 of Leeuwenhook reference). They were able to determine the relatedness of yeast of approximately 500 yeast species by comparing the nucleotide sequence of the D1/D2 domain at the 5′ end of the gene encoding the large ribosomal subunit 26S. In pair-wise comparisons of the D1/D2 nucleotide sequence of S. cerevisiae and the two most distant yeast in the Saccharomyces clade: K. lactis and K. marxianus, yeast from this Glade share greater than 80% identity.

An ancient whole genome duplication (WGD) event occurred during the evolution of hemiascomycete yeast was discovered using comparative genomics tools (Kellis et al 2004 “Proof and evolutionary analysis of ancient genome duplication in the yeast S. cerevisiae.” Nature 428:617-624. Dujon et al 2004 “Genome evolution in yeasts.” Nature 430:35-44. Langkjaer et al 2003 “Yeast genome duplication was followed by asynchronous differentiation of duplicated genes.” Nature 428:848-852. Wolfe and Shields 1997 “Molecular evidence for an ancient duplication of the entire yeast genome.” Nature 387:708-713.) Using this major evolutionary event, yeast can be divided into species that diverged from a common ancestor following the WGD event (termed “post-WGD yeast” herein) and species that diverged from the yeast lineage prior to the WGD event (termed “pre-WGD yeast” herein).

Accordingly, in one embodiment, the yeast microorganism may be selected from a post-WGD yeast genus, including but not limited to Saccharomyces and Candida. The favored post-WGD yeast species include: S. cerevisiae, S. uvarum, S. bayanus, S. paradoxus, S. castelli, and C. glabrata.

In another embodiment, a method provided herein includes a recombinant organism that is a Saccharomyces sensu stricto yeast microorganism. In one aspect, a Saccharomyces sensu stricto yeast microorganism is selected from one of the species: S. cerevisiae, S. cerevisiae, S. kudriavzevii, S. mikatae, S. bayanus, S. uvarum, S. carocanis or hybrids thereof.

In another embodiment, the yeast microorganism may be selected from a pre-whole genome duplication (pre-WBD) yeast genus including but not limited to Saccharomyces, Kluyveromyces, Issatchenkia, Candida, Pichia, Debaryomyces, Hansenula, Pachysolen, Yarrowia and, Schizosaccharomyces. Representative pre-WGD yeast species include: S. kluyveri, K. thermotolerans, K. marxianus, K. waltii, K. lactis, C. tropicalis, P. pastoris, P. anomala, P. stipitis, D. hansenii, H. anomala, P. tannophilis, I. orientalis, Y. lipolytica, and S. pombe.

A yeast microorganism may be either Crabtree-negative or Crabtree-positive. A yeast cell having a Crabtree-negative phenotype is any yeast cell that does not exhibit the Crabtree effect. The term “Crabtree-negative” refers to both naturally occurring and genetically modified organisms. Briefly, the Crabtree effect is defined as the inhibition of oxygen consumption by a microorganism when cultured under aerobic conditions due to the presence of a high concentration of glucose (e.g., 50 g-glucose L⁻¹). In other words, a yeast cell having a Crabtree-positive phenotype continues to ferment irrespective of oxygen availability due to the presence of glucose, while a yeast cell having a Crabtree-negative phenotype does not exhibit glucose mediated inhibition of oxygen consumption.

Accordingly, in one embodiment the yeast microorganism may be selected from a yeast with a Crabtree-negative phenotype including but not limited to the following genera: Kluyveromyces, Pichia, Issatchenkia, Hansenula, and Candida. Crabtree-negative species include but are not limited to: K. lactis, K. marxianus, P. anomala, P. stipitis, H. anomala, I. orientalis, and C. utilis.

In another embodiment, the yeast microorganism may be selected from a yeast with a Crabtree-positive phenotype, including but not limited to Saccharomyces, Kluyveromyces, Zygosaccharomyces, Debaryomyces, Pichia and Schizosaccharomyces. Crabtree-positive yeast species include but are not limited to: S. cerevisiae, S. uvarum, S. bayanus, S. paradoxus, S. casteffi, S. kluyveri, K. thermotolerans, C. glabrat, Z. bailli, Z. rouxii, D. hansenii, P. pastorius, and S. pombe.

Bacterial Microorganisms may be selected from a number of genera, including but not limited to Arthrobacter, Bacillus, Brevibacterium, Clostridium, Corynebacterium, Cyanobacterium, Escherichia, Gluconobacter, Lactobacillus, Nocardia, Pseudomonas, Rhodococcus, Saccharomyces, Shewanella, Streptomyces, Xanthomonas, and Zymomonas. In another embodiment, such hosts are Corynebacterium, Cyanobacterium, E. coli or Pseudomonas. In another embodiment, such hosts are E. coli W3110, E. coli B, Pseudomonas oleovorans, Pseudomonas fluorescens, or Pseudomonas putida.

One exemplary metabolic pathway for the conversion of a carbon source to a C3-C5 alcohol via pyruvate begins with the conversion of glucose to pyruvate via glycolysis. Glycolysis also produces 2 moles of NADH and 2 moles of ATP. Two moles of pyruvate are then used to produce one mole of isobutanol (PCT/US2006/041602, PCT/US2008/053514). Alternative isobutanol pathways have been described in International Patent Application No PCT/US2006/041602 and in Dickinson et al., Journal of Biological Chemistry 273:25751-15756 (1998).

Accordingly, the engineered isobutanol pathway to convert pyruvate to isobutanol can be, but is not limited to, the following reactions:

1. 2 pyruvate→acetolactate+CO₂
2. acetolactate+NAD(P)H→2,3-dihydroxyisovalerate+NAD(P)⁺
3. 2,3-dihydroxyisovalerate→alpha-ketoisovalerate
4. alpha-ketoisovalerate→isobutyraldehyde+CO₂
5. isobutyraldehyde+NADPH→isobutanol+NADP⁺

These reactions are carried out by the enzymes 1) Acetolactate Synthase (ALS), 2) Ketol-acid Reducto-Isomerase (KARI), 3) Dihydroxy-acid dehydratase (DHAD), 4) Keto-isovalerate decarboxylase (KIVD), and 5) an Alcohol Dehydrogenase (ADH).

In another embodiment, the microorganism is engineered to overexpress these enzymes. For example, ALS can be encoded by the alsS gene of B. subtilis, alsS of L. lactis, or the ilvK gene of K. pneumonia. For example, KARI can be encoded by the ilvC genes of E. coli, C. glutamicum, M. maripaludis, or Piromyces sp E2. For example, DHAD can be encoded by the ilvD genes of E. coli, L. lactis, or C. glutamicum, or by the ILV3 gene from S. cerevisiae. KIVD can be encoded by the kivd gene of L. lactis. ADH can be encoded by ADH2, ADH6, or ADH7 of S. cerevisiae, by the adhA gene product of L. lactis, or by an ADH from D. melanogaster.

The microorganism of the invention may be engineered to have increased ability to convert pyruvate to a C3-C5 alcohol, including isobutanol. In one embodiment, the microorganism may be engineered to have increased ability to convert pyruvate to isobutyraldehyde. In another embodiment, the microorganism may be engineered to have increased ability to convert pyruvate to keto-isovalerate. In another embodiment, the microorganism may be engineered to have increased ability to convert pyruvate to 2,3-dihydroxyisovalerate. In another embodiment, the microorganism may be engineered to have increased ability to convert pyruvate to acetolactate.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis.

It is understood that various microorganisms can act as “sources” for genetic material encoding target enzymes suitable for use in a recombinant microorganism provided herein. For example, In addition, genes encoding these enzymes can be identified from other fungal and bacterial species and can be expressed for the modulation of this pathway. A variety of eukaryotic organisms could serve as sources for these enzymes, including, but not limited to, Drosophila spp., including D. melanogaster, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. stipitis, Torulaspora pretoriensis, Schizosaccharomyces spp., including S. pombe, Cryptococcus spp., Aspergillus spp., Neurospora spp., or Ustilago spp. Sources of genes from anaerobic fungi include, but not limited to, Piromyces spp., Orpinomyces spp., or Neocallimastix spp. Sources of prokaryotic enzymes that are useful include, but not limited to, Escherichia. coli, Klebsiella spp., including K. pneumoniae, Zymomonas mobilis, Staphylococcus aureus, Bacillus spp., Clostridium spp., Corynebacterium spp., Pseudomonas spp., Lactococcus spp., Enterobacter spp., and Salmonella spp.

### Methods in General

### Gene Expression

In another embodiment a method of producing a recombinant microorganism that converts a suitable carbon substrate to C3-C5 alcohols such as isobutanol is provided. The method includes transforming a microorganism with one or more recombinant polynucleotides encoding polypeptides that include but are not limited to, for example, ALS, KARI, DHAD, KIVD, ADH and a transhydrogenase. Polynucleotides that encode enzymes useful for generating metabolites including homologs, variants, fragments, related fusion proteins, or functional equivalents thereof, are used in recombinant nucleic acid molecules that direct the expression of such polypeptides in appropriate host cells, such as bacterial or yeast cells. It is understood that the addition of sequences which do not alter the encoded activity of a polynucleotide, such as the addition of a non-functional or non-coding sequence, is a conservative variation of the basic nucleic acid. The “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., concentration or weight), or in terms of affinity or dissociation constants.

Those of skill in the art will recognize that, due to the degenerate nature of the genetic code, a variety of DNA compounds differing in their nucleotide sequences can be used to encode a given amino acid sequence of the disclosure. The native DNA sequence encoding the biosynthetic enzymes described herein are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate embodiments of the disclosure.

The disclosure provides nucleic acid molecules in the form of recombinant DNA expression vectors or plasmids, as described in more detail below, that encode one or more target enzymes. Generally, such vectors can either replicate in the cytoplasm of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) forms.

Provided herein are methods for the expression of one or more of the genes involved in the production of beneficial metabolites and recombinant DNA expression vectors useful in the method. Thus, included within the scope of the disclosure are recombinant expression vectors that include such nucleic acids. The term expression vector refers to a nucleic acid that can be introduced into a host microorganism or cell-free transcription and translation system. An expression vector can be maintained permanently or transiently in a microorganism, whether as part of the chromosomal or other DNA in the microorganism or in any cellular compartment, such as a replicating vector in the cytoplasm. An expression vector also comprises a promoter that drives expression of an RNA, which typically is translated into a polypeptide in the microorganism or cell extract. For efficient translation of RNA into protein, the expression vector also typically contains a ribosome-binding site sequence positioned upstream of the start codon of the coding sequence of the gene to be expressed. Other elements, such as enhancers, secretion signal sequences, transcription termination sequences, and one or more marker genes by which host microorganisms containing the vector can be identified and/or selected, may also be present in an expression vector. Selectable markers, i.e., genes that confer antibiotic resistance or sensitivity, are used and confer a selectable phenotype on transformed cells when the cells are grown in an appropriate selective medium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433), can also be used. For E. coli expression vectors, it is useful to include an E. coli origin of replication, such as from pUC, p1P, p1, and pBR.

Thus, recombinant expression vectors contain at least one expression system, which, in turn, is composed of at least a portion of PKS and/or other biosynthetic gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

Moreover, methods for expressing a polypeptide from a nucleic acid molecule that are specific to yeast microorganisms are well known. For example, nucleic acid constructs that are used for the expression of heterologous polypeptides within Kluyveromyces and Saccharomyces are well known (see, e.g., U.S. Pat. Nos. 4,859,596 and 4,943,529, each of which is incorporated by reference herein in its entirety for Kluyveromyces and, e.g., Gellissen et al., Gene 190(1):87-97 (1997) for Saccharomyces. Yeast plasmids have a selectable marker and an origin of replication, also known as Autonomously Replicating Sequences (ARS). In addition certain plasmids may also contain a centromeric sequence. These centromeric plasmids are generally a single or low copy plasmid. Plasmids without a centromeric sequence and utilizing either a 2 micron (S. cerevisiae) or 1.6 micron (K. lactis) replication origin are high copy plasmids. The selectable marker can be either prototrophic, such as HIS3, TRP1, LEU2, URA3 or ADE2, or antibiotic resistance, such as, bar, ble, hph, or kan.

A nucleic acid of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated nucleic acid molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the polynucleotide by standard techniques, such as site-directed mutagenesis and PCR-mediated mutagenesis. In contrast to those positions where it may be desirable to make a non-conservative amino acid substitutions (see above), in some positions it is preferable to make conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Although the effect of an amino acid change varies depending upon factors such as phosphorylation, glycosylation, intra-chain linkages, tertiary structure, and the role of the amino acid in the active site or a possible allosteric site, it is generally preferred that the substituted amino acid is from the same group as the amino acid being replaced. To some extent the following groups contain amino acids which are interchangeable: the basic amino acids lysine, arginine, and histidine; the acidic amino acids aspartic and glutamic acids; the neutral polar amino acids serine, threonine, cysteine, glutamine, asparagine and, to a lesser extent, methionine; the nonpolar aliphatic amino acids glycine, alanine, valine, isoleucine, and leucine (however, because of size, glycine and alanine are more closely related and valine, isoleucine and leucine are more closely related); and the aromatic amino acids phenylalanine, tryptophan, and tyrosine. In addition, although classified in different categories, alanine, glycine, and serine seem to be interchangeable to some extent, and cysteine additionally fits into this group, or may be classified with the polar neutral amino acids.

### Overexpression of Heterologous Genes

Methods for overexpressing a polypeptide from a native or heterologous nucleic acid molecule are well known. Such methods include, without limitation, constructing a nucleic acid sequence such that a regulatory element promotes the expression of a nucleic acid sequence that encodes the desired polypeptide. Typically, regulatory elements are DNA sequences that regulate the expression of other DNA sequences at the level of transcription. Thus, regulatory elements include, without limitation, promoters, enhancers, and the like. For example, the exogenous genes can be under the control of an inducible promoter or a constitutive promoter. Moreover, methods for expressing a polypeptide from an exogenous nucleic acid molecule in yeast are well known. For example, nucleic acid constructs that are used for the expression of exogenous polypeptides within Kluyveromyces and Saccharomyces are well known (see, e.g., U.S. Pat. Nos. 4,859,596 and 4,943,529, for Kluyveromyces and, e.g., Gellissen et al., Gene 190(1):87-97 (1997) for Saccharomyces). Yeast plasmids have a selectable marker and an origin of replication. In addition certain plasmids may also contain a centromeric sequence. These centromeric plasmids are generally a single or low copy plasmid. Plasmids without a centromeric sequence and utilizing either a 2 micron (S. cerevisiae) or 1.6 micron (K. lactis) replication origin are high copy plasmids. The selectable marker can be either prototrophic, such as HIS3, TRP1, LEU2, URA3 or ADE2, or antibiotic resistance, such as, bar, ble, hph, or kan.

In another embodiment, heterologous control elements can be used to activate or repress expression of endogenous genes. Additionally, when expression is to be repressed or eliminated, the gene for the relevant enzyme, protein or RNA can be eliminated by known deletion techniques.

As described herein, any microorganism within the scope of the disclosure can be identified by selection techniques specific to the particular enzyme being expressed, over-expressed or repressed. Methods of identifying the strains with the desired phenotype are well known to those skilled in the art. Such methods include, without limitation, PCR, RT-PCR, and nucleic acid hybridization techniques such as Northern and Southern analysis, altered growth capabilities on a particular substrate or in the presence of a particular substrate, a chemical compound, a selection agent and the like. In some cases, immunohistochemistry and biochemical techniques can be used to determine if a cell contains a particular nucleic acid by detecting the expression of the encoded polypeptide. For example, an antibody having specificity for an encoded enzyme can be used to determine whether or not a particular microorganism contains that encoded enzyme. Further, biochemical techniques can be used to determine if a cell contains a particular nucleic acid molecule encoding an enzymatic polypeptide by detecting a product produced as a result of the expression of the enzymatic polypeptide. For example, transforming a cell with a vector encoding acetolactate synthase and detecting increased cytosolic acetolactate concentrations compared to a cell without the vector indicates that the vector is both present and that the gene product is active. Methods for detecting specific enzymatic activities or the presence of particular products are well known to those skilled in the art. For example, the presence of acetolactate can be determined as described by Hugenholtz and Starrenburg, Appl. Microbiol. Biotechnol. 38:17-22 (1992).

### Identification of Genes in a Host Microorganism

Any method can be used to identify genes that encode for enzymes with a specific activity. Generally, homologous or analogous genes with similar activity can be identified by functional, structural, and/or genetic analysis. In most cases, homologous or analogous genes with similar activity will have functional, structural, or genetic similarities. Techniques known to those skilled in the art may be suitable to identify homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous genes, proteins, or enzymes, techniques may include, but not limited to, cloning a gene by PCR using primers based on a published sequence of a gene/enzyme or by degenerate PCR using degenerate primers designed to amplify a conserved region among a gene. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity, then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or analogous genes with similar activity, techniques also include comparison of data concerning a candidate gene or enzyme with databases such as BRENDA, KEGG, or MetaCYC. The candidate gene or enzyme may be identified within the above mentioned databases in accordance with the teachings herein. Furthermore, enzymatic activity can be determined phenotypically. For example, ethanol production under fermentative conditions can be assessed. A lack of ethanol production may be indicative of a microorganism lacking an alcohol dehydrogenase capable of reducing acetaldehyde to ethanol.

### Genetic Insertions and Deletions

Any method can be used to introduce a nucleic acid molecule into the chromosomal DNA of a microorganism and many such methods are well known. For example, lithium acetate transformation and electroporation are common methods for introducing nucleic acid into yeast microorganisms. See, e.g., Gietz et al., Nucleic Acids Res. 27:69-74 (1992); Ito et al., J. Bacterol. 153:163-168 (1983); and Becker and Guarente, Methods in Enzymology 194:182-187 (1991).

In an embodiment, the deletion of a gene of interest in a bacterial microorganism, including an E. coli microorganism occurs according to the principle of homologous recombination. According to this embodiment, an integration cassette containing a module comprising at least one marker gene is flanked on either side by DNA fragments homologous to those of the ends of the targeted integration site. After transforming the host microorganism with the cassette by appropriate methods, homologous recombination between the flanking sequences may result in the marker replacing the chromosomal region in between the two sites of the genome corresponding to flanking sequences of the integration cassette. The homologous recombination event may be facilitated by a recombinase enzyme that may be native to the host microorganism or may be heterologous and transiently overexpressed (Datsenko and Wanner, Proc. Natl. Acad. Sci. USA 97, 6640-6645, 2000).

In an embodiment, the integration of a gene of interest into a DNA fragment or target gene of a yeast microorganism occurs according to the principle of homologous recombination. According to this embodiment, an integration cassette containing a module comprising at least one yeast marker gene and/or the gene to be integrated (internal module) is flanked on either side by DNA fragments homologous to those of the ends of the targeted integration site (recombinogenic sequences). After transforming the yeast with the cassette by appropriate methods, a homologous recombination between the recombinogenic sequences may result in the internal module replacing the chromosomal region in between the two sites of the genome corresponding to the recombinogenic sequences of the integration cassette. (Orr-Weaver et al., Proc Natl Acad Sci USA 78:6354-6358 (1981))

In an embodiment, the integration cassette for integration of a gene of interest into a yeast microorganism includes the heterologous gene under the control of an appropriate promoter and terminator together with the selectable marker flanked by recombinogenic sequences for integration of a heterologous gene into the yeast chromosome. In an embodiment, the heterologous gene includes an appropriate native gene desired to increase the copy number of a native gene(s). The selectable marker gene can be any marker gene used in yeast, including but not limited to, HIS3, TRP1, LEU2, URA3, bar, ble, hph, and kan. The recombinogenic sequences can be chosen at will, depending on the desired integration site suitable for the desired application.

Additionally, in an embodiment pertaining to yeast microorganisms, certain introduced marker genes are removed from the genome using techniques well known to those skilled in the art. For example, URA3 marker loss can be obtained by plating URA3 containing cells in FOA (5-fluoro-orotic acid) containing medium and selecting for FOA resistant colonies (Boeke, J. et al, 1984, Mol. Gen. Genet, 197, 345-47).

Integration of all the genes of a metabolic pathway that lead to a product into the genome of the production strain eliminates the need of a plasmid expression system, as the enzymes are produced from the chromosome. The integration of pathway genes avoids loss of productivity over time due to plasmid loss. This is important for long fermentation times and for fermentations in large scale where the seed train is long and the production strain has to go through many doublings from the first inoculation to the end of the large scale fermentation.

Integrated genes are maintained in the strain without selection. This allows the construction of production strains that are free of marker genes which are commonly used for maintenance of plasmids. Production strains with integrated pathway genes can contain minimal amounts of foreign DNA since there are no origins of replication and other non coding DNA necessary that have to be in plasmid based systems. The biocatalyst with integrated pathway genes improves the performance of a production process because it avoids energy and carbon requiring processes. These processes are the replication of many copies of plasmids and the production of non-pathway active proteins like marker proteins in the production strain.

The expression of pathway genes on multi-copy plasmids can lead to overexpression phenotypes for certain genes. These phenotypes can be growth retardation, inclusion bodies, and cell death. Therefore the expression levels of genes on multi copy plasmids has to be controlled effectively by using inducible expression systems, optimizing the time of induction of said expression system, and optimizing the amount of inducer provided. The time of induction has to be correlated to the growth phase of the biocatalyst, which can be followed by measuring of optical density in the fermentation broth.

A biocatalyst that has all pathway genes integrated on its chromosome is far more likely to allow constitutive expression since the lower number of gene copies may avoid overexpression phenotypes.

Plasmids disclosed herein were generally based upon parental plasmids described previously (Lutz, R. & Bujard, H. (1997) Nucleic Acids Research 25(6):1203-1210). Plasmids pGV1698 (SEQ ID NO: 112) and pGV1655 (SEQ ID NO: 109) produce optimized levels of isobutanol pathway enzymes in a production host when compared to other expression systems in the art. Compared to the expression of the isobutanol pathway from pSA55 and pSA69 as described in (WO 2008/098227) BIOFUEL PRODUCTION BY RECOMBINANT MICROORGANISMS, pGV1698 and pGV1655 lead to higher expression of E. coli IlvC and Bacillus subtilis AlsS and lower expression levels for Lactococcus lactis Kivd and E. coli ilvD. These changes are the result of differences in plasmid copy numbers. Also the genes coding for E. coli IlvD and E. coli IlvC were codon optimized for E. coli. This leads to optimized expression of the genes and it also avoids recombination of these genes with their native copies on the E. coli chromosome, thus stabilizing the production strain. The combination of two plasmids with the pSC101 and the ColE1 origin of replication in one cell as realized in a production strain carrying pGV1698 and pGV1655 is known to be more stable than the combination of two plasmids with p15A and ColE1 origins respectively as was used in the prior art (WO 2008/098227—BIOFUEL PRODUCTION BY RECOMBINANT MICROORGANISMS).

### Reduction of Enzymatic Activity

Host microorganisms within the scope of the invention may have reduced enzymatic activity such as reduced alcohol dehydrogenase activity. The term “reduced” as used herein with respect to a particular enzymatic activity refers to a lower level of enzymatic activity than that measured in a comparable host cell of the same species. Thus, host cells lacking alcohol dehydrogenase activity are considered to have reduced alcohol dehydrogenase activity since most, if not all, comparable host cells of the same species have at least some alcohol dehydrogenase activity. Such reduced enzymatic activities can be the result of lower enzyme expression level, lower specific activity of an enzyme, or a combination thereof. Many different methods can be used to make host cells having reduced enzymatic activity. For example, a host cell can be engineered to have a disrupted enzyme-encoding locus using common mutagenesis or knock-out technology. See, e.g., Methods in Yeast Genetics (1997 edition), Adams, Gottschling, Kaiser, and Stems, Cold Spring Harbor Press (1998), Datsenko and Wanner, Proc. Natl. Acad. Sci. USA 97, 6640-6645, 2000.

In addition, certain point-mutation(s) can be introduced which results in an enzyme with reduced activity.

Alternatively, antisense technology can be used to reduce enzymatic activity. For example, host cells can be engineered to contain a cDNA that encodes an antisense molecule that prevents an enzyme from being made. The term “antisense molecule” as used herein encompasses any nucleic acid molecule that contains sequences that correspond to the coding strand of an endogenous polypeptide. An antisense molecule also can have flanking sequences (e.g., regulatory sequences). Thus antisense molecules can be ribozymes or antisense oligonucleotides. A ribozyme can have any general structure including, without limitation, hairpin, hammerhead, or axhead structures, provided the molecule cleaves RNA.

Host cells having a reduced enzymatic activity can be identified using many methods. For example, host cells having reduced alcohol dehydrogenase activity can be easily identified using common methods, which may include, for example, measuring ethanol formation via gas chromatography.

### Increase of Enzymatic Activity

Host microorganisms of the invention may be further engineered to have increased activity of enzymes. The term “increased” as used herein with respect to a particular enzymatic activity refers to a higher level of enzymatic activity than that measured in a comparable yeast cell of the same species. For example, overexpression of a specific enzyme can lead to an increased level of activity in the cells for that enzyme. Increased activities for enzymes involved in glycolysis or the isobutanol pathway would result in increased productivity and yield of isobutanol.

Methods to increase enzymatic activity are known to those skilled in the art. Such techniques may include increasing the expression of the enzyme by increasing plasmid copy number and/or use of a stronger promoter and/or use of activating riboswitches, introduction of mutations to relieve negative regulation of the enzyme, introduction of specific mutations to increase specific activity and/or decrease the K_{M} for the substrate, or by directed evolution. See, e.g., Methods in Molecular Biology (vol. 231), ed. Arnold and Georgiou, Humana Press (2003).

### Microorganism in Detail

### Microorganism Characterized by the Ability to Produce Isobutanol Under Anaerobic Conditions

Economic studies indicate that the aeration of a fermentation process leads to increased operating and capital expenses and thus makes such a fermentation process less desirable compared to a fermentation process that operates under anaerobic conditions. In addition, yield and aeration conditions are closely related. For example, oxygen used as the terminal electron acceptor in respiration leads to undesired loss of carbon in the form of carbon dioxide, resulting in a reduced yield of the target compound.

As exemplified in the examples below, the present inventors have overcome the problem of an oxygen requirement for the production of a fermentation product. For example isobutanol was produced anaerobically at rates, titers and yields comparable to those achieved under micro-aerobic conditions.

Thus, in one embodiment, a modified microorganism may produce said fermentation product under anaerobic conditions, conditions at higher rates, and yields, as compared to a the wild-type or parental microorganism.

In one embodiment, said modified microorganism may be engineered to balance cofactor usage during the production of said fermentation product under anaerobic conditions.

In a specific aspect, a modified microorganism in which cofactor usage is balanced during the production of isobutanol may allow the microorganism to produce said isobutanol under anaerobic conditions at higher rates and yields as compared to a modified microorganism in which the cofactor usage in not balanced during production of isobutanol. One compound to be produced by the recombinant microorganism according to the present invention is isobutanol. However, the present invention is not limited to isobutanol. The invention may be applicable to any metabolic pathway that is imbalanced with respect to cofactor usage. One of skill in the art is able identify pathways that are imbalanced with respect to cofactor usage and apply this invention to provide recombinant microorganisms in which the same pathway is balanced with respect to cofactor usage.

Any method, including the methods described herein may be used to provide a modified microorganism with a metabolic pathway for the production of a target compound in which the cofactor usage is balanced; i.e. said metabolic pathway utilizes the same cofactor that is produced during glycolysis.

In one embodiment, the microorganism may converts glucose, which can be derived from biomass into a target compound under anaerobic conditions with a yield of greater than 75% of theoretical. In another embodiment, the yield is greater than 80% of theoretical. In another embodiment the yield is greater than 85% of theoretical. In another embodiment, the yield is greater than 90% of theoretical. In another embodiment, the yield is greater than 95% of theoretical. In another embodiment, the yield is greater than 97% of theoretical. In another embodiment the yield is greater than 98% of theoretical. In yet another embodiment, the yield is greater than 99% of theoretical. In still another embodiment, the yield is approximately 100% of theoretical

In one aspect, the microorganism may convert glucose, which can be derived from biomass into isobutanol under anaerobic conditions with a yield of greater than 50% of theoretical. In one embodiment, the yield is greater than 60% theoretical. In another embodiment, the yield is greater than 70% of theoretical. In yet another embodiment the yield is greater than 80% of theoretical. In yet another embodiment, the yield is greater than 85% of theoretical. In another embodiment, the yield is greater than 90% of theoretical. In yet another embodiment, the yield is greater than 95% of theoretical. In yet another embodiment, the yield is greater than 97% of theoretical. In yet another embodiment the yield is greater than 98% of theoretical. In yet another embodiment, the yield is greater than 99% of theoretical. In still another embodiment, the yield is approximately 100% of theoretical.

It is understood that while in the present disclosure the yield is exemplified for glucose as a carbon source, the invention can be applied to other carbon sources and the yield may vary depending on the carbon source used. One skilled in the art can calculate yields on various carbon sources. Other carbon sources, such as including but not limited to galactose, mannose, xylose, arabinose, sucrose, lactose, may be used. Further, oligomers or polymers of these and other sugars may be used as a carbon source.

### Microorganism Characterized by an Increased Product Yield

Economic studies indicate that the predominant factor accounting for the production cost for commodity chemicals and fuels from fermentation processes is attributed to the feedstock cost. In fact, as much as 60% of the variable cash operating costs or more may be attributable to feedstock costs. An important measure of the process economics is therefore the product yield. For a biocatalyst to produce a biofuel most economically, a single product is desired. Extra products reduce primary product yield increasing capital and operating costs, particularly if those extra, undesired products, or byproducts have little or no value. Extra products or byproducts also require additional capital and operating costs to separate these products from the product or biofuel of interest or may require additional cost for disposal.

As exemplified in the examples below, the present inventors have shown that, achieving cofactor balance increases the yield of fermentation products as compared to wild-type or parental organisms.

In an embodiment, a microorganism is provided in which cofactor usage is balanced during the production of a fermentation product and the microorganism produces the fermentation product at a higher yield compared to a modified microorganism in which the cofactor usage in not balanced.

In a specific aspect of the present invention, a microorganism is provided in which cofactor usage is balanced during the production of isobutanol and the microorganism produces isobutanol at a higher yield compared to a modified microorganism in which the cofactor usage in not balanced.

One compound to be produced by the recombinant microorganism according to the present invention is isobutanol. However, the present invention is not limited to isobutanol. The invention may be applicable to any microorganism comprising a metabolic pathway that leads to an imbalance with respect to cofactor usage. One of skill in the art is able to identify microorganisms comprising metabolic pathways that lead to an imbalance with respect to cofactor usage and apply this invention to provide recombinant microorganisms in which the microorganism comprising the same metabolic pathway is balanced with respect to cofactor usage.

Any method, including the methods described herein may be used to provide a modified microorganism with a metabolic pathway for the production of a target compound in which the cofactor usage is balanced; i.e. said metabolic pathway utilizes the same cofactor that is produced during glycolysis.

In one embodiment, the microorganism may convert glucose, which can be derived from biomass into a target compound with a yield of greater than 75% of theoretical. In another embodiment, the yield is greater than 80% of theoretical. In another embodiment the yield is greater than 85% of theoretical. In another embodiment, the yield is greater than 90% of theoretical. In another embodiment, the yield is greater than 95% of theoretical. In another embodiment, the yield is greater than 97% of theoretical. In another embodiment the yield is greater than 98% of theoretical. In yet another embodiment, the yield is greater than 99% of theoretical. In still another embodiment, the yield is approximately 100% of theoretical

In one aspect, the microorganism may convert glucose, which can be derived from biomass into isobutanol with a yield of greater than 75% of theoretical. In one embodiment, the yield is greater than 80% of theoretical. In one embodiment the yield is greater than 85% of theoretical. In another embodiment, the yield is greater than 90% of theoretical. In yet another embodiment, the yield is greater than 95% of theoretical. In yet another embodiment, the yield is greater than 97% of theoretical. In yet another embodiment the yield is greater than 98% of theoretical. In yet another embodiment, the yield is greater than 99% of theoretical. In still another embodiment, the yield is approximately 100% of theoretical.

It is understood that while in the present disclosure the yield is exemplified for glucose as a carbon source, the invention can be applied to other carbon sources and the yield may vary depending on the carbon source used. One skilled in the art can calculate yields on various carbon sources. Other carbon sources, such as including but not limited to galactose, mannose, xylose, arabinose, sucrose, lactose, may be used. Further, oligomers or polymers of these and other sugars may be used as a carbon source.

### Microorganism Characterized by Balancing Cofactor Usage

The ideal production microorganism produces a desirable product at close to theoretical yield. For example the ideal isobutanol producing organism produces isobutanol according to the following equation:

1 glucose→isobutanol+2 CO₂+H₂O

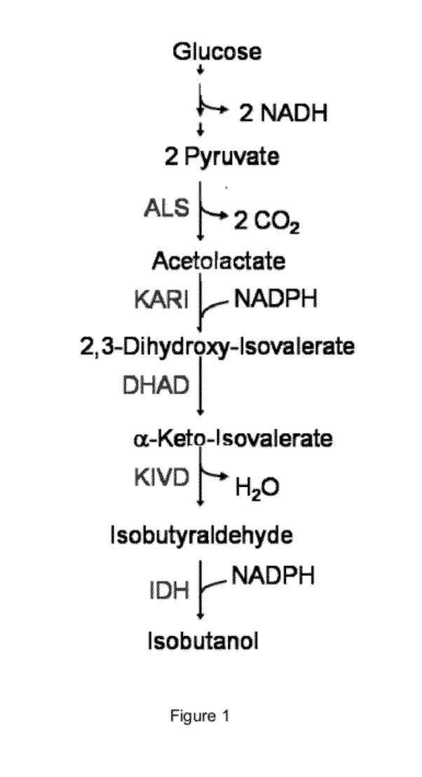

Accordingly, 66% of the glucose carbon results in isobutanol, while 33% is lost as CO₂. In exemplary metabolic pathways for the conversion of pyruvate to isobutanol described by Atsumi et al. (Atsumi et al., Nature, 2008 Jan. 3; 451(7174):86-9, which is herein incorporated by reference; International Patent Application No PCT/US2008/053514, which is herein incorporated by reference) two of the five enzymes used to convert pyruvate into isobutanol according to the metabolic pathway outlined in FIG. 1 require the reduced cofactor nicotinamide adenine dinucleotide phosphate (NADPH). NADPH is produced only sparingly by the cell—the reduced cofactor nicotinamide adenine dinucleotide (NADH) is the preferred equivalent. Respiration is required to produce NADPH in the large quantities required to support high-level production of isobutanol.

Even If competing pathways can be eliminated or reduced in activity by metabolic engineering, yield is limited to about 83% of theoretical. Carbon loss to carbon dioxide (CO₂) remains the main limitation on yield in the aforementioned metabolic pathway for the production of isobutanol. Reducing the oxygen uptake rate (OUR) of the cells should decrease the loss of carbon to CO₂ because it decreases the metabolic flux through the CO₂-generating tricarboxylic acid (TCA) cycle and/or pentose phosphate pathway (PPP). However, a modified microorganism utilizing the aforementioned metabolic pathway for the production of isobutanol exhibits drastically decreased specific productivity under conditions where the OUR is decreased and isobutanol production under anaerobic conditions may not be possible.

The decreased yield and the loss of productivity upon O₂ limitation indicate that the strain uses one or more metabolic pathways to generate the NADPH needed to support isobutanol production. In a modified cell utilizing the aforementioned metabolic pathway the production of isobutanol from glucose results in an imbalance between the cofactors reduced during glycolysis and the cofactors oxidized during the conversion of pyruvate to isobutanol. While glycolysis produces two moles of NADH, the isobutanol pathway consumes two moles of NADPH. This leads to a deficit of two moles of NADPH and overproduction of two moles of NADH per isobutanol molecule produced, a state described henceforth as cofactor imbalance.

The terms “cofactor balance” or “balanced with respect to cofactor usage” refer to a recombinant microorganism comprising a metabolic pathway converting a carbon source to a fermentation product and a modification that leads to the regeneration of all redox cofactors within the recombinant microorganism producing said fermentation product from a carbon source and wherein the re-oxidation or re-reduction of said redox cofactors does not require the pentose phosphate pathway, the TCA cycle or the generation of additional fermentation products.

Stated another way, the terms “cofactor balance” or “balanced with respect to cofactor usage” can refer to an advantageous modification that leads to the regeneration of all redox cofactors within the recombinant microorganism producing a fermentation product from a carbon source and wherein said re-oxidation or re-reduction of all redox cofactors does not require the production of byproducts or co-products.

Stated another way, the terms “cofactor balance” or “balanced with respect to cofactor usage” can refer to an advantageous modification that leads to the regeneration of all redox cofactors within the recombinant microorganism producing a fermentation product from a carbon source under anaerobic conditions and wherein the production of additional fermentation products is not required for re-oxidation or re-reduction of redox cofactors.

Stated another way, the terms “cofactor balance” or “balanced with respect to cofactor usage” can refer to an advantageous modification that leads to the regeneration of all redox cofactors within the recombinant microorganism producing a fermentation product from a carbon source and wherein said modification increases production of said fermentation product under anaerobic conditions compared to the parental or wild type microorganism and wherein additional fermentation products are not required for the regeneration of said redox cofactors.

The cell has several options for resolving a cofactor imbalance. One is to change the relative fluxes going from glucose through glycolysis and through the pentose phosphate pathway (PPP). For each glucose molecule metabolized through the PPP, two moles of NADPH are generated in addition to the two moles of NADH that are generated through glycolysis (a total of 4 reducing equivalents). Therefore, use of the PPP results in the generation of excess reducing equivalents since only two moles are consumed during the production of isobutanol. Under anaerobic conditions, and without an alternate electron acceptor, the cell has no way to reoxidize or regenerate these extra cofactors to NADP⁺ and metabolism thus stops. The excess reducing equivalents must instead be utilized for energy production through aerobic respiration which is only possible under aerobic conditions or for the production of byproducts. Another result of the flux through the PPP is that one additional molecule of CO₂ is lost per molecule of glucose consumed, which limits the yield of isobutanol that can be achieved under aerobic conditions.

Another way the cell can generate NADPH is via the TCA cycle. Flux through the TCA cycle results in carbon loss through CO₂ and in production of NADH in addition to the NADPH required for the isobutanol pathway. The NADH would have to be utilized for energy production through respiration under aerobic conditions (and without an alternate electron acceptor) or for the production of byproducts. In addition, the TCA cycle likely is not functional under anaerobic conditions and is therefore unsuitable for the production of stoichiometric amounts of NADPH in an anaerobic isobutanol process.

An economically competitive isobutanol process requires a high yield from a carbon source. Lower yield means that more feedstock is required to produce the same amount of isobutanol. Feedstock cost is the major component of the overall operating cost, regardless of the nature of the feedstock and its current market price. From an economical perspective, this is important because the cost of isobutanol is dependent on the cost of the biomass-derived sugars. An increase in feedstock cost results in an increase in isobutanol cost. Thus, it is desirable to utilize NADH-dependent enzymes for the conversion of pyruvate to isobutanol.

An enzyme is “NADH-dependent” if it catalyzes the reduction of a substrate coupled to the oxidation of NADH with a catalytic efficiency that is greater than the reduction of the same substrate coupled to the oxidation of NADPH at equal substrate and cofactor concentrations.

Thus, in one embodiment of the invention, a microorganism is provided in which cofactor usage is balanced during the production of a fermentation product.

In a specific aspect, a microorganism is provided in which cofactor usage is balanced during the production of isobutanol, in this case, production of isobutanol from pyruvate utilizes the same cofactor that is produced during glycolysis.

In another embodiment, a microorganism is provided in which cofactor usage is balanced during the production of a fermentation product and the microorganism produces the fermentation product at a higher yield compared to a modified microorganism in which the cofactor usage in not balanced.

In a specific aspect, a microorganism is provided in which cofactor usage is balanced during the production of isobutanol and the microorganism produces isobutanol at a higher yield compared to a modified microorganism in which the cofactor usage in not balanced.

In yet another embodiment, a modified microorganism in which cofactor usage is balanced during the production of a fermentation product may allow the microorganism to produce said fermentation product under anaerobic conditions at higher rates, and yields as compared to a modified microorganism in which the cofactor usage in not balanced during production of a fermentation product.

In a specific aspect, a modified microorganism in which cofactor usage is balanced during the production of isobutanol may allow the microorganism to produce isobutanol under anaerobic conditions at higher rates, and yields as compared to a modified microorganism in which the cofactor usage is not balanced during production of isobutanol.

One compound to be produced by the recombinant microorganism according to the present invention is isobutanol. However, the present invention is not limited to isobutanol. The invention may be applicable to any metabolic pathway that is imbalanced with respect to cofactor usage. One skilled in the art is able to identify pathways that are imbalanced with respect to cofactor usage and apply this invention to provide recombinant microorganisms in which the same pathway is balanced with respect to cofactor usage. One skilled in the art will recognize that the identified pathways may be of longer or shorter length, contain more or fewer genes or proteins, and require more or fewer cofactors than the exemplary isobutanol pathway. Further, one skilled in the art will recognize that in certain embodiments, such as a recombinant microbial host that produces an excess of NADPH, certain embodiments of the present invention may be adapted to convert NADPH to NADH.

### Microorganism Characterized by Providing Cofactor Balance Via Overexpression of a Transhydrogenase

Conversion of glucose to pyruvate via glycolysis in E. coli leads to the production of two moles of NADH. A metabolic pathway that converts pyruvate to a target product that consumes either two moles of NADPH or one mole of NADH and one mole of NADPH leads to cofactor imbalance. For example, the isobutanol metabolic pathway that converts glucose to two moles of pyruvate via glycolysis to 1 mole of isobutanol generates two moles of NADH and consumes two moles of NADPH and thus is imbalanced with respect to cofactor usage.

The different ways in which the cell can provide NADPH to the isobutanol pathway show that utilization of the TCA cycle as well as the PPP has to be avoided to maximize the yield of the isobutanol process. Loss of CO₂ as a byproduct in isobutanol producing microorganism described in the prior art (Atsumi et al., Nature, 2008 Jan. 3; 451(7174):86-9; International Patent Application No PCT/US2008/053514; International Patent Application No PCT/US2006/041602) indicates that either or both of these two yield-limiting pathways are currently active.

A Nicotinamide dinucleotide transhydrogenase (hereinafter may be referred to simply as “transhydrogenase”) that catalyzes the interconversion of NADH and NADPH as disclosed herein may be used to provide cofactor balance in a metabolic pathway for the production of a target compound that is otherwise imbalanced with respect to cofactor usage and thus decrease the yield loss to CO₂ in such a pathway (FIG. 2)

A preferred transhydrogenase under conditions in which the reduced cofactor NADPH is limiting is one that preferentially catalyzes the conversion of NADH to NADPH. For example, membrane-bound transhydrogenases have been described in bacteria that catalyze this reaction. Membrane bound transhydrogenases require energy in form of proton translocation to catalyze the reaction. As long as there is enough energy available to maintain the proton gradient across the cell membrane a transhydrogenase may thus be used to balance an otherwise imbalanced metabolic pathway. However, in some circumstances, a transhydrogenase that catalyzes the conversion of NADPH to NADH may be preferred. However, a preferred transhydrogenase under conditions in which the reduced cofactor NADH is limiting is one that preferentially catalyzes the conversion of NADPH to NADH.

The expression and specific activity of an endogenously expressed membrane-bound transhydrogenase might not be sufficient to maintain the high metabolic flux through the metabolic pathway for the production of a fermentation product (e.g. for isobutanol) that is required in a commercial process.

Thus, in one embodiment, the insufficient activity of the membrane-bound transhydrogenase may be compensated by overexpression of the coding genes of a membrane bound transhydrogenase.

In a preferred embodiment, the E. coli pntA (SEQ ID NO: 1) and pntB genes (SEQ ID NO: 3), encoding for the PntA (SEQ ID NO: 2) and PntB (SEQ ID NO: 4) enzymes respectively or homologs thereof may be overexpressed. These genes have been overexpressed in E. coli before for characterization of the enzyme (Clarke, D. M. and P. D. Bragg, Journal of Bacteriology, 1985. 162(1): p. 367-373) and have been used to regenerate NADPH cofactor in the production of chiral alcohols from ketones using a whole cell biocatalyst (Weckbecker, A. and W. Hummel, Biotechnology Letters, 2004. 26(22): p. 1739-1744.) or to increase production of biosynthesized products that rely on NADPH-dependent biosynthetic pathways (U.S. Pat. No. 5,830,716).

In one embodiment, the E. coli pntAB operon (SEQ ID NO: 1 and SEQ ID NO: 3) is expressed in the presence of the isobutanol pathway. The E. coli pntAB operon may be cloned on a medium copy plasmid (p15A origin of replication) under the control of the LtetOI promoter, for example pGV1685 (SEQ ID NO: 111). The high level expression of membrane proteins can lead to the buildup of toxic intermediates and to inclusion bodies. Thus, in another embodiment, different copy numbers of the E. coli pntAB operons may be tested to find the optimum expression level of this membrane transhydrogenase.

In another embodiment, the E. coli pntAB operon may be integrated into the chromosome of the microorganism. For example, E. coli pntAB may be integrated into the E. coli genome.

In one aspect of the present invention, the pntAB operon may be integrated into the sthA locus of E. coli or the corresponding locus in another microorganism. The sthA gene codes for the soluble transhydrogenase of E. coli and has previously been shown to be utilized by the cell for the conversion of NADPH to NADH. To avoid the generation of a futile cycle E. coli pntAB may be integrated at the sthA site, thus removing the sthA gene and eliminating this reverse reaction.

The E. coli pntAB operon may be integrated into a wild-type E. coli W3110 and then transduced into a recombinant microorganism that produces a product via a metabolic pathway that is imbalanced with respect to cofactor usage. For example, the E. coli pntAB operon may be integrated into an isobutanol producing strain in which the isobutanol pathway is integrated into the chromosome.

For example the E. coli pntAB operon may be integrated into the isobutanol pathway strain GEVO1859 which has the pathway genes Bs_alsS1 and Ec_ilvC_coEc integrated into the pflB site and has Ll_kivd1 and Ec_ilvD_coEc genes integrated into the adhE site. All genes may be under the control of the LlacOI promoter.

The soluble E. coli transhydrogenase coded by sthA has been shown to be utilized by the cell for the conversion of NADPH to NADH. However overexpression of sthA was demonstrated to increase the yield of poly(3-hydroxybutyrate) production in E. coli. These results indicate that if a pathway is present in E. coli that consumes NADPH effectively, the soluble transhydrogenase can function in the direction of NADPH production. The advantages of using SthA as opposed to E. coli PntAB are that the soluble protein might be easier to overexpress and that this enzyme is energy independent. The sthA gene may be cloned into pGV1685, replacing E. coli pntAB. Decisive for the success of this approach is the affinity of E. coli IlvC (KARI enzyme) for its cofactor and the steady state concentrations of NADH and NADPH in the cell that allow SthA to run “backwards” or in the direction of converting NADH to NADPH. It is to be expected that the concentration of the reduced cofactor NADPH has to be low in order for SthA to supply this cofactor. If this concentration is low enough to limit the activity of E. coli IlvC and therefore the flux through the isobutanol pathway then this approach is not suitable for the isobutanol production strain without further modifications. These modifications could be identification of a KARI with a lower K_{M} for NADPH, or mutagenesis and directed evolution to increase the affinity of E. coli IlvC for its cofactor.

This approach may be used to provide cofactor balance in a metabolic pathway otherwise imbalanced with respect to cofactor usage if the steady state concentrations of NADH and NADPH in the cell are appropriate to allow SthA to run “backwards” or in the direction of converting NADH to NADPH. It is to be expected that the concentration of the reduced cofactor NADPH has to be low in order for SthA to supply this cofactor.

This embodiment may enable higher yields of a fermentation product in a microorganism. Further, this embodiment may enable economical anaerobic production of a fermentation product, which was not possible without the teachings of this embodiment. Further, this embodiment may enable aerobic production of a fermentation product at higher yield, which was not possible without the teachings of this embodiment.

### Microorganism Characterized by Providing Cofactor Balance Via Overexpression of an NADPH-Dependent GAPDH

Conversion of glucose to pyruvate via glycolysis in E. coli leads to the production of two moles of NADH. A metabolic pathway that converts pyruvate to a target product that consumes either two moles of NADPH or one mole of NADH and one mole of NADPH leads to cofactor imbalance. For example, the isobutanol metabolic pathway that converts glucose to two moles of pyruvate via glycolysis to 1 mole of isobutanol generates two moles of NADH and consumes two moles of NADPH and thus is imbalanced with respect to cofactor usage.

GAPDH catalyzes the conversion of glyceraldehyde 3-phosphate (GAP) to 1,3-diphosphate glycerate as part of glycolysis. For example, in E. coli GAPDH is encoded by gapA which is NADH-dependent and is active in glycolysis as well as in gluconeogenesis [DellaSeta, F., et al., Characterization of Escherichia coli strains with gapA and gapB genes deleted. Journal of Bacteriology, 1997. 179(16): p. 5218-5221.]. GAPDH proteins from other organisms vary in their cofactor requirements.

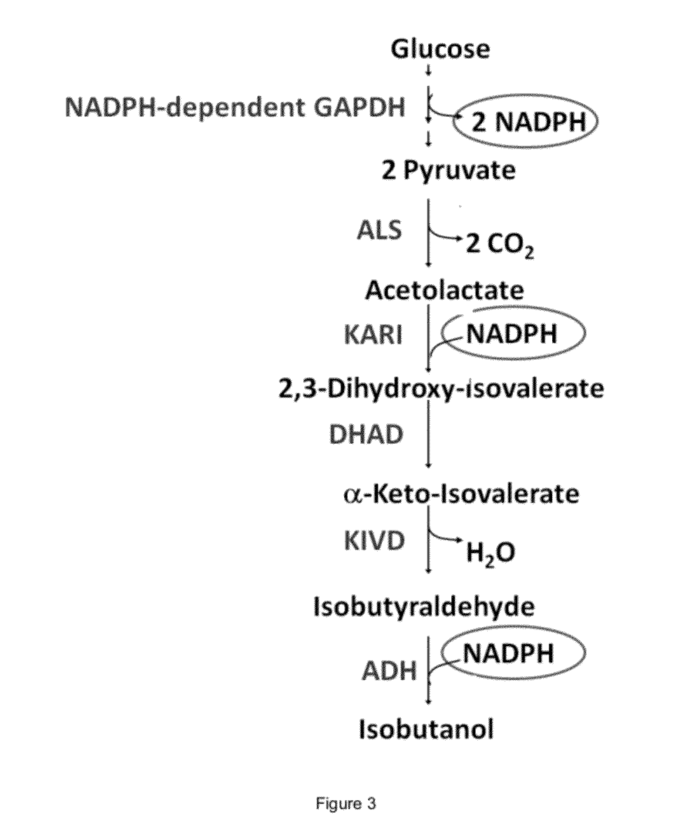

Thus in an embodiment, a recombinant microorganism that produces a compound may express a GAPDH is that uses the same cofactor as the fermentative pathway for the production of said compound. For example, in case of an isobutanol biosynthetic pathway that consumes two moles of NADPH per mole of pyruvate an NADPH-dependent GAPDH may be utilized to provide a metabolic pathway that is balanced with respect to cofactor usage (FIG. 3). In such an embodiment, it may also be desirable to increase the concentration of NADPH in the cell by overexpression of other enzymes for the metabolic synthesis of NADPH cofactor. In other embodiments, it may also be desirable to increase the concentration of NADPH in the cell by overexpression of other enzymes for the metabolic synthesis of NADPH cofactor.

Thus, such an NADPH-dependent GAPDH may be expressed in a recombinant microorganism. NADPH-dependent GAPDH enzymes may be identified by analysis with an in vitro enzyme assay. Further, some NADPH-dependent GAPDH enzymes may be identified by analysis of protein identity, similarity, or homology. Further, genes that encode NADPH-dependent GAPDH enzymes may be identified by analysis of gene identity, similarity, or homology.

One NADPH-dependent GAPDH according to the present invention with reported high activity with NADPH is Gdp1 from Kluyveromyces lactis [Verho, R., et al., Identification of the first fungal NADP-GAPDH from Kluyveromyces lactis. Biochemistry, 2002. 41(46): p. 13833-13838.]. Gdp1 has been expressed in Saccharomyces cerevisiae to improve ethanol fermentations on xylose as a substrate [Verho, R., et al., Engineering redox cofactor regeneration for improved pentose fermentation in Saccharomyces cerevisiae. Applied and Environmental Microbiology, 2003. 69(10): p. 5892-5897.] Expression of Gdp1 improved the yield of the fermentation from 18 to 23% and from 24 to 41% when it was coupled to a zwf1 deletion which forces more flux through glycolysis. Purified Gdp1 was shown in the literature to be as active with NAD+ as it is with NADP+. Thus, the intracellular concentrations and more importantly the redox ratio of the cofactors in a recombinant microorganism according to the present invention will dictate which cofactor is used in glycolysis.

Another NADPH accepting GAPDH is found in Clostridium acetobutylicum and is coded by the gene gapC. Additional homologs of NADPH-dependent GAPDH enzymes may be found in thermotolerant bacteria. Other alternatives of such GAPDH enzymes are those found in cyanobacteria.

A different class of enzymes that can be used to generate NADPH from glucose during glycolysis is comprised of the NADP+-dependent GAPDH (non-phosphorylating). Such enzymes are designated as GapN. However, use of this enzyme results in a loss of one ATP per pyruvate produced. Thus, the production of one NADPH is coupled to a reduction of ATP yield by 1 ATP.

To provide cofactor balance in a recombinant microorganism via an NADPH-dependent GAPDH, it may be necessary to deactivate the native NADH-dependent GAPDH. For example, in the host strain E. coli the gapA gene may be deleted.

Another way to force the cell to produce NADPH with GDP1 is the elimination of flux through the PPP. This can be accomplished by deletion of the gene that encodes 6-Phosphogluconate dehydrogenase or decreasing the activity of 6-Phosphogluconate dehydrogenase. For example, in E. coli 6-Phosphogluconate dehydrogenase is encoded by zwf. The mutation of zwf eliminates flux through the PPP and may force the microorganism to utilize glycolysis in which the heterologously expressed GAPDH will utilize the cofactor NADP+ instead of NADH.

Alternatively, cofactor imbalance in a recombinant microorganism Alternatively, cofactor imbalance in a recombinant microorganism that produces a fermentation product may be alleviated by engineering the native GAPDH to accept NADPH as cofactor. A crystal structure is available from the Palinurus versicolor GAPDH which can be used to model the structures of GDP1, GapA (E. coli) and other GAPDH enzymes with different cofactor specificities. It is known that an aspartate residue in the NAD binding site is conserved among the NAD dependent GAPDHs. This residue is replaced by asparagine in GDP1.

Additional target amino acids may be found using sequence alignments and structure modeling for site directed mutagenesis. The gapA gene can be mutated using saturation mutagenesis or random mutagenesis according to protein engineering methods known to those skilled in the art. The library of mutant genes may be transformed into microorganisms carrying a zwf deletion and expressing a metabolic pathway that is imbalanced with respect to cofactor usage pathway genes. Mutant enzymes that are NADPH-dependent may be identified in those microorganism that grow on a growth medium. In certain embodiments, it may not be necessary to delete the zwf gene. Alternate genes known to one skilled in the art may be deleted from the organism that in effect inhibits flux through the pentose phosphate pathway.

This embodiment may enable higher yields of a fermentation product in a microorganism. Further, this embodiment may enable anaerobic production of a fermentation product, which was not possible without the teachings of this embodiment. Further, this embodiment may enable anaerobic production of a fermentation product at higher yield, which was not possible without the teachings of this embodiment.

### Microorganism Characterized by Providing Cofactor Balance Via a Transhydrogenase Cycle

Conversion of glucose to pyruvate via glycolysis in E. coli leads to the production of two moles of NADH. A metabolic pathway that converts pyruvate to a target product that consumes either two moles of NADPH or one mole of NADH and one mole of NADPH leads to cofactor imbalance. For example, the isobutanol metabolic pathway that converts glucose to two moles of pyruvate via glycolysis to 1 mole of isobutanol generates two moles of NADH and consumes two moles of NADPH and thus is imbalanced with respect to cofactor usage.

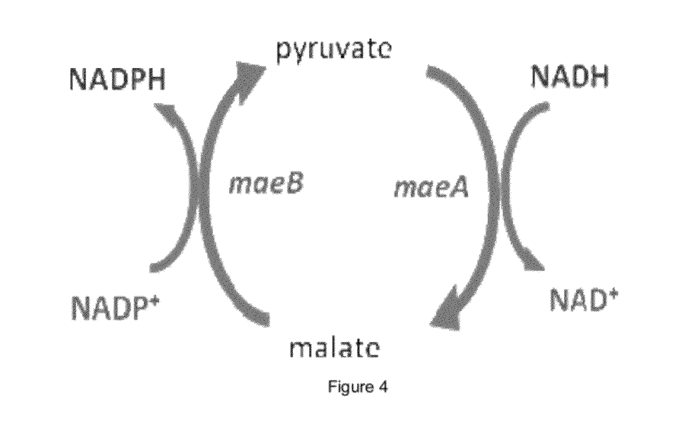

This cofactor imbalance may be resolved using two dehydrogenase enzymes that catalyze the same reaction but use different cofactors. One example for such a pair of enzymes are the malic enzymes MaeA and MaeB. MaeA is NADH-dependent and MaeB is NADPH-dependent and both catalyze the conversion of malate to pyruvate [Bologna, F. P., C. S. Andreo, and M. F. Drincovich, Escherichia coli malic enzymes: Two isoforms with substantial differences in kinetic properties, metabolic regulation, and structure. Journal of Bacteriology, 2007. 189(16): p. 5937-5946.]. The reaction catalyzed by each of these two enzymes is reversible. The kinetics of the two malic enzymes and the different concentrations and redox ratios of the cofactors they use might allow the NADH-dependent enzyme to run in the oxidative direction while the NADPH-dependent enzyme catalyses the reductive direction of the same conversion. In effect the enzymes would catalyze the interconversion of pyruvate and malate coupled to the consumption of NADH and the generation of NADPH (FIG. 4).

Thus the two malic enzymes may function like a transhydrogenase. This cofactor conversion cycle is dependent on the redox ratios of the cofactors which depends on the kinetics of the enzymes in an metabolic pathway that is imbalanced with respect to cofactor, for example the isobutanol pathway enzyme E. coli Ilvc as well as GapA and the malic enzymes. Homologs of malic enzymes can be identified by those skilled in the art. Those enzymes may be used which show kinetic properties favoring the oxidative conversion with NAD+ as cofactor and the reductive conversion with NADPH. The E. coli enzymes may to perform these reactions but enzymes with more favorable kinetics may increase the performance of the cofactor conversion.

This embodiment may enable higher yields of a fermentation product in a microorganism. Further, this embodiment may enable anaerobic production of a fermentation product, which was not possible without the teachings of this embodiment. Further, this embodiment may enable anaerobic production of a fermentation product at higher yield, which was not possible without the teachings of this embodiment.

### Microorganism Characterized by Providing Cofactor Balance Via Metabolic Transhydrogenation Via Ppc or Pyc

Conversion of glucose to pyruvate via glycolysis in E. coli leads to the production of two moles of NADH. A metabolic pathway that converts pyruvate to a target product that consumes either two moles of NADPH or one mole of NADH and one mole of NADPH leads to cofactor imbalance. For example, the isobutanol metabolic pathway that converts glucose to two moles of pyruvate via glycolysis to 1 mole of isobutanol generates two moles of NADH and consumes two moles of NADPH and thus is imbalanced with respect to cofactor usage.

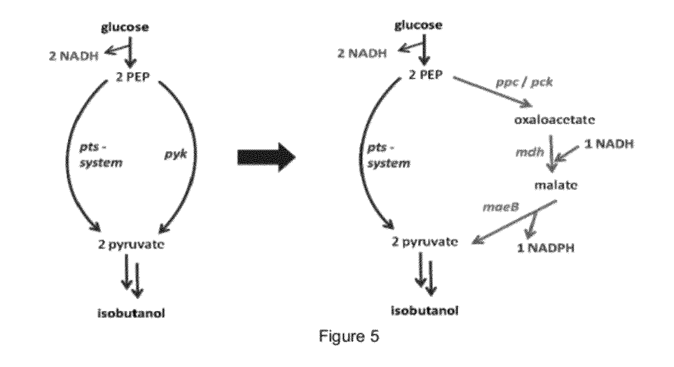

To resolve this cofactor imbalance the metabolic flux may be diverted to allow the conversion of at least one mole of NADH into NADPH. Looking at the stoichiometric network in E. coli points to a pathway that allows such a conversion of cofactors (FIG. 5).

Flux from PEP to pyruvate can be replaced by flux from PEP to oxaloacetate, to malate, to pyruvate. To redirect the flux in such a way the native conversion from PEP to pyruvate has to be removed from the network by deletion of the genes coding for pyruvate kinase (pykA, pykF). The other enzymes required are phosphoenolpyruvate carboxylase (Ppc) or phosphoenolpyruvate carboxykinase (Pck) for the conversion of PEP to oxaloacetate, malate dehydrogenase (mdh) for the conversion of oxaloacetate to malate and MaeB for the conversion of malate to pyruvate. The choice whether to use ppc or pck for the conversion of PEP to oxaloacetate depends on the energy load of the isobutanol production strain. With the deletion of Pyk the ATP yield of the strain is reduced if Ppc is used. If Pck is used instead the ATP yield is the same as when the flux goes from PEP to pyruvate using Pyk. Under production condition the strain will only need limited amounts of ATP for cell maintenance. This energy requirement might be lower than the two ATP per glucose generated by glycolysis. By overexpressing ppc, pck or both enzymes the energy yield of the conversion of PEP to pyruvate can be varied between one and two moles of ATP.

The native expression levels of some or all of the enzymes used in the above described conversion from PEP to pyruvate is expected to be insufficient to sustain the high glycolytic flux necessary in the isobutanol production strain. As an example the expression level of mdh is reduced in the presence of glucose and it is further reduced two-fold under anaerobic conditions. Therefore these enzymes may be overexpressed. To allow conversion of 50% of the NADH generated through glycolysis to NADPH the NADH-dependent malic enzyme MaeA may be deleted. Further the enzyme Mqo was reported to catalyze the conversion of malate to oxaloacetate and may be deleted to allow maximum flux in the opposite direction. The thermodynamic equilibrium of the conversion of malate to oxaloacetate lies on the malate side and Mdh catalyzes the reduction of oxaloacetate under anaerobic respiration and under fermentative conditions.

Flux through the PPP may be avoided by adding the deletion of zwf to the strain which eliminates glucose 6-phosphate 1-dehydrogenase the first committed step of the oxidative PPP.

This embodiment may enable higher yields of a fermentation product in a microorganism. Further, this embodiment may enable anaerobic production of a fermentation product, which was not possible without the teachings of this embodiment. Further, this embodiment may enable anaerobic production of a fermentation product at higher yield, which was not possible without the teachings of this embodiment.

### Yeast Microorganism Characterized by Providing Cofactor Balance

The aforementioned methods to provide cofactor balance are generally applicable to many microorganisms, including yeast microorganisms. Specifically, however, in yeast, metabolic transhydrogenation may accomplished by introduction of NADPH dependent malic enzyme into yeast. If the conversion of phosphoenol pyruvate to pyruvate by pyruvate kinase is disrupted then the carbon flux can go through a pyruvate kinase bypass that goes from PEP to oxaloacetate to malate and from there to pyruvate. The conversion of oxaloacetate to malate by Mdh consumes one NADH and the conversion of malate to pyruvate by the heterologous malic enzyme produces one NADPH. NADPH dependent malic enzymes are common in bacteria and one example is E. coli MaeB. If the NADPH cofactor is needed in the mitochondria the malic enzyme expression can be directed into this organelle instead of the cytoplasm by addition of mitochondrial targeting sequence to the N-terminus or C-terminus of the gene. Also, the yeast enzyme Mae1, which is physiologically localized in the mitochondria can be overexpressed. Malate as well as pyruvate is shuttled across the mitochondrial membranes enabling the pyruvate bypass to effectively convert one cytoplasmic NADH into a mitochondrial NADPH. In yeast the complete carbon flux can be diverted in this way since there is no phosphotransferase (pts) system for glucose import and all PEP generated by glycolysis is available. However, one ATP is lost per NADPH produced through the yeast pyruvate kinase bypass.

Yeast do not have transhydrogenases. The heterologous expression of bacterial, plant or other eukaryotic transhydrogenases in yeast can be used to provide cofactor balance. The transhydrogenases that natively convert NADH to NADPH are generally membrane proteins that use the proton motive force to drive the reaction they are catalyzing. Bacterial transhydrogenases are in the cell membrane while plant and mammalian transhydrogenases are located in the inner mitochondrial membrane. For the heterologous transhydrogenase expression these enzymes can be targeted either to the cytoplasmic membrane or to the mitochondrial membrane in yeast. To achieve this leader sequences have to be added to the heterologous proteins. The mechanisms of membrane targeting are well understood and the direction of normally cytosolic proteins to the mitochondrium has been demonstrated. These targeting mechanisms are well conserved throughout the eukaryotes, which was demonstrated by the use of plant mitochondrial targeting sequences in yeast. Eukaryotic transhydrogenases are expressed in yeast with their native targeting and sorting sequences. Bacterial transhydrogenases are fused to mitochondrial targeting and membrane sorting sequences that have been characterized in yeast membrane proteins.

An alternative approach for the production of NADPH is the use of biosynthetic pathway enzymes. An NADH kinase could phosphorylate NADH to NADPH. Then the NADP+ needs to be dephosphorylated to NAD+ to maintain NAD+ pool. This can be carried out by an NADP phosphatase.

### Microorganisms Characterized by Providing Cofactor Balance Via Engineered Enzymes

Conversion of one mole of glucose to two moles of pyruvate via glycolysis leads to the production of two moles of NADH. A metabolic pathway that converts pyruvate to a target product that consumes either two moles of NADPH or one mole of NADH and one mole of NADPH leads to cofactor imbalance. One example of such a metabolic pathway is the isobutanol metabolic pathway described by Atsumi et al. (Atsumi et al., 2008, Nature 451(7174): 86-9) which converts two moles of pyruvate to one mole of isobutanol. In this five enzyme pathway, two enzymes are dependent upon NADPH: (1) KARI and (2) ADH, encoded by the E. coli ilvC and E. coli yqhD, respectively.

To resolve this cofactor imbalance, the present invention provides a recombinant microorganism in which the NADPH-dependent enzymes KARI and ADH are replaced with enzymes that preferentially depend on NADH (i.e. KARI and ADH enzymes that are NADH-dependent).

To further resolve this cofactor imbalance, the present invention in another embodiment provides recombinant microorganisms wherein the NADH-dependent KARI and ADH enzymes are overexpressed.

In one aspect, such enzymes may be identified in nature. In an alternative aspect, such enzymes may be generated by protein engineering techniques including but not limited to directed evolution or site-directed mutagenesis.

In one embodiment, the two NADPH-dependent enzymes within an isobutanol biosynthetic pathway that converts pyruvate to isobutanol may be replaced with ones that utilize NADH. These two enzymes may be an NADH-dependent KARI (NKR) and an NADH-dependent alcohol dehydrogenase.

In another embodiment, two NADH-dependent enzymes that catalyze the same reaction as the NADPH-dependent enzymes are overexpressed. These two enzymes may be an NADH-dependent KARI (NKR) and an NADH-dependent alcohol dehydrogenase.

In one aspect, NADH-dependent KARI and ADH enzymes are identified in nature. In another aspect, the NADPH-dependent KARI and ADH enzymes may be engineered using protein engineering techniques including but not limited to directed evolution and site-directed mutagenesis.

There exist two basic options for engineering NADH-dependent alcohol dehydrogenases or ketol-acid reductoisomerases: (1) increase the NADH-dependent activity of an NADPH-dependent enzyme that is active towards the substrate of interest and/or (2) increase the activity of an NADH-dependent enzyme that is not sufficiently active towards the substrate of interest.

### NADH-Dependent KARI Enzymes

As shown in FIG. 1, the ketol-acid reductoisomerase (KARI) enzyme of the isobutanol biosynthetic pathway as disclosed by Atsumi et al (Atsumi et al., 2008, Nature 451(7174): 86-9, herein incorporated by reference in its entirety), requires the cofactor nicotinamide dinucleotide phosphate (NADPH) to convert acetolactate to 2,3-dihydroxyisovalerate. However, under anaerobic conditions, NADPH is produced only sparingly by the cell—nicotinamide adenine dinucleotide (NADH) is the preferred equivalent. Therefore, oxygen is required to produce NADPH in the large quantities to support high-level production of isobutanol. Thus, the production of isobutanol is feasible only under aerobic conditions and the maximum yield that can be achieved with this pathway is limited. Accordingly, KARI enzymes that preferentially utilize NADH rather than NADPH are desirable.

Other biosynthetic pathways utilize KARI enzymes for the conversion of acetolactate to 2-3-dihydroxyisovalerate. For example, KARI enzymes convert acetolactate to 2-3-dihydroxyisovalerate as part of the biosynthetic pathway for the production of 3-methyl-1-butanol (Atsumi et al., 2008, Nature 451(7174): 86-9, herein incorporated by reference in its entirety).

Yet other biosynthetic pathways utilize KARI to convert 2-aceto-2-hydroxy-butyrate to 2,3-dihydroxy-3-methylvalerate. This reaction is part of the biosynthetic pathway for the production of 2-methyl-1-butanol. (Atsumi et al., 2008, Nature 451(7174): 86-9, herein incorporated by reference in its entirety).

As used herein, the term “KARI” or “KARI enzyme” or “ketol-acid reductoisomerase” are used interchangeably herein to refer to an enzyme that catalyzes the conversion of acetolactate to 2,3-dihydroxyisovalerate and/or the conversion of 2-aceto-2-hydroxy-butyrate to 2,3-dihydroxy-3-methylvalerate. Moreover, these terms can be used interchangeably herein with the terms “acetohydroxy acid isomeroreductase” and “acetohydroxy acid reductoisomerase.”

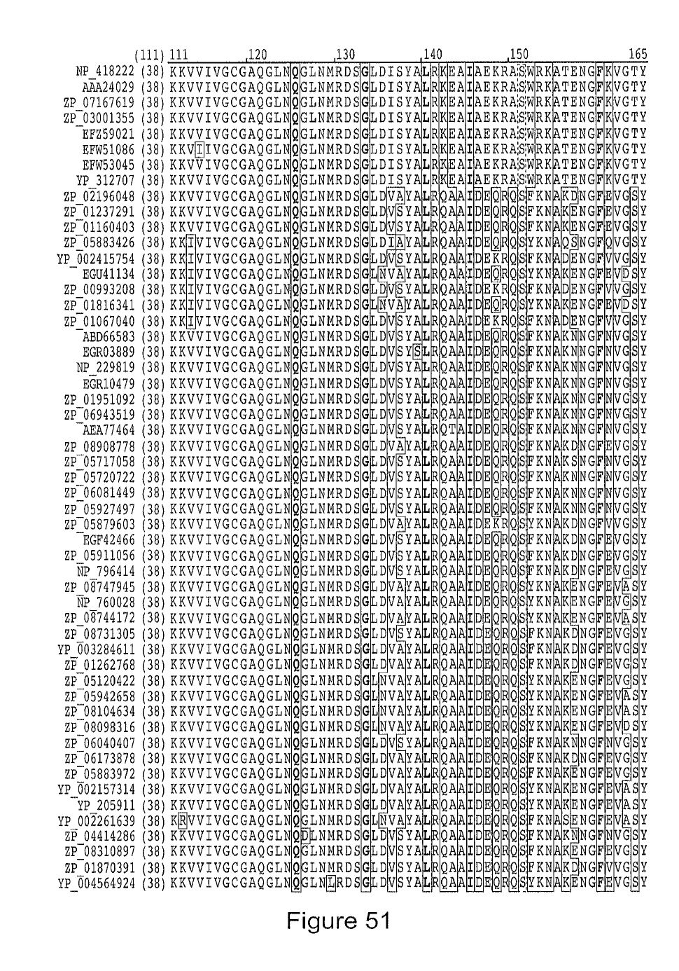

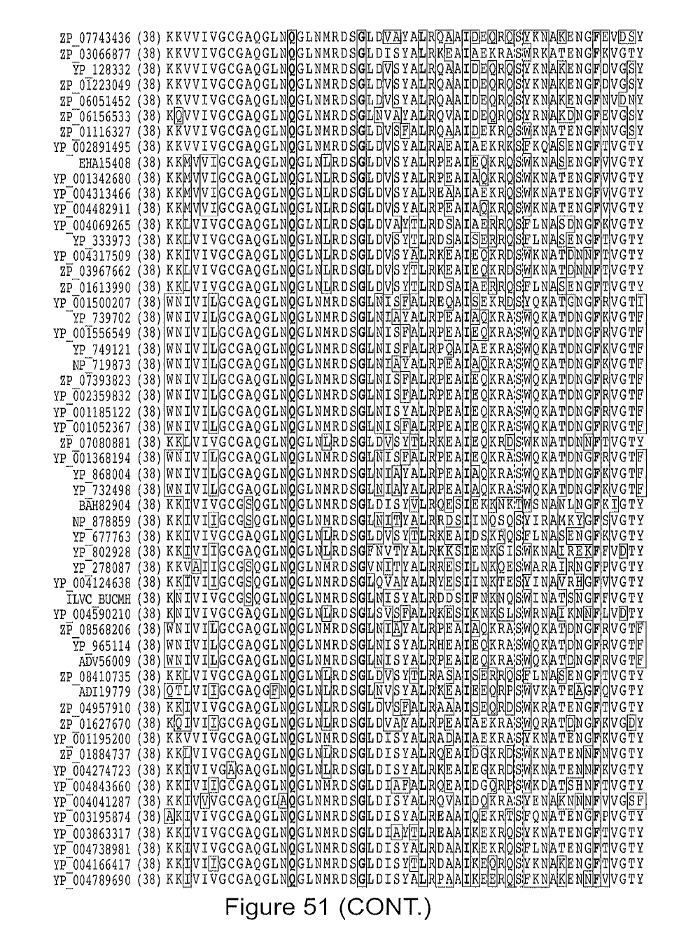

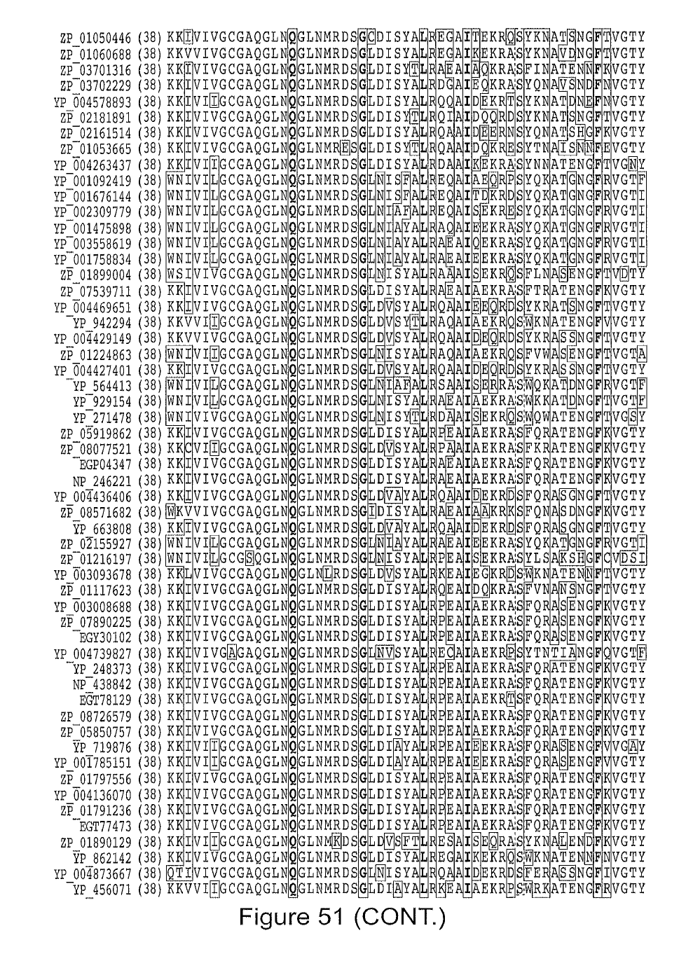

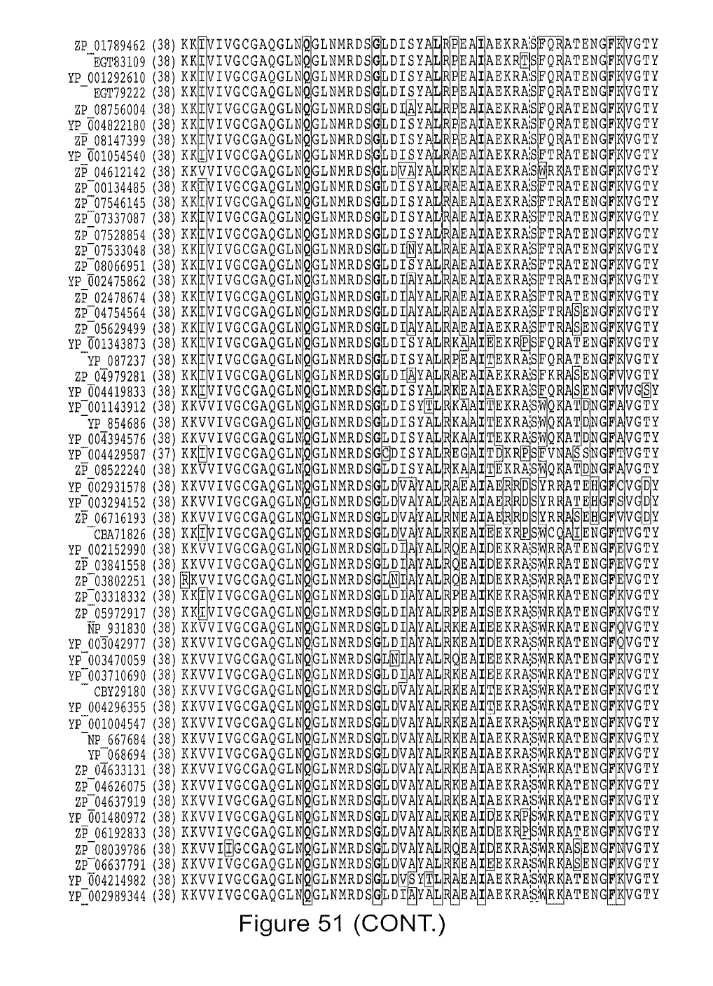

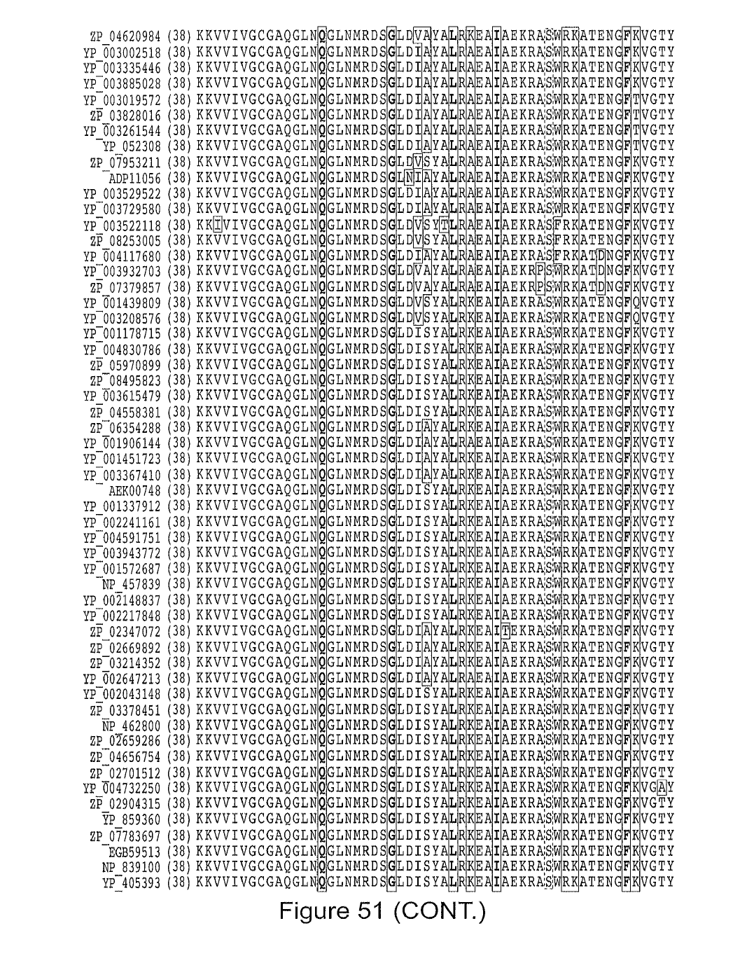

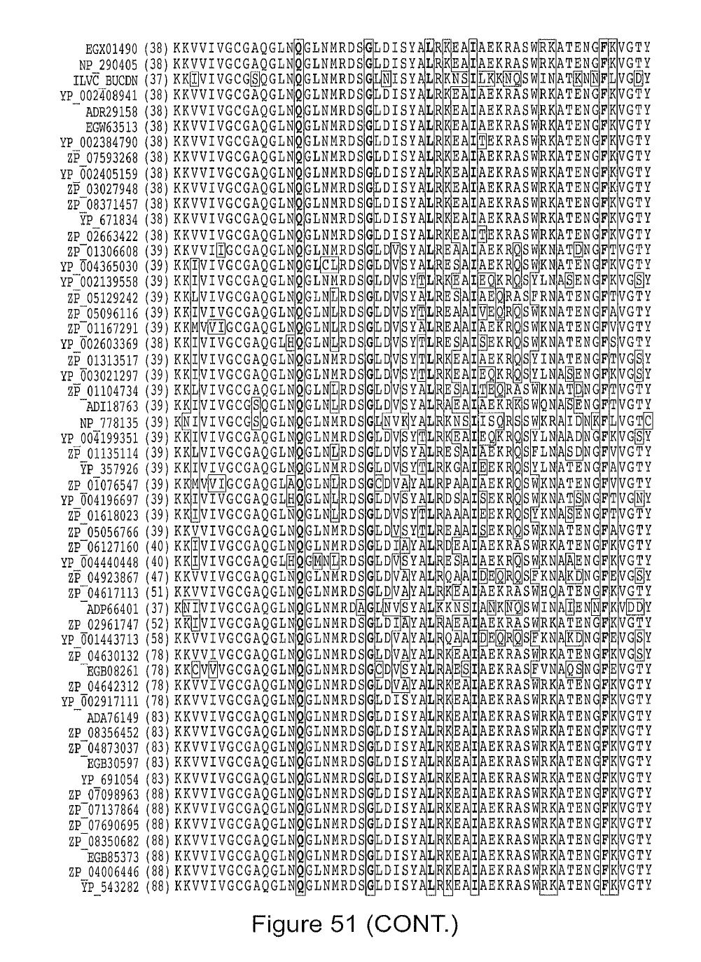

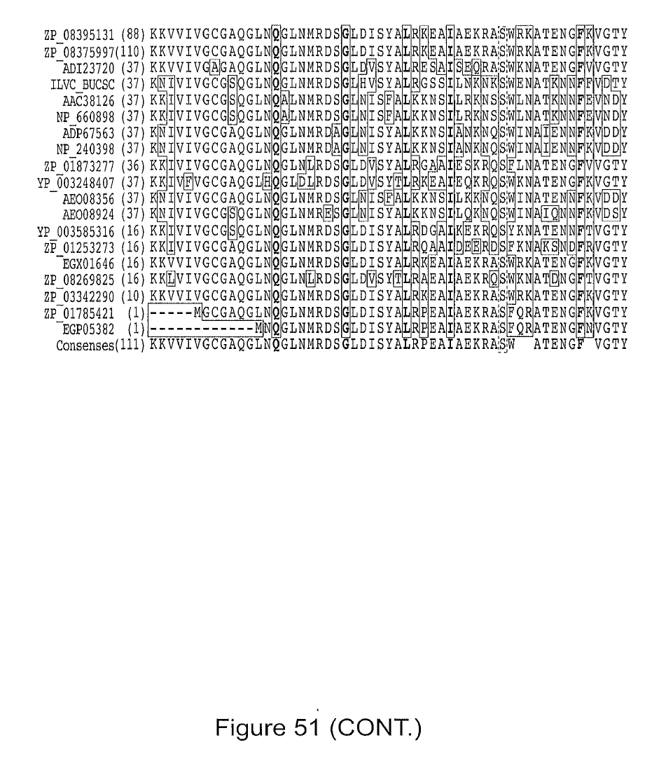

There are two classes of KARI enzymes known in the art: (1) short-form KARIs and (2) long-form KARIs. As used herein, the short-form versions of KARI are generally referred to as “Class I ketol-acid reductoisomerases” and typically have less than 415 amino acid residues. These “Class I ketol-acid reductoisomerases” are distinct from the long-form KARIs. As used herein, the long-form versions of KARI are generally referred to as “class II ketol-acid reductoisomerases” and typically have more than 415 amino acid residues. An alignment of available class II ketol-acid reductoisomerases is illustrated in FIG. 51. These class II ketol-acid reductoisomerases are further depicted in Table 44, and their corresponding sequences are found in the attached sequence listing as SEQ ID NOs: 13 and 331-676.

Enzymes for use in the compositions and methods of the invention include any enzyme having the ability to convert acetolactate to 2,3-dihydroxyisovalerate and/or the ability to convert 2-aceto-2-hydroxy-butyrate to 2,3-dihydroxy-3-methylvalerate. Such enzymes include, but are not limited to, the E. coli ilvC gene product and the S. cerevisiae ilv5 gene product, and the KARI enzyme from Piromyces sp, Buchnera aphidicola, Spinacia oleracea, Oryza sativa, Chlamydomonas reinhardtii, Neurospora crassa, Schizosaccharomyces pombe, Laccaria bicolor, Ignicoccus hospitalis, Picrophilus torridus, Acidiphilium cryptum, Cyanobacteria/Synechococcus sp., Zymomonas mobilis, Bacteroides thetaiotaomicron, Methanococcus maripaludis, Vibrio fischeri, Shewanella sp, Gramella forsetti, Psychromonas ingrhamaii, and Cytophaga hutchinsonii.

There are two distinct categories of KARI enzymes known in the art: (1) KARI enzymes which are dependent upon the use of NADPH as a cofactor, categorized into enzyme classification (EC) number 1.1.1.86; and (2) KARI enzymes which are dependent upon the use of NADH as a cofactor (i.e., an NADH-dependent KARI or “NKR”.)

As noted above, unmodified KARI enzymes are known by the EC number 1.1.1.86. Once modified to be NADH-dependent, the NADH-dependent KARI enzyme no longer belongs to EC number 1.1.1.86, which specifically requires that the enzyme use NADPH to catalyze the conversion of acetolactate to 2,3-dihydroxyisovalerate. Sequences of unmodified KARI enzymes are available from a vast array of microorganisms, including, but not limited to, Escherichia coli (GenBank Nos: NP_{—}418222 and NC_{—}000913, Saccharomyces cerevisiae (GenBank Nos: NP_{—}013459 and NC_{—}001144, Methanococcus maripaludis (GenBank Nos: CAF30210 and BX957220, and Bacillus subtilis (GenBank Nos: CAB14789 and Z99118) and the KARI enzymes from Piromyces sp (GenBank No: CAA76356), Buchnera aphidicola (GenBank No: AAF13807), Spinacia oleracea (GenBank Nos: Q01292 and CAA40356), Oryza sativa (GenBank No: NP_{—}001056384) Chlamydomonas reinhardtii (GenBank No: XP_{—}001702649), Neurospora crassa (GenBank No: XP_{—}961335), Schizosaccharomyces pombe (GenBank No: NP_{—}001018845), Laccaria bicolor (GenBank No: XP_{—}001880867), Ignicoccus hospitalis (GenBank No: YP_{—}001435197), Picrophilus torridus (GenBank No: YP_{—}023851), Acidiphilium cryptum (GenBank No: YP_{—}001235669), Cyanobacteria/Synechococcus sp. (GenBank No: YP_{—}473733), Zymomonas mobilis (GenBank No: YP_{—}162876), Bacteroides thetaiotaomicron (GenBank No: NP_{—}810987), Methanococcus maripaludis (GenBank No: YP_{—}001097443), Vibrio fischeri (GenBank No: YP_{—}205911), Shewanella sp (GenBank No: YP_{—}732498), Gramella forsetti (GenBank No: YP_{—}862142), Psychromonas ingrhamaii (GenBank No: YP_{—}942294), and Cytophaga hutchinsonii (GenBank No: YP_{—}677763).

As will be understood by one of ordinary skill in the art, modified KARI enzymes may be obtained by recombinant or genetic engineering techniques that are routine and well-known in the art. Mutant KARI enzymes can, for example, be obtained by mutating the gene or genes encoding the KARI enzyme of interest by site-directed or random mutagenesis. Such mutations may include point mutations, deletion mutations and insertional mutations. For example, one or more point mutations (e.g., substitution of one or more amino acids with one or more different amino acids) may be used to construct mutant KARI enzymes of the invention.

Ketol-acid reductoisomerase catalyzes the reduction of acetolactate to 2,3-dihydroxyisovalerate. The two-step reaction involves an alkyl migration and a ketone reduction that occurs at a single active site on the enzyme without dissociation of any reaction intermediates. The unmodified, wild-type versions of KARI are NADPH-dependent. The cofactor specificity may be expanded or switched so that it will utilize both cofactors and preferentially NADH during the production of isobutanol. In an exemplary embodiment, the cofactor specificity may be altered such that the KARI enzyme exclusively uses NADH during the production of isobutanol. A study published in 1997 (Rane, M. J. and K. C. Calvo, Archives of Biochemistry and Biophysics, 1997. 338(1): p. 83-89) describes a supposed cofactor-switched KARI quadruplet variant of the E. coli ilvC gene product with mutations R68D, K69L, K75V and R76D). However, in-house studies indicate that although the ratio NADH/NADPH was 2.5, the specific activity of this variant on NADH was actually worse than wild-type (Table 25), rendering this enzyme not suited for the purpose of this disclosure.

### Modified or Mutated KARI Enzymes

In accordance with the invention, any number of mutations can be made to the KARI enzymes, and in a preferred aspect, multiple mutations can be made to result in an increased ability to utilize NADH for the conversion of acetolactate to 2,3-dihydroxyisovalerate. Such mutations include point mutations, frame shift mutations, deletions, and insertions, with one or more (e.g., one, two, three, or four, etc.) point mutations preferred.

Mutations may be introduced into the KARI enzymes of the present invention using any methodology known to those skilled in the art. Mutations may be introduced randomly by, for example, conducting a PCR reaction in the presence of manganese as a divalent metal ion cofactor. Alternatively, oligonucleotide directed mutagenesis may be used to create the mutant KARI enzymes which allows for all possible classes of base pair changes at any determined site along the encoding DNA molecule. In general, this technique involves annealing an oligonucleotide complementary (except for one or more mismatches) to a single stranded nucleotide sequence coding for the KARI enzyme of interest. The mismatched oligonucleotide is then extended by DNA polymerase, generating a double-stranded DNA molecule which contains the desired change in sequence in one strand. The changes in sequence can, for example, result in the deletion, substitution, or insertion of an amino acid. The double-stranded polynucleotide can then be inserted into an appropriate expression vector, and a mutant or modified polypeptide can thus be produced. The above-described oligonucleotide directed mutagenesis can, for example, be carried out via PCR.

The invention further includes homologous KARI enzymes which are 5%, 10%, 20%, 30%, 40%, 50%, 60%, 70%, 75%, 80%, 85%, 90%, 95%, 96%, 97%, 98%, or 99% identical at the amino acid level to a wild-type KARI enzyme (e.g., encoded by the Ec_ilvC gene or S. cerevisiae ilv5 gene) and exhibit an increased ability to utilize NADH for the conversion of acetolactate to 2,3-dihydroxyisovalerate. Also included within the invention are KARI enzymes which are 50%, 60%, 70%, 75%, 80%, 85%, 90%, 95%, 96%, 97%, 98%, or 99% identical at the amino acid level to a KARI enzyme comprising the amino acid sequence set out in SEQ ID NO: 13 and exhibit an increased ability to utilize NADH for the conversion of acetolactate to 2,3-dihydroxyisovalerate. The invention also includes nucleic acid molecules which encode the above described KARI enzymes.

The invention also includes fragments of KARI enzymes which comprise at least 50, 100, 150, 200, 250, 300, 350, 400, 450, 500, 550, or 600 amino acid residues and retain one or more activities associated with KARI enzymes. Such fragments may be obtained by deletion mutation, by recombinant techniques that are routine and well-known in the art, or by enzymatic digestion of the KARI enzyme(s) of interest using any of a number of well-known proteolytic enzymes. The invention further includes nucleic acid molecules which encode the above described mutant KARI enzymes and KARI enzyme fragments.

By a protein or protein fragment having an amino acid sequence at least, for example, 50% “identical” to a reference amino acid sequence it is intended that the amino acid sequence of the protein is identical to the reference sequence except that the protein sequence may include up to 50 amino acid alterations per each 100 amino acids of the amino acid sequence of the reference protein. In other words, to obtain a protein having an amino acid sequence at least 50% identical to a reference amino acid sequence, up to 50% of the amino acid residues in the reference sequence may be deleted or substituted with another amino acid, or a number of amino acids up to 50% of the total amino acid residues in the reference sequence may be inserted into the reference sequence. These alterations of the reference sequence may occur at the amino (N-) and/or carboxy (C-) terminal positions of the reference amino acid sequence and/or anywhere between those terminal positions, interspersed either individually among residues in the reference sequence and/or in one or more contiguous groups within the reference sequence. As a practical matter, whether a given amino acid sequence is, for example, at least 50% identical to the amino acid sequence of a reference protein can be determined conventionally using known computer programs such as those described above for nucleic acid sequence identity determinations, or using the CLUSTAL W program (Thompson, J. D., et al., Nucleic Acids Res. 22:4673 4680 (1994)).

In one aspect, amino acid substitutions are made at one or more of the above identified positions (i.e., amino acid positions equivalent or corresponding to A71, R76, S78, or Q110 of E. coli IlvC). Thus, the amino acids at these positions may be substituted with any other amino acid including Ala, Asn, Arg, Asp, Cys, Gln, Glu, Gly, His, Ile, Leu, Lys, Met, Phe, Pro, Ser, Thr, Trp, Tyr, and Val. A specific example of a KARI enzyme which exhibits an increased ability to utilize NADH includes an E. coli IlvC KARI enzyme in which (1) the alanine at position 71 has been replaced with a serine, (2) the arginine at position 76 has been replaced with an aspartic acid, (3) the serine at position 78 has been replaced with an aspartic acid, and/or (4) the glutamine at position 110 has been replaced with valine.

In another aspect, the present application provides modified class II KARI enzymes, wherein said modified class II KARI enzymes are derived from unmodified class II KARI enzymes that have been engineered to comprise an amino acid substitution corresponding to amino acid residue Serine 78 of the E. coli KARI (SEQ ID NO: 13), and wherein said modified class II KARI exhibits an increased ability to use NADH as a cofactor to catalyze the conversion of acetolactate to 2,3-dihydroxyisovalerate as compared to a corresponding unmodified class II KARI. In one embodiment, the amino acid residue corresponding to the Serine 78 of E. coli KARI (SEQ ID NO: 13) is replaced with an amino acid residue selected from the group consisting of aspartic acid and glutamic acid. In further embodiments, the modified class II KARI enzymes may independently or additionally comprise an amino acid substitution corresponding to one or more amino acid residues selected from the group consisting of alanine 71, arginine 76, and glutamine 110 of the E. coli KARI (SEQ ID NO: 13). In some embodiments, the unmodified class II KARI is derived from a genus selected from the group consisting of Escherichia, Shigella, Yersinia, Klebsiella, Vibrio, Providencia, Haemophilus, Shewanella, and Salmonella. In further embodiments, the unmodified class II KARI comprises SEQ ID NO: 13. In yet further embodiments, the unmodified class II KARI comprises an amino acid sequence selected from the group consisting of SEQ ID NOs: 331-676

Polypeptides having the ability to convert acetolactate to 2,3-dihydroxyisovalerate and/or 2-aceto-2-hydroxy-butyrate to 2,3-dihydroxy-3-methylvalerate for use in the invention may be isolated from their natural prokaryotic or eukaryotic sources according to standard procedures for isolating and purifying natural proteins that are well-known to one of ordinary skill in the art (see, e.g., Houts, G. E., et al., J. Virol. 29:517 (1979)). In addition, polypeptides having the ability to convert acetolactate to 2,3-dihydroxyisovalerate and/or 2-aceto-2-hydroxy-butyrate to 2,3-dihydroxy-3-methylvalerate may be prepared by recombinant DNA techniques that are familiar to one of ordinary skill in the art (see, e.g., Kotewicz, M. L., et al., Nucl. Acids Res. 16:265 (1988); Soltis, D. A., and Skalka, A. M., Proc. Natl. Acad. Sci. USA 85:3372 3376 (1988)).

In accordance with the invention, one or more mutations may be made in any KARI enzyme of interest in order to increase the ability of the enzyme to utilize NADH, or confer other properties described herein upon the enzyme, in accordance with the invention. Such mutations include point mutations, frame shift mutations, deletions and insertions. Preferably, one or more point mutations, resulting in one or more amino acid substitutions, are used to produce KARI enzymes having an enhanced or increased ability to utilize NADH, particularly to facilitate the conversion of acetolactate to 2,3-dihydroxyisovalerate and/or the conversion of 2-aceto-2-hydroxy-butyrate to 2,3-dihydroxy-3-methylvalerate. In a preferred aspect of the invention, one or more mutations at positions equivalent or corresponding to position A71 (e.g., A71S), R76 (e.g., R76D), S78 (e.g. S78D), and/or Q110 (e.g. Q110V) and/or D146 (e.g. D146G), and/or G185 (e.g. G185R) and/or K433 (e.g. K433E) of the E. coli IlvC KARI enzyme may be made to produce the desired result in other KARI enzymes of interest.

The corresponding positions of the KARI enzymes identified herein (e.g. E. coli IlvC may be readily identified for other KARI enzymes by one of skill in the art. Thus, given the defined region and the assays described in the present application, one with skill in the art can make one or a number of modifications which would result in an increased ability to utilize NADH, particularly for the conversion of acetolactate to 2,3-dihydroxyisovalerate, in any KARI enzyme of interest. Residues to be modified in accordance with the present invention may include those described in Examples 14, 15, and 16.

In a preferred embodiment, the modified or mutated KARI enzymes have from 1 to 4 amino acid substitutions in amino acid regions involved in cofactor specificity as compared to the wild-type KARI enzyme proteins. In other embodiments, the modified or mutated KARI enzymes have additional amino acid substitutions at other positions as compared to the respective wild-type KARI enzymes. Thus, modified or mutated KARI enzymes may have at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 different residues in other positions as compared to the respective wild-type KARI enzymes. As will be appreciated by those of skill in the art, the number of additional positions that may have amino acid substitutions will depend on the wild-type KARI enzyme used to generate the variants. Thus, in some instances, up to 50 different positions may have amino acid substitutions.

The nucleotide sequences for several KARI enzymes are known. For instance, the sequences of KARI enzymes are available from a vast array of microorganisms, including, but not limited to, Escherichia coli (GenBank No: NP_{—}418222), Saccharomyces cerevisiae (GenBank Nos: NP_{—}013459, Methanococcus maripaludis (GenBank No: YP_{—}001097443), Bacillus subtilis (GenBank Nos: CAB14789), and the KARI enzymes from Piromyces sp (GenBank No: CAA76356), Buchnera aphidicola (GenBank No: AAF13807), Spinacia oleracea (GenBank Nos: Q01292 and CAA40356), Oryza sativa (GenBank No: NP_{—}001056384) Chlamydomonas reinhardtii (GenBank No: XP_{—}001702649), Neurospora crassa (GenBank No: XP_{—}961335), Schizosaccharomyces pombe (GenBank No: NP_{—}001018845), Laccaria bicolor (GenBank No: XP_{—}001880867), Ignicoccus hospitalis (GenBank No: YP_{—}001435197), Picrophilus torridus (GenBank No: YP_{—}023851), Acidiphilium cryptum (GenBank No: YP_{—}001235669), Cyanobacteria/Synechococcus sp. (GenBank No: YP_{—}473733), Zymomonas mobilis (GenBank No: YP_{—}162876), Bacteroides thetaiotaomicron (GenBank No: NP_{—}810987), Methanococcus maripaludis (GenBank No: YP_{—}001097443), Vibrio fischeri (GenBank No: YP_{—}205911), Shewanella sp (GenBank No: YP_{—}732498), Gramella forsetti (GenBank No: YP_{—}862142), Psychromonas ingrhamaii (GenBank No: YP_{—}942294), and Cytophaga hutchinsonii (GenBank No: YP_{—}677763).

### Improved NADH-Dependent Activity

In one aspect, the NADH-dependent activity of the modified or mutated KARI enzyme is increased.

In an exemplary embodiment, the catalytic efficiency of the modified or mutated KARI enzyme is improved for the cofactor NADH. Preferably, the catalytic efficiency of the modified or mutated KARI enzyme is improved by at least about 5% as compared to the wild-type or parental KARI for NADH. More preferably the catalytic efficiency of the modified or mutated KARI enzyme is improved by at least about 15% as compared to the wild-type or parental KARI for NADH. More preferably, the catalytic efficiency of the modified or mutated KARI enzyme is improved by at least about 25% as compared to the wild-type or parental KARI for NADH. More preferably, the catalytic efficiency of the modified or mutated KARI enzyme is improved by at least about 50% as compared to the wild-type or parental KARI for NADH. More preferably, the catalytic efficiency of the modified or mutated KARI enzyme is improved by at least about 75% as compared to the wild-type or parental KARI for NADH. More preferably, the catalytic efficiency of the modified or mutated KARI enzyme is improved by at least about 100% as compared to the wild-type or parental KARI for NADH. More preferably, the catalytic efficiency of the modified or mutated KARI enzyme is improved by at least about 300% as compared to the wild-type or parental KARI for NADH. More preferably, the catalytic efficiency of the modified or mutated KARI enzyme is improved by at least about 500% as compared to the wild-type or parental KARI for NADH. More preferably, the catalytic efficiency of the modified or mutated KARI enzyme is improved by at least about 1000% as compared to the wild-type or parental KARI for NADH. More preferably, the catalytic efficiency of the modified or mutated KARI enzyme is improved by at least about 5000% as compared to the wild-type or parental KARI for NADH.

In another exemplary embodiment, the catalytic efficiency of the modified or mutated KARI enzyme with NADH is increased with respect to the catalytic efficiency of the wild-type or parental enzyme with NADPH. Preferably, the catalytic efficiency of the modified or mutated KARI enzyme is at least about 10% of the catalytic efficiency of the wild-type or parental KARI enzyme for NADPH. More preferably, the catalytic efficiency of the modified or mutated KARI enzyme is at least about 25% of the catalytic efficiency of the wild-type or parental KARI enzyme for NADPH. More preferably, the catalytic efficiency of the modified or mutated KARI enzyme is at least about 50% of the catalytic efficiency of the wild-type or parental KARI enzyme for NADPH. More preferably, the catalytic efficiency of the modified or mutated KARI enzyme is at least about 75%, 85%, 95% of the catalytic efficiency of the wild-type or parental KARI enzyme for NADPH.

In yet another exemplary embodiment, the K_{M} of the KARI enzyme for NADH is decreased relative to the wild-type or parental enzyme. A change in K_{M} is evidenced by at least a 5% or greater increase or decrease in K_{M} compared to the wild-type KARI enzyme. In certain embodiments, modified or mutated KARI enzymes of the present invention may show greater than 10 times decreased K_{M} for NADH compared to the wild-type or parental KARI enzyme. In certain embodiments, modified or mutated KARI enzymes of the present invention may show greater than 30 times decreased K_{M} for NADH compared to the wild-type or parental KARI enzyme.

In yet another exemplary embodiment, the k_{cat} of the KARI enzyme with NADH is increased relative to the wild-type or parental enzyme. A change in k_{cat} is evidenced by at least a 5% or greater increase or decrease in K_{M} compared to the wild-type KARI enzyme. In certain embodiments, modified or mutated KARI enzymes of the present invention may show greater than 50% increased k_{cat} for NADH compared to the wild-type or parental KARI enzyme. In certain embodiments, modified or mutated KARI enzymes of the present invention may show greater than 100% increased k_{cat} for NADH compared to the wild-type or parental KARI enzyme. In certain embodiments, modified or mutated KARI enzymes of the present invention may show greater than 200% increased k_{cat} for NADH compared to the wild-type or parental KARI enzyme.

### Cofactor Switch

In preferred embodiments, the cofactor specificity of the modified or mutated KARI enzyme is altered such that there is a cofactor switch from NADPH to NADH. In other words, these modified or mutated KARI enzymes will have an increase in NADH-dependent activity and a substantially simultaneous decrease in NADPH dependent activity. Thus, the methods of the present invention can be used to change the cofactor preference from NADPH to NADH.

“Cofactor specificity” is a measure of the specificity of an enzyme for one cofactor over another. Thus, the methods of the present invention may be used to alter the cofactor preference of the target enzyme, such that the preference for the less favored cofactor is increased by 20%, 50%, 100%, 300%, 500%, 1000%, up to 2000%. For example, a number of reductase enzymes have been described that favor NADPH over NADH (see WO 02/22526; WO 02.29019; Mittl, P R., et al., (1994) Protein Sci., 3: 1504 14; Banta, S., et al., (2002) Protein Eng., 15:131 140; all of which are hereby incorporated by reference in their entirety). As the availability of NADPH is often limiting, both in vivo and in vitro, the overall activity of the target protein is often limited. For target proteins that prefer NADPH as a cofactor, it would be desirable to alter the cofactor specificity of the target protein (e.g. a KARI enzyme) to a cofactor that is more readily available, such as NADH.

In a preferred embodiment, the cofactor specificity of the KARI enzyme is switched. By “switched” herein is meant, that the cofactor preference (in terms of catalytic efficiency (k_{cat}/K_{M}) of the KARI enzyme is changed to another cofactor Preferably, in one embodiment, by switching cofactor specificity, activity in terms of catalytic efficiency (k_{cat}/K_{M}) with the cofactor preferred by the wild-type KARI enzyme is reduced, while the activity with the less preferred cofactor is increased. This can be achieved, for example by increasing the k_{cat} for less preferred cofactor over the preferred cofactor or by decreasing K_{M} for the less preferred cofactor over the preferred cofactor or both.

In a preferred embodiment, the KARI enzyme is modified or a mutated to become NADH-dependent. The term “NADH-dependent” refers to the property of an enzyme to preferentially use NADH as the redox cofactor. An NADH-dependent enzyme has a higher catalytic efficiency (k_{cat}/K_{M}) with the cofactor NADH than with the cofactor NADPH as determined by in vitro enzyme activity assays. Accordingly, the term “NADPH-dependent” refers to the property of an enzyme to preferentially use NADPH as the redox cofactor. An NADPH dependent enzyme has a higher catalytic efficiency (k_{cat}/K_{M}) with the cofactor NADPH than with the cofactor NADH as determined by in vitro enzyme activity assays.

In a preferred embodiment, the catalytic efficiency of the KARI enzyme for NADH is enhanced relative to the catalytic efficiency with NADPH. The term “catalytic efficiency” describes the ratio of the rate constant k_{cat} over the Michaelis-Menten constant K_{M}. In one embodiment, the invention is directed to a modified or mutated KARI enzyme that exhibits at least about a 1:10 ratio of catalytic efficiency (k_{cat}/K_{M}) with NADH over catalytic efficiency with NADPH. In another embodiment, the modified or mutated KARI enzyme exhibits at least about a 1:1 ratio of catalytic efficiency (k_{cat}/K_{M}) with NADH over catalytic efficiency with NADPH. In yet another embodiment, the modified or mutated KARI enzyme exhibits at least about a 10:1 ratio of catalytic efficiency (k_{cat}/K_{M}) with NADH over catalytic efficiency with NADPH. In yet another embodiment, the modified or mutated KARI enzyme exhibits at least about a 100:1 ratio of catalytic efficiency (k_{cat}/K_{M}) with NADH over catalytic efficiency with NADPH. In an exemplary embodiment, the modified or mutated KARI enzyme exhibits at least about a 100:1 ratio of catalytic efficiency (k_{cat}/K_{M}) with NADH over catalytic efficiency with NADPH.

In a preferred embodiment, the K_{M} of the KARI enzyme for NADH is decreased relative to the K_{M} of the KARI enzyme for NADPH. In one embodiment, the invention is directed to a modified or mutated KARI enzyme that exhibits at least about a 10:1 ratio of K_{M} for NADH over K_{M} for NADPH. In one embodiment, the invention is directed to a modified or mutated KARI enzyme that exhibits at least about a 1:1 ratio of K_{M} for NADH over K_{M} for NADPH. In a preferred embodiment, the invention is directed to a modified or mutated KARI enzyme that exhibits at least about a 1:10 ratio of K_{M} for NADH over K_{M} for NADPH. In yet another embodiment, the invention is directed to a modified or mutated KARI enzyme that exhibits at least about a 1:20, 1:100, 1:1000 ratio of K_{M} for NADH over K_{M} for NADPH.

In another preferred embodiment, the k_{cat} of the KARI enzyme with NADH is increased relative to k_{cat} with NADPH. In certain embodiments, modified or mutated KARI enzymes of the present invention may show greater than 0.8:1 ratio of k_{cat} with NADH over k_{cat} with NADPH. In certain embodiments, modified or mutated KARI enzymes of the present invention may show greater than 1:1 ratio of k_{cat} with NADH over k_{cat} with NADPH. In a preferred embodiments, modified or mutated KARI enzymes of the present invention may show greater than 10:1 ratio of k_{cat} with NADH over k_{cat} with NADPH. In certain embodiments, modified or mutated KARI enzymes of the present invention may show greater than 100:1 ratio of k_{cat} with NADH over k_{cat} with NADPH

### Identification of Corresponding Amino Acid Substitutions in Homologous Enzymes

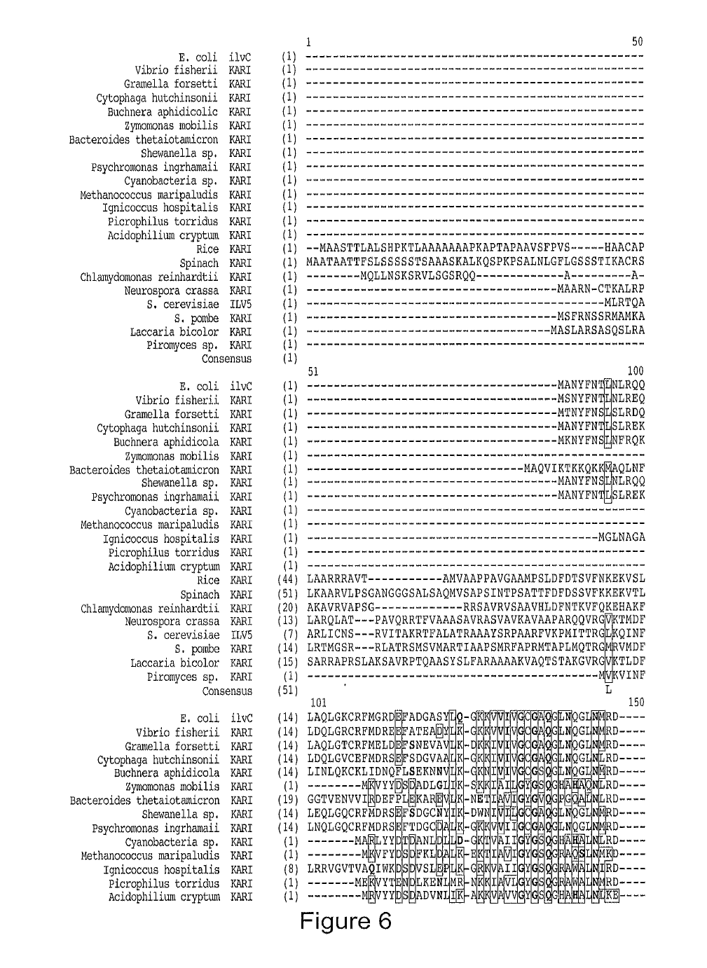

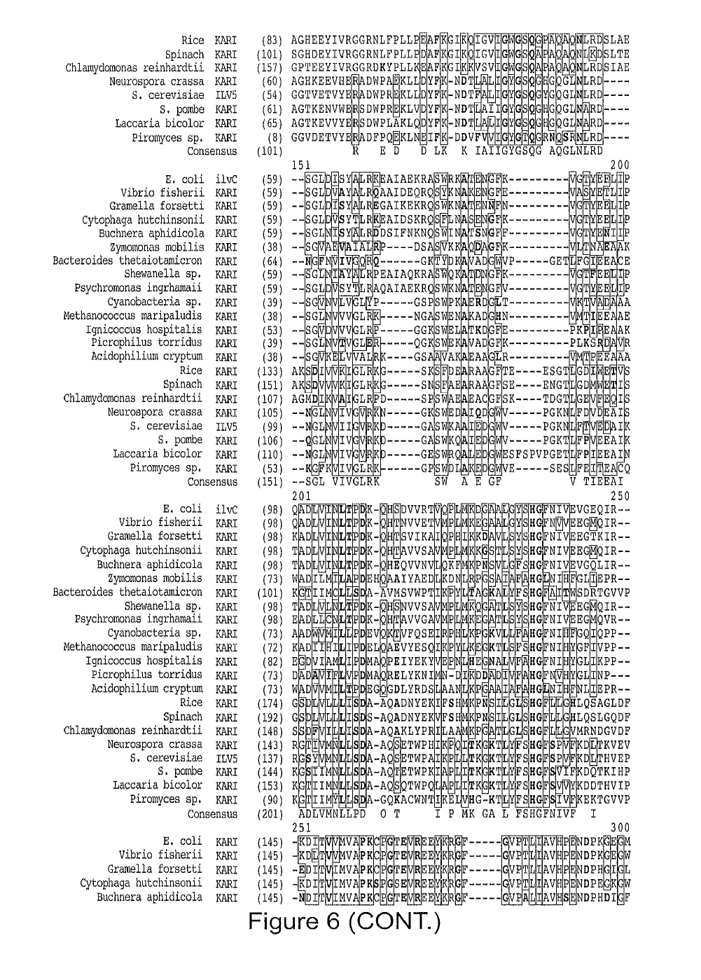

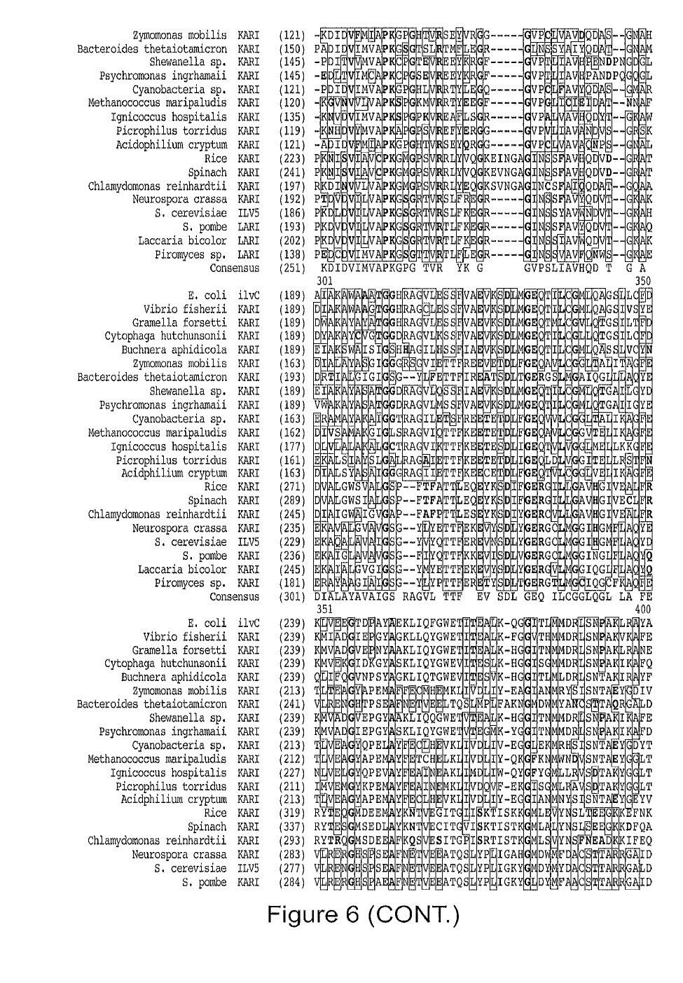

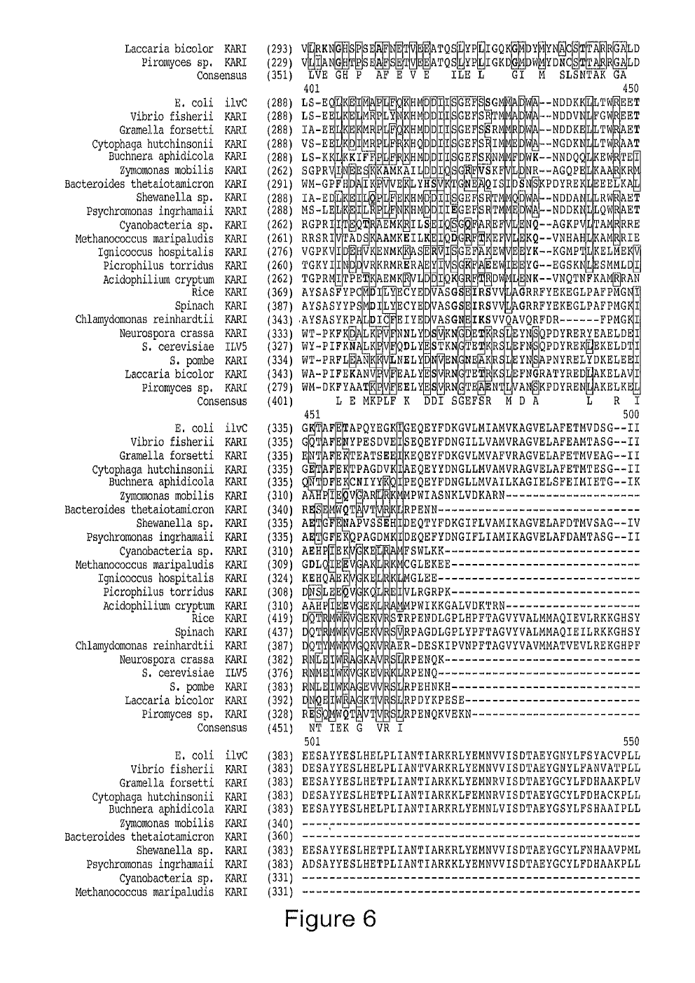

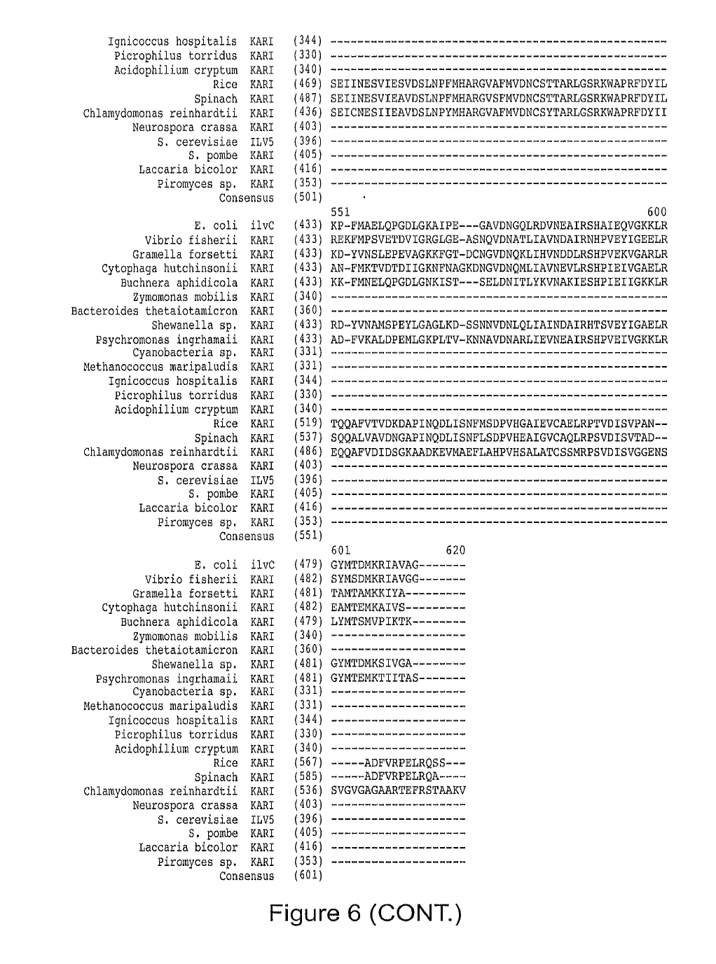

An amino acid sequence alignment of 22 KARIs (including E. coli IlvC, spinach KARI and rice KARI) was performed (FIG. 6). Some KARIs aligned with the E. coli KARI sequence at amino acid positions 71, 76, 78, and 110 and this allows to conclude that the beneficial mutations found for E. coli KARI confer the same effects in these KARI enzymes. Other sequences show deletions at about these positions and the sequence alignment is not sufficient to make any predictions.

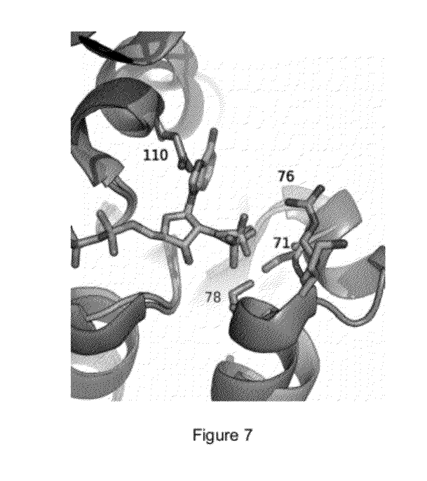

A structure alignment of E. coli KARI (PDB ID NO. 1YRL) with rice KARI (PDB ID NO. 3FR8) as a representative of the shorter loop group was performed (FIG. 7). The sites of useful mutations in the E. coli context corresponded reasonably well with specific residues in the context of the shorter loop: Ser165, Lys166, and Ser167. Ser165 of (corresponding to A71 in E. coli) therefore may be substituted with aspartate. A charge reversal at position K166 (corresponding to position R76D) may yield the same result. Ser167 may correspond to Ser78 and a mutation to aspartate may be beneficial Mutations at Q110 may be transferable in all 22 KARIs aligned.

In the case of D146 (e.g. D146G), G185 (e.g. G185R), and K433 (e.g. K433E), surface charge changes took place. Glycine at position 185 and Lysine at position 433 are highly conserved among other KARIs. These mutations may therefore be transferable to other KARIs with a similar effect. Aspartate at position 146 is not as highly conserved.

In the case of class II KARI enzymes, an amino acid sequence alignment of over 300 class II KARIs is illustrated in FIG. 51. As this alignment demonstrates, class II KARIs share a conserved serine residue at the position corresponding to Serine 78 residue of the E. coli KARI of SEQ ID NO: 13. As demonstrated in Example 27, the beneficial mutation at the Serine 78 residue in the E. coli KARI is transferable to other class II KARI enzymes, including, but not limited to, class II KARI enzymes derived from a genus selected from the group consisting of Escherichia, Shigella, Yersinia, Klebsiella, Vibrio, Providencia, Haemophilus, Shewanella, and Salmonella.

### NADH-Dependent ADH Enzymes

Several alcohol dehydrogenases may be suitable candidates for conversion into an NADH-dependent isobutyraldehyde dehydrogenase. Among the preferred enzymes for conversion are S. cerevisiae ADH1, Zymomonas mobilis ADHII, E. coli YqhD, herein referred to as Ec_YqhD, and S. cerevisiae ADH7.

As described in the prior art in PCT/US2008/053514, the S. cerevisiae ADH2 gene is expected to be functionally expressed from pSA55 and required for catalyzing the final step of the isobutanol biosynthetic pathway, namely the conversion of isobutyraldehyde to isobutanol. Thus, no isobutanol should be produced with the plasmid combination lacking ADH2 as adhE is deleted in JCL260. However, as exemplified in Example 10, the results of a fermentation using a strain without overexpression of any gene encoding an enzyme with ADH activity for the conversion of isobutyraldehyde to isobutanol showed that overexpression of an ADH enzyme is not required for isobutanol production in E. coli. In fact, isobutanol production for the system lacking ADH2 was higher than for the system with ADH2 expression. Volumetric productivity and titer showed 42% increase, specific productivity showed 18% increase and yield 12% increase. This suggests strongly that a native E. coli dehydrogenase is responsible for the conversion of isobutyraldehyde to isobutanol.

Surprisingly, this last step of the isobutanol biosynthetic pathway was found to be carried out by a native E. coli dehydrogenase in the aforementioned strains, as exemplified in Example 11: Approximately ˜80% of the isobutyraldehyde reduction activity is due to Ec_YqhD under certain culture conditions. Available literature on Ec_YqhD suggests that while it does prefer long-chain alcohols, it also utilizes NADPH (versus NADH) (Perez, J. M., et al., Journal of Biological Chemistry, 2008. 283(12): p. 7346-7353).

Switching the cofactor specificity of an NADPH-dependent alcohol dehydrogenase may be complicated by the fact that cofactor binding induces a conformational change, resulting in an anhydrous binding pocket that facilitates hydride transfer from the reduced cofactor to the aldehyde (Leskovac, V., S. Trivic, and D. Pricin, Fems Yeast Research, 2002. 2: p. 481-494; Reid, M. F. and C. A. Fewson., Critical Reviews in Microbiology, 1994. 20(1): p. 13-56). Mutations that are beneficial for binding NADH may have deleterious effects with respect to this conformational change.

Alternatively, isobutyraldehyde reduction activity of an NADH-dependent enzyme with little native activity towards this substrate may be increased. This approach has the advantages that (1) several specialized enzymes exist in nature that are highly active under fermentative conditions, (2) the binding sites of several of these enzymes are known, (3) mutational studies indicate that substrate specificity can easily be altered to achieve high activity on a new substrate.

Several alcohol dehydrogenase enzymes may be suitable candidates for conversion into an NADH-dependent isobutyraldehyde dehydrogenase: S. cerevisiae ADH1 and Zymomonas mobilis ADHII are NADH-dependent enzymes responsible for the conversion of acetaldehyde to ethanol under anaerobic conditions. These enzymes are highly active. The substrate specificity for these enzymes has been analyzed (Leskovac, V., S. Trivic, and D. Pricin, Fems Yeast Research, 2002. 2: p. 481-494; Rellos, P., J. Ma, and R. K. Scopes, Protein Expression and Purification, 1997. 9: p. 89-90), the amino acid residues comprising the substrate binding pocket are known (Leskovac, V., S. Trivic, and D. Pricin, Fems Yeast Research, 2002. 2: p. 481-494; Rellos, P., J. Ma, and R. K. Scopes, Protein Expression and Purification, 1997. 9: p. 89-90), and attempts to alter the substrate specificity by mutation have revealed that the substrate specificity can be altered (Rellos, P., J. Ma, and R. K. Scopes, Protein Expression and Purification, 1997. 9: p. 89-90; Green, D. W., H. Suns, and B. V. Plapp, Journal of Biological Chemistry, 1993. 268(11): p. 7792-7798). Ec_YqhD and S. cerevisiae ADH7 are NADPH-dependent enzymes whose physiological functions are not as well understood. Ec_YqhD has been implicated in the protection of the cell from peroxide-derived aldehydes (Perez, J. M., et al., Journal of Biological Chemistry, 2008. 283(12): p. 7346-7353). The substrate specificity of both enzymes is understood, and amino acids lining the substrate binding pocket are known (Perez, J. M., et al., Journal of Biological Chemistry, 2008. 283(12): p. 7346-7353). Based on the known amino acid residues implicated in substrate binding (S. cerevisiae ADH1, Z. mobilis ADHII) or the cofactor binding site (Ec_yqhD), sites with the highest likelihood of affecting desired enzyme features such as substrate specificity or cofactor specificity may be mutated to generate the desired function.

One approach to increase activity of enzymes with NADH as the cofactor may be saturation mutagenesis with NNK libraries at each of the residues that interact with the cofactor. These libraries may be screened for activity in the presence of NADPH and NADH in order to identify which single mutations contribute to increased activity on NADH and altered specificity for NADH over NADPH. Combinations of mutations at aforementioned residues may be investigated by any method. For example, a combinatorial library of mutants may be designed based on the results of the saturation mutagenesis studies. For example, a combinatorial library of mutants may be designed including only those mutations that do not lead to decrease in NADH-dependent activity.

Another approach to increase the NADH-dependent activity of the enzyme is to perform saturation mutagenesis of a first amino acid that interacts with the cofactor, then isolate the mutant with the highest activity using NADH as the cofactor, then perform saturation mutagenesis of a second amino acid that interacts with the cofactor, and so on. Similarly, a limited number of amino acids that interact with the cofactor may be targeted for randomization simultaneously and then be screened for improved activity with NADH as the cofactor. The selected, best mutant can then be subjected to the same procedure again and this approach may be repeated iteratively until the desired result is achieved.

Another approach is to use random oligonucleotide mutagenesis to generate diversity by incorporating random mutations, encoded on a synthetic oligonucleotide, into the cofactor binding region of the enzyme. The number of mutations in individual enzymes within the population may be controlled by varying the length of the target sequence and the degree of randomization during synthesis of the oligonucleotides. The advantages of this more defined approach are that all possible amino acid mutations and also coupled mutations can be found.

If the best variants from the experiments described above are not sufficiently active with NADH as the cofactor, directed evolution via error-prone PCR may be used to obtain further improvements. Error-prone PCR mutagenesis of the first domain containing the cofactor binding pocket may be performed followed by screening for ADH activity with NADH and/or increased specificity for NADH over NADPH as the cofactor.

Surprisingly, alcohol dehydrogenase enzymes that are not known to catalyze the reduction of isobutyraldehyde to isobutanol were identified that catalyze this reaction. Thus, in another aspect, such an alcohol dehydrogenase may be encoded by an NADH-dependent 1,3-propanediol dehydrogenase. In yet another aspect, such an alcohol dehydrogenase may be encoded by an NADH-dependent 1,2-propanediol dehydrogenase. Preferred enzymes of this disclosure include enzymes listed in Table 1. These enzymes exhibit NADH-dependent isobutyraldehyde reduction activity, measured as Unit per minute per mg of crude cell lysate (U min⁻¹ mg⁻¹) that is approximately six-fold to seven-fold greater than the corresponding NADPH-dependent isobutyraldehyde reduction activity (Tables 2 and 23).

In addition to exhibiting increased activity with NADH as the cofactor as compared to the NADPH, alcohol dehydrogenases of the present invention may further be more active as compared to the native E. coli alcohol dehydrogenase Ec_YqhD. In particular, alcohol dehydrogenases of the present invention may exhibit increased activity and/or decreased K_{M} values with NADH as the cofactor as compared to Ec_YqhD with NADPH as the cofactor. Exemplary enzymes that exhibit greater NADH-dependent alcohol dehydrogenase activity than the NADPH-dependent alcohol dehydrogenase activity are listed in Table 1; activity values are listed in Table 2 and Table 23.

TABLE 1
ADH genes tested in the following fermentations, and rationale for inclusion of each

| Gene Name | SEQ ID NO | Accession Number | Rationale for inclusion |
|---|---|---|---|
| Drosophila | 60 (nucleotide | NT_033779, | NADH-dependent, broad |
| melanogaster | sequence) | REGION: | substrate specificity, well- |
| ADH | 61 (amino acid | 14615555 . . . 14618902 | expressed in bacterial |
| | sequence) | | expression systems. Different |
| | | | class of enzyme versus others |
| | | | tested (short-chain, non-metal |
| | | | binding) |
| Lactococcus | 66 (nucleotide | | NADH-dependent alcohol |
| lactis adhA | sequence) | | dehydrogenase with activity |
| | 67 (amino acid | | using isobutyraldehyde as the |
| | sequence) | | substrate (Atsumi et al., Appl. |
| | | | Microbiol. Biotechnol., 2009, |
| | | | DOI 10.1007/s00253-009- |
| | | | 2085-6) |
| Klebsiella | 62 (nucleotide | NC_011283 | NADH-utilizing 1,2-propanediol |
| pneumoniae | sequence) | | dehydrogenase |
| dhaT | 63 (amino acid | | |
| | sequence) | | |
| Escherichia | 64 (nucleotide | NC_000913.2 | Homolog of K. pneumoniae |
| coli fucO | sequence) | (2929887 . . . 2931038, | dhaT, NADH-dependent 1,3- |
| | 65 (amino acid | complement) | propanediol dehydrogenase |
| | sequence) | | |

TABLE 2
Kinetic parameters for the conversion of isobutyraldehyde to isobutanol by
Ec_YqhD, Ec_FucO, Dm_Adh, and Kp_DhaT

| | | NADH | | NADPH | |
|---|---|---|---|---|---|
| | | | Activity | | Activity |
| | | | (U/min⁻¹ mg⁻¹ | | (U/min⁻¹ mg⁻¹ |
| | | K_{M} | crude | K_{M} | crude |
| Plasmid | Adh | (mM) | lysate) | (mM) | lysate) |
| pGV1705-A | Ec_YqhD | n.d. | n.d. | 0.25 | 0.09 |
| pGV1748-A | Ec_FucO | 0.8 | 0.23 | 0.2 | 0.04 |
| pGV1749-A | Dm_Adh | 0.9 | 6.60 | 2.7 | 1.70 |
| pGV1778-A | Kp_DhaT | 1.3 | 0.56 | 0.6 | 0.08 |

Alcohol dehydrogenases of the present disclosure may also be utilized in metabolically-modified microorganisms that include recombinant biochemical pathways useful for producing additional alcohols such as 2-methyl-1-butanol, 3-methyl-1-butanol, 2-phenylethanol, 1-propanol, or 1-butanol via conversion of a suitable substrate by a modified microorganism.

Microorganisms producing such compounds have been described (PCT/US2008/053514). For example, these alcohols can be 1-propanol, 1-butanol, 2-methyl-1-butanol, 3-methyl-1-butanol or 2-phenylethanol and are generally produced from a metabolite comprising a 2-keto acid. In some aspects, the 2-keto acid includes 2-ketobutyrate, 2-ketovalerate, 2-keto-3-methylvalerate, 2-keto-4-methyl-pentanoate, or phenylpyruvate. The 2-ketoacid is converted to the respective aldehyde by a 2-ketoacid decarboxylase. For example, 2-ketobutyrate is converted to 1-propanal, 2-ketovalerate is converted to 1-butanal, 2-keto-3-methylvalerate is converted to 2-methyl-1-butanol, 2-keto-4-methyl-pentanoate is converted to 3-methyl-1-butanal, and phenylpyruvate is converted to phenylethanal by a 2-ketoacid decarboxylase. Thus, the recombinant microorganism includes elevated expression or activity of a 2-keto-acid decarboxylase, as compared to a parental microorganism. The 2-keto-acid decarboxylase may be encoded by kivd from Lactococcus lactis, or homologs thereof. The 2-keto-acid decarboxylase can be encoded by a polynucleotide derived from a gene selected from kivd from L. lactis, or homologs thereof.

In earlier publications (PCT/US2008/053514, Atsumi et al., Nature, 2008 Jan. 3; 451(7174):86-9), only NADPH-dependent alcohol dehydrogenases are described that convert the aforementioned aldehyde to an alcohol. In particular, S. cerevisiae Adh2p is described that converts the aldehyde to the respective aldehyde.

Thus, in one embodiment of this disclosure, a microorganism is provided in which the cofactor dependent final step for the conversion of the aldehyde to the respective alcohol is catalyzed by an NADH-dependent alcohol dehydrogenase. In particular, NADH-dependent alcohol dehydrogenases are disclosed that catalyze the reduction aldehydes to alcohols, for example, of 1-propanal to 1 propanol, 1-butanal to 1-butanol, 2-methyl-1-butanal to 2-methyl-1-butanol, 3-methyl-1-butanal to 3-methyl-1-butanol, or phenylethanal to phenylethanol.

In a specific aspect, such an alcohol dehydrogenase may be encoded by the Drosophila melanogaster alcohol dehydrogenase Dm_Adh or homologs thereof. In another specific aspect, such an alcohol dehydrogenase may be encoded by the Lactococcus lactis alcohol dehydrogenase Ll_AdhA (SEQ ID NO: 67), as described by Atsumi et al. (Atsumi et al., Appl. Microbiol. Biotechnol., 2009, DOI 10.1007/s00253-009-2085-6) or homologs thereof.

Surprisingly, alcohol dehydrogenase enzymes that are not known to catalyze the reduction of isobutyraldehyde to isobutanol were identified that catalyze this reaction. Thus, in another aspect, such an alcohol dehydrogenase may be encoded by an NADH-dependent 1,3-propanediol dehydrogenase. In yet another aspect, such an alcohol dehydrogenase may be encoded by an NADH-dependent 1,2-propanediol dehydrogenase. Preferred enzymes of this disclosure include enzymes listed in Table 1.

In another embodiment, a method of producing an alcohol is provided. The method includes providing a recombinant microorganism provided herein; culturing the microorganism of in the presence of a suitable substrate or metabolic intermediate and under conditions suitable for the conversion of the substrate to an alcohol; and detecting the production of the alcohol. In various aspects, the alcohol is selected from 1-propanol, 1-butanol, 2-methyl 1-butanol, 3-methyl 1-butanol, and 2-phenylethanol. In another aspect, the substrate or metabolic intermediate includes a 2-keto acid-derived aldehyde, such as 1-propanal, 1-butanal, 2-methyl-1-butanal, 3-methyl-1-butanal, or phenylethanal.

Recombinant Host Cells Comprising a NADH-Dependent KARI and/or ADH Enzymes

In an additional aspect, the present invention is directed to recombinant host cells (i.e. metabolically “engineered” or “modified” microorganisms) comprising NADH-dependent KARI and/or ADH enzymes of the invention. Recombinant microorganisms provided herein can express a plurality of additional heterologous and/or native target enzymes involved in pathways for the production of beneficial metabolites such as isobutanol from a suitable carbon source.

Accordingly, metabolically “engineered” or “modified” microorganisms are produced via the introduction of genetic material (i.e. a NADH-dependent KARI and/or ADH enzymes) into a host or parental microorganism of choice, thereby modifying or altering the cellular physiology and biochemistry of the microorganism. Through the introduction of genetic material and/or the modification of the expression of native genes the parental microorganism acquires new properties, e.g. the ability to produce a new, or greater quantities of, an intracellular metabolite. As described herein, the introduction of genetic material and/or the modification of the expression of native genes into a parental microorganism results in a new or modified ability to produce beneficial metabolites such as isobutanol. The genetic material introduced into and/or the genes modified for expression in the parental microorganism contains gene(s), or parts of genes, coding for one or more of the enzymes involved in a biosynthetic pathway for the production of isobutanol and may also include additional elements for the expression and/or regulation of expression of these genes, e.g. promoter sequences.

Recombinant microorganisms provided herein may also produce metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate), an intermediate (e.g., 2-ketoisovalerate), or an end product (e.g., 1-propanol, 1-butanol, isobutanol, 2-methyl-1-butanol, 3-methyl-1-butanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

Exemplary metabolites include glucose, pyruvate, 1-propanol, 1-butanol, isobutanol, 2-methyl-1-butanol, and 3-methyl-1-butanol.

The metabolite 1-propanol can be produced by a recombinant microorganism engineered to express or over-express a metabolic pathway that converts pyruvate to 1-propanol. An exemplary metabolic pathway that converts pyruvate to 1-propanol has been described in WO/2008/098227 and by Atsumi et al. (Atsumi et al., 2008, Nature 451(7174): 86-9), the disclosures of which are herein incorporated by reference in their entireties. In a preferred embodiment, metabolic pathway comprises a KARI and/or an ADH enzyme of the present invention.

The metabolite 1-butanol can be produced by a recombinant microorganism engineered to express or over-express a metabolic pathway that converts pyruvate to 3-methyl-1-butanol. An exemplary metabolic pathway that converts pyruvate to 3-methyl-1-butanol has been described in WO/2008/098227 and by Atsumi et al. (Atsumi et al., 2008, Nature 451(7174): 86-9), the disclosures of which are herein incorporated by reference in their entireties. In a preferred embodiment, metabolic pathway comprises a KARI and/or an ADH enzyme of the present invention.

The metabolite isobutanol can be produced by a recombinant microorganism engineered to express or over-express a metabolic pathway that converts pyruvate to isobutanol. An exemplary metabolic pathway that converts pyruvate to isobutanol may be comprised of a acetohydroxy acid synthase (ALS) enzyme encoded by, for example, alsS from B. subtilis, a ketolacid reductoisomerase (KARI) of the present invention, a dihydroxy-acid dehydratase (DHAD), encoded by, for example ilvD from E. coli, a 2-keto-acid decarboxylase (KIVD) encoded by, for example kivd from L. lactis, and an alcohol dehydrogenase (ADH) of the present invention.

The metabolite 3-methyl-1-butanol can be produced by a recombinant microorganism engineered to express or over-express a metabolic pathway that converts pyruvate to 3-methyl-1-butanol. An exemplary metabolic pathway that converts pyruvate to 3-methyl-1-butanol has been described in WO/2008/098227 and by Atsumi et al. (Atsumi et al., 2008, Nature 451(7174): 86-9), the disclosures of which are herein incorporated by reference in their entireties. In a preferred embodiment, metabolic pathway comprises a KARI and/or an ADH enzyme of the present invention.

The metabolite 2-methyl-1-butanol can be produced by a recombinant microorganism engineered to express or over-express a metabolic pathway that converts pyruvate to 2-methyl-1-butanol. An exemplary metabolic pathway that converts pyruvate to 2-methyl-1-butanol has been described in WO/2008/098227 and by Atsumi et al. (Atsumi et al., 2008, Nature 451(7174): 86-9), the disclosures of which are herein incorporated by reference in their entireties. In a preferred embodiment, metabolic pathway comprises a KARI and/or an ADH enzyme of the present invention.

The disclosure identifies specific genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutation and silent mutations. Such modified or mutated polynucleotides and polypeptides can be screened for expression of a functional enzyme using methods known in the art. In addition, homologs of enzymes useful for generating metabolites are encompassed by the microorganisms and methods provided herein.

### Method of Using Microorganism for Anaerobic Isobutanol Fermentation

In a method to produce a target compound from a carbon source at high yield a modified microorganism subject to this invention is cultured in an appropriate culture medium containing a carbon source.

An exemplary embodiment provide a method for producing isobutanol comprising a modified microorganism of the invention in a suitable culture medium containing a carbon source that can be converted to isobutanol by the microorganism of the invention.

In certain embodiments, the method further includes isolating said target compound from the culture medium. For example, isobutanol may be isolated from the culture medium by any method, in particular a method known to those skilled in the art, such as distillation, pervaporation, or liquid-liquid extraction.

This invention is further illustrated by the following examples that should not be construed as limiting. The contents of all references, patents, and published patent applications cited throughout this application, as well as the Figures and the Sequence Listing, are incorporated herein by reference for all purposes.

### EXAMPLES

The following provides examples that demonstrate that microorganisms modified to resolve a cofactor imbalance produce a target compound at higher yield under conditions that include anaerobic conditions. One compound to be produced by the recombinant microorganism according to the present invention is isobutanol. The present invention is not limited to isobutanol. The invention may be applicable to any metabolic pathway that is imbalanced with respect to cofactor usage. One skilled in the art is able identify pathways that are imbalanced with respect to cofactor usage and apply this invention to provide recombinant microorganisms in which the same pathway is balanced with respect to cofactor usage.

### Sample Preparation

Generally, samples (2 mL) from fermentation experiments performed in shake flasks were stored at 4° C. for later substrate and product analysis. Prior to analysis, samples were centrifuged at 14,000×g for 10 min. The supernatant was filtered through a 0.2 μm filter. Analysis of substrates and products was performed using authentic standards (>99%, obtained from Sigma-Aldrich), and a 5-point calibration curve (with 1-pentanol as an internal standard for analysis by gas chromatography).

### Determination of Optical Density

The optical density of the yeast cultures was determined at 600 nm using a DU 800 spectrophotometer (Beckman-Coulter, Fullerton, Calif., USA). Samples were diluted as necessary to yield an optical density of between 0.1 and 0.8.

### Gas Chromatography

Analysis of volatile organic compounds, including ethanol and isobutanol was performed on a HP 5890 gas chromatograph fitted with an HP 7673 Autosampler, a DB-FFAP column (J&W; 30 m length, 0.32 mm ID, 0.25 μM film thickness) or equivalent connected to a flame ionization detector (FID). The temperature program was as follows: 200° C. for the injector, 300° C. for the detector, 100° C. oven for 1 minute, 70° C./minute gradient to 235° C., and then hold for 2.5 min.

Analysis was performed using authentic standards (>99%, obtained from Sigma-Aldrich), and a 5-point calibration curve with 1-pentanol as the internal standard.

### High Performance Liquid Chromatography

Analysis of glucose and organic acids was performed on a HP-1100 High Performance Liquid Chromatography system equipped with an Aminex HPX-87H Ion Exclusion column (Bio-Rad, 300×7.8 mm) or equivalent and an H⁺ cation guard column (Bio-Rad) or equivalent. Organic acids were detected using an HP-1100 UV detector (210 nm, 8 nm 360 nm reference) while glucose was detected using an HP-1100 refractive index detector. The column temperature was 60° C. This method was Isocratic with 0.008N sulfuric acid in water as mobile phase. Flow was set at 0.6 mL/min. Injection size was 20 μL and the run time was 30 minutes.

### Molecular Biology and Bacterial Cell Culture

Standard molecular biology methods for cloning and plasmid construction were generally used, unless otherwise noted (Sambrook, J., Russel, D. W. Molecular Cloning, A Laboratory Manual. 3 ed. 2001, Cold Spring Harbor, N.Y.: Cold Spring Harbor Laboratory Press).

Standard recombinant DNA and molecular biology techniques used in the Examples are well known in the art and are described by Sambrook, J., Russel, D. W. Molecular Cloning, A Laboratory Manual. 3 ed. 2001, Cold Spring Harbor, N.Y.: Cold Spring Harbor Laboratory Press; and by T. J. Silhavy, M. L. Bennan, and L. W. Enquist, Experiments with Gene Fusions, Cold Spring Harbor Laboratory Press, Cold Spring Harbor, N.Y. (1984) and by Ausubel, F. M. et al., Current Protocols in Molecular Biology, pub. by Greene Publishing Assoc. and Wiley-Interscience (1987).

General materials and methods suitable for the routine maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds.), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989).

Preparation of Electrocompetent E. coli Cells and Transformation

The acceptor strain culture was grown in SOB-medium (Sambrook, J., Russel, D. W. Molecular Cloning, A Laboratory Manual. 3 ed. 2001, Cold Spring Harbor, N.Y.: Cold Spring Harbor Laboratory Press) to an OD₆₀₀ of about 0.6 to 0.8. The culture was concentrated 100-fold, washed once with ice cold water and 3 times with ice cold 10% glycerol. The cells were then resuspended in 150 μL of ice-cold 10% glycerol and aliquoted into 50 μL portions. These aliquots were used immediately for standard transformation or stored at −80° C. These cells were transformed with the desired plasmid(s) via electroporation. After electroporation, SOC medium (Sambrook, J., Russel, D. W. Molecular Cloning, A Laboratory Manual. 3 ed. 2001, Cold Spring Harbor, N.Y.: Cold Spring Harbor Laboratory Press) was immediately added to the cells. After incubation for an hour at 37° C. the cells were plated onto LB-plates containing the appropriate antibiotics and incubated overnight at 37° C.

Transformation of S. cerevisiae Cells

S. cerevisiae strains were transformed by the Lithium Acetate method (Gietz et al., Nucleic Acids Res. 27:69-74 (1992): Cells from 50 mL YPD cultures (YPGaI for valine auxotrophs) were collected by centrifugation (2700 rcf, 2 minutes, 25° C.) once the cultures reached an OD₆₀₀ of 1.0. The cells were washed cells with 50 mL sterile water and collected by centrifugation at 2700 rcf for 2 minutes at 25° C. The cells were washed again with 25 mL sterile water and collected cells by centrifugation at 2700 rcf for 2 minutes at 25° C. The cells were resuspended in 1 mL of 100 mM lithium acetate and transferred to a 1.5 mL eppendorf tube. The cells were collected cells by centrifugation for 20 sec at 18,000 rcf, 25° C. The cells were resuspended cells in a volume of 100 mM lithium acetate that was approximately 4× the volume of the cell pellet. A mixture of DNA (final volume of 15 μl with sterile water), 72 μl 50% PEG, 10 μl 1 M lithium acetate, and 3 μl denatured salmon sperm DNA was prepared for each transformation. In a 1.5 mL tube, 15 μl of the cell suspension was added to the DNA mixture (85 μl), and the transformation suspension was vortexed with 5 short pulses. The transformation was incubated at 30 minutes at 30° C., followed by incubation for 22 minutes at 42° C. The cells were collected by centrifugation for 20 sec at 18,000 rcf, 25° C. The cells were resuspended in 100 μl SOS (1 M sorbitol, 34% (v/v) YP (1% yeast extract, 2% peptone), 6.5 mM CaCl₂) or 100 μl YP (1% yeast extract, 2% peptone) and spread over an appropriate selective plate.

Sporulation of Diploid S. cerevisiae and Germination to Obtain Haploids

Random spore analysis was used to identify desired haploid segregants of relevant diploid strains. Diploid strains were sporulated by pre-culturing in YPD for 24 hrs and then transferring the cells into 5 mL of sporulation medium (1% wt/vol potassium acetate). After 4-5 days, the culture was examined microscopically for the presence of visible spore-containing asci. To the 5 mL sporulation culture, 0.5 mL of 1 mg/mL Zymolyase-T (Seikagaku Biobusiness, Tokyo, Japan) and 10 μL of β-mercaptoethanol were added, and the cells were incubated overnight at 30° C. while shaking slowly (60 rpm). The next day, 5 mL of 1.5% IGEPAL-CA-630 [reference] were added and the mixture incubated on ice for 15 minutes. The cell suspension was then sonicated (3 rounds, 30 seconds per round, 50% power) with 2 minutes on ice between sonications. The suspension was centrifuged (1200×g, 10 min), the liquid poured off, 5 mL of 1.5% IGEPAL-CA-630 (Sigma-Aldrich Co., St. Louis, Mo.) were added, and the centrifugation and resuspension step repeated once more. The cell suspension was again sonicated as described above, after which it was centrifuged and washed as described above except that instead of IGEPAL, sterile water was used to resuspend the cells. The cells were finally resuspended in 1 mL of sterile water, and 0.1 mL of a 1:10, 1:100, 1:100, and 1:10,000 dilution of the initial 1 mL cell suspension were plated onto SCE-Trp, Leu, Ura (for full-pathway integrants strains) or SCD-Trp, Ura (for partial-pathway integrant strains) media and the plates incubated at 30° C. until colonies appeared (typically, 4-5 days).

### Yeast Colony PCR

Colony PCR was carried out using the FailSafe mix (Epicentre Biotechnologies, Madison, Wis.). Specifically, 15 L of FailSafe Mix “E” were combined with 13 μL sterile water, 0.35 μL of each primer (from a 100 μM solution), and 0.6 μL FailSafe polymerase. For template, a small dab of yeast cells sufficient to just turn the solution turbid was swirled into each individual reaction mixture. The PCR reactions were incubated as follows: 1 cycle of 94° C.×2 min; 40 cycles of 94° C.×15 sec, 53° C.×15 sec, 72° C.×1 min; 1 cycle of 72° C.×8 min.

qRT-PCR

Performed by isolating RNA, synthesizing cDNA by reverse transcription and performing qPCR using protocols described below.

### RNA Isolation for Reverse Transcription (RT)

3 ml YPD cell cultures were incubated at 30° C., 250 RPM until they reached OD₆₀₀'s of 0.7 to 1.5. 2 OD₆₀₀'s (e.g. 1 mL of a culture at 20D₆₀₀) of cells were then harvested from each culture in 1.5 ml tubes by centrifugation at full speed in a microfuge for 2 minutes. The cell pellet was stored overnight at −20° C. RNA was isolated using the YeaStar RNAKit™ (Zymo Research Corp. Orange, Calif. 92867 USA). Following the protocol provided with the kit, cells were resuspended in 80 μl of YR Digestion Buffer and 5 μl of Zymolyase™. The pellet was completely resuspended by repeated pipetting. The suspension was incubated at 37° C. for 60 minutes. 160 μl of YR Lysis Buffer was added to the suspension, which was then mixed thoroughly by vortexing. The mixture was centrifuged at >4,000×g for 2 minutes in the microfuge, and the supernatant was transferred to a Zymo-Spin Column in a Collection Tube. The column was centrifuged at >10,000×g for 1 minute in the microfuge. To the column, 200 μl RNA Wash Buffer was added, and the column was centrifuged for 1 minute at 14,000 RPM in the microfuge. The flow-through was discarded and 200 μl RNA Wash Buffer was added to the column. The column was centrifuged for 1 minute at >10,000×g. The Zymo-Spin Column was transferred to a new RNase-free 1.5 ml centrifuge tube, and 60 μl of DNase/RNase-Free Water was added directly to the column membrane. The RNA was eluted by centrifugation for 30 seconds at >10,000×g in the microfuge.

cDNA Synthesis (Reverse Transcription) for qPCR

Using the gScript™ cDNA SuperMix kit provided by Quanta Biosciences™ (Gaithersburg, Md.), cDNA was prepared following the protocol provided with the kit. First, the concentration of RNA was measured for the preparations from each transformant candidate and control strain. A final solution of 300 ng of RNA in sterile water was prepared in a volume of 16 μl in 0.2 ml PCR tube (RNase-free). To each sample, 4 μl of qScript cDNA Supermix was added. The reactions were incubated on a thermocycler for 5 minutes at 25° C., 30 minutes at 42° C., and 5 minutes at 85° C.

qPCR:

Each reaction contained: 10 μL of PerfeCTa™ SYBR® Green SuperMix kit (Quanta Biosciences™ Gaithersburg, Md.), 1 μl of cDNA, 1 μl of a 5 μM (each) mix of forward and reverse primers and 8 μl of sterile water. Each reaction was assembled in a well of a 0.2 ml 96-well plate, and a clear plastic sheet was carefully (to avoid the introduction of warped surface or fingerprints or smudges) and firmly placed over the 96-well plate. The reactions were incubated in an Eppendorf Mastercycler ep thermocycler (Eppendorf, Hamburg, Germany) using the following conditions: 95° C. for 2 minutes, 40 cycles of 95° C. for 15 seconds and 60° C. for 45 seconds, 95° C. for 15 seconds, 60° C. for 15 seconds, and a 20 minute slow ramping up of the temperature until it reaches 95° C. Finally, it was incubated at 95° for 15 seconds. The fluorescence emitted by the SYBR dye was measured at the 60° C. incubation step during each of the 40 cycles, as well as during the ramping up to 95° C. for melting curve analysis of the primer sets.

Construction of E. coli Strains

GEVO1385 was constructed by integrating the Z1 module into the chromosome of JCL260 by P1 transduction from the strain E. coli W3110, Z1 (Lutz, R, Bujard, H Nucleic Acids Research (1997) 25, 1203-1210).

GEVO1399: The gene zwf was deleted according to the standard protocol for gene deletion using the Wanner method (Datsenko, K. and Wanner, B. One-step Inactivation of chromosomal genes in Escherichia coli K-12 using PCR products. PNAS 2000). Primers 73 and 74 were used to amplify the Kan resistance cassette from pKD13. The linear PCR product was transformed into E. coli W3110 pKD46 electro competent cells and the knock-out of zwf was verified by PCR. Lysate of the new strain (E. coli W3110, Δzwf::FRT::Kan::FRT) was prepared and the knock-out was transferred into JCL260 by P1 transduction. Removal of the Kan resistance cassette from this strain using transient expression of FLP recombinase yielded GEVO1399.

GEVO1608: The gene Ec_yqhD (SEQ ID NO: 68) was deleted according to the standard protocol for gene deletion using the Wanner method (Datsenko, K and Wanner, B, “One-step Inactivation of chromosomal genes in Escherichia coli K-12 using PCR products,” PNAS 2000, 97:6640-6645). Primers 1155 and 1156 were used to amplify the Kan resistance cassette from pKD13. The linear PCR product was transformed into E. coli W3110 pKD46 electro competent cells and the knock-out of Ec_yqhD was verified by PCR. A lysate of the new strain (E. coli W3110, ΔyqhD::FRT::Kan::FRT) was prepared and the knock-out was transferred into JCL260 by P1 transduction yielding GEVO1608.

GEVO1745: Removal of the Kan resistance cassette from GEVO1608 using transient expression of FLP recombinase yielded GEVO1745.

GEVO1748 and GEVO1749 are derivatives of JCL260. For the construction of GEVO1748, PLlacO1:LI_kivd1::Ec_ilvD_coEc was integrated into the ilvC locus on the E. coli chromosome. In particular primers 869 and 1030 were used to amplify the kanamycin resistance cassette (Kan) from pKD13, and primers 1031 and 1032 were used to amplify PLlacO1:LI_kivd1::Ec_ilvD_coEc from pGV1655 (SEQ ID NO: 109). For the construction of GEVO1749 PLlacO1:LI_kivd1::Ec_ilvD_coEc was integrated into the adhE locus on the E. coli chromosome. In particular primers 50 and 1030 were used to amplify the kanamycin resistance cassette from pKD13, and primers 1031 and 1205 were used to amplify PLlacO1:LI_kivd1::Ec_ilvD_coEc from pGV1655 (SEQ ID NO: 109). Afterwards, SOE (splicing by overlap extension) (Horton, R M, Cai, Z L, Ho, S N, et al. Biotechniques Vol. 8 (1990) pp 528) reactions were done to connect the gene expression cassettes to the resistance cassette using primers 1032 and 869 for the ilvC locus and primers 1205 and 50 for the adhE locus. The linear PCR products were transformed into W3110 pKD46 electro competent cells and the knock ins of PLlacO1:LI_kivd1::Ec_ilvD_coEc::FRT::Kan::FRT were verified by PCR. The knock ins were further verified by sequencing. Lysates of the new strains E. coli W3110, ΔilvC::PLlacO1:LI_kivd1::Ec_ilvD_coEc::FRT::Kan::FRT) and E. coli W3110, ΔadhE::PLlacO1:LI_kivd1:: Ec_ilvD_coEc::FRT::Kan::FRT) were prepared and the knock ins were transferred to JCL260 by P1 transduction. Removal of the Kan resistance cassette from this strain using expression of FLP recombinase yielded GEVO1748 and GEVO1749.

GEVO1725, GEVO1750, GEVO1751: The gene maeA was deleted according to the standard protocol for gene deletion using the Wanner method (Datsenko, K. and Wanner, B. One-step Inactivation of chromosomal genes in Escherichia coli K-12 using PCR products. PNAS 2000). Primers 116 and 117 were used to amplify the Kan resistance cassette from pKD13. The linear PCR product was transformed into E. coli W3110 pKD46 electro competent cells and the knock-out of maeA was verified by PCR. Lysate of the new strain (E. coli W3110, ΔmaeA::FRT::Kan::FRT) was prepared and the knock-out was transferred into JCL260 by P1 transduction. The Kan resistance cassette was removed from this strain using transient expression of FLP recombinase. The resulting strain was transduced with the Z1 cassette yielding GEVO1750, and the same strain was transduced with a lysate conferring a pykA deletion. The pykA deletion lysate was prepared from W3110, ΔpykA::FRT::Kan::FRT, which was created using homologous recombination according to the Wanner method using primers 1187 and 1188 for the amplification of the Kan cassette from pKD13. The Kan resistance cassette was removed from this strain using transient expression of FLP recombinase. The resulting strain was transduced with a lysate conferring a pykF deletion. The pykF deletion lysate was prepared from W3110, ΔpykF::FRT::Kan::FRT, which was created using homologous recombination according to the Wanner method using primers 1191 and 1192 for the amplification of the Kan cassette from pKD13. Removal of the Kan resistance cassette from this strain using transient expression of FLP recombinase yielded GEVO1725. For the construction of GEVO1751 strain GEVO1725 was transduced with a lysate of W3110Z1. The resulting strain was GEVO1751.

For the construction of GEVO1777 ilvC was deleted according to the standard protocol for gene deletion using the Wanner method. Primers 868 and 869 were used to amplify the Kan resistance cassette from pKD13. The linear PCR product was transformed into E. coli W3110 pKD46 electro competent cells and the knock-out of ilvC was verified by PCR. The Kan resistance cassette was removed from this strain using transient expression of FLP recombinase. The resulting strain was transduced with the Z1 cassette yielding GEVO1777.

GEVO1780 was constructed by transforming JCL260 with plasmids pGV1655 (SEQ ID NO: 109) and pGV1698 (SEQ ID NO: 112).

GEVO1844: An E. coli sthA deletion strain was obtained from the Keio collection and the deletion of sthA was verified. The sthA deletion was transferred to GEVO1748 by P1 phage transduction and after removal of the Kan resistance cassette by transient expression of FLP recombinase the resulting strain GEVO1844 was verified for the sthA deletion.

GEVO1846 was constructed by transforming strain GEVO1748 with plasmids pGV1745 (SEQ ID NO: 117) and pGV1698 (SEQ ID NO: 112).

GEVO1859 was constructed according to the standard protocol for gene integration using the Wanner method (Datsenko, K. and Wanner, B. One-step Inactivation of chromosomal genes in Escherichia coli K-12 using PCR products. PNAS 2000). Primers 1219 and 1485 were used to amplify PLlacO1::Bs_alsS1::Ec_ilvC_coEc from pGV1698 (SEQ ID NO: 112). Primers 1218 and 1486 were used to amplify the Kan resistance cassette from pKD13. SOE (splicing by overlap extension) was used to combine the two pieces to one integration cassette. The linear PCR product was transformed into E. coli W3110 pKD46 electro competent cells and the knock-in of PLlacO1::Bs_alsS1::Ec_ilvC_coEc::FRT::Kan::FRT into the pflB locus was verified by PCR. The knock-in was further verified by sequencing. Lysate of the new strain (E. coli W3110, ΔpflB:: PLlacO1::Bs_alsS1::Ec_ilvC_coEc::FRT::Kan::FRT) was prepared and the knock-in was transferred into GEVO1749 by P1 transduction. Removal of the Kan resistance cassette from this strain using transient expression of FLP recombinase yielded GEVO1859.

GEVO1886 was constructed according to the standard protocol for gene integration using the Wanner method (Datsenko, K. and Wanner, B. One-step Inactivation of chromosomal genes in Escherichia coli K-12 using PCR products. PNAS 2000). Primers 1562 and 1539 were used to amplify PLlacO1::pntAB from pGV1745 (SEQ ID NO: 117). Primers 1479 and 1561 were used to amplify the Kan resistance cassette from pKD13. SOE was used to combine the two pieces to one integration cassette. The linear PCR product was transformed into E. coli W3110 pKD46 electro competent cells and the knock-in of PLlacO1::pntAB::FRT::Kan::FRT into the sthA locus was verified by PCR. The knock-in was further verified by sequencing. Lysate of the new strain (E. coli W3110, ΔsthA:: PLlacO1::pntAB::FRT::Kan::FRT) was prepared and the knock-in was transferred into GEVO1859 by P1 transduction. Removal of the Kan resistance cassette from this strain using transient expression of FLP recombinase yielded GEVO1886.

GEVO1993 is a derivative of GEVO1748. For the construction of GEVO1993, PLlacO1::Bs_alsS1 was integrated into the pta locus on the E. coli chromosome. In particular primers 1526 and 474 were used to amplify the kanamycin resistance cassette (Kan) from pKD13, and primers 1563 and 1527 were used to amplify PLlacO1::Bs_alsS1 from pGV1698. Afterwards, SOE (splicing by overlap extension) reactions were done to connect the gene expression cassette to the resistance cassette using primers 1563 and 474. The linear PCR products were transformed into E. coli W3110 pKD46 electro competent cells and the knock-ins of PLlacO1::Bs_alsS1::FRT::Kan::FRT were verified by PCR. The knock-ins were further verified by sequencing. Lysate of the new strain E. coli W3110, Δpta::PLlacO1::Bs_alsS1::FRT::Kan::FRT was prepared and the knock-in was transferred to GEVO1748 by P1 transduction yielding GEVO1993. The integration into the pta locus in GEVO1993 was verified by PCR.

Construction of Saccharomyces cerevisiae Strains

A PDC deletion variant S. cerevisiae, GEVO2302, was evolved so that it does not have the requirement for a two-carbon molecule and has a growth rate similar to the parental strain on glucose.

GEVO1186 is S. cerevisiae CEN.PK2

GEVO1803 was made by transforming GEVO1186 with the 6.7 kb pGV1730 (SEQ ID NO: 116) (contains S. cerevisiae TRP1 marker and the CUP1 promoter-driven Bs_alsS2) that had been linearized by digestion with NruI. Completion of the digest was confirmed by running a small sample on a gel. The digested DNA was then purified using Zymo Research DNA Clean and Concentrator and used in the transformation. Trp+ clones were confirmed for the correct integration into the PDC1 locus by colony PCR using primer pairs 1440+1441 and 1442+1443 for the 5′ and 3′ junctions, respectively. Expression of Bs_alsS2 was confirmed by qRT-PCR using primer pairs 1323+1324.

GEVO2107 was made by transforming GEVO1803 with linearized, Hpal-digested pGV1914 (SEQ ID NO: 119). Correct integration of pGV1914 at the PDC6 locus was confirmed by analyzing candidate Ura+ colonies by colony PCR using primers 1440 plus 1441, or 1443 plus 1633, to detect the 5′ and 3′ junctions of the integrated construct, respectively. Expression of all transgenes were confirmed by qRT-PCR using primer pairs 1321 plus 1322, 1587 plus 1588, and 1633 plus 1634 to examine Bs_alsS2, LI_kivd2_coEc, and Dm_ADH transcript levels, respectively.

GEVO2158 was made by transforming GEVO2107 with NruI-digested pGV1936 (SEQ ID NO: 120). Correct integration of pGV1936 at the PDC5 locus was confirmed by analyzing candidate Ura+, Leu+ colonies by colony PCR using primers primers 1436 plus 1437, or 1595 plus 1439, to detect the 5′ and 3′ junctions of the integrated construct, respectively. Expression of all transgenes were confirmed by qRT-PCR using primer pairs 1321 plus 1322, 1597 plus 1598, 1566 plus 1567, 1587 plus 1588, 1633 plus 1634, and 1341 plus 1342 to examine levels of Bs_alsS2, Ec_ilvC_coSc^{Q110V}, Sc_ilv3ΔN, LI_kivd2_coEc, Dm_ADH, and ACT1, respectively.

GEVO2302 was constructed by sporulating GEVO2158. Haploid spores were prepared for random spores analysis (as described above), and the spores were plated onto SCE-Trp,Leu,Ura medium (14 g/L Sigma™ Synthetic propout Media supplement (includes amino acids and nutrients excluding histidine, tryptophan, uracil, and leucine), 6.7 g/L Difco™ Yeast Nitrogen Base without amino acids. 0.076 g/L histidine and 25 mL/L 100% ethanol). Candidate colonies were patched onto SCE-Trp, Leu, Ura plates (Plate version of the above medium was prepared using 20 g/L agar) and then replica plated onto YPD (10 g/L yeast extract, 20 g/L peptone, 20 g/L glucose) and YPE (10 g/L yeast extract, 20 g/L peptone, 25 mL/L 100% ethanol) plates. Patches that grew on YPE but failed to grow on YPD were further analyzed by colony PCR to confirm mating type (and, hence, their status as haploid). Several verified haploid candidates were further analyzed for transgene expression by qRT-PCR. GEVO2302 contains the full isobutanol pathway with ALS, KARI, DHAD, KIVD, and ADH being encoded by Bs_alsS2, Ec_ilvC_coSc^{Q110V}, Sc_ilv3ΔN, LI_kivd2_coEc, Dm_ADH, respectively.

GEVO2710, GEVO2711, GEVO2712 and GEVO2799 are C2-independent, glucose de-repressed derivatives of GEVO2302, which were constructed via chemostat evolution: A DasGip fermentor vessel was sterilized and filled with 1 L of YNB+histidine medium (Yeast Nitrogen Base+histidine, containing per liter of distilled water: 6.7 g YNB without amino acids from Difco and 0.076 g histidine; the medium was adjusted to pH 5 by adding a few drops of HCL or KOH) and contained 2% w/v ethanol. The vessel was installed and all probes were calibrated according to DasGip instructions. The vessel was also attached to an off-gas analyzer of the DasGip system, as well as to a mass spectrometer. Online measurements of oxygen, carbon dioxide, isobutanol, and ethanol were taken throughout the experiment. The two probes that were inside the vessel measured pH and dissolved oxygen levels at all times. A medium inlet and an outlet were also set up on the vessel. The outlet tube was placed at a height just above the 1 L level, and the pump rate was set to maximum. This arrangement helped maintain the volume in the vessel at 1 L. Air was sparged into the fermentor at 12 standard liters per hour (slph) at all times. The temperature of the vessel was held constant at 30.0° C. and the agitation rate was set at a minimum of 500 rpm, with a cascade control to adjust the agitation to maintain 50% dissolved oxygen in the culture. The off-gas was analyzed for CO₂, O₂, ethanol and isobutanol concentrations. The amount of carbon dioxide (X_{CO2}) and oxygen (X_{O2}) levels in the off-gas were used to assess the metabolic state of the cells. An increase in X_{CO2} levels and decrease in X_{O2} levels indicated an increase in growth rate and glucose consumption rate. The ethanol levels were monitored to ensure that there was no contamination, either from other yeast cells or from potential revertants of the mutant strain because the S. cerevisiae PDC triple-mutant (GEVO2302) does not produce ethanol. The minimum pH in the vessel was set to 5, and a base control was set up to pump in potassium hydroxide into the vessel when the pH dropped below 5.

GEVO2302 was inoculated into 10 ml of YNB+histidine medium with 2% w/v ethanol as the carbon source. The culture was incubated at 30° C. overnight with shaking. The overnight culture was used to inoculate the DasGip vessel. Initially, the vessel was run in batch mode, to build up a high cell density. When about a cell biomass of OD₆₀₀=8 was reached, the vessel was switched to chemostat mode and the dilution of the culture began. The medium pumped into the vessel was YNB+histidine with 6.357 g/L glucose and 0.364 g/L of acetate (5% carbon equivalent). The initial dilution rate was set to 0.06 h⁻¹ to avoid washout.

After the culture in the chemostat was stabilized at the 0.06 h⁻¹ dilution rate, the concentration of acetate was slowly decreased. This was achieved by using a two pump system, effectively producing a gradient pumping scheme. Initially pump A was pumping YNB+histidine medium with 10 g/L glucose at a rate of 35.5 mL/h and pump B was pumping YNB+histidine medium with only 1 g/L acetate at a rate of 20.3 mL/h. The total acetate going into the vessel was 0.364 g/L. Then, over a period of 5 days, the rate of pump B was slowly decreased and the rate of pump A was increased so that the combined rate of feeding increased from 56 mL/h to 74 ml/h. Over this period, the rate of pump B was finally reduced to 0, resulting in no (0 g/L) acetate addition to the chemostat. The glucose feed to the chemostat over this period was increased from 6.4 g/L to 10 g/L and the evolved strain was able to grow on glucose only.

Evolution of the strain for growth on increased glucose concentration was performed by slowly increasing the concentration of glucose in the chemostat with the evolved strain that no longer required a 2-carbon supplement. The concentration of glucose in the feed medium was increased from 10 g/L to 38 g/L over a period of 31 days. This was achieved by using a two pump system, effectively producing a gradient pumping scheme. Initially pump A was pumping YNB+histidine medium with 10 g/L glucose at a rate of 35.2 mL/h and pump B was pumping YNB+histidine medium with 15 g/L glucose at a rate of 32.9 mL/h. The total glucose going into the vessel was 12.4 g/L. Then, over a period of 18 days, the medium reservoirs for pump A and pump B were replaced with reservoirs containing increased concentrations of glucose until the reservoir for pump A contained 80 g/L glucose and the reservoir for pump B contained 100 g/L glucose. During this period, the combined rate of feeding maintained a dilution rate of 0.04 h⁻¹. At the end of this period, the rate of pump A was finally reduced to 0, resulting in a feed of 100 g/L glucose. This dilution rate resulted in a biomass of OD₆₀₀=4.8 at this glucose concentration and increasing the dilution rate to 0.09 h⁻¹ over a period of 4 days lowered the biomass to an OD₆₀₀=2.6. The dilution rate was lowered to 0.03 h⁻¹ and gradually raised to 0.04 h⁻¹ at 100 g/L glucose feed to raise the biomass to an OD₆₀₀=4.4 over a period of 5 days. The glucose feed was then lowered by replacing the medium reservoir for pump A with a reservoir containing 0 g/L glucose, pumping initially at a rate of 33.4 ml/h, and pumping the 100 g/L glucose feed from pump B at 2.4 ml/h. This resulted in a dilution rate of 0.04 h⁻¹, a glucose feed of 6.7 g/L and a biomass of OD₆₀₀=6.0. Over a period of 4 days, the glucose concentration in the feed was gradually increased to 37.8 g/L by increasing the rate of pump B and decreasing the rate of pump A while maintaining a dilution rate of 0.04 h⁻¹ and resulting in a biomass under these conditions of an OD₆₀₀=6.6 and a glucose level in the chemostat of 18.8 g/L.

Evolution of the strain for increased growth rate was performed by slowly increasing the dilution rate in the chemostat with the evolved strain that no longer required a 2-carbon supplement and could grow with a feed of 37.8 g/L glucose with a glucose level in the chemostat of 18.8 g/L. Over a period of 13 days, the dilution rate was gradually increased from 0.04 h⁻¹ to 0.14 h⁻¹ by alternately increasing the rates of pump A and pump B to maintain a glucose feed concentration of 21-24 g/L glucose. A biomass of OD₆₀₀=1.6 to an OD₆₀₀=2.0 was maintained at dilution rates of 0.13 h⁻¹ to 0.14 h⁻¹.

Over the period of evolution, a sample was occasionally removed from the vessel directly. Samples were analyzed for glucose, acetate, and pyruvate using HPLC. Samples were plated onto YNB+histidine medium with 2% w/v ethanol as carbon source, YNB+histidine medium with different glucose concentrations (5 g/L, 10 g/L, 15 g/L, 20 g/L, 25 g/L and 50 g/L glucose), and YPD medium (containing 10 g/L yeast extract, 20 g/L peptone and 20 g/L dextrose) agar plates (plates contain the indicated medium+20 g/L agar). OD₆₀₀ measurements were taken regularly to make sure the chemostat did not wash out. Freezer stocks of samples of the culture were made regularly for future characterization of the strains.

The chemostat with the evolved strain that no longer required a 2-carbon supplement and could grow with a feed of 37.8 g/L glucose with a glucose level in the chemostat of 18.8 g/L and could grow at a dilution rate>0.13 h⁻¹ was maintained for another 23 days with varying dilution rates from 0.07 h⁻¹ to 0.11 h⁻¹ to allow further evolution for improved growth rate. At the end of this period, a sample from the chemostat was plated onto YNB+histidine medium with 50 g/L glucose agar plates and allowed to form colonies at 30° C. Ten colonies were picked for further characterization and re-streaked onto YNB+histidine medium with 50 g/L glucose agar plates for purification. None of these 10 evolved strains isolated from the chemostat sample grew when streaked onto SC-histidine medium (Synthetic complete medium lacking histidine, containing per liter of distilled water: 6.7 g YNB without amino acids from Difco, 100 ml of a solution of 14 g Yeast Synthetic prop-out Medium Supplements without histidine, leucine, tryptophan and uracil from Sigma dissolved in 1 L water, 20 ml of a solution of 3.8 g/L tryptophan, 20 ml of a solution of 19 g/L leucine and 40 ml of a solution of 1.9 g/L uracil) containing 20 g/L glucose plates but did grow on SC-leucine medium (Synthetic complete medium lacking leucine, containing per liter of distilled water: 6.7 g YNB without amino acids from Difco, 100 ml of a solution of 14 g Yeast Synthetic prop-out Medium Supplements without histidine, leucine, tryptophan and uracil from Sigma dissolved in 1 L water, 20 ml of a solution of 3.8 g/L tryptophan, 20 ml of a solution of 3.8 g/L histidine and 40 ml of a solution of 1.9 g/L uracil) containing 20 g/L glucose plates, indicating that they were still auxotrophic for histidine.

To characterize growth of the evolved strains, single colonies from each of the 10 evolved isolates purified on YNB+histidine medium with 50 g/L glucose agar plates were inoculated into 3 ml of YNB+histidine medium with 50 g/L glucose and YPD medium in 14 ml round-bottom snap-cap tubes and incubated overnight at 30° C. as a pre-culture. The next day the pre-cultures were used to inoculate 5 ml of the same medium as the pre-cultures in 50 ml conical plastic screw-cap centrifuge tubes to an OD₆₀₀ of 0.01. The cultures were incubated shaking upright at 250 rpm at 30° C. and sampled periodically for OD₆₀₀ measurement. Growth rates were calculated from plots of the OD₆₀₀ measurements vs. time of incubation. Evolved isolates GEVO2710, GEVO2711, GEVO2712 and GEVO2799 were selected because of high growth rates in both YNB+histidine medium with 50 g/L glucose and YPD medium.

GEVO2792 is a C2-independent, PDC-minus S. cerevisiae strain carrying a control plasmid encoding no genes for an isobutanol metabolic pathway. To generate this strain, GEVO2710 was transformed with plasmid pGV2020 (SEQ ID NO: 121).

GEVO2844 is a C2-independent, PDC-minus S. cerevisiae strain carrying a control plasmid encoding no genes for an isobutanol metabolic pathway. To generate this strain, GEVO2799 was transformed with plasmid pGV2020 (SEQ ID NO: 121).

GEVO2847 is a C2-independent, PDC-minus S. cerevisiae strain carrying a partially NADH-utilizing isobutanol metabolic pathway. To generate this strain, GEVO2799 was transformed with plasmid pGV2082 (SEQ ID NO: 122), carrying the genes encoding NADPH-dependent KARI and the NADH-dependent ADH, Ec_ilvC_coSc^{Q110V}(SEQ ID NO: 24), and Dm_ADH (SEQ ID NO: 60), respectively.

GEVO2848 is a C2-independent, PDC-minus S. cerevisiae strain carrying a partially NADH-utilizing isobutanol metabolic pathway. To generate this strain, GEVO2799 was transformed with plasmid pGV2227 (SEQ ID NO: 123), carrying the genes encoding NADPH-dependent KARI and the NADH-dependent ADH, Ec_ilvC_coSc^{Q110V} (SEQ ID NO: 24), and LI_adhA (SEQ ID NO: 66), respectively.

GEVO2849 is a C2-independent, PDC-minus S. cerevisiae strain carrying an NADH-utilizing isobutanol metabolic pathway. To generate this strain, GEVO2799 was transformed with plasmid pGV2242 (SEQ ID NO: 125), carrying the genes encoding NADH-dependent KARI and ADH, Ec_ilvC_coSc^{P2D1} (SEQ ID NO: 39) and LI_adhA (SEQ ID NO: 66), respectively.

GEVO2851 is a C2-independent, PDC-minus S. cerevisiae strain carrying a partially NADH-utilizing isobutanol metabolic pathway. To generate this strain, GEVO2711 was transformed with plasmid pGV2227 (SEQ ID NO: 123), carrying the genes encoding NADPH-dependent KARI and the NADH-dependent ADH, Ec_ilvC_coSc^{Q110V} (SEQ ID NO: 24), and LI_adhA (SEQ ID NO: 66), respectively.

GEVO2852 is a C2-independent, PDC-minus S. cerevisiae strain carrying an NADH-utilizing isobutanol metabolic pathway. To generate this strain, GEVO2711 was transformed with plasmid pGV2242 (SEQ ID NO: 125), carrying the genes encoding NADH-dependent KARI and ADH, Ec_ilvC_coSc^{P2D1} (SEQ ID NO: 39) and LI_adhA (SEQ ID NO: 66), respectively.

GEVO2854 is a C2-independent, PDC-minus S. cerevisiae strain carrying a partially NADH-utilizing isobutanol metabolic pathway. To generate this strain, GEVO2710 was transformed with plasmid pGV2082 (SEQ ID NO: 122), carrying the genes encoding NADPH-dependent KARI and the NADH-dependent ADH, Ec_ilvC_coSc^{Q110V}, and Dm_ADH (SEQ ID NO: 60), respectively.

GEVO2855 is a C2-independent, PDC-minus S. cerevisiae strain carrying a partially NADH-utilizing isobutanol metabolic pathway. To generate this strain, GEVO2710 was transformed with plasmid pGV2227 (SEQ ID NO: 123), carrying the genes encoding NADPH-dependent KARI and the NADH-dependent ADH Ec_ilvC_coSc^{Q110V}, and LI_adhA (SEQ ID NO: 66), respectively.

GEVO2856 is a C2-independent, PDC-minus S. cerevisiae strain carrying an NADH-utilizing isobutanol metabolic pathway. To generate this strain, GEVO2710 was transformed with plasmid pGV2242 (SEQ ID NO: 125), carrying the genes encoding NADH-dependent KARI and ADH, Ec_ilvC_coSc^{P2D1} (SEQ ID NO: 39) and LI_adhA (SEQ ID NO: 66), respectively.

Construction of E. coli Expression Plasmids

pGV1631: The adh2 gene was cut out of plasmid pSA55 using appropriate restriction enzymes. Re-ligation yielded plasmid pGV1631 featuring only LI_kivd1 (SEQ ID NO: 45) under the control of the PLlacOI promoter. The plasmid was verified by sequencing prior to its use.

pGV1705A: The Ec_yqhD gene (SEQ ID NO: 68) contained on plasmid pGV1705 was cloned into plasmid pGV1711 (SEQ ID NO: 113) using the primers XX3 and XX4. These primers added additional sequences surrounding the ADH coding sequence. Specifically, the 5′-end of the PCR product contains an EcoRI site, a BamHI site, a RBS (aggaga), a 7 nucleotide space sequence, and the initiating ATG codon. The 3′ end of the product, following the stop codon, contains a NotI site followed by an AvrII site. The amplified product was digested with EcoRI and NotI and ligated into pGV1711 (SEQ ID NO: 113) which had been cut with both EcoRI and AvrII and gel purified to generate plasmid pGV1705-A,

ADH genes, whether PCR amplified or ordered as synthetic DNA sequences were cloned into plasmid pGV1716 (SEQ ID NO: 114), a derivative of plasmid pGV1698 carrying an in vitro-synthesized gene for S. cerevisiae ADH2, codon-optimized for expression in E. coli (=“ADH2co”). ADH2co gene was amplified from plasmid pGV1527 in a PCR reaction using KOD polymerase (Novagen, Gibbstown, N.J.) and primers 1296 and 1297. These primers add additional sequences surrounding the ADH2co coding sequence. Specifically, the 5′-end of the PCR product contains a SalI site, a BamHI site, an RBS (aggaga), a 7 nucleotide space sequence, and the initiating ATG codon. The 3′ end of the product, following the stop codon, contains a NotI site followed by a SalI site. The amplified product was digested SalI and was ligated into pGV1698 (SEQ ID NO: 112) which had been cut with SalI and gel purified. DNA constructs were analyzed by multiple restriction digests, and also by DNA sequencing to confirm integrity and to correct construction. Oligonucleotides 1220 and 1365 were used as primers in standard DNA sequencing reactions to sequence all of the aforementioned clones.

Plasmid pGV1748, which contains the ORF for Ec_fucO (SEQ ID NO: 64) expressed under the control of the IPTG-inducible promoter PLlacO1, was generated by amplifying the Ec_fucO gene in a PCR reaction, using primers 1470 and 1471 and E. coli genomic DNA as a template. The ˜1.2 kb PCR product so generated was digested with BamHI plus NotI, purified using a Zymo Research DNA Gel Extraction kit (Zymo Research, Orange, Calif.) according to manufacturer's protocol, and ligated into the vector pGV1716 (SEQ ID NO: 114) which had been digested with BamHI plus NotI and purified using a Zymo Research DNA Gel Extraction kit (Zymo Research, Orange, Calif.).

Plasmid pGV1748-A: The Ec_fucO gene contained on plasmid pGV1748 was cloned into plasmid pGV1711 (SEQ ID NO: 113) using the primers XX1 and XX2. These primers add additional sequences into the vector backbone upstream of the AvrII restriction site and downstream of the EcoRI restriction site. Specifically, the 5′-end of the PCR product contains a NotI site followed by an AvrII site and the 3′ end of the product, contains an AgeI site followed by an EcoRI site. The amplified product was digested with AgeI and NotI and ligated with the similarly digested pGV1711 to generate plasmid 1748-A.

Plasmid pGV1749, which contains the ORF for Dm_ADH (SEQ ID NO: 60) expressed under the control of the IPTG-inducible promoter PLlacO1, was generated by amplifying the Dm_ADH gene in a PCR reaction, using primers 1469 and 1364 and the clone RH54514 (Drosophila Genome Resource Center) as a template. The ˜0.8 kb PCR product was digested with BglII plus NotI, was purified using a Zymo Research DNA Gel Extraction kit according to manufacturer's protocol, and was ligated into the vector pGV1716 (SEQ ID NO: 114) which had been digested with BamHI plus NotI and purified using a Zymo Research DNA Gel Extraction kit.

Plasmid pGV1749-A: The Dm_ADH gene (SEQ ID NO: 60) contained on plasmid pGV1749 was cloned into plasmid pGV1711 (SEQ ID NO: 113) using the primers XX1 and XX2. These primers add additional sequences into the vector backbone 5′ of the AvrII restriction site and 3′ of the EcoRI restriction site. Specifically, the 5′-end of the PCR product contains a NotI site followed by an AvrII site and the 3′ end of the product, contains an AgeI site followed by an EcorI site. The amplified product was digested with AgeI and NotI and ligated with the product of the ADH gene similarly digested with AgeI and NotI to generate plasmid pGV1749-A.

Plasmid pGV1778, which contains the ORF for Kp_dhaT (SEQ ID NO: 62) expressed under the control of the IPTG-inducible promoter PLlacO1, was generated by excising the Kp_dhaT gene from an in vitro synthesized plasmid (generated by DNA2.0, Menlo Park, Calif.) by digestion with BamHI plus NotI. The released 1.16 kb fragment was purified using a Zymo Research DNA Gel Extraction kit according to manufacturer's protocol, and was ligated into the vector pGV1716 (SEQ ID NO: 114) which had been digested with BamHI plus NotI and purified using a Zymo Research DNA Gel Extraction kit.

Plasmid pGV1778-A: The Kp_dhaT gene (SEQ ID NO: 62) contained on plasmid pGV1778 was cloned into plasmid pGV1711 (SEQ ID NO: 113) using the primers XX1 and XX2. These primers add additional sequences into the vector backbone 5′ of the AvrII restriction site and 3′ of the EcoRI restriction site. Specifically, the 5′-end of the PCR product contains a NotI site followed by an AvrII site and the 3′ end of the product, contains an Agel site followed by an EcoRI site. The amplified product was digested with AgeI and NotI and ligated with the product of the ADH gene similarly digested with AgeI and NotI to generate plasmid pGV1778-A.

Plasmids pGV1655 (SEQ ID NO: 109) and pGV1711 (SEQ ID NO: 113) have been described previously. Briefly, pGV1655 is a low-copy, Kan^{R}-selected plasmid that expresses E. coli Ec_ilvD_coEc (SEQ ID NO: 51) and LI_kivd1 (SEQ ID NO: 41) under the control of the PLlac promoter.

Plasmid pGV1938 was constructed by inserting the gene coding for Ec_IlvC_coEc^{S78D} into pGV1711 (SEQ ID NO: 113). The KARI variant gene was amplified with primers Not_in_for and AvrII_in_rev introducing the 5′ NotI and the 3′ AvrII restriction sites, DpnI digested for 1 h at 37° C., and then cleaned up using the Zymo PCR clean up kit. The fragment and the vector pGV1711 were restriction digested with NotI and AvrII and run out on a 1% agarose gel. After cutting out the fragments, they were cleaned up using the Freeze'n'Squeeze and pellet paint procedure. Ligation was performed with the rapid ligation kit from Roche according to the manufacturer's instructions.

Plasmid pGV1939 was generated using primers XX3 and XX4 to amplify the Ec_fucO gene from plasmid pGV1748-A. The forward primer adds a new RBS (aggaga), a 7 nucleotide space sequence, and the initiating ATG codon. The amplified product was digested with EcoRI and NotI and ligated with the similarly digested pGV1711 (SEQ ID NO: 113) to generate plasmid pGV1939 containing the modified RBS.

The genes coding for KARI variants Ec_ilvC_coEc^{his6} (SEQ ID NO: 14), Ec_ilvC_coEc^{S78D-his6} (SEQ ID NO: 16), Ec_ilvC_coEc^{6E6-his6} (SEQ ID NO: 32) and Ec_ilvC_coEc^{2H10-his6} (SEQ ID NO: 30) were cloned into pGV1939 generating plasmids pGV1925, pGV1927, pGV1975 and pGV1976, respectively using primers NotI_in_for and AvrII_in_rev. The PCR products were DpnI digested for 1 h and cleaned over a 1% agarose gel. After a sequential restriction digestion of vector and insert with NotI for 1 h followed by 1 h with AvrII, ligation was performed using rapid ligase (Roche). Ligation mixture was desalted using the Zymo PCR clean up kit and used to transform E. coli DH5α. DNA constructs were analyzed by restriction digests, and also by DNA sequencing to confirm integrity and correct construction. Primers pETup and KARIpETrev were used as primers in standard DNA sequencing reactions to sequence pET22b(+) derivatives, primer seq_ilvc_pGV was used to sequence pGV1925, pGV1927, pGV1975 and pGV1976.

Construction of Saccharomyces cerevisiae Expression Plasmids

pGV1824: The gene coding for Ec_IlvC (SEQ ID NO: 13) was codon optimized for S. cerevisiae and synthesized (DNA2.0, Menlo Park, Calif.), resulting in Ec_ilvC_coSc (SEQ ID NO: 12). To generate pGV1824, the Ec_ilvC_coSc gene was excised from plasmid pGV1774 using BglII and XhoI. Plasmid pGV1662 was digested with SalI and BamHI. The pGV1662 vector backbone and Ec_ilvC_coSc insert were ligated using standard methods resulting in plasmid pGV1824 containing the gene Ec_ilvC_coSc.

pGV1914 (SEQ ID NO: 119) is a yeast integrating vector (YIp) that utilizes the S. cerevisiae URA3 gene as a selection marker and contains homologous sequence for targeting the HpaI-digested, linearized plasmid for integration at the PDC6 locus of S. cerevisiae. This plasmid does not carry a yeast replication origin, thus is unable to replicate episomally. This plasmid carries the Dm_ADH (SEQ ID NO: 60) and LI_kivd2_coEc (SEQ ID NO: 48) genes, expressed under the control of the S. cerevisiae TDH3 and TEF1 promoters, respectively. pGV1914 was generated in two steps. First, the Dm_ADH-containing E. coli expression plasmid pGV1749 was digested with SalI plus NotI, and the 0.78 kb fragment containing the Dm_ADH ORF released by digestion was gel purified and ligated into pGV1635, which had been digested with XhoI plus NotI and gel purified. Plasmid pGV1635 is a yeast expression plasmid which has as its salient feature a TDH3 promoter followed by several restriction enzyme recognition sites, into which the Dm_ADH sequence was cloned as described above. A correct recombinant plasmid was named pGV1913. In the second step of pGV1914 construction, pGV1913 was digested with BamHI plus NotI and the 1.45 kb fragment, containing the TDH3 promoter-Dm_ADH ORF sequence was gel purified and ligated into pGV1733, which had been digested with BamHI plus NotI and similarly gel purified, yielding pGV1914. Thus, the ScADH7 ORF in pGV1733 is replaced by the Dm_ADH ORF in the pGV1914, both under the control of the TDH3 promoter; both plasmids also contain the P_{TEF1}-LI_kivd2_coEc cassette as well as the URA3 selection marker and ScPDC6 5′ and 3′ regions suitable for homologous recombination targeting following linearization of the plasmid with HpaI.

pGV1936 (SEQ ID NO: 120) is a yeast integrating vector (YIp) that utilizes the S. cerevisiae LEU2 gene as a selection marker and contains homologous sequence for targeting the linearized (by HpaI digestion) plasmid for integration at the PDC5 locus of S. cerevisiae. This plasmid does not carry a yeast replication origin, thus is unable to replicate episomally. This plasmid carries the Ec_ilvC_coSc^{Q110V} (SEQ ID NO: 24) mutant (i.e. codon optimized for expression in S. cerevisiae) and S. cerevisiae ILV3ΔN genes, expressed under the control of the S. cerevisiae TDH3 and TEF1 promoters, respectively. pGV1936 was constructed using an SOE PCR method that amplified the Ec_ilvC_coSc gene while simultaneously introducing the nucleotide changes coding for a Q110V mutation. Specifically, primers 1624 and 1814 were used to amplify a portion of plasmid pGV1774 containing the Ec_ilvC_coSc gene; primers 1813 and 1798 were used to amplify a portion of plasmid pGV1824 that also contained the Ec_ilvC_coSc gene. The two separate PCR products were gel purified, eluted in 15 μL, and 3 μL of each were used as a template along with primers 1624 and 1798. The resulting PCR product was digested with XhoI plus NotI and ligated into pGV1765 that had been digested with XhoI plus NotI, yielding pGV1936. Candidate clones of pGV1936 were confirmed by sequencing, using primers 350, 1595, and 1597.

pGV1994: Mutations found in variant Ec_IlvC^{6E6-his6} were introduced into pGV1824 by SOE PCR. The 5′ PCR used primers 1898 and 2037 and the 3′ PCR used primers 1893 and 2036. Each of these primer pairs were used with pGV1894 as the template in two separate PCR reactions. The product was used in a second PCR with the end primers 1898 and 1893 to yield a final PCR product. This final PCR product has a 5′ SalI restriction site and 3′ BglII followed by NotI restriction sites. These were cloned into pGV1662 using the SalI and NotI site and yielding plasmid pGV1994 which carries Ec_ilvC_coSc^{6E6} (SEQ ID NO: 35).

pGV2020 (SEQ ID NO: 121) is an empty G418 resistant 2-micron yeast vector that was generated by removing the LI_kivd2_coEc sequence from pGV2017. This was carried out by amplifying the TDH3 promoter from pGV2017 using primers 1926 and 1927, digesting with SalI and NotI and cloning into the same sites of pGV2017.

pGV2082 (SEQ ID NO: 122) is a G418 resistant yeast 2-micron plasmid for the expressions of Ec_ilvC_coSc^{Q110V} (SEQ ID NO: 24), LI_ilvD_coSc (SEQ ID NO: 54), LI_kivd2_coEc (SEQ ID NO: 48), and Dm_ADH (SEQ ID NO: 60). A fragment carrying the PGK1 promoter, LI_kivd2_coEc and a short region of the PDC1 terminator sequence was obtained by cutting pGV2047 with AvrII and NcoI. This fragment was treated with Klenow to generate blunt ends then cloned into pGV2044 that had been digested with EcoRI and SbfI and the overhangs filled in with Klenow. This construction replaced the CUP1 promoter and the Bs_alsS1_coSc (SEQ ID NO: 6) in pGV2044 with the PGK1 promoter and LI_kivd2_coEc.

pGV2193: The Ec_IlvC variant encoded by Ec_ilvC_coSc^{6E6-his6} (SEQ ID NO: 33) encoded on pGV2241 (SEQ ID NO: 124) served as template for error-prone PCR using primers pGV1994ep_for and pGV1994ep_rev yielding variant Ec_IlvC^{P2D1-his6} (SEQ ID NO: 38) which is encoded by Ec_ilvC_coSc^{P2D1-his6} (SEQ ID NO: 37) on construct pGV2193.

pGV2227 (SEQ ID NO: 123) is a G418 resistant yeast 2-micron plasmid for the expressions of Ec_ilvC_coSc^{Q110V} (SEQ ID NO: 24), LI_ilvD_coSc (SEQ ID NO: 54), LI_kivd2_coEc (SEQ ID NO: 48), and LI_adhA (SEQ ID NO: 66). pGV2227 is a derivative of pGV2201 where the BamHI and XhoI sites at the 3′ end of the LI_adhA were removed and replaced with an AvrII site. This construction was carried out by cloning into the NheI-MluI sites of pGV2202a fragment carrying the 3′ end of the LI_adhA sequence, an AvrII site, and the 5′ part of the CYC1 terminator. This fragment was generated by SOE PCR combining a PCR product using primers 2091 and 2352 with pGV2201 as template and a PCR product using primers 2353 and 772 with pGV2201 as template. The sequences of primers 2352 and 2353 overlap and introduce an AvrII site. This SOE PCR product was digested with NheI and MluI for cloning into pGV2201.

pGV2238: The Ec_IlvC variant encoded by Ec_ilvC_coSc^{P2D1-his6} (SEQ ID NO: 37) encoded on pGV2193 served as parent for an additional error-prone PCR round using the same primers as described before on template DNA pGV2193 yielding an improved KARI variant named Ec_IlvC^{P2D1-A1-his6} (SEQ ID NO: 42) which is encoded by the gene Ec_ilvC_coSc^{P2D1-A1-his6} (SEQ ID NO: 41) on plasmid pGV2238.

pGV2241 (SEQ ID NO: 124): The gene Ec_ilvC_coSc^{6E6} (SEQ ID NO: 35) was his-tagged using primers pGV1994_ep_for and 1994hisrev, cleaned with the Zymo PCR clean up kit (Zymo Research), NotI and SalI digested, and ligated into similarly digested pGV1994, resulting in construct pGV2241 coding for Ec_ilvC_coSc^{6E6-his6} (SEQ ID NO: 33).

pGV2242 (SEQ ID NO: 125) is a G418 resistant yeast 2-micron plasmid for the expressions of Ec_ilvC_coSc^{P2D1} (SEQ ID NO: 39), LI_ilvD_coSc (SEQ ID NO: 54), LI_kivd2_coEc (SEQ ID NO: 48), and LI_adhA (SEQ ID NO: 66). This plasmid was generated by cloning the SalI-BspEI fragment of pGV2193 carrying the region encoding for Ec_IlvC with the relevant mutations for the Ec_ilvC_coSc^{P2D1} allele into the XhoI-BspEI sites of pGV2227 (SEQ ID NO: 123).

TABLE 3
Strains disclosed herein

| Strain No. | Description |
|---|---|
| GEVO1186 | S. cerevisiae CEN.PK2 (MATa/α ura3/ura3 leu2/leu2 his3/his3 trp1/trp1 |
| | PDC1/PDC1 PDC5/PDC5 PDC6/PDC6) |
| GEVO1385 | E. coli BW25113, ΔldhA-fnr::FRT, ΔadhE::FRT, Δfrd::FRT, Δpta::FRT, |
| | ΔpflB::FRT, F′ (laclq+), attB::(Sp⁺ laclq⁺ tetR⁺) |
| GEVO1399 | E. coli BW25113, ΔldhA-fnr::FRT, ΔadhE::FRT, Δfrd::FRT, Δpta::FRT, |
| | pflB::FRT, Δzwf::FRT F′ (laclq+) |
| GEVO1608 | E. coli BW25113, ΔldhA-fnr::FRT, ΔadhE::FRT, Δfrd::FRT, ΔpflB::FRT, |
| | Δpta::FRT, ΔyqhD::FRT-Kan-FRT, F′ (laclq+) |
| GEVO1725 | E. coli BW25113, ΔldhA-fnr::FRT, ΔadhE::FRT, Δfrd::FRT, Δpta::FRT, |
| | ΔpflB::FRT, ΔmaeA::FRT, ΔpykA::FRT, ΔpykF::FRT, F′ (laclq+) |
| GEVO1745 | E. coli BW25113, ΔldhA-fnr::FRT, ΔadhE::FRT, Δfrd::FRT, ΔpflB::FRT, |
| | Δpta::FRT, ΔyqhD::FRT |
| GEVO1748 | E. coli BW25113, ΔldhA-fnr::FRT, ΔadhE::FRT, Δfrd::FRT, Δpta::FRT, |
| | pflB::FRT, F′ (laclq+), ΔilvC::PLlacO1::Ll_kivd1::Ec_ilvD_coEc::FRT |
| GEVO1749 | E. coli BW25113, ΔldhA-fnr::FRT, Δfrd::FRT, Δpta::FRT, pflB::FRT, F′ |
| | (laclq+), ΔadhE::[PLlacO1::Ll_kivd1::Ec_ilvD_coEc::FRT] |
| GEVO1750 | E. coli BW25113, ΔldhA-fnr::FRT, ΔadhE::FRT, Δfrd::FRT, Δpta::FRT, |
| | ΔpflB::FRT, ΔmaeA::FRT, F′ (laclq+), attB::(Sp+ laclq+ tetR+) |
| GEVO1751 | E. coli BW25113, ΔldhA-fnr::FRT, ΔadhE::FRT, Δfrd::FRT, Δpta::FRT, |
| | ΔpflB::FRT, ΔmaeA::FRT, ΔpykA::FRT, ΔpykF::FRT, F′ (laclq+), attB::(Sp+ |
| | laclq+ tetR+) |
| GEVO1777 | E. coli W3110, ΔilvC::FRT, attB::(Sp+ laclq+ tetR+) |
| GEVO1780 | JCL260 transformed with pGV1655 and pGV1698 |
| GEVO1803 | S. cerevisiae CEN.PK2, MATa/alpha ura3/ura3 leu2/leu2 his3/his3 trp1/trp1 |
| | pdc1::Bs_alsS2, TRP1/PDC1 |
| GEVO1844 | E. coli BW25113, Δ(ldhA-fnr::FRT) ΔadhE::FRT Δfrd::FRT Δpta::FRT |
| | ΔpflB::FRT ΔilvC::P_{LlacO1}::Ll_kivd1::Ec_ilvD_coEc::FRT ΔsthA::FRT |
| GEVO1846 | E. coli BW25113, ΔldhA-fnr::FRT, ΔadhE::FRT, Δfrd::FRT, Δpta::FRT, |
| | pflB::FRT, F′ (laclq+), ΔilvC::PLlacO1::Ll_kivd1::Ec_ilvD_coEc::FRT, |
| | pGV1745, pGV1698 |
| GEVO1859 | E. coli BW25113, ΔldhA-fnr::FRT, Δfrd::FRT, Δpta::FRT, F′ (laclq+), |
| | ΔadhE::[pLlacO1::Ll_kivd1::Ec_ilvD_coEc::FRT], |
| | pflB::[pLlacO1::Bs_alsS1::Ec_ilvC_coEc::FRT] |
| GEVO1886 | E. coli BW25113, ΔldhA-fnr::FRT, Δfrd::FRT, Δpta::FRT, F′ (laclq+), |
| | ΔadhE::[pLlacO1::Ll_kivd1::Ec_ilvD_coEc::FRT], ΔpflB::[pLlacO1::Bs_alsS1 |
| | ::Ec_ilvC_coEc::FRT] ΔsthA::[pLlacO1::pntA::pntB::FRT] |
| GEVO1993 | E. coli BW25113, ΔldhA-fnr::FRT, ΔadhE::FRT, Δfrd::FRT, DpflB::FRT, F′ |
| | (laclq+), ΔilvC::PLlacO1::Ll_kivd1::Ec_ilvD_coEc::FRT, |
| | Δpta::PLlacO1::Bs_alsS1, FRT::KAN::FRT |
| GEVO2107 | S. cerevisiae CEN.PK2, MATa/alpha ura3/ura3 leu2/leu2 his3/his3 trp1/trp1 |
| | pdc1::Bs_alsS2, TRP1/PDC1 pdc6::{ScTEF1p-Ll_kivd2_coEcScTDH3p- |
| | Dm_ADH URA3}/PDC6 |
| GEVO2158 | S. cerevisiae CEN. PK2; MATa/α ura3/ura3 leu2/leu2 his3/his3 trp1/trp1 |
| | pdc1::Bs_alsS2, TRP1/PDC1 pdc5::{ScTEF1prom-Sc_ILV3ΔN ScTDH3prom- |
| | Ec_ilvC_coSc^{Q110V} LEU2}/PDC5 pdc6::{ScTEF1p-Ll_kivd2_coEc ScTDH3p- |
| | Dm_ADH URA3}/PDC6 |
| GEVO2302 | S. cerevisiae CEN.PK2; MATa ura3 leu2 his3 trp1 pdc1::Bs_alsS2, TRP1 |
| | pdc5::{P_{TEF1}:Sc_ILV3ΔN P_{TDH3}:Ec_ilvC_coSc^{Q110V} LEU2} pdc6::{P_{TEF1}: |
| | Ll_kivd2_coEc P_{TDH3}:Dm_ADH URA3} |
| GEVO2710 | S. cerevisiae CEN.PK2; MATa ura3 leu2 his3 trp1 pdc1::{P_{CUP1}- |
| | Bs_alsS2, TRP1} pdc5::{P_{TEF1}:Sc_ILV3ΔN P_{TDH3}:Ec_ilvC_coSc^{Q110V}, LEU2} |
| | pdc6::{P_{TEF1}: Ll_kivd2_coEc P_{TDH3}:Dm_ADH, URA3}, evolved for C2 |
| | supplement-independence, glucose tolerance and faster growth |
| GEVO2711 | S. cerevisiae CEN.PK2; MATa ura3 leu2 his3 trp1 pdc1::{P_{CUP1}- |
| | Bs_alsS2, TRP1} pdc5::{P_{TEF1}:Sc_ILV3ΔN P_{TDH3}:Ec_ilvC_coSc^{Q110V}, LEU2} |
| | pdc6::{P_{TEF1}: Ll_kivd2_coEc P_{TDH3}:Dm_ADH, URA3}, evolved for C2 |
| | supplement-independence, glucose tolerance and faster growth |
| GEVO2712 | S. cerevisiae CEN.PK2; MATa ura3 leu2 his3 trp1 pdc1::{P_{CUP1}- |
| | Bs_alsS2, TRP1} pdc5::{P_{TEF1}:Sc_ILV3ΔN P_{TDH3}:Ec_ilvC_coSc^{Q110V}, LEU2} |
| | pdc6::{P_{TEF1}: Ll_kivd2_coEc P_{TDH3}:Dm_ADH, URA3}, evolved for C2 |
| | supplement-independence, glucose tolerance and faster growth |
| GEVO2799 | S. cerevisiae CEN.PK2; MATa ura3 leu2 his3 trp1 pdc1::{P_{CUP1}- |
| | Bs_alsS2, TRP1} pdc5::{P_{TEF1}:Sc_ILV3ΔN P_{TDH3}:Ec_ilvC_coSc^{Q110V}, LEU2} |
| | pdc6::{P_{TEF1}: Ll_kivd2_coEc P_{TDH3}:Dm_ADH, URA3}, evolved for C2 |
| | supplement-independence, glucose tolerance and faster growth |
| GEVO2792 | GEVO2710 transformed with pGV2020 |
| GEVO2844 | GEVO2799 transformed with pGV2020 |
| GEVO2847 | GEVO2799 transformed with pGV2082 |
| GEVO2848 | GEVO2799 transformed with pGV2227 |
| GEVO2849 | GEVO2799 transformed with pGV2242 |
| GEVO2851 | GEVO2711 transformed with pGV2227 |
| GEVO2052 | GEVO2711 transformed with pGV2242 |
| GEVO2854 | GEVO2710 transformed with pGV2082 |
| GEVO2855 | GEVO2710 transformed with pGV2227 |
| GEVO2856 | GEVO2710 transformed with pGV2242 |
| GEVO5001 | S. cerevisiae CEN.PK2, Δpdc1 Δpdc5 Δpdc6 expressing an isobutanol |
| | pathway (ALS, KARI, DHAD, KIVD, ADH) |
| GEVO5002 | GEVO5001 P_{TEF1}:NADH kinase P_{TDH3}:NADP⁺ phosphatase HPH |
| GEVO5003 | GEVO5001, P_{TDH3}:Kl_GDP1 HPH |
| GEVO5004 | GEVO5001 P_{TEF1}:ess:pntA P_{TDH3}:ess:pntB HPH |
| GEVO5005 | GEVO5001 P_{TEF1}:mts:pntA P_{TDH3}:mts:pntB HPH |
| GEVO5006 | GEVO5001 P_{ADH1}:PYC1 P_{TEF1}:MDH2 P_{TDH3}:maeB HPH |
| E. coli BL21 | Lucigen Corporation (Middleton, WI) |
| (DE3) | |
| E. coli | Lutz, R. and Bujard, H, Nucleic Acids Research (1997) 25 1203-1210 |
| DH5αZ1 | |
| JCL260* | E. coli BW25113, ΔldhA-fnr::FRT, ΔadhE::FRT, Δfrd::FRT, ΔpflB::FRT, |
| | Δpta::FRT, F′ (lacIq+) |

*These strains are described in PCT/US2008/053514

TABLE 4
Plasmids disclosed herein

| | | SEQ ID | |
|---|---|---|---|
| GEVO No. | FIG. | NO | Genotype or Reference |
| pKD13 | n/a | | Datsenko, K and Wanner, B. PNAS 2000, 97: 6640-5 |
| pKD46 | n/a | | Datsenko, K and Wanner, B. PNAS 2000, 97: 6640-5 |
| pSA55* | n/a | | pLlacO1::Ll_kivd1::ADH2, ColE1, Amp |
| pSA69* | n/a | | pLlacO1::Bs_alsS1::Ec_ilvC::Ec_ilvD, p15A, Kan |
| pET22b(+) | n/a | | Novagen, Gibbstown, NJ |
| pET22b[ilvCco] | n/a | | Novagen, Gibbstown, NJ |
| pGV1102 | | 101 | P_{TEF1}-HA-tag-MCS-T_{CYC1}, URA3,2-micron, bla, pUC-ori |
| pGV1323 | | 102 | |
| pGV1485 | | 103 | PLlacO1::Ll_kivd1::ADH2, pSC101, Km |
| pGV1490 | | 104 | pLtetO1:: p15A, Cm |
| pGV1527 | | | PLtetO1::Ll_kivd1_coEc::S. cerevisiae ADH2 ColE1, bla |
| pGV1572 | | 105 | PLlacO1::empty, p15A, Cm^{R} |
| pGV1573 | | 106 | PLlacO1::GDP1, p15A, Cm^{R} |
| pGV1575 | | 107 | PLlacO1::gapC, p15A, Cm^{R} |
| pGV1609 | | 108 | PLlacO1::Bs_alsS1::ilvC::Ec_ilvD, p15A, Cm |
| pGV1631 | | | PLlacO1::Ll_kivd1, ColE1, Amp |
| pGV1655 | | 109 | PLlacO1::Ll_kivd1::Ec_ilvD_coEc,, pSC101, Km |
| pGV1661 | | 110 | pLtetO1::maeB::ppc::mdh, p15A, Cm |
| pGV1662 | | | |
| pGV1685 | | 111 | PLtetO1::pntAB, p15A, Cm |
| pGV1698 | | 112 | PLlacO1::Bs_alsS1::ilvC, bla, ColE1 ORI |
| pGV1705-A | | | PLlacO1::Ec_yqhD bla, ColE1 ORI |
| pGV1711 | | 113 | PLlacO1::(no ORF) bla, ColE1 ORI |
| pGV1716 | | 114 | PLlacO1::Bs_alsS1::Saccharomyces cerevisiae |
| | | | ADH2::ilvC bla, ColE1 ORI |
| pGV1720 | | 115 | pLlacO1::empty, pSC101, Km |
| pGV1730 | | 116 | P_{CUP1}-Bs_alsS2-PDC1 3′ region-PDC1 5′ region, TRP1, |
| | | | bla, pUC ori |
| pGV1745 | | 117 | pLlacO1::pntAB, pSC101, Km |
| pGV1748 | | | PLlacO1::Bs_alsS1::Ec_fucO::Ec_ilvC_coEc bla, ColE1 |
| | | | ORI |
| pGV1748-A | | | PLlacO1::Ec_fucO:: bla, ColE1 ORI |
| pGV1749 | | | PLlacO1:: Bs_alsS1::Dm_ADH: Ec_ilvC_coEc bla, |
| | | | ColE1 ORI |
| pGV1749-A | | | PLlacO1::Dm_ADH:: bla, ColE1 ORI |
| pGV1772 | | | pLtetO1::maeB::pck::mdh, p15A, Cm |
| pGV1777 | | 118 | PLlacO1::Ec_ilvC_coEc, bla, ColE1 ORI |
| pGV1778 | | | PLlacO1:: Bs_alsS1::Kp_dhaT::Ec_ilvC_coEc bla, ColE1 |
| | | | ORI |
| pGV1778-A | | | PLlacO1::Kp_dhaT::bla, ColE1 ORI |
| pGV1824 | | | P_{TEF1}::Ec_ilvC_coSc:T_{CYC1}, pUC ORI, URA3, 2μ ORI, bla |
| pGV1914 | | 119 | P_{TEF1}:Ll_kivd2: P_{TDH3}:Dm_ADH PDC6 5′,3′ targeting |
| | | | homology URA3 pUC ori bla(ampR) |
| pGV1925 | | | pLlacO1::Ec_fucO ::Ec_ilvC_coEc::bla, ColE1 ORI |
| pGV1927 | | | pLlacO1::Ec_fucO::Ec_ilvC_coEc^{S78D} bla, ColE1 ORI |
| pGV1936 | | 120 | P_{TEF1}:Sc_ILV3ΔN P_{TDH3}:Ec_ilvC_coSc^{Q110V} PDC5 5′,3′ |
| | | | targeting homology LEU2 |
| pGV1938 | | | pLlac01::ilvC_coS78D bla, ColE1 ORI |
| pGV1939 | | | pLlacO1::E. coli fucO bla, ColE1 ORI |
| pGV1975 | | | pLlacO1::Ec_fucO::Ec_ilvC_coEc^{6E6} bla, ColE1 ORI |
| pGV1976 | | | pLlacO1::Ec_fucO::Ec_ilvC_coEc^{2H10} bla, ColE1 ORI |
| pGV1994 | | | P_{TEF1}::Ec_ilvC_coSc^{6E6}:T_{CYC1}, bla, pUC ORI, URA3, 2μ |
| | | | ORI |
| pGV2020 | | 121 | P_{Sc}_{_{—}}_{TEF1}, P_{Sc}_{_{—}}_{TPI1}, P_{Sc}_{_{—}}_{TPI1}G418^{R}, AP^{r}, 2μ - Vector Control |
| pGV2082 | | 122 | P_{TEF1}-Ll_ilvD_coSc-P_{TDH3}-Ec_ilvC_coSc^{Q110V}-P_{TPI1}- |
| | | | G418R-P_{PGK1}-Ll_kivd2_coEc-PDC1-3′region-P_{ENO2}- |
| | | | Dm_ADH 2μ bla, pUC-ori |
| pGV2193 | | | P_{TEF1}::Ec_ilvC_coSc^{P2D1-his6}:T_{CYC1}, bla, pUC ORI, URA3, |
| | | | 2μ ORI |
| pGV2227 | | 123 | P_{TEF1}-Ll_ilvD_coSc-P_{TDH3}-Ec_ilvC_coSc^{Q110V}-P_{TPI1}- |
| | | | G418R-P_{PGK1}-Ll_kivd2_coEc-PDC1-3′region-P_{ENO2}- |
| | | | Ll_adhA 2μ bla, pUC-ori |
| pGV2238 | | | P_{TEF1}::Ec_ilvC_coSc^{P2D1-A1-his6}:T_{CYC1}, bla, pUC ORI, |
| | | | URA3, 2μ ORI. |
| pGV2241 | | 124 | P_{TEF1}::Ec_ilvC_coSc^{6E6-his6}:T_{CYC1}, bla, pUC ORI, URA3, |
| | | | 2μ ORI. |
| pGV2242 | | 125 | P_{TEF1}-Ll_ilvD_coSc-P_{TDH3}-Ec_ilvC_coSc^{P2D1}-P_{TPI1}- |
| | | | G418R-P_{PGK1}-Ll_kivd2_coEc-PDC1-3′region-P_{ENO2}- |
| | | | Ll_adhA 2μ bla, pUC-ori |
| pGV6000 | | | P_{TEF1}:NADH kinase P_{TDH3}:NADP⁺ phosphatase HPH |
| pGV6001 | | | P_{TDH3}:Kl_GDP1 HPH |
| pGV6002 | | | P_{TEF1}:ess:pntA P_{TDH3}:ess:pntB HPH |
| pGV6003 | | | P_{TEF1}:mts:pntA P_{TDH3}:mts:pntB HPH |
| pGV6004 | | | P_{ADH1}:PYC1 P_{TEF1}:MDH2 P_{TDH3}:maeB HPH |

*These plasmids are described in PCT/US2008/053514

TABLE 5
Amino acid and nucleotide sequences of enzymes and
genes disclosed herein

| | | | Corresponding |
|---|---|---|---|
| | | Gene | Protein |
| Enz. | Source | (SEQ ID NO) | (SEQ ID NO) |
| pntA | E. coli | E. coli pntA | E. coli PntA |
| | | (SEQ ID NO: 1) | (SEQ ID NO: 2) |
| pntB | E. coli | E. coli pntB | E. coli PntB |
| | | (SEQ ID NO: 3) | (SEQ ID NO: 4) |
| ALS | B. subtilis | Bs_alsS1 | Bs_AlsS1 |
| | | (SEQ ID NO: 5) | (SEQ ID NO: 7) |
| | | Bs_alsS1_coSc | |
| | | (SEQ ID NO: 6) | |
| | | Bs_alsS2 | Bs_AlsS2 |
| | | (SEQ ID NO: 8) | (SEQ ID NO: 9) |
| KARI | E. coli | Ec_ilvC | Ec_IlvC |
| | | (SEQ ID NO: 10) | (SEQ ID NO: 13) |
| | | Ec_ilvC_coEc | |
| | | (SEQ ID NO: 11) | |
| | | Ec_ilvC_coSc | |
| | | (SEQ ID NO: 12) | |
| | | Ec_ilvC_coEc^{his6} | Ec_IlvC^{his6} |
| | | (SEQ ID NO: 14) | (SEQ ID NO: 15) |
| | | Ec_ilvC_coEc^{S78D-his6} | Ec_IlvC^{S78D-his6} |
| | | (SEQ ID NO: 16) | (SEQ ID NO: 17) |
| | | Ec_ilvC_coEc^{S78D} | Ec_IlvC^{S78D} |
| | | (SEQ ID NO: 18) | (SEQ ID NO: 19) |
| | | Ec_ilvC_coEc^{Q110A-his6} | Ec_IlvC^{Q110A-his6} |
| | | (SEQ ID NO: 20) | (SEQ ID NO: 21) |
| | | Ec_ilvC_coEc^{Q110V-his6} | Ec_IlvC^{Q110V-his6} |
| | | (SEQ ID NO: 22) | (SEQ ID NO: 23) |
| | | Ec_ilvC_coSc^{Q110V} | Ec_IlvC^{Q110V} |
| | | (SEQ ID NO: 24) | (SEQ ID NO: 25) |
| | | Ec_ilvC_coEc^{B8-his6} | Ec_IlvC^{B8-his6} |
| | | (SEQ ID NO: 26) | (SEQ ID NO: 27) |
| | | Ec_ilvC_coEc^{B8A/1S-his6} | Ec_IlvC^{B8A/1S-his6} |
| | | (SEQ ID NO: 28) | (SEQ ID NO: 29) |
| | | Ec_ilvC_coEc^{2H10-his6} | Ec_IlvC^{2H10-his6} |
| | | (SEQ ID NO: 30) | (SEQ ID NO: 31) |
| | | Ec_ilvC_coEc^{6E6-his6} | Ec_IlvC^{6E6-his6} |
| | | (SEQ ID NO: 32) | (SEQ ID NO: 34) |
| | | Ec_ilvC_coSc^{6E6-his6} | |
| | | (SEQ ID NO: 33) | |
| | | Ec_ilvC_coSc^{6E6} | Ec_IlvC^{6E6} |
| | | (SEQ ID NO: 35) | (SEQ ID NO: 36) |
| | | Ec_ilvC_coSc^{P2D1-his6} | Ec_IlvC^{P2D1-his6} |
| | | (SEQ ID NO: 37) | (SEQ ID NO: 38) |
| | | Ec_ilvC_coSc^{P2D1} | Ec_IlvC^{P2D1} |
| | | (SEQ ID NO: 39) | (SEQ ID NO: 40) |
| | | Ec_ilvC_coSc^{P2D1-A1-his6} | Ec_IlvC^{P2D1-A1-his6} |
| | | (SEQ ID NO: 41) | (SEQ ID NO: 42) |
| | | Ec_ilvC_coSc^{P2D1-A1} | Ec_IlvC^{P2D1-A1} |
| | | (SEQ ID NO: 43) | (SEQ ID NO: 44) |
| KIVD | L. lactis | Ll_Kivd1 | Ll_kivd1 |
| | | (SEQ ID NO: 45) | (SEQ ID NO: 47) |
| | | Ll_Kivd1_coEc | |
| | | (SEQ ID NO: 46) | |
| | | Ll_kivd2_coEc | Ll_Kivd2 |
| | | (SEQ ID NO: 48) | (SEQ ID NO: 49) |
| DHAD | E. coli | Ec_ilvD | Ec_IlvD |
| | | (SEQ ID NO: 50) | (SEQ ID NO: 52) |
| | | Ec_ilvD_coEc | |
| | | (SEQ ID NO: 51) | |
| | L. lactis | Ll_ilvD_coSc | Ll_IlvD |
| | | (SEQ ID NO: 54) | (SEQ ID NO: 55) |
| | S. cerevisiae | Sc_ILV3 | Sc_Ilv3 |
| | | (SEQ ID NO: 56) | (SEQ ID NO: 57) |
| | | Sc_ILV3ΔN | Sc_Ilv3ΔN |
| | | (SEQ ID NO: 58) | (SEQ ID NO: 59) |
| ADH | D. melanogaster | Dm_ADH | Dm_Adh |
| | | (SEQ ID NO: 60) | (SEQ ID NO: 61) |
| | K. pneumoniae | Kp_dhaT | Kp_DhaT |
| | | (SEQ ID NO: 62) | (SEQ ID NO: 63) |
| | E. coli | Ec_fucO | Ec_FucO |
| | | (SEQ ID NO: 64) | (SEQ ID NO: 65) |
| | L. lactis | Ll_adhA | Ll_AdhA |
| | | (SEQ ID NO: 66) | (SEQ ID NO: 67) |
| | E. coli | Ec_yqhD | Ec_YqhD |
| | | (SEQ ID NO: 68) | (SEQ ID NO: 69) |

TABLE 6
Primers sequences disclosed herein

| No. (SEQ ID NO) | Sequence (listed as 5′ to 3′) |
|---|---|
| XX1 | CGCACCGGTTTTCTCCTCTTTAATGAATTCGGTCAGTGCGTCCTGC |
| (SEQ ID NO: 201) | |
| XX2 | GCGGCCGCCCTAGGGCGTTCGGCTGCGGCGAGCGGT |
| (SEQ ID NO: 202) | |
| XX3 | CGCGAATTCGGATCCGAGGAGAAAATAGTTATGAACAACTTTAATCTGCACAC |
| (SEQ ID NO: 203) | CCC |
| XX4 | GCGCCTAGGGCGGCCGCTTAGCGGGCGGCTTCGTATATACGG |
| (SEQ ID NO: 204) | |
| 50 | GCAGTTTCACCTTCTACATAATCACGACCGTAGTAGGTATCATTCCGGGGATC |
| (SEQ ID NO: 205) | CGTCGACC |
| 73 | CTGGCTTAAGTACCGGGTTAGTTAACTTAAGGAGAATGACGTGTAGGCTGGA |
| (SEQ ID NO: 206) | GCTGCTTC |
| 74 | CTCAAACTCATTCCAGGAACGACCATCACGGGTAATCATCATTCCGGGGATCC |
| (SEQ ID NO: 207) | GTCGACC |
| 116 | CAGCGTTCGCTTTATATCCCTTACGCTGGCCCTGTACTGCTGGAAGTGTAGG |
| (SEQ ID NO: 208) | CTGGAGCTGCTTC |
| 117 | TTCGGCTTGCCAGAAATTATCGTCAATGGCCTGTTGCAGGGCTTCATTCCGG |
| (SEQ ID NO: 209) | GGATCCGTCGACC |
| 350 | CTTAAATTCTACTTTTATAGTTAGTC |
| (SEQ ID NO: 210) | |
| 474 | CAAAGCTGCGGATGATGACGAGATTACTGCTGCTGTGCAGACTGAATTCCGG |
| (SEQ ID NO: 211) | GGATCCGTCGACC |
| 772 | AGGAAGGAGCACAGACTTAG |
| (SEQ ID NO: 212) | |
| 868 | CACAACATCACGAGGAATCACCATGGCTAACTACTTCAATACACGTGTAGGCT |
| (SEQ ID NO: 213) | GGAGCTGCTTC |
| 869 | CTTAACCCGCAACAGCAATACGTTTCATATCTGTCATATAGCCGCATTCCGGG |
| (SEQ ID NO: 214) | GATCCGTCGACC |
| 1030 | GTCGGTGAACGCTCTCCTGAGTAGGGTGTAGGCTGGAGCTGCTTC |
| (SEQ ID NO: 215) | |
| 1031 | GAAGCAGCTCCAGCCTACACCCTACTCAGGAGAGCGTTCACCGAC |
| (SEQ ID NO: 216) | |
| 1032 | CACAACATCACGAGGAATCACCATGGCTAACTACTTCAATACACCACGAGGCC |
| (SEQ ID NO: 217) | CTTTCGTCTTCACCTC |
| 1155 | CCCAACCCGCATTCTGTTTGGTAAAGGCGCAATCGCTGGTTTACGGTGTAGG |
| (SEQ ID NO: 218) | CTGGAGCTGCTTC |
| 1156 | CAATCGCGGCGTCAATACGCTCATCATCGGAACCTTCAGTGATGTATTCCGG |
| (SEQ ID NO: 219) | GGATCCGTCGACC |
| 1187 | CGGATAAAGTTCGTGAGATTGCCGCAAAACTGGGGCGTCATGTGGGTGTAGG |
| (SEQ ID NO: 220) | CTGGAGCTGCTTC |
| 1188 | CAGACATCAAGTAACCTTTATCGCGCAGCAGATTAACCGCTTCGCATTCCGGG |
| (SEQ ID NO: 221) | GATCCGTCGACC |
| 1191 | GGCACTCACGTTGGGCTGAGACACAAGCACACATTCCTCTGCACGGTGTAGG |
| (SEQ ID NO: 222) | CTGGAGCTGCTTC |
| 1192 | GCACCAGAAACCATAACTACAACGTCACCTTTGTGTGCCAGACCGATTCCGG |
| (SEQ ID NO: 223) | GGATCCGTCGACC |
| 1205 | GTTATCTAGTTGTGCAAAACATGCTAATGTAGCCACCAAATCCACGAGGCCCT |
| (SEQ ID NO: 224) | TTCGTCTTCACCTC |
| 1218 | GCTCACTCAAAGGCGGTAATACGTGTAGGCTGGAGCTGCTTC |
| (SEQ ID NO: 225) | |
| 1219 | GAAGCAGCTCCAGCCTACACGTATTACCGCCTTTGAGTGAGC |
| (SEQ ID NO: 226) | |
| 1220 | CGTAGAATCACCAGACCAGC |
| (SEQ ID NO: 227) | |
| 1296 | TTTTGTCGACGGATCCAGGAGACAACATTATGTCTATTCCAGAAACTCAAAAA |
| (SEQ ID NO: 228) | GCG |
| 1297 | TTTTGTCGACGCGGCCGCTTATTTAGAGGTGTCCACCACGTAACGG |
| (SEQ ID NO: 229) | |
| 1321 | AATCATATCGAACACGATGC |
| (SEQ ID NO: 230) | |
| 1322 | TCAGAAAGGATCTTCTGCTC |
| (SEQ ID NO: 231) | |
| 1323 | ATCGATATCGTGAAATACGC |
| (SEQ ID NO: 232) | |
| 1324 | AGCTGGTCTGGTGATTCTAC |
| (SEQ ID NO: 233) | |
| 1341 | TGCTGAAAGAGAAATTGTCC |
| (SEQ ID NO: 234) | |
| 1342 | TTTCTTGTTCGAAGTCCAAG |
| (SEQ ID NO: 235) | |
| 1364 | TTTTGCGGCCGCTTAGATGCCGGAGTCCCAGTGCTTG |
| (SEQ ID NO: 236) | |
| 1365 | AGTTGTTGACGCAGGTTCAGAG |
| (SEQ ID NO: 237) | |
| 1436 | AAATGACGACGAGCCTGAAG |
| (SEQ ID NO: 238) | |
| 1437 | GACCTGACCATTTGATGGAG |
| (SEQ ID NO: 239) | |
| 1439 | CAATTGGCGAAGCAGAACAAG |
| (SEQ ID NO: 240) | |
| 1469 | TTTTAGATCTAGGAGATACCGGTATGTCGTTTACTTTGACCAACAAG |
| (SEQ ID NO: 241) | |
| 1440 | ATCGTACATCTTCCAAGCATC |
| (SEQ ID NO: 242) | |
| 1441 | AATCGGAACCCTAAAGGGAG |
| (SEQ ID NO: 243) | |
| 1442 | AATGGGCAAGCTGTTTGCTG |
| (SEQ ID NO: 244) | |
| 1443 | TGCAGATGCAGATGTGAGAC |
| (SEQ ID NO: 245) | |
| 1470 | TTTTGGATCCAGGAAATAGATCTATGATGGCTAACAGAATGATTCTGAACG |
| (SEQ ID NO: 246) | |
| 1471 | TTTTGCGGCCGCTTACCAGGCGGTATGGTAAAGCTC |
| (SEQ ID NO: 247) | |
| 1479 | CCGATAGGCTTCCGCCATCGTCGGGTAGTTAAAGGTGGTGTTGAGTGTAGGC |
| (SEQ ID NO: 248) | TGGAGCTGCTTC |
| 1485 | GCCTTTATTGTACGCTTTTTACTGTACGATTTCAGTCAAATCTAACACGAGGCC |
| (SEQ ID NO: 249) | CTTTCGTCTTCACCTC |
| 1486 | AAGTACGCAGTAAATAAAAAATCCACTTAAGAAGGTAGGTGTTACATTCCGGG |
| (SEQ ID NO: 250) | GATCCGTCGACC |
| 1526 | TCGACGAGGAGACAACATTGTGTAGGCTGGAGCTGCTTC |
| (SEQ ID NO: 251) | |
| 1527 | GAAGCAGCTCCAGCCTACACAATGTTGTCTCCTCGTCGA |
| (SEQ ID NO: 252) | |
| 1539 | CCATTCTGTTGCTTTTATGTATAAGAACAGGTAAGCCCTACCATGGAGAATTGT |
| (SEQ ID NO: 253) | GAGCGGATAAC |
| 1561 | GCAATCCTGAAAGCTCTGTAACATTCCGGGGATCCGTCGACC |
| (SEQ ID NO: 254) | |
| 1562 | GGTCGACGGATCCCCGGAATGTTACAGAGCTTTCAGGATTGC |
| (SEQ ID NO: 255) | |
| 1563 | CAAATCGGCGGTAACGAAAGAGGATAAACCGTGTCCCGTATTATTCACGAGG |
| (SEQ ID NO: 256) | CCCTTTCGTCTTCACCTC |
| 1566 | TCCCACCCAATCAAGGCCAACG |
| (SEQ ID NO: 257) | |
| 1567 | TCCACCTGGTGCCAATGAACCG |
| (SEQ ID NO: 258) | |
| 1587 | CGGCTGCCAGAACTCTACTAACTG |
| (SEQ ID NO: 259) | |
| 1588 | GCGACGTCTACTGGCAGGTTAAT |
| (SEQ ID NO: 260) | |
| 1595 | CAACCTGGTGATTTGGGGAAG |
| (SEQ ID NO: 261) | |
| 1597 | GAATGATGGCAGATTGGGCA |
| (SEQ ID NO: 262) | |
| 1598 | TATTGTGGGGCTGTCTCGAATG |
| (SEQ ID NO: 263) | |
| 1624 | CCCTCATGTTGTCTAACGG |
| (SEQ ID NO: 264) | |
| 1633 | TCCGTCACTGGATTCAATGCCATC |
| (SEQ ID NO: 265) | |
| 1634 | TTCGCCAGGGAGCTGGTGAA |
| (SEQ ID NO: 266) | |
| 1798 | GCAAATTAAAGCCTTCGAGCG |
| (SEQ ID NO: 267) | |
| 1926 | TTTTTGTCGACGGATCCAGTTTATCATTATCAATACTCG |
| (SEQ ID NO: 268) | |
| 1927 | TTTTGCGGCCGCAGATCTCTCGAGTCGAAACTAAGTTCTGGTGTT |
| (SEQ ID NO: 269) | |
| 2091 | CTTTTCTTCCCTTGTCTCAATC |
| (SEQ ID NO: 270) | |
| 2352 | GACTCGACCTAGGTTATTTAGTAAAATCAATGACCATTC |
| (SEQ ID NO: 271) | |
| 2353 | CTAAATAACCTAGGTCGAGTCATGTAATTAGTTATGTC |
| (SEQ ID NO: 272) | |
| KARIpETfor | ATTCATATGGCGAATTATTTCAACACTCTG |
| (SEQ ID NO: 273) | |
| KARIpETrev | TAATCTCGAGGCCAGCCACCGCGATGCG |
| (SEQ ID NO: 274) | |
| pETup | ATGCGTCCGGCGTAGA |
| (SEQ ID NO: 275) | |
| seq_ilvC_pGV | GCGGCCGCGTCGACGAGGAGACAACATTATGGCGA |
| (SEQ ID NO: 276) | |
| pGV1994ep_for | CGGTCTTCAATTTCTCAAGTTTCAGTTTCATTTTTCTTGTTCTATTACAAC |
| (SEQ ID NO: 277) | |
| pGV1994ep_rev | CTAACTCCTTCCTTTTCGGTTAGAGCGGATGTGGG |
| (SEQ ID NO: 278) | |
| Not_in_for | CCTCTA GAAATAATTTGCGGCCGCGTTAAGAAGGAGATATACATATG |
| (SEQ ID NO: 279) | |
| AvrII_in_rev | CCGAACGCCCTAGGTCAGTGGTGGTGGTGGTGGTGCTCGAG |
| (SEQ ID NO: 280) | |
| R68DK69Lfor | TAGCTATGCGCTGGACCTGGAGGCTATC |
| (SEQ ID NO: 281) | |
| R68DK69Lrev | GATAGCCTCCAGGTCCAGCGCATAGCTA |
| (SEQ ID NO: 282) | |
| K75VR76Dfor | AGGCTATCGCGGAAGTTGACGCTAGCTG |
| (SEQ ID NO: 283) | |
| K75VR76Drev | CAGCTAGCGTCAACTTCCGCGATAGCCT |
| (SEQ ID NO: 284) | |
| R69NNKfor | TAGCTATGCGCTGCGCNNKGAGGCTATC |
| (SEQ ID NO: 285) | |
| R69NNKrev | GATAGCCTCMNNGCGCAGCGCATAGCTA |
| (SEQ ID NO: 286) | |
| K75NNKfor | AGGCTATCGCGGAANNKCGTGCTAGCTG |
| (SEQ ID NO: 287) | |
| K75NNKrev | CAGCTAGCACGMNNTTCCGCGATAGCCT |
| (SEQ ID NO: 288) | |
| R76NNKfor | AGGCTATCGCGGAAAAANNKGCTAGCTGGC |
| (SEQ ID NO: 289) | |
| R76NNKrev | GCCAGCTAGCMNNTTTTTCCGCGATAGCCT |
| (SEQ ID NO: 290) | |
| R68NNK_for | TAGCTATGCGCTGNNKAAGGAGGCTATC |
| (SEQ ID NO: 291) | |
| R68NNK_rev | GATAGCCTCCTTMNNCAGCGCATAGCTA |
| (SEQ ID NO: 292) | |
| S78NNK_for | GCGGAAAAACGTGCTNNKTGGCGCAAGGCTACT |
| (SEQ ID NO: 293) | |
| 578NNK_rev | AGTAGCCTTGCGCCAMNNAGCACGTTTTTCCGC |
| (SEQ ID NO: 294) | |
| A71NNK_for | GCGCTGCGCAAGGAGNNKATCGCGGAAAAAC |
| (SEQ ID NO: 295) | |
| A71NNK_rev | GTTTTTCCGCGATMNNCTCCTTGCGCAGCGC |
| (SEQ ID NO: 296) | |
| Gln110NNK_for | CTGACCCCAGATAAANNKCATAGCGACGTTG |
| (SEQ ID NO: 297) | |
| Gln110NNK_rev | CAACGTCGCTATGMNNTTTATCTGGGGTCAG |
| (SEQ ID NO: 298) | |
| seq_ilvC_pGV | GCGGCCGCGTCGACGAGGAGACAACATTATGGCGA |
| (SEQ ID NO: 299) | |
| Q110Qfor | GACCCCAGATAAACAACATAGCGACGTTGTT |
| (SEQ ID NO: 300) | |
| Q110Qrev | AACAACGTCGCTATGTTGTTTATCTGGGGTC |
| (SEQ ID NO: 301) | |
| Q110Afor | GACCCCAGATAAAGCACATAGCGACGTTGTT |
| (SEQ ID NO: 302) | |
| Q110Arev | AACAACGTCGCTATGTGCTTTATCTGGGGTC |
| (SEQ ID NO: 303) | |
| Q110Vfor | GACCCCAGATAAAGTACATAGCGACGTTGTT |
| (SEQ ID NO: 304) | |
| Q110Vrev | AACAACGTCGCTATGTACTTTATCTGGGGTC |
| (SEQ ID NO: 305) | |
| R68A71recombfor | GCTATGCGCTGCKAAAGGAGDCAATCGCGG |
| (SEQ ID NO: 306) | |
| R68A71recombrev | CCGCGATTGHCTCCTTTMGCAGCGCATAGC |
| (SEQ ID NO: 307) | |
| R76S78recombfor | GAAAAACGTGCTAGCTGGCGCAAGGCTACT |
| (SEQ ID NO: 308) | |
| R76S78recombrev | AGTAGCCTTGCGCCAGCTAGCACGTTTTTC |
| (SEQ ID NO: 309) | |
| G76S78recombfor | GAAAAAGGTGCTAGCTGGCGCAAGGCTACT |
| (SEQ ID NO: 310) | |
| G76S78recombrev | AGTAGCCTTGCGCCAGCTAGCACCTTTTTC |
| (SEQ ID NO: 311) | |
| S76S78recombfor | GAAAAAAGTGCTAGCTGGCGCAAGGCTACT |
| (SEQ ID NO: 312) | |
| S76S78recombrev | AGTAGCCTTGCGCCAGCTAGCACTTTTTTC |
| (SEQ ID NO: 313) | |
| T76S78recombfor | GAAAAAACTGCTAGCTGGCGCAAGGCTACT |
| (SEQ ID NO: 314) | |
| T76S78recombrev | AGTAGCCTTGCGCCAGCTAGCAGTTTTTTC |
| (SEQ ID NO: 315) | |
| D76S78recombfor | GAAAAAGATGCTAGCTGGCGCAAGGCTACT |
| (SEQ ID NO: 316) | |
| D76S78recombrev | AGTAGCCTTGCGCCAGCTAGCATCTTTTTC |
| (SEQ ID NO: 317) | |
| R76D78recombfor | GAAAAACGTGCTGACTGGCGCAAGGCTACT |
| (SEQ ID NO: 318) | |
| R76D78recombrev | AGTAGCCTTGCGCCAGTCAGCACGTTTTTC |
| (SEQ ID NO: 319) | |
| G76D78recombfor | GAAAAAGGTGCTGACTGGCGCAAGGCTACT |
| (SEQ ID NO: 320) | |
| G76D78recombrev | AGTAGCCTTGCGCCAGTCAGCACCTTTTTC |
| (SEQ ID NO: 321) | |
| S76D78recombfor | GAAAAAAGTGCTGACTGGCGCAAGGCTACT |
| (SEQ ID NO: 322) | |
| S76D78recombrev | AGTAGCCTTGCGCCAGTCAGCACTTTTTTC |
| (SEQ ID NO: 323) | |
| T76D78recombfor | GAAAAAACTGCTGACTGGCGCAAGGCTACT |
| (SEQ ID NO: 324) | |
| T76D78recombrev | AGTAGCCTTGCGCCAGTCAGCAGTTTTTTC |
| (SEQ ID NO: 325) | |
| D76D78recombfor | GAAAAAGATGCTGACTGGCGCAAGGCTACT |
| (SEQ ID NO: 326) | |
| D76D78recombrev | AGTAGCCTTGCGCCAGTCAGCATCTTTTTC |
| (SEQ ID NO: 327) | |
| 1994hisrev | TGACTCGAGCGGCCGCGGATCCTTAGTGGTGGTGGTGGTGGTGTCCTGCCA |
| (SEQ ID NO: 328) | CTGCA |
| pGV1994ep_for | CGGTCTTCAATTTCTCAAGTTTCAGTTTCATTTTTCTTGTTCTATTACAAC |
| (SEQ ID NO: 329) | |
| pGV1994ep_rev | CTAACTCCTTCCTTTTCGGTTAGAGCGGATGTGGG |
| (SEQ ID NO: 330) | |

### Example 1

### Low-Level Anaerobic Production of Isobutanol

This example illustrates that a modified microorganism which is engineered to overexpress an isobutanol producing pathway produces a low amount of isobutanol under anaerobic conditions.

Overnight cultures of GEVO1859 were started from glycerol stocks stored at −80° C. of previously transformed strains. These cultures were started in 3 mL M9 minimal medium (Miller, J.H. A Short Course in Bacterial Genetics: A laboratory manual and handbook for Escherichia coli and related bacteria. 1992. Cold Spring Harbor Laboratory Press, Cold Spring Harbor, N.Y.), supplemented with 10 g/L yeast extract, 10 μM ferric citrate and trace metals, containing 8.5% glucose and the appropriate antibiotics in snap cap tubes about 14 h prior to the start of the fermentation. Isobutanol fermentations were then carried out in screw cap flasks containing 20 mL of the same medium that was inoculated with 0.2 mL of the overnight culture. The cells were incubated at 37° C./250 rpm until the strains had grown to an OD₆₀₀ of 0.6-0.8 and were then induced with Isopropyl β-D-1-thiogalactopyranoside at 1 mM final concentration.

Three hours after induction the cultures were either kept under the current conditions (micro-aerobic conditions) or shifted to anaerobic conditions by loosening the cap of the flasks and placing the flasks into to a Coy Laboratory Products Type B Vinyl anaerobic chamber (Coy Laboratory Products, Grass Lakes, Mich.) through an airlock in which the flasks were cycled three times with nitrogen and vacuum, and then filled with the a hydrogen gas mix (95% Nitrogen, 5% Hydrogen).

Once the flasks were inside the anaerobic chamber, the flasks were closed again and incubated without shaking at 30° C. The flasks in the anaerobic chamber were swirled twice a day. Samples (2 mL) were taken at the time of the shift and at 24 h and 48 h after inoculation, spun down at 22,000 g for 1 min to separate the cell pellet from the supernatant and stored frozen at −20° C. until analysis. The samples were analyzed using High performance liquid chromatography (HPLC) and gas chromatography (GC).

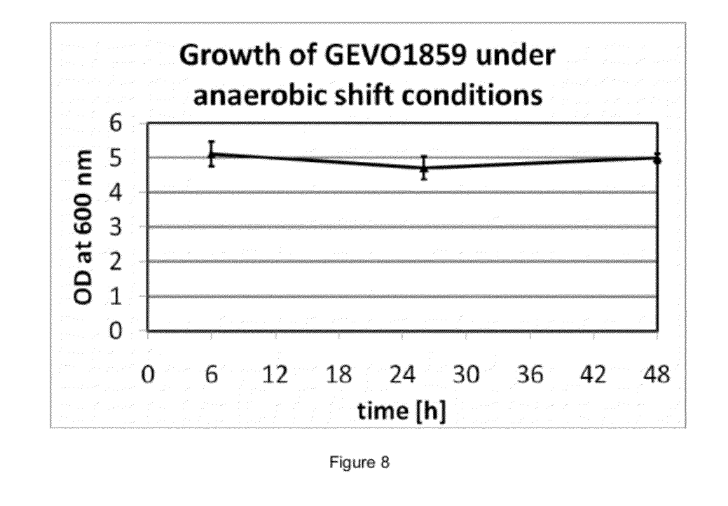

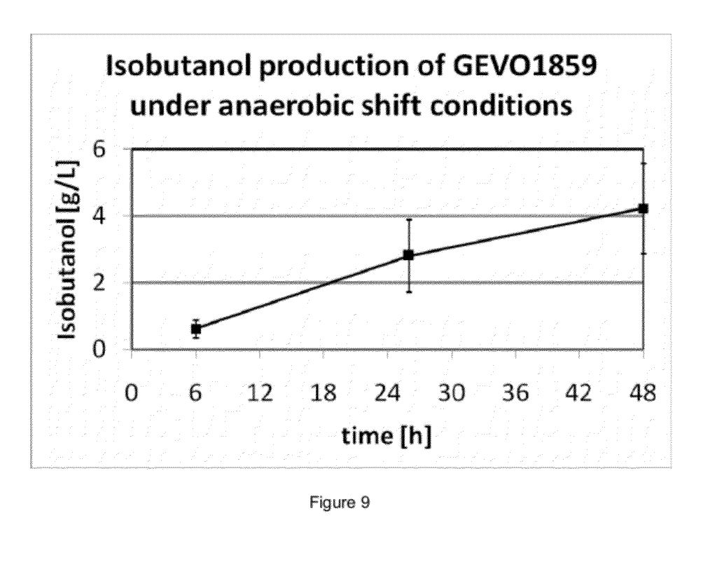

GEVO1859 was run in triplicate. Stable OD values can be observed for all strains under anaerobic shift conditions over the course of the fermentation (FIG. 8). The complete pathway integrant strain showed low-level anaerobic isobutanol production over the course of the fermentation (FIG. 9, Table 7).

TABLE 7
Volumetric productivity, specific productivity titer and yield
reached in an anaerobic fermentation for the tested strains
and plasmid systems

| | | | Specific | | | | | |
|---|---|---|---|---|---|---|---|---|
| | | | Productivity | | | | | |
| | Volumetric | | [g/L/ | | | | | |
| | Productivity | | h/ | | Titer | | Yield | |
| Samples | [g/L/h] | ± | OD] | ± | [g/L] | ± | [g/g] | ± |
| GEVO1859 | 0.088 | 0.028 | 0.019 | 0.005 | 4.22 | 1.35 | 0.140 | 0.029 |

In the period from 6 h to 48 h, i.e. under anaerobic conditions GEVO1859 demonstrated limited production of isobutanol (Table 8).

TABLE 8
Volumetric productivity, specific productivity titer and yield reached in the
period from 6 to 48 h for the tested strain

| | | Volumetric | | Specific | | | | | |
|---|---|---|---|---|---|---|---|---|---|
| | | Productivity | | Productivity | | Titer | | Yield | |
| Samples | Condition | [g/L/h] | ± | [g/L/h/OD] | ± | [g/L] | ± | [g/g] | ± |
| GEVO1859 | Micro- | 0.266 | 0.010 | 0.040 | 0.004 | 11.2 | 0.4 | 0.33 | 0.016 |
| | aerobic | | | | | | | | |
| GEVO1859 | Anaerobic | 0.086 | 0.026 | 0.019 | 0.005 | 3.60 | 1.1 | 0.14 | 0.032 |

### Example 2

### Determination of Transhydrogenase Activity

This example illustrates that an isobutanol producing microorganism which carries a plasmid for the expression of the E. coli PntAB transhydrogenase (SEQ ID NO: 2 and SEQ ID NO: 4) contains increased transhydrogenase activity.

A fermentation was performed with a strain expressing the tet repressor (GEVO1385) and carrying the plasmids pGV1655 (SEQ ID NO: 109) and pGV1698 (SEQ ID NO: 112) for expression of the isobutanol pathway. The E. coli transhydrogenase PntAB was expressed from a third plasmid pGV1685 (SEQ ID NO: 111), which contained the E. coli pntAB genes under control of the PLtet promoter. The appropriate empty vector control carries the plasmid pGV1490 (SEQ ID NO: 104).

GEVO1385 was transformed with pGV1698, pGV1655, and either pGV1685 or pGV1490. Transformed cells were plated on LB-plates containing the appropriate antibiotics and the plates were incubated overnight at 37° C. Overnight cultures were started in 3 mL EZ-Rich Defined Medium (Neidhardt, F. C., P. L. Bloch, and D. F. Smith. 1974, Culture medium for enterobacteria, J. Bacteriol. 119:736-47) containing 5% glucose and the appropriate antibiotics in snap cap tubes about 14 h prior to the start of the fermentation. Isobutanol fermentations were then carried out in EZ-Rich containing 5% glucose and the appropriate antibiotics. 250 mL screw cap flasks with 20 mL EZ-Rich containing 5% glucose and the appropriate antibiotics were inoculated with 1% of the grown overnight culture. The cells were incubated at 37° C./250 rpm until the strains were grown to an OD₆₀₀ of 0.6-0.8 and these strains were then induced with Isopropyl β-D-1-thiogalactopyranoside (IPTG (Gold BioTechnology, Inc, I2481C100) 1 mM) and anhydrotetracycline (aTc (Sigma, 37919-100 mg) 100 ng/mL). Samples were taken of the medium 48 h after inoculation. 15 mL of cell culture from each flask were centrifuged at 5,000×g for 5 min to separate the cell pellet from the supernatant. The cell pellets were stored frozen at −80° C. until analysis. The cultures grew to a comparable OD in this experiment.

To confirm that the transhydrogenase was actually expressed from the plasmids and to assess their enzymatic activity levels, enzyme assays were done with lysates prepared from the fermentation cultures. Frozen cell pellets were thawed on ice. The pellets were resuspended in 1.2 mL lysis buffer (50 mM potassium phosphate buffer at pH 7.5, MgCl₂ 2 mM). The suspensions were sonicated on ice for twice 2 min. The transhydrogenase enzyme assay was done in potassium phosphate buffer (50 mM pH 7.5, MgCl₂ 2 mM, 1 mM acetylpyridine-AD, 0.5 mM NADPH). The assay was run at 25° C. in a 96 well plate. Absorbance at 375 nm was followed in a kinetic assay format. To measure PntAB activity lysates were not cleared by centrifugation. The activity obtained for the samples featuring over-expressed E. coli pntAB show at least a 10 fold increase in transhydrogenase activity (Table 9).

TABLE 9
Shown are the enzymatic activities of the independent E. coli pntAB
overexpressing strains and the amount of isobutanol production that would
be supported by that activity calculated from Vₘₐₓ values obtained from
the enzyme assay

| | | | | | specific activity |
|---|---|---|---|---|---|
| | | | protein | | [u/mg |
| | average | stdev. | conc. | units in | (total cell |
| Samples | Vmax | Vmax | [mg/mL] | reaction | protein)] |
| pntAB-1 | 33.81 | 3.87 | 1.17 | 0.0010 | 0.1646 |
| pntAB-2 | 45.06 | 1.51 | 1.89 | 0.0013 | 0.1355 |
| empty vector-1 | 2.24 | 0.21 | 0.89 | 0.0001 | 0.0142 |
| empty vector-2 | −0.01 | 2.00 | 0.71 | 0.0000 | −0.0001 |

### Example 3

### Overexpression of pntAB Improves Isobutanol Fermentation Performance

This example illustrates that overexpression of a transhydrogenase, exemplified by the E. coli pntAB operon (SEQ ID NO: 1 and SEQ ID NO: 3) on a low copy plasmid improves isobutanol production under micro-aerobic conditions.

GEVO1748 was transformed with plasmids pGV1698 (SEQ ID NO: 112) and one of either pGV1720 (SEQ ID NO: 115) (control) or pGV1745 (SEQ ID NO: 117) (E. coli pntAB).

The aforementioned strains were plated on LB-plates containing the appropriate antibiotics and incubated overnight at 37° C. Overnight cultures were started in 3 mL EZ-Rich medium (Neidhardt, F. C., P. L. Bloch, and D. F. Smith. 1974. Culture medium for enterobacteria. J. Bacteriol. 119:736-47) containing 5% glucose and the appropriate antibiotics in snap cap tubes about 14 h prior to the start of the fermentation. Isobutanol fermentations were then carried out in EZ-Rich Medium containing 5% glucose and the appropriate antibiotics. 250 mL screw cap flasks with 20 mL EZ-Rich medium containing 5% glucose and the appropriate antibiotics were inoculated with 1% of the grown overnight culture. The cells were incubated at 37° C./250 rpm until they reached an OD₆₀₀ of 0.6-0.8 followed by induction with Isopropyl β-D-1-thiogalactopyranoside (IPTG, 1 mM) and anhydrotetracycline (aTc, 100 ng/mL). Samples (2 mL) were taken 24 h and 48 h post inoculation, centrifuged at 22,000×g for 1 min and stored frozen at −20° C. until via Gas Chromatography (GC) and High Performance Liquid Chromatography (HPLC). Fermentations were run with two biological replicates.

All cultures grew to an OD of 5.5 to 6.5. Volumetric productivity and titer were improved by 45%, specific productivity even by 51%. Yield was improved by 8% (Table 10).

TABLE 10
Overexpression of E. coli pntAB improves isobutanol fermentation
performance

| | Volumetric | | Specific | | | | | |
|---|---|---|---|---|---|---|---|---|
| | Productivity | | Productivity | | Titer | | Yield | |
| Strain | [g/L/h] | ± | [g/L/h/OD] | ± | [g/L] | ± | [g/g] | ± |
| GEVO1748 + | 0.205 | 0.001 | 0.035 | 0.001 | 9.86 | 0.04 | 0.311 | 0.001 |
| pGV1698 + | | | | | | | | |
| pGV1720 | | | | | | | | |
| GEVO1748 + | 0.298 | 0.006 | 0.053 | 0.003 | 14.29 | 0.28 | 0.337 | 0.001 |
| pGV1698 + | | | | | | | | |
| pGV1745 | | | | | | | | |

### Example 4

### Overexpression of pntAB Enables Anaerobic Isobutanol Production

This example illustrates that overexpression of a transhydrogenase, exemplified by the E. coli pntAB operon product (SEQ ID NO: 2 and SEQ ID NO: 4), improves anaerobic isobutanol production. This is surprising because it was previously not known that isobutanol could be produced anaerobically. In addition, this result was achieved without modifying the isobutanol biosynthetic pathway itself.

GEVO1748 was transformed with plasmids pGV1698 (SEQ ID NO: 112) and pGV1720 (SEQ ID NO: 115) (control) or pGV1745 (SEQ ID NO: 117) (E. coli pntAB).

Overnight cultures of the aforementioned strains were started from glycerol stocks stored at −80° C. of previously transformed strains. These cultures were started in 3 mL M9 minimal medium (Miller, J.H. A Short Course in Bacterial Genetics: A laboratory manual and handbook for Escherichia coli and related bacteria. 1992. Cold Spring Harbor Laboratory Press, Cold Spring Harbor, N.Y.), supplemented with 10 g/L yeast extract, 10 μM ferric citrate and trace metals, containing 8.5% glucose and the appropriate antibiotics in snap cap tubes about 14 h prior to the start of the fermentation. Isobutanol fermentations were then carried out in 250 mL screw cap flasks containing 20 mL of the same medium that was inoculated with 0.2 mL of the overnight culture. The cells were incubated at 37° C./250 rpm until the strains had grown to an OD₆₀₀ of 0.6-0.8 and were then induced with Isopropyl β-D-1-thiogalactopyranoside at 1 mM final concentration.

Three hours after induction the cultures were shifted to anaerobic fermentation conditions by loosening the cap of the flasks and placing the flasks into to a Coy Laboratory Products Type B Vinyl anaerobic chamber (Coy Laboratory Products, Grass Lakes, Mich.) through an airlock in which the flasks were cycled three times with nitrogen and vacuum, and then filled with the a hydrogen gas mix (95% Nitrogen, 5% Hydrogen). Once the flasks were inside the anaerobic chamber, the flasks were closed again and incubated without shaking at 30° C. Inside the chamber, an anaerobic atmosphere (<5 ppm oxygen) was maintained through the hydrogen gas mix (95% Nitrogen, 5% Hydrogen) reacting with a palladium catalyst to remove oxygen. The flasks in the anaerobic chamber were swirled twice a day. Samples (2 mL) were taken at the time of the shift and at 24 h and 48 h after inoculation, spun down at 22,000×g for 1 min to separate the cell pellet from the supernatant and stored frozen at −20° C. until analysis. The samples were analyzed using High performance liquid chromatography (HPLC) and gas chromatography (GC). All experiments for the E. coli pntAB-expressing strain were performed in duplicate while the control strain was only run in a single experiment.

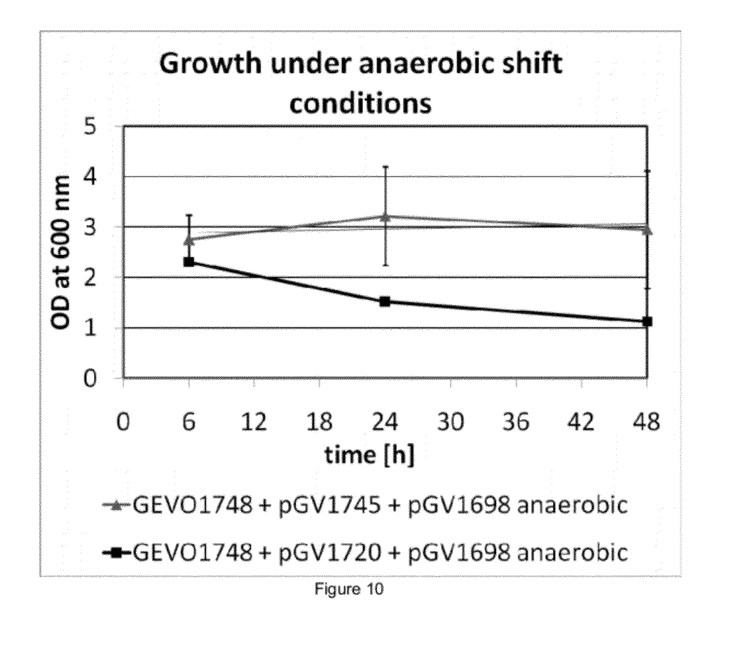

At the time of shifting the cultures to anaerobic conditions all samples had an OD₆₀₀ ranging between 2.3 and 3.3. All samples featuring an overexpressed E. coli pntAB operon (pGV1745) increased in OD₆₀₀ from 6 h to 24 h by 0.2-1.1, all samples lacking pntAB (pGV1720) decreased in OD₆₀₀ by 0.5-1.2 (FIG. 10), indicating that overexpression of E. coli pntAB is beneficial under anaerobic conditions.

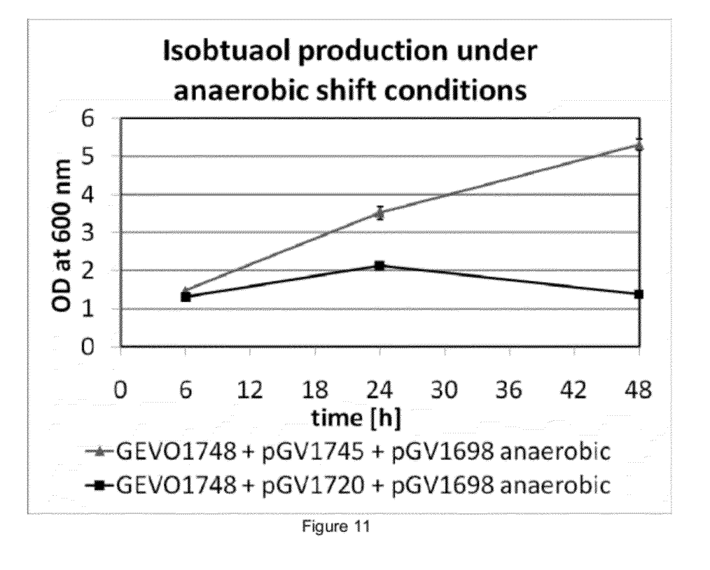

Furthermore, pntAB over-expression is beneficial for anaerobic isobutanol production. All samples featuring E. coli PntAB continued isobutanol production under anaerobic conditions until the fermentation was stopped at 48 hours whereas the samples lacking E. coli PntAB did not produce isobutanol between 24 and 48 hours (FIG. 11)

In the strain overexpressing E. coli pntAB, volumetric productivity and titer are increased 2.4-fold, specific productivity by 85% and yield by 9% (Table 11).

TABLE 11
Shown are the results for volumetric productivity, specific productivity titer
and yield reached in an anaerobic fermentation for the tested strains and plasmid
systems after 48 h

| | Volumetric | | Specific | | | | | |
|---|---|---|---|---|---|---|---|---|
| | Productivity | | Productivity | | Titer | | Yield | |
| Samples | [g/L/h] | ± | [g/L/h/OD] | ± | [g/L] | ± | [g/g] | ± |
| GEVO1748 + | 0.047 | | 0.022 | | 2.24 | | 0.279 | |
| pGV1720 + | | | | | | | | |
| pGV1698 | | | | | | | | |
| GEVO1748 + | 0.111 | 0.002 | 0.041 | 0.012 | 5.32 | 0.10 | 0.304 | 0.004 |
| pGV1745 + | | | | | | | | |
| pGV1698 | | | | | | | | |

In the period from 6 h to 48 h, (i.e. under anaerobic conditions), GEVO1748 transformed with plasmids pGV1698 and pGV1745 (carrying E. coli pntAB) demonstrated significantly higher productivity, titer, and yield of isobutanol compared to the control strain carrying pGV1720 (without E. coli pntAB) (Table 12).

TABLE 12
Shown are the results for volumetric productivity, specific productivity titer
and yield reached in the period from 6 to 48 h for the tested strains and plasmid
systems

| | Volumetric | | Specific | | | | | |
|---|---|---|---|---|---|---|---|---|
| | Productivity | | Productivity | | Titer | | Yield | |
| samples | [g/L/h] | ± | [g/L/h/OD] | ± | [g/L] | ± | [g/g] | ± |
| GEVO1748 + | 0.029 | | 0.014 | | 1.21 | | 0.171 | |
| pGV1720 + | | | | | | | | |
| pGV1698 | | | | | | | | |
| GEVO1748 + | 0.096 | 0.003 | 0.035 | 0.015 | 4.01 | 0.15 | 0.246 | 0.002 |
| pGV1745 + | | | | | | | | |
| pGV1698 | | | | | | | | |

### Example 5

### Chromosomal Integration of pntAB Improves Anaerobic Isobutanol Production

This example illustrates that overexpression of a transhydrogenase, exemplified by the E. coli pntAB operon product (SEQ ID NO: 2 and SEQ ID NO: 4), from the chromosome improves isobutanol production under anaerobic conditions compared to the case in which E. coli pntAB is expressed from a low copy plasmid. This strain reaches the same titer aerobically as anaerobically.

Overnight cultures of GEVO1846, GEVO1859, GEVO1886 were started from glycerol stocks stored at −80° C. of previously transformed strains. These cultures were started in 3 mL M9 minimal medium (Miller, J.H. A Short Course in Bacterial Genetics: A laboratory manual and handbook for Escherichia coli and related bacteria. 1992. Cold Spring Harbor Laboratory Press, Cold Spring Harbor, N.Y.), supplemented with 10 g/L yeast extract, 10 μM ferric citrate and trace metals, containing 8.5% glucose and the appropriate antibiotics in snap cap tubes about 14 h prior to the start of the fermentation. Isobutanol fermentations were then carried out in screw cap flasks containing 20 mL of the same medium that was inoculated with 0.2 mL of the overnight culture. The cells were incubated at 37° C./250 rpm until the strains had grown to an OD₆₀₀ of 0.6-0.8 and were then induced with Isopropyl β-D-1-thiogalactopyranoside at 1 mM final concentration.

Three hours after induction the cultures were either kept under the current conditions (micro-aerobic conditions) or shifted to anaerobic conditions by loosening the cap of the flasks and placing the flasks into to a Coy Laboratory Products Type B Vinyl anaerobic chamber (Coy Laboratory Products, Grass Lakes, Mich.) through an airlock in which the flasks were cycled three times with nitrogen and vacuum, and then filled with the a hydrogen gas mix (95% Nitrogen, 5% Hydrogen). Once the flasks were inside the anaerobic chamber, the flasks were closed again and incubated without shaking at 30° C. The flasks in the anaerobic chamber were swirled twice a day. Samples (2 mL) were taken at the time of the shift and at 24 h and 48 h after inoculation, spun down at 22,000×g for 1 min to separate the cell pellet from the supernatant and stored frozen at −20° C. until analysis. The samples were analyzed using High performance liquid chromatography (HPLC) and gas chromatography (GC). All experiments were performed in duplicate.

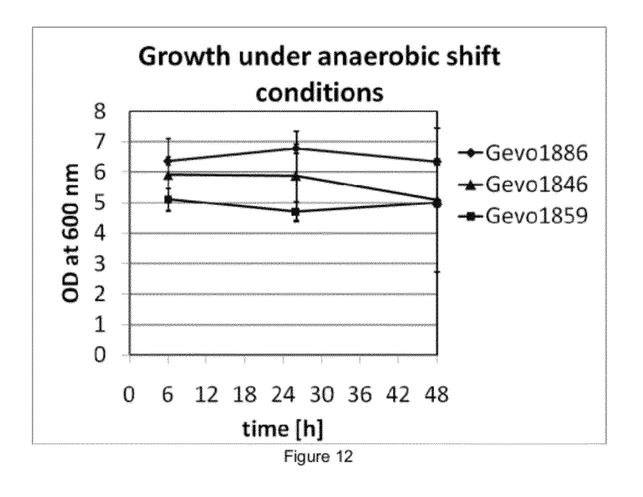

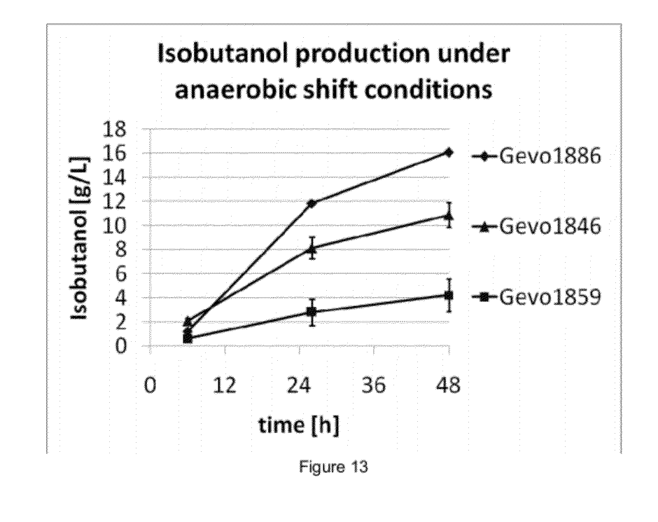

GEVO1886, GEVO1859 and GEVO1846 were run in parallel. Each strain was run in triplicate. Stable OD values can be observed for all strains under anaerobic shift conditions over the course of the fermentation (FIG. 12). The over-expression of E. coli pntAB in the complete pathway integrant strain again showed improvement for isobutanol production over the course of the fermentation (FIG. 13).

Compared to the complete pathway integrant strain without E. coli pntAB knock-in (GEVO1859), volumetric productivity and titer are increased 3.8-fold, specific productivity is increased 2.8-fold and the yield is 2.2-fold higher in GEVO1886. In addition, GEVO1886 shows superior performance compared to the plasmid system strain (GEVO1846) under anaerobic conditions. Volumetric productivity and titer are increased by 48%, specific productivity is increased by 18% and yield is 12% higher (Table 13).

TABLE 13
Shown are the results for volumetric productivity, specific
productivity titer and yield reached in an anaerobic fermentation
for the tested strains and plasmid systems

| | | | Specific | | | | | |
|---|---|---|---|---|---|---|---|---|
| | | | Productivity | | | | | |
| | Volumetric | | [g/L/ | | | | | |
| | Productivity | | h/ | | Titer | | Yield | |
| Samples | [g/L/h] | ± | OD] | ± | [g/L] | ± | [g/g] | ± |
| GEVO1886 | 0.335 | 0.002 | 0.053 | 0.001 | 16.08 | 0.08 | 0.307 | 0.004 |
| GEVO1859 | 0.088 | 0.028 | 0.019 | 0.005 | 4.22 | 1.35 | 0.140 | 0.029 |
| GEVO1846 | 0.227 | 0.021 | 0.045 | 0.005 | 10.88 | 1.01 | 0.274 | 0.003 |

The performance numbers in the period from 6 to 48 demonstrate that most of isobutanol production occurred under anaerobic conditions. Highest values for yield and specific productivity were reached by the strain featuring the complete pathway integration and the E. coli pntAB knock-in (GEVO1886) under anaerobic conditions. In addition this strain reached the highest values for volumetric productivity and titer under both conditions anaerobic and micro-aerobic (Table 14).

TABLE 14
Shown are the results for volumetric productivity, specific productivity titer
and yield reached in the period from 6 to 48 h for the tested strains and plasmid
systems

| | | Volumetric | | Specific | | | | | |
|---|---|---|---|---|---|---|---|---|---|
| | | Productivity | | Productivity | | Titer | | Yield | |
| Samples | Condition | [g/L/h] | ± | [g/L/h/OD] | ± | [g/L] | ± | [g/g] | ± |
| GEVO1886 | Micro- | 0.355 | 0.004 | 0.042 | 0.001 | 14.9 | 0.2 | 0.33 | 0.012 |
| | aerobic | | | | | | | | |
| GEVO1859 | Micro- | 0.266 | 0.010 | 0.040 | 0.004 | 11.2 | 0.4 | 0.33 | 0.016 |
| | aerobic | | | | | | | | |
| GEVO1846 | Micro- | 0.344 | 0.007 | 0.051 | 0.004 | 14.4 | 0.3 | 0.33 | 0.005 |
| | aerobic | | | | | | | | |
| GEVO1886 | Anaerobic | 0.355 | 0.008 | 0.056 | 0.001 | 14.9 | 0.1 | 0.35 | 0.004 |
| GEVO1859 | Anaerobic | 0.086 | 0.026 | 0.019 | 0.005 | 3.60 | 1.1 | 0.14 | 0.032 |
| GEVO1846 | Anaerobic | 0.209 | 0.019 | 0.041 | 0.004 | 8.79 | 0.8 | 0.27 | 0.006 |

The performance numbers in the period from 6 to 48 demonstrate that most of isobutanol production occurred under anaerobic conditions. Highest values for yield and specific productivity were reached by the strain featuring the complete pathway integration and the E. coli pntAB knock-in (GEVO1886) under anaerobic conditions.

### Example 6

### Anaerobic Batch Fermentation of GEVO1886 and GEVO1859

This example illustrates that an engineered microorganism which overexpresses a transhydrogenase, exemplified by the E. coli pntAB gene product (SEQ ID NO: 2 and SEQ ID NO: 4), from the chromosome produces isobutanol at a higher rate, titer and productivity compared to the a strain that does not overexpress a transhydrogenase. This is surprising because the increase in rate, titer, and productivity was achieved without modifying the isobutanol biosynthetic pathway itself.

Overnight cultures were started in 250 mL Erlenmeyer flasks with strain GEVO1886 and strain GEVO1859 cells from fresh streak plates with a 40 mL volume of M9 medium (Miller, J.H. A Short Course in Bacterial Genetics: A laboratory manual and handbook for Escherichia coli and related bacteria. 1992. Cold Spring Harbor Laboratory Press, Cold Spring Harbor, N.Y.) containing 85 g/L glucose, 20 g/L yeast extract, 20 μM ferric citrate, trace metals, an additional 1 g/L NH₄Cl, an additional 1 mM MgSO₄ and an additional 1 mM CaCl₂ and at a culture OD₆₀₀ of 0.02 to 0.05. The overnight cultures were grown for approximately 14 hours at 30° C. at 250 rpm.

Some of the overnight cultures were then transferred to 400 mL DasGip fermenter vessels containing about 200 mL of M9 medium (Miller, J.H. A Short Course in Bacterial Genetics: A laboratory manual and handbook for Escherichia coli and related bacteria. 1992. Cold Spring Harbor Laboratory Press, Cold Spring Harbor, N.Y.) containing 85 g/L glucose, 20 g/L yeast extract, 20 μM ferric citrate, trace metals, an additional 1 g/L NH₄Cl, an additional 1 mM MgSO₄ and an additional 1 mM CaCl₂ to achieve a starting cell concentration by optical density at 600 nm of 0.1. The vessels were attached to a computer control system to monitor and control pH at 6.5 through addition of base, temperature at 30° C., dissolved oxygen, and agitation. The vessels were agitated, with a minimum agitation of 200 rpm and agitation was varied to maintain a dissolved oxygen content of about 50% using a 12 sL/h air sparge until the OD₆₀₀ was about 1.0. The vessels were then induced with 1 mM IPTG.

After continuing growth for 3 hrs, the dissolved oxygen content was decreased to 0% with 200 rpm agitation and 2.5 sL/h sparge with nitrogen (N₂) gas. Measurement of the fermenter vessel off-gas for isobutanol and ethanol was performed throughout the experiment by passage of the off-gas stream through a mass spectrometer. Continuous measurement of off-gas concentrations of carbon dioxide and oxygen were also measured by a DasGip off-gas analyzer throughout the experiment. Samples were aseptically removed from the fermenter vessel throughout the experiment and used to measure OD₆₀₀, glucose concentration by HPLC, and isobutanol concentration in the broth by GC. Each strain was run in three independent fermentations.

Strain GEVO1886 reached an average isobutanol total titer of 21.6 g/L. The average yield of the fermentation, calculated when the titer of isobutanol was between 1 g/L and 15 g/L, was 88% of theoretical. The average productivity of the fermentation was 0.4 g/L/h. As described in Example 5, GEVO1886 performs at least equally well in terms of isobutanol productivity, titer, yield under anaerobic and aerobic conditions.

By comparison, strain GEVO1859 reached an average isobutanol total titer of 1.8 g/L. The average yield of the fermentation was 56% of theoretical, and the average productivity of the fermentation was 0.02 g/l/h.

### Example 7

### PntAB Overexpression Rescues a zwf-Deletion Phenotype

This example illustrates that a strain that has a growth defect and does not produce isobutanol because of the deletion in a native pathway that reduces the strains ability to produce the redox cofactor NADPH can surprisingly be rescued by overexpression of E. coli pntAB.

Overnight cultures of GEVO1399 transformed with plasmids pSA55, pGV1609 (SEQ ID NO: 108), and pGV1745 (SEQ ID NO: 117) and GEVO1399 transformed with plasmids pSA55, pGV1609, and pGV1720 (SEQ ID NO: 115) were started from glycerol stock cultures stored at −80° C. in 3 mL fermentation medium (M9 minimal medium according to Miller (Miller, J.H. A Short Course in Bacterial Genetics: A laboratory manual and handbook for Escherichia coli and related bacteria. 1992. Cold Spring Harbor Laboratory Press, Cold Spring Harbor, N.Y.), supplemented with 10 g/L yeast extract, 10 μM ferric citrate and trace metals) containing 8.5% glucose and the appropriate antibiotics in snap cap tubes about 14 h prior to the start of the fermentation.

Isobutanol fermentations were then carried out in fermentation medium containing 8.5% glucose and the appropriate antibiotics. Two 250 mL screw cap flasks with 20 mL fermentation medium containing 8.5% glucose and the appropriate antibiotics were inoculated with 1% of each grown overnight culture. The cells were incubated at 37° C./250 rpm until the strains were grown to an OD₆₀₀ of 0.6-0.8 and were then induced with Isopropyl β-D-1-thiogalactopyranoside at 1 mM final concentration. Three hours after induction one flask per overnight culture was shifted to anaerobic fermentation conditions. This was done by loosening the cap of the flasks and introducing the flasks into the anaerobic chamber. Once the flasks were flushed with oxygen free atmosphere (while going through the airlock), the flasks were closed again and incubated without shaking at 30° C. in the anaerobic chamber. The flasks in the anaerobic chamber were swirled twice a day. Samples were taken from the medium at the time of the shift and at 24 h and 48 h after inoculation, spun down at 22,000×g for 1 min to separate the cell pellet from the supernatant and stored frozen at −20° C. until analysis. The samples were analyzed using High performance liquid chromatography (HPLC) and gas chromatography (GC).

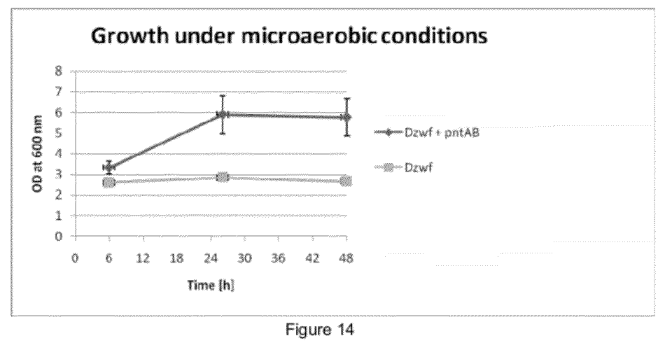

The strain lacking zwf without E. coli pntAB grew to an OD of about 3, whereas the samples featuring E. coli pntAB reached OD values of about 5-6. This OD was not significantly different from normal growth and thus the over-expression of E. coli pntAB rescues the zwf growth phenotype (FIG. 14).

Isobutanol production was rescued under micro-aerobic conditions by the overexpression of E. coli pntAB. Volumetric productivity and titer are improved 7.4 fold, specific productivity was improved 3.3 fold and yield 2.5 fold (Table 15).

TABLE 15
Volumetric productivity, specific productivity titer and yield in a micro-
aerobic fermentation for the tested strains and plasmid systems

| | Volumetric | | Specific | | | | | |
|---|---|---|---|---|---|---|---|---|
| | Productivity | | Productivity | | Titer | | Yield | |
| Samples | [g/L/h] | ± | [g/L/h/OD] | ± | [g/L] | ± | [g/g] | ± |
| GEVO1399 + | 0.170 | 0.001 | 0.030 | 0.003 | 8.18 | 0.02 | 0.248 | 0.012 |
| pGV1745 + pSA55 + | | | | | | | | |
| pGV1609 | | | | | | | | |
| GEVO1399 + | 0.023 | 0.004 | 0.009 | 0.002 | 1.10 | 0.18 | 0.100 | 0.013 |
| pGV1720 + pSA55 + | | | | | | | | |
| pGV1609 | | | | | | | | |

For the anaerobic shift experiment the same trend was observed as under micro-aerobic conditions. Isobutanol production was rescued by the over-expression of E. coli pntAB. Volumetric productivity and titer are improved 3.4 fold, specific productivity was improved 2.1 fold and yield by 43% (Table 16).

TABLE 16
Volumetric productivity, specific productivity titer and yield in an anaerobic
fermentation for the tested strains and plasmid systems

| | Volumetric | | Specific | | | | | |
|---|---|---|---|---|---|---|---|---|
| | Productivity | | Productivity | | Titer | | Yield | |
| Samples | [g/L/h] | ± | [g/L/h/OD] | ± | [g/L] | ± | [g/g] | ± |
| GEVO1399 + | 0.125 | 0.038 | 0.035 | 0.003 | 6.00 | 1.84 | 0.297 | 0.008 |
| pGV1745 + | | | | | | | | |
| pSA55 + pGV1609 | | | | | | | | |
| GEVO1399 + | 0.037 | 0.001 | 0.017 | 0.001 | 1.78 | 0.04 | 0.207 | 0.005 |
| pGV1720 + | | | | | | | | |
| pSA55 + pGV1609 | | | | | | | | |

### Example 8

### sthA Does not Contribute to Improvement in Anaerobic Isobutanol Production

This example illustrates that an isobutanol production strain with a deletion of the soluble transhydrogenase sthA produces low amounts of isobutanol anaerobically. This shows that the introduction of the sthA deletion does not provide cofactor balance to the isobutanol production strain and does not enable anaerobic isobutanol production above the levels seen for strains without redox engineering. The deletion of sthA has no significant effect on anaerobic performance of a production strain that overexpresses E. coli pntAB.

GEVO1748 and GEVO1844 were transformed with plasmids pGV1698 (SEQ ID NO: 112) and one of either pGV1720 (SEQ ID NO: 115) (control) or pGV1745 (SEQ ID NO: 117) (E. coli pntAB).

Overnight cultures of the strains to be tested were started either using fresh transformants (for all combinations featuring strain GEVO1844) or using frozen stocks (all other samples). The cultures were started in 3 mL fermentation medium (M9 minimal medium according to Miller (Miller, J.H. A Short Course in Bacterial Genetics: A laboratory manual and handbook for Escherichia coli and related bacteria. 1992. Cold Spring Harbor Laboratory Press, Cold Spring Harbor, N.Y.), supplemented with 10 g/L yeast extract, 10 μM ferric citrate and trace metals) containing 8.5% glucose and the appropriate antibiotics in snap cap tubes about 14 h prior to the start of the fermentation.

Isobutanol fermentations were then carried out in fermentation medium containing 8.5% glucose and the appropriate antibiotics. Two 250 mL screw cap flasks with 20 mL fermentation medium containing 8.5% glucose and the appropriate antibiotics were inoculated with 1% of each grown overnight culture. The cells were incubated at 37° C./250 rpm until the strains were grown to an OD₆₀₀ of 0.6-0.8 and were then induced with Isopropyl β-D-1-thiogalactopyranoside at 1 mM final concentration. Three hours after induction the flasks were shifted to anaerobic fermentation conditions. This was done by loosening the cap of the flasks and introducing the flasks into the anaerobic chamber. Once the flasks were flushed with oxygen free atmosphere (while going through the airlock), the flasks were closed again and incubated without shaking at 30° C. in the anaerobic chamber. The flasks in the anaerobic chamber were swirled twice a day. Samples were taken of the medium at the time of the shift and at 24 h and 48 h after inoculation, spun down at 22,000×g for 1 min to separate the cell pellet from the supernatant and stored frozen at −20° C. until analysis. The samples were analyzed using High performance liquid chromatography (HPLC) and gas chromatography (GC).

Strain GEVO1844 showed similar isobutanol production compared to non redox cofactor engineered strain GEVO1748 (Table 17).

TABLE 17
Shown are the results for volumetric productivity, specific productivity titer
and yield reached in an anaerobic fermentation for the tested strains and
plasmid systems

| | Volumetric | | Specific | | | | | |
|---|---|---|---|---|---|---|---|---|
| | Productivity | | Productivity | | Titer | | Yield | |
| Samples | [g/L/h] | ± | [g/L/h/OD] | ± | [g/L] | ± | [g/g] | ± |
| GEVO1844 + | 0.039 | 0.004 | 0.036 | 0.006 | 1.89 | 0.20 | 0.236 | 0.025 |
| pGV1720 + pGV1698 | | | | | | | | |
| (i.e. ΔsthA without | | | | | | | | |
| PntAB) | | | | | | | | |
| GEVO1748 + | 0.047 | | 0.022 | | 2.24 | | 0.279 | |
| pGV1720 + pGV1698 | | | | | | | | |
| (i.e. Control without | | | | | | | | |
| PntAB) | | | | | | | | |
| GEVO1844 + | 0.127 | 0.004 | 0.033 | 0.002 | 6.11 | 0.19 | 0.310 | 0.007 |
| pGV1745 + pGV1698 | | | | | | | | |
| (i.e. ΔsthA with | | | | | | | | |
| PntAB) | | | | | | | | |
| GEVO1748 + | 0.111 | 0.002 | 0.041 | 0.012 | 5.32 | 0.10 | 0.304 | 0.004 |
| pGV1745 + pGV1698 | | | | | | | | |
| (i.e. control with | | | | | | | | |
| PntAB) | | | | | | | | |

The strains with the sthA deletion exhibited similar isobutanol production compared to the strains without the sthA deletion. This was independent on the presence or absence of overexpression of E. coli pntAB. It can thus be concluded that the sthA deletion has no significant effect on isobutanol production.

### Example 9

### pntAB in Yeast

This example illustrates an isobutanol producing yeast which is engineered to express a transhydrogenase.

Yeast strain, GEVO5001, which is deficient in pyruvate decarboxylase activity and expresses the isobutanol biosynthetic pathway is further engineered to express a transhydrogenase. The E. coli pntA (SEQ ID NO: 1) and pntB (SEQ ID NO: 3) genes are expressed in yeast with the modifications of (1) N-terminal addition of amino acids to target the proteins to the plasma membrane (export signal sequence (ess)) and (2) N-terminal modifications to target the proteins to the mitochondrial outer membrane (mitochondrial targeting sequence (mts)). pGV6002 is a yeast integration plasmid that carries versions of pntA and pntB with modifications to target them to the plasma membrane. pGV6003 is a yeast integration plasmid that carries versions of pntA and pntB with modifications to target them to the mitochondrial outer membrane. In both cases, the pntA and pntB genes are under the control of the strong constitutive promoters from TEF1 and TDH3, respectively. pGV6002 and pGV6003 are linearized and transformed into GEVO5001 to generate GEVO5004 and GEVO5005, respectively. Expression of pntA and pntB is confirmed by qRT-PCR and once confirmed; GEVO5004 and GEVO5005 are used in fermentations for the production of isobutanol.

### Example 10

### Native E. coli Alcohol Dehydrogenase Activity Converts Isobutyraldehyde to Isobutanol

This example illustrates that native E. coli alcohol dehydrogenase activity converts isobutyraldehyde to isobutanol.

Strain JCL260 transformed with pGV1631 and pSA69 (strain without S. cerevisiae ADH2) and JCL260 transformed with pSA55 and pSA69 (strain with S. cerevisiae ADH2) were plated onto LB-plates containing the appropriate antibiotics and incubated overnight at 37° C. Plates were taken out of the incubator and kept at room temperature until further use. Overnight cultures were started in 3 mL EZ-Rich medium containing 7.2% glucose and the appropriate antibiotics in snap cap tubes about 14 hours prior to the start of the fermentation. Isobutanol fermentations were then carried out in EZ-Rich defined medium containing 7.2% glucose and the appropriate antibiotics. Screw cap flasks with 20 mL EZ-Rich medium containing 7.2% glucose and the appropriate antibiotics were inoculated with 1% of the grown overnight culture. The cells were incubated at 37° C./250 rpm until they were grown to an OD₆₀₀ of 0.6-0.8 and induced with Isopropyl β-D-1-thiogalactopyranoside (IPTG, 1 mM).

After induction the cells were incubated at 30° C./250 rpm. Samples were taken from the medium before induction, and 24 and 48 hours after inoculation, spun down at 22,000×g for 1 min to separate the cell pellet from the supernatant and stored frozen at −20° C. until analysis.

The ADH2 gene product is expected to be functionally expressed from pSA55 and required for isobutanol production. Thus, no isobutanol should be produced with the plasmid combination lacking ADH2 as adhE is deleted in JCL260. However, isobutanol production for the system lacking ADH2 was higher than for the system with ADH2 expression. Table 18 shows the results for the isobutanol fermentation comparing the pathway including Adh2 expression with the exact same system excluding Adh2 expression. Both systems feature Bs_AlsS1, Ec_IlvC and Ec_ilvD expressed from the same medium copy plasmid and LI_Kivd1 expressed from a high copy plasmid. Volumetric productivity and titer showed 42% increase, specific productivity 18% and yield 12% increase. This suggests strongly that a native E. coli dehydrogenase is responsible for the conversion of isobutyraldehyde to isobutanol. and that Adh2 is not expressed and not necessary for isobutanol production in E. coli.

TABLE 18
Isobutanol fermentation with and without Adh2 expression

| | Volumetric | | Specific | | | | | |
|---|---|---|---|---|---|---|---|---|
| | Productivity | | Productivity | | Titer | | Yield | |
| samples | [g/L/h] | ± | [g/L/h/OD] | ± | [g/L] | ± | [g/g] | ± |
| without Adh2 | 0.175 | 0.006 | 0.039 | 0.003 | 8.40 | 0.26 | 0.207 | 0.009 |
| with Adh2 | 0.123 | 0.004 | 0.033 | 0.001 | 5.88 | 0.17 | 0.185 | 0.004 |

### Example 11

### Identification of Native ADH

This example illustrates that the native E. coli alcohol dehydrogenase is encoded by the Ec_yqhD gene (SEQ ID NO: 68).

Several E. coli genes predicted or known to code for alcohol dehydrogenases were knocked out of strain JCL260 to determine whether any of them are involved in isobutyraldehyde reduction. Fermentations were carried out with GEVO1608 and with JCL260, each transformed with plasmids pGV1609 (SEQ ID NO: 108) and pGV1631 by electroporation. Single colonies were grown and two colonies from each strain were started in a 3 mL overnight culture, with appropriate antibiotics. Each 250 mL fermentation flask was filled with 20 mL of EZ-Rich medium (Neidhardt, F. C., P. L. Bloch, and D. F. Smith. 1974. Culture medium for enterobacteria. J. Bacteriol. 119:736-47) supplemented with 5% glucose, Ampicillin (100 mg/mL), and Chloramphenical (100 mg/mL).

The cell densities of the overnight cultures were normalized and 2% inoculum was added to each fermentation flask and incubated at 270 rpm/37° C. The cultures were induced with 20 μL 0.1 M IPTG after they reached an OD₆₀₀ of 0.6-0.8 at which time the temperature was lowered to 30° C. Samples were taken from the medium before induction, and 24 hours after inoculation, spun down at 22,000×g for 1 min to separate the cell pellet from the supernatant and stored frozen at −20° C. until analysis. A second fermentation was performed in the same way with the best candidate, GEVO1608 containing the yqhD deletion, and samples were taken at 24 and 48 hours.

While both GEVO1608 and JCL260 grew to similar cell densities, GEVO1608 produced ˜80% less isobutanol than the control strain (Table 19), indicating that the Ec_yqhD gene product is primarily responsible for isobutyraldehyde reduction.

TABLE 19
Specific Productivity and Titer of Fermentation

| Strain | Plasmids | Time | Titer (g/L) |
|---|---|---|---|
| GEVO1608 | pGV1609, pGV1631 | 24 h | 0.33 |
| JCL260 | pGV1609, pGV1631 | 24 h | 2.45 |
| GEVO1608 | pGV1609, pGV1631 | 48 h | 0.83 |
| JCL260 | pGV1609, pGV1631 | 48 h | 4.00 |

### Example 12

### Overexpression of NADH-Dependent Alcohol Dehydrogenase and Propanediol Dehydrogenases

This example demonstrates that overexpression of an NADH-dependent alcohol dehydrogenase or propanediol dehydrogenases increases isobutanol production.

Relevant E. coli strains were transformed with the appropriate plasmids (Table 20).

TABLE 20
Plasmid and strain combinations used in isobutanol fermentations

| # | Plasmid 1 | Plasmid 2 | Strain | Comments |
|---|---|---|---|---|
| 1 | pGV1655 | pGV1698 | GEVO1745 | No ADH on plasmid |
| 2 | pGV1655 | pGV1698 | JCL260 | GEVO1780 |
| 3 | pGV1655 | pGV1748 | GEVO1745 | Ec_fucO |
| 4 | pGV1655 | pGV1749 | GEVO1745 | Dm_ADH |
| 5 | pGV1655 | pGV1778 | GEVO1745 | Kp_dhaT |

Following transformation, the strains were plated on LB-plates containing the appropriate antibiotics and incubated overnight at 37° C. Overnight cultures were started in 3 mL EZ-Rich medium (Neidhardt, F. C., P. L. Bloch, and D. F. Smith. 1974. Culture medium for Enterobacteria. J. Bacteriol. 119:736-47) containing 8% glucose and the appropriate antibiotics in snap cap tubes about 14 h prior to the start of the fermentation. Isobutanol fermentations were then carried out in EZ-Rich Medium containing 8% glucose and the appropriate antibiotics. Screw cap flasks with 25 mL EZ-Rich medium containing 8% glucose and the appropriate antibiotics were inoculated with a sufficient volume of the grown overnight culture to obtain a starting OD₆₀₀ of 0.1. The cells were incubated at 37° C./250 rpm until they reached an OD₆₀₀ of 0.6-0.8 followed by induction with Isopropyl β-D-1-thiogalactopyranoside (IPTG, 1 mM). After induction, cultures were capped, sealed and placed in 30° C. shaker, 225 rpm to start fermentation. Samples (2 mL) were taken 24 h and 48 h post induction, centrifuged at 22,000×g for 1 min and the supernatant stored at 4° C. until analyzed. Prior to analysis, the supernatants were filtered and then analyzed via Gas Chromatography and High Performance Liquid Chromatography. All experiments were carried out in triplicate.

Results are presented in Table 21, below. Expression of either 1,2-propanediol dehydrogenase Ec_fucO or 1,3-propanediol dehydrogenase Kp_dhaT significantly and reproducibly increases titer in the ΔyqhD background of strain GEVO1745. Expression of Dm_ADH enhances titer and yield of the fermentations in the ΔyqhD background of strain GEVO1745.

TABLE 21
Summary of isobutanol titer, and yield data from fermentations after 48
hours

| # | Comments | titer [g/L] | ± | Yield [% theor.] | ± |
|---|---|---|---|---|---|
| 1 | no ADH | 1.91 | 0.50 | 38.5 | 10.30 |
| 2 | GEVO1780 | 3.39 | 0.15 | 65.0 | 2.83 |
| 3 | Ec_FucO | 6.30 | 0.10 | 79.9 | 1.79 |
| 4 | Dm_Adh | 4.86 | 0.29 | 67.0 | 4.54 |
| 5 | Kp_DhaT | 6.22 | 0.16 | 75.3 | 2.04 |

### Example 13

### Characterization of Alcohol Dehydrogenases

This example demonstrates that the alcohol dehydrogenases Ec_FucO (SEQ ID NO: 65), Kp_DhaT (SEQ ID NO: 63), and Dm_Adh (SEQ ID NO: 61) catalyze the NADH-dependent reduction of isobutyraldehyde.

E. coli strain GEVO1745 was transformed by electroporation with one of plasmids pGV1705-A, pGV1748-A, pGV1749-A, or pGV1778-A. 50 mL of TB medium (23.1 g/L KH2PO4, 125.4 g/L K2HPO4, 12 g/L Bacto-tryptone, 24 g/L yeast extract, 4 ml/L glycerol) were inoculated to an initial OD₆₀₀ of 0.2 using a 3 mL overnight LB culture of a single colony. The 50 mL culture was allowed to grow for 3-4 hrs at 250 rpm and 37° C. Protein expression was induced at an OD₆₀₀ of 2-2.5 by the addition of IPTG to a final concentration of 1 mM. After the addition of IPTG, protein expression was allowed to continue for 20-24 hours at 225 rpm and 25° C.

Alcohol dehydrogenase (ADH) activity was assayed kinetically by monitoring the decrease in NAD(P)H concentration by measuring the absorbance at 340 nm. A reaction buffer was prepared containing 0.1 M potassium phosphate, 0.4 mM NAD(P)H, 10 mM isobutyraldehyde, 1 mM DTT, and 1 mM PMSF. Cell pellets were resuspended in 0.1 M potassium phosphate buffer containing 1 mM DTT and 1 mM PMSF at one fifth of the culture volume, i.e. 10 mL resuspension buffer for cell pellet from a 50 mL culture. The resuspended cells were lysed by sonication for 1 min with a 50% duty cycle. The reaction was initiated by the addition of 0.5 mL of the reaction buffer to 0.5 mL of clarified lysate in a cuvette. Dilution of the clarified lysate was necessary for ADHs that were highly active. A substrate free control was conducted using reaction buffer without the addition of aldehyde.

Kinetic parameters were determined for Ec_YqhD, Ec_FucO, Dm_Adh, and Kp_DhaT (Table 22).

TABLE 22
Kinetic parameters for the conversion of isobutyraldehyde to
isobutanol by Ec_YqhD, Ec_FucO, Dm_Adh, and Kp_DhaT

| | | NADH | | NADPH | |
|---|---|---|---|---|---|
| | | | Activity | | Activity |
| | | | (U/min⁻¹ | | (U/min⁻¹ |
| | | K_{M} | mg⁻¹ crude | K_{M} | mg⁻¹ crude |
| Plasmid | ADH | (mM) | lysate) | (mM) | lysate) |
| pGV1705-A | Ec_YqhD | n.d. | n.d. | 0.25 | 0.09 |
| pGV1748-A | Ec_FucO | 0.8 | 0.23 | 0.2 | 0.04 |
| pGV1749-A | Dm_Adh | 0.9 | 6.60 | 2.7 | 1.70 |
| pGV1778-A | Kp_DhaT | 1.3 | 0.56 | 0.6 | 0.08 |

The kinetic properties of the LI_AdhA enzyme were described by Atsumi et al. (Atsumi et al., Appl. Microbiol. Biotechnol., 2009, DOI 10.1007/s00253-009-2085-6), and are shown in Table 23.

TABLE 23
Kinetic parameters for Ll_AdhA (Atsumi et al., Appl. Microbiol. Biotechnol.,
2009, DOI 10.1007/s00253-009-2085-6)

| | | NADH | | | NADPH | | |
|---|---|---|---|---|---|---|---|
| | | K_{M} | | | K_{M} | | |
| ADH | Substrate | (mM) | k_{cat} (s⁻¹) | Kcat/K_{M} | (mM) | k_{cat} (s⁻¹) | Kcat/K_{M} |

| | | | | | |
|---|---|---|---|---|---|
| Ll_AdhA | Acetaldehyde | 0.5 | 10 | 20.9 | n.d.^{a} |
| Ll_AdhA | isobutyraldehyde | 9.1 | 6.6 | 0.8 | |

^{a}did not show any detectably activity when tested with NADPH as a cofactor

### Example 14

### KARI Engineering by Saturation Mutagenesis

Construction of KARI-containing plasmids: Standard molecular biology procedures (Sambrook and Russell, Molecular Cloning, A Laboratory Manual, 3^{rd} Edition, Vol. 3, 2001) were utilized to make plasmid pGV1711 (SEQ ID NO: 113) (pLIacO1::(no ORF) bla, ColE1 ORI). Plasmid pGV1711 is a high-copy, AmpR vector that serves as an “empty vector” control, i.e. it contains no open reading frames under the control of the PLIac promoter. The E. coli KARI gene Ec_ilvC (SEQ ID NO: 10) was codon optimized for E. coli resulting in gene Ec_ilvC_coEc (SEQ ID NO: 11)

The codon optimized gene Ec_ilvC_coEc was cloned into pET22b(+) using primers KARIpETfor and KARIpETrev introducing a 5′ NdeI and a 3′ XhoI restriction site and a C-terminal his₆-tag, resulting in plasmid pET22b[ilvCco] carrying Ec_ilvC_coEc^{his6}(SEQ ID NO: 14).

DNA constructs were analyzed by restriction digests, and also by DNA sequencing to confirm integrity and correct construction. Primers pETup and KARIpETrev were used as primers in standard DNA sequencing reactions to sequence pET22b(+) derivatives.

Construction of NNK libraries: NNK libraries were constructed using site directed mutagenesis overlap extension (SOE) PCR. First, the fragments containing the mutations were created allowing for at least 15 by of overlap using KARIpET_for and KARIpET_rev and the respective NNK primers listed in Table 6 (SEQ ID NO 285 through SEQ ID NO 298). After digesting traces of template DNA with DpnI, the fragments were separated on a 1% TAE agarose gel, extracted, and the PCR products were precipitated using pellet paint (Novagen). The clean products were used as templates in a subsequent assembly PCR. The PCR product was cleaned up (Zymo Research, Orange, Calif.), restriction digested with NdeI and XhoI for 1.5 h at 37° C., cleaned on a 1% agarose gel, and ligated into pET22b(+).

Site directed mutagenesis mutants were generated as described above. The successful mutagenesis was confirmed by DNA sequencing.

Cell growth and protein expression in shake flasks: Flasks containing 25 mL of Luria-Bertani (LB) medium (10 g tryptone, 10 g NaCl, 5 g yeast extract) with ampicillin (final concentration 0.1 mg/mL) were inoculated to an initial OD₆₀₀ of 0.1 using 0.25 mL overnight LB culture of a single colony. The 25 mL LB expression culture was allowed to grow for 3-4 h at 250 rpm and 37° C. Protein expression was induced at OD₆₀₀ of 1 by the addition of IPTG to a final concentration of 0.5 mM. Protein expression was allowed to continue for 20-24 h at 225 rpm and 25° C. Cells were harvested at 5300×g and 4° C. for 10 min and the cell pellets were frozen at −20° C. until further use.

Cell growth and protein expression in microplates: In order to grow and express KARI variants in deep well plates, sterile toothpicks were used to pick single colonies into shallow 96 well plates filled with 300 μl LBₐₘₚ. 75 μl of these overnight cultures were used to inoculate deep well plates filled with 600 μl of LBₐₘₚ per well. The plates were grown at 37° C. and 210 rpm for 4 h. One hour before induction with IPTG (final concentration 0.5 mM), the temperature of the incubator was reduced to 25° C. After induction, growth and expression continued for 20 h at 25° C. and 210 rpm. Cells were harvested at 5300×g and 4° C. and stored at −20° C.

KARI cuvette assay: KARI activity was assayed kinetically by monitoring the decrease in NAD(P)H concentration by measuring the absorbance at 340 nm. A reaction buffer was prepared containing 250 mM potassium phosphate pH 7, 1 mM DTT and 10 mM MgCl₂. Cell pellets were resuspended (0.25 g wet weight/mL buffer) in 250 mM potassium phosphate (KPi) buffer containing 1 mM DTT and 10 mM MgCl₂. The resuspended cells were lysed by sonication for 1 min with a 50% duty cycle and pelleted at 11000×g and 4° C. for 15 min. A reaction mixture consisting of 910 μl reaction buffer, 50 μl acetolactate, and 20 μl lysate was prepared in a cuvette. The reaction was initiated by addition of 20 μL of 10 mM NAD(P)H. A substrate free control was conducted using reaction buffer without the addition of acetolactate.

KARI high-throughput assay: Frozen cell pellets were thawed at room temperature for 20 min and then 100 μL of lysis buffer (250 mM Kpi, 750 mg/L lysozyme, 10 mg/L DNaseI, pH 7) were added. Plates were vortexed to resuspend the cell pellets. After a 30 min incubation at 37° C., plates were centrifuged at 5300×g and 4° C. for 10 min. 20 μL of the resulting crude extract were transferred into assay plates (flat bottom, Rainin) using a liquid handling robot. 10 mL assay buffer per plate were prepared (250 mM Kpi, pH 7, 500 μL acetolactate, 1 mM DTT, 10 mM NAD(P)H, and 10 mM MgCl₂) and 90 μL thereof were added to each well to start the reaction. The depletion of NAD(P)H was monitored at 340 nm in a plate reader (TECAN) over 1.5 min.

Purification of KARI: Cell pellets used for purification were resuspended in purification buffer A (20 mM Tris, 20 mM imidazol, 100 mM NaCl, 10 mM MgCl₂, pH 7.4). KARI was purified by IMAC (Immobilized metal affinity chromatography) over a 1 ml Histrap High Performance (histrap HP) column pre-charged with Nickel (GE Healthcare) using an Akta FPLC system (GE Healthcare). The column was equilibrated with four column volumes (cv) of buffer A. After injecting the crude extract, the column was washed with buffer A for 2 cv, followed by a wash step with a mixture of 10% elution buffer B (20 mM Tris, 300 mM imidazol, 100 mM NaCl, 10 mM MgCl₂, pH 7.4) for 5 cv. KARI variants were eluted at 40% buffer B and stored at 4° C.

Homology modeling was performed with pymol and x-ray structures of E. coli KARI (PDB ID: 1YRL) and spinach KARI (PDB ID: 1YVE), the latter containing NADPH co-crystallized.

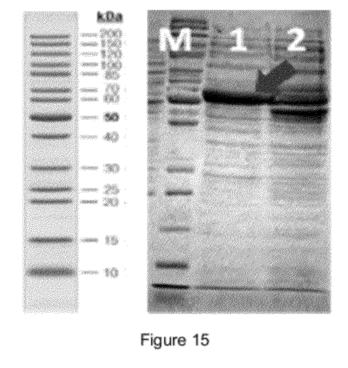

A KARI expression construct (pGV1777 (SEQ ID NO: 118)) (pLIacO1::Ec_ilvC_coEc::bla, ColE1 ORI) was tested in E. coli strain GEVO1777 and yielded KARI activity in lysates. On this plasmid, the ilvC gene was not his-tagged and therefore no purification was attempted. In order to obtain higher expression levels for a high-throughput screen (HTS) in 96-well plate format, ilvC_co was sub-cloned into pET22b(+). This plasmid also ads a his-tag to the C-terminus of the protein to facilitate purification. E. coli BL21 (DE3) (Lucigen, Middleton, Wis.) cells were transformed with pET22[ilvCco] and protein expression was performed in LB medium with ampicillin at 25° C. SDS PAGE analysis (FIG. 15) shows a comparison of crude extracts of BL21 (DE3) and GEVO1777 expressing KARI.

Table 24 shows the specific activities in U/mg of KARI in lysates of GEVO1777 and BL21(DE3) being 15-fold higher in BL21 crude extract, mirroring the results shown in the SDS PAGE.

TABLE 24
Specific Activities of KARI in U/mg Expressed in GEVO1777 and
BL21 (DE) measured with NADPH

| Strain/Construct | U/mg Crude Extract |
|---|---|
| pGV1777 in GEVO1777 | 0.03 |
| pET22b[ilvCco] in BL21 (DE3) | 0.45 |

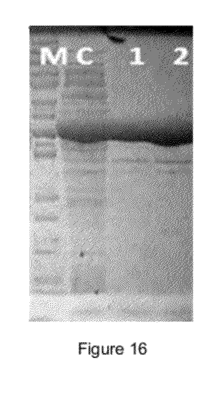

Purification of his-tagged KARI expressed from pET22[ilvCco] in BL21(DE3) cells was first performed over a linear gradient to determine the proper amount of imidazol to elute KARI. Then, a step gradient was implemented and the protein was eluted at 40% elution buffer B (140 mM imidazol). A SDS PAGE documented the purity of the enriched protein (FIG. 16).

A quadruplet E. coli IlvC mutant (R68D:K69L:K75V:R76D), which was described previously by Rane and coworkers (Rane et al., 1997, Arch Biochem Biophys 338: 83-89) was constructed using the respective primers listed in Table 6 (SEQ ID NO: 281 through SEQ ID NO 284) and cloned into pET22b(+) as described, but did not yield the cofactor switch that was described in the paper, although the ratio NADH/NADPH was 2.5 (wild-type 0.08). In fact, the specific activity of the quadruplet mutant on NADH was even worse than wild-type (Table 25), suggesting this mutant enzyme is not suited for the aforementioned aims.

TABLE 25
Comparison of specific activities from purified Ec_IlvC^{his6} and
purified IlvC^{quadruplet-his6} quadruplet in U/mg measured on NAD(P)H

| | U/mg with | U/mg with | NADH/ |
|---|---|---|---|
| Variant | NADH | NADPH | NADPH |
| Ec_IlvC^{his6} | 0.03 | 1 | 0.08 |
| IlvC^{quadruplet-his6} | 0.45 | 0.02 | 2.5 |

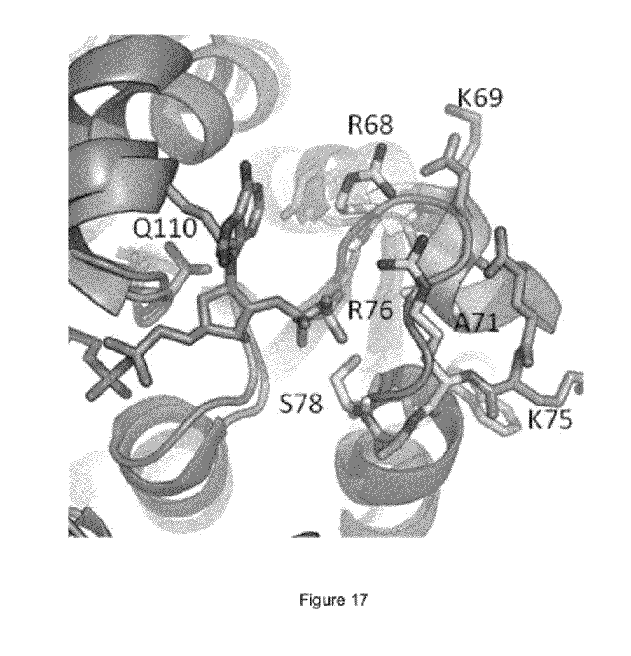

Since the quadruplet KARI mutant did not yield the promised activity, the Ec_ilvC_coEc^{his6} gene (SEQ ID NO: 14) was used as starting point for engineering a cofactor switch. A structure alignment of E. coli KARI with spinach KARI was generated (FIG. 17) because spinach KARI was co-crystallized with NADPH. The position of the cofactor in the spinach KARI structure was in good agreement with the NADPH phosphate group in the E. coli KARI structure. Based on this, amino acid residues R68, A71, R76, S78, and Q110 seemed likely to be interacting with NADPH and therefore were chosen as targets in a site saturation mutagenesis experiment. Only residues R68 and R76 were found in the aforementioned quadruplet mutant. Residues K69 and K75 seemed less likely to be involved in cofactor binding.

Five individual site saturation libraries were generated and electro-competent E. coli BL21(DE3) cells were transformed with the desalted ligation mixtures. 88 clones of each library were screened for NAD(P)H depletion at 340 nm in microplates. Clones with an improved NADH/NADPH consumption ratio while maintaining or increasing their NADH activity were chosen for a rescreen. Variants that passed the rescreen were sequenced, expressed in shake flasks, purified, and characterized.

The first screening round resulted in several improved variants in terms of their specific activity on NADH (and NADPH for most of them) (Table 26). The first variant to favor NADH over NADPH was Ec_IlvC^{S78D-his6} which showed a specific activity for NADH that equals the specific activity of Ec_IlvC^{his6} for NAPDH (1 U/mg). Table 26 shows the variants resulting from the first round of site saturation mutagenesis compared to the parent Ec_IlvC^{his6}. All proteins were purified over a histrap column.

TABLE 26
Specific Activities for NADH and NADPH in U/mg

| | U/mg | U/mg | NADH/ |
|---|---|---|---|
| Variant | NADH | NADPH | NADPH |
| No mutation | 0.08 | 1 | 0.08 |
| (Ec_IlvC^{his6}) | | | |
| Ec_IlvC^{R68L-his6} | 0.27 | 1.15 | 0.23 |
| Ec_IlvC^{A71T-his6} | 0.48 | 1.81 | 0.27 |
| Ec_IlvC^{A71S-his6} | 0.57 | 2.65 | 0.22 |
| Ec_IlvC^{R76G-his6} | 0.64 | 2.73 | 0.23 |
| Ec_IlvC^{R76S-his6} | 0.59 | 1.51 | 0.39 |
| Ec_IlvC^{R76T-his6} | 0.25 | 1 | 0.25 |
| Ec_IlvC^{R76D-his6} | 0.26 | 0.69 | 0.38 |
| Ec_IlvC^{S78D-his6} | 1 | 0.61 | 1.64 |
| Ec_IlvC^{Q110A-his6} | 0.85 | 2 | 0.43 |
| Ec_IlvC^{Q110V-his6} | 0.93 | 2 | 0.47 |

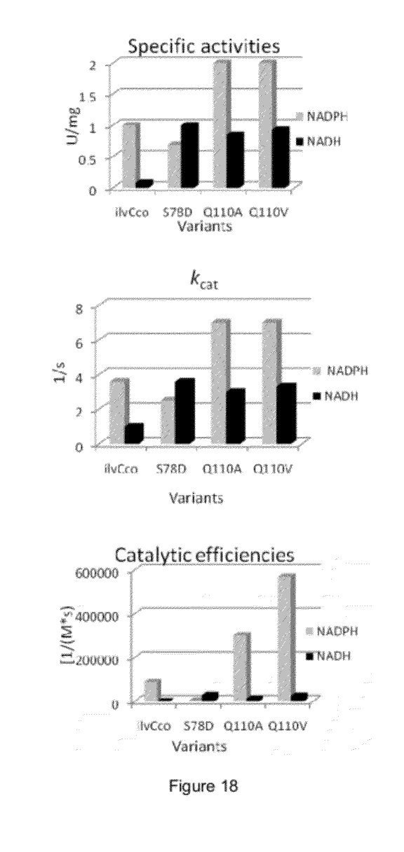

The three best variants Ec_IlvC^{S78D-his6}, Ec_IlvC^{Q110A-his6}, and Ec_IlvC^{Q110V-his6} were characterized according to their specific activities [U/mg], k_{cat} values [s⁻¹], catalytic efficiencies [M⁻¹*s⁻¹] (FIG. 18), and K_{M} values (Table 27).

TABLE 27
K_{M} values of Ec_IlvC^{his6} compared to three variants resulting
from the site saturation library

| | K_{M} [mM] | K_{M} [mM] |
|---|---|---|
| Variant | NADPH | NADH |
| Ec_IlvC^{his6} | 41 | 1075 |
| Ec_IlvC^{S78D-his6} | 658 | 130 |
| Ec_IlvC^{Q110V-his6} | 13 | 135 |
| Ec_IlvC^{Q110A-his6} | 24 | 277 |

All three variants were improved compared to the parent Ec_IlvC^{his6}. Ec_IlvC^{S78D-his6} was the first variant to show an actual preference of NADH over NADPH, while variants Ec_IlvC^{Q110A-his6} and Ec_IlvC^{Q110V-his6} showed drastic improvements in their overall catalytic efficiencies (FIG. 18). Table 28 contains a comparison of the K_{M} values of Ec_IlvC^{his6} with the three best variants resulting from the site saturation mutagenesis library on both cofactors. All variants showed improved K_{M} values on NADH. While Ec_IlvC^{Q110V-his6} and Ec_IlvC^{Q110A-his6} had improved K_{M} values on NADPH compared to wild-type, the K_{M} value of variant Ec_IlvC^{S78D-his6} on NADPH was decreased 16-fold from 1075 μM to 130 μM. The catalytic efficiencies on NADH were greatly improved as well. Ec_IlvC^{his6} showed 1,000 M⁻¹*^{s-1}, while Ec_IlvC^{S78D-his6} yielded 27,600 M⁻¹*^{s-1}.

TABLE 28
Catalytic efficiencies [M⁻¹*s⁻¹] for Ec_IlvC^{his6} and variants
Ec_IlvC^{Q110V-his6}, Ec_IlvC^{Q110A-his6}, and Ec_IlvC^{S78D-his6} on
NADPH

| | | | (k_{cat}/K_{M} with |
|---|---|---|---|
| | | | NADH)/(k_{cat}/K_{M} of |
| | k_{cat}/K_{M} | k_{cat}/K_{M} | Ec_IlvC^{his6} with |
| | with NADH | with NADH | NADPH) |
| Variant | [M⁻¹ * s⁻¹] | [M⁻¹ * s⁻¹] | [%] |
| Ec_IlvC^{his6} | 1000 | 87300 | 1% |
| Ec_IlvC^{Q110V-his6} | 24800 | 569000 | 28% |
| Ec_IlvC^{Q110A-his6} | 11063 | 301800 | 13% |
| Ec_IlvC^{S78D-his6} | 27600 | 3770 | 32% |

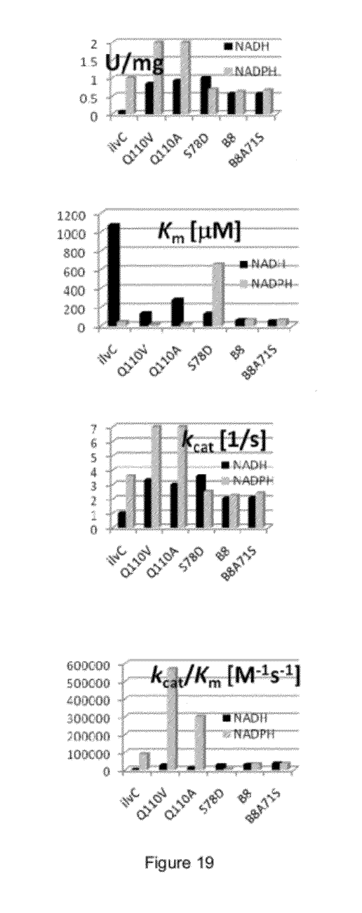

As a next step, the gene encoding variant Ec_IlvC^{Q110V-his6} (SEQ ID NO: 23) was used as template to generate individual combinations of the mutation Q110V with other mutations: R68L, A71T, A71S, R76G, R76S, R76T, S78D, and R76D. After screening the variants as described above, the most promising ones were expressed, purified, and characterized. Table 29 lists the K_{M} values in μM on NADPH and NADH for Ec_IlvC^{his6}, Ec_IlvC^{Q110V-his6}, and variants of Ec_IlvC^{Q110V-his6}. Variant Ec_IlvC^{B8-his6} containing amino acid mutations Q110V and S78D, showed the same K_{M} value for NADH and for NADPH with 65 μM. The A71S mutation was introduced into Ec_IlvC^{B8-his6} resulting in a variant Ec_IlvC^{B8A71S-his6}, which yielded 44% catalytic efficiency on NADH compared to the catalytic efficiency of wild-type KARI on NADPH (FIG. 19 and Table 30).

TABLE 29
K_{M} values for Ec_IlvC^{his6}, Ec_IlvC^{Q110V-his6}, and variants of
Ec_IlvC^{Q110V-his6} on NADPH and on NADH

| | K_{M} for NADPH | K_{M} for NADH |
|---|---|---|
| Variant | [mM] | [mM] |
| Ec_IlvC^{his6} | 41 | 1075 |
| Ec_IlvC^{Q110V-his6} | 13 | 135 |
| Ec_IlvC^{Q110VA71T-his6} | 37 | 80 |
| Ec_IlvC^{Q110VA71S-his6} | 30 | 70 |
| Ec_IlvC^{Q110VR76G-his6} | 47 | 87 |
| Ec_IlvC^{Q110VR76S-his6} | n.d. | 223 |
| Ec_IlvC^{B8-his6} | 65 | 65 |

TABLE 30
Catalytic efficiencies [M⁻¹ * s⁻¹] for wild-type Ec_IlvC^{his6} and
variants Ec_IlvC^{Q110V-his6}, Ec_IlvC^{Q110A-his6}, and Ec_IlvC^{S78D-his6} on
NAD(P)H compared to Ec_IlvC^{B8-his6} and Ec_IlvC^{B8A71S-his6}

| | | | (k_{cat}/K_{M} with |
|---|---|---|---|
| | | | NADH)/(k_{cat}/ |
| | k_{cat}/K_{M} with | k_{cat}/K_{M} with | K_{M} of Ec_IlvC^{his6} |
| | NADH | NADH | with NADPH) |
| Variant | [M⁻¹ * s⁻¹] | [M⁻¹ * s⁻¹] | [%] |
| Ec_IlvC^{his6} | 1000 | 87300 | 1% |
| Ec_IlvC^{Q110V-his6} | 24800 | 569000 | 28% |
| Ec_IlvC^{Q110A-his6} | 11063 | 301800 | 13% |
| Ec_IlvC^{S78D-his6} | 27600 | 3770 | 32% |
| Ec_IlvC^{B8-his6} | 31775 | 34188 | 36% |
| Ec_IlvC^{B8A71S-his6} | 38330 | 37459 | 44% |

### Example 15

### KARI Engineering by Recombination

The codon optimized gene Ec_ilvC_coEc^{his6} (SEQ ID NO: 14) and libraries thereof were cloned into pET22b(+) using primers KARIpETfor and KARIpETrev (Table 6). DNA constructs were analyzed by restriction digests, and also by DNA sequencing to confirm integrity and correct construction. Primers pETup and KARIpETrev (Table 6) were used as primers in standard DNA sequencing reactions to sequence pET22b(+) derivatives.

The recombination library was constructed using SOE PCR introducing mutations found at the five targeted sites while allowing for wild-type sequence as well. The first fragments were generated using degenerate primers R68A71 recombfor and R68A71 recombrev which covered the gene sequence coding for the region at amino acid positions 68/71 (Table 6). After assembling the long and the short fragment, the assembly product was DpnI digested for 1 h, separated on an agarose gel, freeze'n'squeeze (BioRad, Hercules, Calif.) treated, and finally pellet painted (Novagen, Gibbstown, N.J.). The clean assembly product served as template for the second round of SOE PCR introducing mutations at amino acid positions 76/78 using the following primers: R68A71recombfor, R68A71recombrev, R76S78recombfor, R76S78recombrev, G76S78recombfor, G76S78recombrev, S76S78recombfor, S76S78recombrev, T76S78recombfor, T76S78recombrev, D76S78recombfor, D76S78recombrev, R76D78recombfor, R76D78recombrev, G76D78recombfor, G76D78recombrev, S76D78recombfor, S76D78recombrev, T76D78recombfor, T76D78recombrev, D76D78recombfor, D76D78recombrev (Table 6). The mixture of primers was used, since degenerate codons would have expanded the library size immensely. Again, the assembly product served as template to complete the recombination library with amino acid position 110. The same procedure was applied as described for the first two rounds of SOE PCR. Primers used were again a mixture prepared out of equimolar concentrations of Q110Qfor, Q110Qrev, Q110Afor, Q110Arev, Q110Vfor, and Q110Vrev. After all sites were recombined, the insert was restriction digested with NdeI and XhoI, ligated into pET22b(+), and electro-competent BL21(D3) (Lucigen, Middleton, Wis.) were transformed. In order to oversample the library by approximately five-fold, one thousand clones were picked and cultured as described below. In order to check for possible biases (i.e. certain mutations occurring more frequently than others), 20 clones were randomly chosen for DNA sequence analysis.

As described in Example 14, the first screening round identified several individual point mutations within the KARI cofactor binding region that either improved NADH-dependent activity or were at least neutral (i.e. had neither a beneficial nor deleterious effect). These mutations, along with the wild-type amino acid residue are listed in Table 31.

TABLE 31
Amino Acid Mutations Included in the Recombinatorial Library

| | | | Total # |
|---|---|---|---|
| Amino Acid | | Neutral or beneficial | (including |
| Position | Wild-type | mutations identified | wild-type) |
| 68 | R | L | 2 |
| 71 | A | T, S | 3 |
| 76 | R | G, S, T, D | 5 |
| 78 | S | D | 2 |
| 110 | Q | A, V | 3 |

A complete recombination library was constructed allowing for all beneficial and some neutral mutations (and including the wild-type residues) at each of the five sites. The total number of unique combinations was 180.

Generating all mutations using a single primer would result in a large library of ˜4,000. Thus, the present inventors built the library stepwise in three SOE reactions using primers mixed in equimolar amounts for each of three SOE reactions:
SOE 1: R68/A71, R68/T71, R68/S71, L68/A71, L68/T71, L68/S71
SOE 2: A76/S78, G76/S78, S76/S78, T76/S78, D76/S78, A76/D78, G76/D78, S76/D78, T76/D78, D76/D78,
SOE 3: Q110, A110, V110

First, mutations at amino acid sites 68 and 71 were introduced into the Ec_ilvC_coEc^{his6} gene, followed by mutations at site 76 and finally, by mutations at site 110. After the library had been generated, it was ligated into pET22b(+). The resulting plasmid library was used to transform E. coli BL21(DE3) electro-competent cells. Cells were grown in 96-well plates according to the protocol for cell growth and protein expression in microplates as described in Example 14. The KARI enzyme activity of each of 1,000 individual transformants was determined using the high-throughput assay as described in Example 14.

Only 20% of the enzymes of the recombination library were active on NADH. After screening 1,000 clones using the NADH depletion assay at 340 nm, 26 KARI variants were selected for a rescreen by the high-throughput assay described in Example 14 and eight thereof were expressed in 25 ml LBₐₘₚ medium in shake flasks according to the protocol for cell growth and protein expression in shake flasks as described in Example 14, purified according to the protocol for purification of KARI enzymes as described in Example 14, and NAD(P)H depletion at 340 nm was measured again. Two candidates Ec_IlvC^{2H10-his6} (containing the amino acid substitutions A71S, R76D, S78D, and Q110A) and Ec_IlvC^{6E6-his6} (containing the amino acid substitutions A71S, R76D, S78D, and Q110V) showed good specific activity on NADH and were only marginally active on NADPH. The other six variants showed lower specific activities on NADH (ranging from 0.44-0.55 U/mg) compared to the two favored variants Ec_IlvC^{2H10-his6} and Ec_IlvC^{6E6-his6} and higher specific activities on NADPH (0.72-2.62 U/mg). The K_{M} values of variants Ec_IlvC^{2H10-his6} and Ec_IlvC^{6E6-his6} were measured and the catalytic efficiencies were calculated.

The kinetic parameters of the recombination variants and previously described KARI mutants are shown in Table 32. Both variants found in the recombination library showed an almost complete switch in cofactor preference from NADPH to NADH. The K_{M} values of the mutants on NADH rival the K_{M} value of KARI Ec_IlvC^{his6} on NADPH (44.2 and 31.6 μM on NADH vs. 41 μM for Ec_IlvC^{his6} on NADPH). The catalytic efficiencies of Ec_IlvC^{2H10-his6} and Ec_IlvC^{6E6-his6} on NADH (60322 and 74045 M⁻¹*s⁻¹, respectively) came very close to the catalytic efficiency of Ec_IlvC^{his6} on NADPH (87300 M⁻¹*s⁻¹). The mutants described herein exhibit a complete reversal in cofactor specificity and the NADH-dependent activity approaches the NADPH-dependent activity of the wild-type enzyme. The best variant exhibited 85% activity (in terms of k_{cat}/K_{M}) on NADH compared to wild-type activity on NADPH.

TABLE 32
Kinetic parameters of Ec_IlvC^{his6}, two of the enzymes described previously
(Ec_IlvC^{B8-his6} and Ec_IlvC^{B8A71S-his6}), as well as the two mutants
Ec_IlvC^{2H10-his6} and Ec_IlvC^{6E6-his6}

| | U/mg | | K_{M} [μM] | | k_{cat} [^{s-1}] | | k_{cat}/K_{M} [M⁻¹ * s⁻¹] | |
|---|---|---|---|---|---|---|---|---|
| Variant | NADH | NADPH | NADH | NADPH | NADH | NADPH | NADH | NADPH |
| Ec_IlvC^{his6} | 0.08 | 1.00 | 1,075 | 41 | 1.0 | 3.6 | 1,000 | 87,300 |
| Ec_IlvC^{B8-his6} | 0.57 | 0.62 | 65 | 65 | 2.0 | 2.2 | 31,775 | 34,188 |
| Ec_IlvC^{B8A71S-his6} | 0.57 | 0.66 | 53.5 | 63.4 | 2.0 | 2.4 | 38,330 | 37,459 |
| Ec_IlvC^{2H10-his6} | 0.74 | 0.17 | 44.2 | 568 | 2.6 | 0.61 | 60,322 | 1,078 |
| Ec_IlvC^{6E6-his6} | 0.65 | 0.07 | 31.6 | 653 | 2.3 | 0.2 | 74,045 | 386 |

The above data demonstrates the effects brought on by the beneficial mutations at positions 71 and 110. Moreover, aspartic acids at positions 76 and 78 electrostatically repel the phosphate of NADPH. It is noted that the electrostatic attraction of arginine to the NADPH phosphate is lost when R76 is mutated to an aspartic acid residue.

### Example 16

### KARI Engineering by Random Mutagenesis in Yeast

The following example demonstrates increases in specific, NADH-dependent KARI activity.

Methods: Plasmid pGV2241 (SEQ ID NO: 124) carrying the Ec_ilvC_coSc^{6E6-his6} gene (SEQ ID NO: 33) served as template for generating the first error-prone PCR library using forward primer pGV1994ep_for and reverse primer pGV1994_rev. These primers are specific to the backbone pGV1102 (SEQ ID NO: 101) and bind 50 by upstream and downstream of the KARI insert to create an overlap for homologous recombination in yeast. Generally, three different MnCl₂ concentrations were tested (100, 200, and 300 μM MnCl₂) and the PCR compositions are summarized in Table 33.

TABLE 33
PCR set up for different concentrations of MnCl₂ that were tested. The
final volumes were 100 μL and amounts of ingredients are in μL

| | final MnCl₂ concentration [μM] | | | | |
|---|---|---|---|---|---|
| | 100 | 150 | 200 | 250 | 300 |
| Template | 1 | 1 | 1 | 1 | 1 |
| primer forward | 2 | 2 | 2 | 2 | 2 |
| primer reverse | 2 | 2 | 2 | 2 | 2 |
| dNTP's | 4 | 4 | 4 | 4 | 4 |
| Taq buffer | 10 | 10 | 10 | 10 | 10 |
| MgCl₂ | 28 | 28 | 28 | 28 | 28 |
| Taq polymerase | 1.6 | 1.6 | 1.6 | 1.6 | 1.6 |
| MnCl₂ (1 mM stock) | 10 | 15 | 20 | 25 | 30 |
| PCR grade water | 41.4 | 36.4 | 31.4 | 26.4 | 21.4 |

The temperature profile was the following: 95° C. 3 min initial denaturation, 95° C. 30 s denaturation, 55° C. 30 s annealing, 72° C. 2 min elongation, 25 cycles, 5 min final elongation at 72° C.

The PCR products were checked on a 1% analytical TAE agarose gel, DpnI digested for 1 h at 37° C. to remove traces of template DNA, and then cleaned up using a 1% preparative TAE agarose gel. The agarose pieces containing the PCR products were put into Freeze ‘n’ Squeeze tubes (BIORAD, catalog #732-6166) and frozen for 10 min at −20° C. Then, they were spun down at room temperature and 10,000 rpm to “squeeze” the buffer with the soluble DNA out of the agarose mesh. The volume of the eluted DNA/buffer mixture was estimated and then subjected to the pellet paint procedure (Novagen, catalog #69049-3), which was performed according to the manufacturer's manual. The dried pink DNA pellets were resuspended in 50 μL PCR grade water. In the meantime, the restriction digest of the backbone pGV1102 (SEQ ID NO: 101) was performed as follows: 10 μL of DNA, 32 μL PCR grade water, 5 μL NEB buffer 3 (10×), 2 μL NotI, and 1 μL SalI. After an incubation time of 3 h at 37° C., the digest was run out on an agarose gel and then pellet painted as described above. After determining the DNA concentration of cut vector and insert, 500 ng of each were mixed together, precipitated with pellet paint, and resuspended in 6 μL of PCR grade water. This mixture can be prepared a day before the transformation.

In the evening before the planned transformation, YPD medium (10 g/L yeast extract, 20 g/L peptone, 20 g/L glucose) was inoculated with a single colony of GEVO1186 and incubated at 30° C. and 250 rpm over night. The next morning, a 20 mL YPD culture was started in a 250 ml Erlenmeyer flask without baffles with the overnight culture at an OD₆₀₀ of 0.1. This culture was incubated at 30° C. and 250 rpm until it reached an OD₆₀₀ of 1.3-1.5. When the culture had reached the desired OD₆₀₀, 200 μL of freshly prepared sterile-filtered Tris-DTT (0.39 g 1,4-dithiothreitol per 1 mL of 1 M Tris, pH 8.0) were added and the culture was allowed to incubate at 30° C. and 250 rpm for another 15 min. The cells were then pelleted at 4° C. and 2,500×g for 3 min. After removing the supernatant, the pellet was resuspended in 10 mL of ice-cold buffer E and spun down again as described above. Then, the cell pellet was resuspended in 1 mL of sterile-filtered ice-cold buffer E (1.2 g Tris base, 92.4 g glucose, and 0.2 g MgCl₂ per 1 L deionized water, adjusted to pH 7.5) and spun down one more time as before. After removal of the supernatant with a pipette, 200 μL of ice-cold buffer E (1.2 g/L Tris, 92.4 g/L glucose, and 0.2 g/L MgCl₂, pH 7.5) were added and the pellet was gently resuspended. The 6 μL of insert/backbone mixture were split in half and added to 50 μL of electrocompetent GEVO1186 cells. The DNA/cell mixtures were transferred into 0.2 cm electroporation cuvettes (BioRad) and electroporated without a pulse controller at 0.54 kV and 25 μF. 1 mL of pre-warmed YPD medium was added immediately and the transformed cells were allowed to regenerate at 30° C. and 250 rpm in 15 mL round bottom culture tubes (Falcon). After 1 hour, the cells were spun down at 4° C. and 2,500×g for 3 min, and the pellets were resuspended in 1 mL pre-warmed SD-URA medium (1.7 g/L yeast nitrogen base, 5 g/L ammonium sulfate, 20 g/L glucose, with casamino acids but without uracil (CSM-URA). Different amounts of transformed cells were plated on SD-URA agar plats plates and incubated at 30° C. for 1.5 days or until the colonies were large enough to be picked with sterile toothpicks.

Single yeast colonies were picked with sterile toothpicks into shallow 96-well plates containing 300 μL of SC-URA medium (6.7 g/L Difco™ Yeast Nitrogen Base, 14 g/L Sigma™ Synthetic propout Media supplement (includes amino acids and nutrients excluding histidine, tryptophan, uracil, and leucine), 10 g/L casamino acids, 20 g/L glucose, 0.018 g/L adenine hemisulfate, and 0.076 g/L tryptophan) per well. Each plate encompassed 88 wells with variants, four wells with parent, three wells with GEVO1886 carrying pGV1102 as background control, and one well with medium only, which served as a sterility control. The plates were incubated at 250 rpm and 30° C. in a humidified plate shaker (Kuhner) over night. On the next morning, 50 μL of the overnight culture were transferred into 600 μL SC-URA medium in 96 well deep well plates (2 mL capacity per well). The cultures were allowed to grow for another 8 h at the same conditions, before they were spun down at 4° C. and 5000 rpm for 5 min. The supernatants were removed and the pellets were frozen at −20° C. until they were screened for activity as described in Example 14 above.

Improved variants were expressed and purified from GEVO1186. 20 mL SC-URA medium overnight cultures were grown at 30° C. and 250 rpm in 250 mL flasks and were then used to inoculate 96 well deep well plates on the next morning. 50 μL of the overnight cultures were transferred into 600 μL SC-URA medium per well. The plates were then grown at 30° C. and 250 rpm in a humidified plate shaker for 8 h. In order to the harvest, the cultures were transferred into 50 mL Falcon tubes and then spun down at 4° C. and 5,000 rpm for 10 min. The pellets were frozen until they were processed and purified as described in Example 14 above.

Results: Two rounds of error-prone PCR and screening were carried out. The libraries (˜2400 clones per library) were screened using the KARI high-throughput assay. KARI variants that exhibited an improved activity compared to their parent (total of 88 variants) were picked and rescreened in triplicate and five clones were selected for sequencing and purification. In the first round variant Ec_IlvC^{P2D1-his6} (SEQ ID NO: 38), encoded by Ec_ilvC_coSc^{P2D1-his6} (SEQ ID NO: 37) was identified carrying the following mutations: D146G and G185R. This variant served as parent for the second round of error-prone PCR and screening which yielded variant Ec_IlvC^{P2D1-A1-his6} (SEQ ID NO: 42), encoded by Ec_ilvC_coSc^{P2D1-A1-his6} (SEQ ID NO: 41) with one additional mutation (K433E). The biochemical properties were determined and are summarized in Table 34. A two-fold improvement of the specific activity in lysate and in the purified enzyme was observed after two rounds of error-prone PCR.

TABLE 34
Comparison of the biochemical properties of the parent Ec_IlvC^{6E6-his-6} with
the variants found in round 1 (Ec_IlvC^{P2D1-his6}) and 2 (Ec_IlvC^{P2D1-A1-his6}).
The variants were purified before characterization

| | U/mg | | K_{M} [μM] | | k_{cat} [^{s-1}] | | k_{cat}/K_{M} [M⁻¹ * s⁻¹] | |
|---|---|---|---|---|---|---|---|---|
| Variant | NADH | NADPH | NADH | NADPH | NADH | NADPH | NADH | NADPH |
| Ec_IlvC^{6E6-his6} | 0.69 | | 39 | | 2.4 | | 63,000 | |
| Ec_IlvC^{P2D1-his6} | 0.92 | 0.15 | 40 | 1432 | 3.3 | 0.54 | 82,650 | 377 |
| Ec_IlvC^{P2D1-A1-his6} | 1.2 | 0.15 | 26 | >1432 | 4.3 | 0.54 | 167,687 | <377 |

### Example 17

### NADH-Dependent Anaerobic Isobutanol Production

This example illustrates that an isobutanol producing microorganism which is engineered to carry NADH-dependent KARI and ADH enzymes produces isobutanol at higher yield compared to strains engineered to carry NADPH-dependent KARI and ADH enzymes. These strains also acquire the ability to produce isobutanol anaerobically.

A first set of anaerobic fermentations with isobutanol producing strains according to Table 35 were performed. Strain GEVO1993 is an E. coli strain in which the native ilvC gene was deleted and the other three steps of the isobutanol pathway (Bs_alsS1, Ec_ilvD_coEc and LI_kivd1) were integrated into the chromosome.

TABLE 35
Strain/Plasmid combinations described herein.

| | | | | Cofactor |
|---|---|---|---|---|
| | | | | usage of the |
| | | | | isobutanol |
| Plasmid | Strain | KARI gene | ADH gene | pathway |
| pGV1777 | GEVO1993 | Ec_ilvC_coEc | Ec_yqhD | NADPH/ |
| | | | (native) | NADPH |
| pGV1925 | GEVO1993 | Ec_ilvC_coEc | Ec_fucO | NADPH/ |
| | | | | NADH |
| pGV1938 | GEVO1993 | Ec_ilvC_coEc^{S78D} | Ec_yqhD | NADH/ |
| | | | (native) | NADPH |
| pGV1927 | GEVO1993 | Ec_ilvC_coEc^{S78D} | Ec_fucO | NADH/ |
| | | | | NADH |

Overnight cultures of the GEVO1993 transformed with pGV1777 (SEQ ID NO: 118), pGV1925, pGV1938, or pGV1927 were started from individual colonies of previously transformed strains. These cultures were started in 3 mL M9 minimal medium (Miller, J.H. A Short Course in Bacterial Genetics: A laboratory manual and handbook for Escherichia coli and related bacteria. 1992. Cold Spring Harbor Laboratory Press, Cold Spring Harbor, N.Y.), supplemented with 10 g/L yeast extract, 10 μM ferric citrate and trace metals, containing 8.5% glucose and the appropriate antibiotics in snap cap tubes about 14 h prior to the start of the fermentation. Isobutanol fermentations were then carried out in screw cap flasks containing 20 mL of the same medium that was inoculated with 0.2 mL of the overnight culture. The cells were incubated at 37° C./250 rpm until the strains had grown to an OD₆₀₀ of 0.6-0.8 and were then induced with Isopropyl β-D-1-thiogalactopyranoside at 1 mM final concentration.

Three hours after induction the cultures were shifted to anaerobic fermentation conditions by loosening the cap of the flasks and placing the flasks into to a Coy Laboratory Products Type B Vinyl anaerobic chamber (Coy Laboratory Products, Grass Lakes, Mich.) through an airlock in which the flasks were cycled three times with nitrogen and vacuum, and then filled with the a hydrogen gas mix (95% Nitrogen, 5% Hydrogen). Once the flasks were inside the anaerobic chamber, the flasks were closed again and incubated without shaking at 30° C. Inside the chamber, an anaerobic atmosphere (<5 ppm oxygen) was maintained through the hydrogen gas mix (95% Nitrogen, 5% Hydrogen) reacting with a palladium catalyst to remove oxygen. The flasks in the anaerobic chamber were swirled twice a day. Samples (2 mL) were taken at the time of the shift and at 21 h and 45 h after shifting to anaerobic conditions, spun down at 22,000 g for 1 min to separate the cell pellet from the supernatant and stored frozen at −20° C. until analysis. The samples were analyzed using High performance liquid chromatography (HPLC) and gas chromatography GC. All experiments were performed in triplicate.

The OD₆₀₀ values of the cultures were similar amongst the three replicates. Notably, after 45 h, GEVO1993+pGV1927 (i.e. expressing NADH-dependent KARI and ADH) produced isobutanol at approximately twice the volumetric productivity, specific productivity, and titer. Surprisingly the theoretical yield increased from about 70% of theoretical to 96% of theoretical. Expressing only one NADH-dependent enzyme with the other enzyme being NADPH-dependent did not have an effect (Table 36).

TABLE 36
45 h performance parameters

| | | Vol. | | Spec. | | Anaerobic | | | |
|---|---|---|---|---|---|---|---|---|---|
| | | Productivity | | Productivity | | Yield^{a} | | Titer | |
| Sample | KARI/ADH | [g/L/h] | ± | [g/L/h/OD] | ± | % theor. | ± | [g/L] | ± |
| GEVO1993 + | Ec_IlvC/ | 0.044 | 0.019 | 0.018 | 0.003 | 72 | 3 | 2.4 | 1.0 |
| pGV1777 | Ec_YqhD | | | | | | | | |
| GEVO1993 + | Ec_IlvC/ | 0.031 | 0.002 | 0.017 | 0.003 | 55 | 4 | 1.9 | 0.1 |
| pGV1925 | Ec_FucO | | | | | | | | |
| GEVO1993 + | Ec_IlvC^{S78D}/ | 0.040 | 0.015 | 0.021 | 0.002 | 78 | 10 | 2.1 | 0.9 |
| pGV1938 | Ec_YqhD | | | | | | | | |
| GEVO1993 + | Ec_IlvC^{S78D}/ | 0.078 | 0.006 | 0.030 | 0.003 | 96 | 5 | 3.8 | 0.2 |
| pGV1927 | Ec_FucO | | | | | | | | |

^{a}The anaerobic yield is calculated by dividing the isobutanol produced from time of anaerobic shift until 45 hours after the shift by the amount of glucose consumed during this time period

A second set of anaerobic fermentations with isobutanol producing strains according to Table 37 were performed to demonstrate that the of improved KARI variants correlates with an improvement of isobutanol production under anaerobic conditions.

TABLE 37
Strain/Plasmid combinations used for the second set of anaerobic fermentations.

| | | | | | | KARI |
|---|---|---|---|---|---|---|
| | | | | ADH | KARI | (k_{cat}/K_{M,NADH})/ |
| # | Plasmid | Strain | KARI gene | gene | k_{cat}/K_{M,NADH} | (k_{cat}/K_{M,NADPH}) |
| 1 | pGV1927 | GEVO1993 | Ec_ilvC_coEc^{S78D} | Ec_fucO | 27,600 | 7 |
| 2 | pGV1976 | GEVO1993 | Ec_ilvC_coEc^{2H10} | Ec_fucO | 60,300 | 56 |
| 3 | pGV1975 | GEVO1993 | Ec_ilvC_coEc^{6E6} | Ec_fucO | 74,000 | 192 |

The experiment was carried out as described above except that the cell cultures were induced at an OD₆₀₀ of 0.8-1.0 instead of 0.6-0.8 and shifted to anaerobic conditions at and OD OD₆₀₀ of 4.0-6.0 instead of 3 hours after induction. In addition, samples were taken at the time of the anaerobic shift and 24 h and 48 h after induction (i.e. 20 h and 44 h after the anaerobic shift, respectively).

44 hours after shift to anaerobic fermentation conditions, the trend for volumetric and specific productivity is the same as observed 20 hours after shift to anaerobic conditions: strains carrying improved KARI variants Ec_IlvC^{2H10} and Ec_IlvC^{6E6} produced isobutanol at higher volumetric and specific productivity as well as yield compared to strains carrying KARI variant Ec_IlvC^{S78D} (Table 38).

TABLE 38
44 h performance parameters

| | | Vol. | | Spec. | | anaerobic | | | |
|---|---|---|---|---|---|---|---|---|---|
| | | Productivity | | Productivity | | Yield^{a} | | Titer | |
| Sample | KARI/ADH | [g/L/h] | ± | [g/L/h/OD] | ± | % theor. | ± | [g/L] | ± |
| GEVO1993 + | Ec_IlvC^{S78D}/ | 0.215 | 0.005 | 0.037 | 0.002 | 79 | 12 | 10.9 | 0.3 |
| pGV1927 | Ec_FucO | | | | | | | | |
| GEVO1993 + | Ec_IlvC^{2H10}/ | 0.274 | 0.008 | 0.047 | 0.002 | 107 | 15 | 13.0 | 0.6 |
| pGV1976 | Ec_FucO | | | | | | | | |
| GEVO1993 + | Ec_IlvC^{6E6}/ | 0.270 | 0.032 | 0.047 | 0.005 | 97 | 2 | 12.5 | 1.5 |
| pGV1975 | Ec_FucO | | | | | | | | |

^{a}The anaerobic yield is calculated by dividing the isobutanol produced from time of anaerobic shift until 44 hours after the shift by the amount of glucose consumed during this time period

### Example 18

### NADH-Dependent Anaerobic Isobutanol Production in Yeast

This example illustrates that isobutanol producing yeast microorganisms engineered to carry NADH-dependent KARI and ADH enzymes produce isobutanol at higher yields compared to isobutanol producing yeast microorganisms engineered to carry NADPH-dependent KARI and/or ADH enzymes. These strains also produce isobutanol anaerobically.

Cultures of GEVO2710, GEVO2711 and GEVO2799 transformed with pGV2227 (SEQ ID NO: 123) or pGV2242 (SEQ ID NO: 125) and cultures of GEVO2710, and GEVO2799 transformed with pGV2020 (SEQ ID NO: 121) or pGV2082 (SEQ ID NO: 122) were started from individual colonies of previously transformed and purified strains. These cultures were started in 14 ml round-bottom snap-cap test tubes containing 3 ml of YPD medium supplemented with 0.2 g/L G418 antibiotic, and1% (v/v) of a stock solution containing 3 g/L ergosterol and 66 g/L Tween 80 dissolved in ethanol. The snap-cap test tubes were not closed completely so that air would vent in/out of the tubes. After growth for about 10 hours at 30° C. shaking at 250 rpm, these cultures were added to 47 ml of the same medium in 250 ml non-baffled flasks with sleeve closures and incubated for about 14 hours at 30° C. shaking at 250 rpm. Isobutanol fermentations were then carried out after harvesting the cells from the 50 ml cultures by centrifugation, and resuspending the cell pellets in f 50 ml of the same medium in 250 ml non-baffled flasks to an initial optical density (OD₆₀₀) of 3-6.

Anaerobic fermentations were carried out by inoculating flasks with screw-cap closures as above and placing the flasks with loose caps into to a Coy Laboratory Products Type B Vinyl anaerobic chamber (Coy Laboratory Products, Grass Lakes, Mich.) through an airlock in which the flasks were cycled three times with nitrogen and vacuum, and then filled with a hydrogen gas mix (95% Nitrogen, 5% Hydrogen). The flasks were moved inside the anaerobic chamber from the airlock and the screw-caps on the flasks were closed inside the anaerobic chamber. Inside the chamber, an anaerobic atmosphere (<5 ppm oxygen) was maintained through the hydrogen gas mix (95% Nitrogen, 5% Hydrogen) reacting with a palladium catalyst to remove oxygen. The flasks were then removed from the anaerobic chamber and incubated outside the anaerobic chamber at 30° C. shaking at 75 rpm. Samples (2 ml) were taken at the beginning of the incubation of the anaerobic fermentations and after 24 hours, 48 hours and 72 hours of incubation. The samples taken at the beginning of the incubation were taken before moving the flasks into the anaerobic chamber. The 24 hour and 48 hour samples were taken by moving the flasks into the anaerobic chamber through the airlock as above, opening the flasks in the anaerobic chamber to remove the samples, re-closing the flasks in the anaerobic chamber and removing the flasks from the anaerobic chamber for continued incubation. The 72 hour samples were taken outside of the anaerobic chamber because these were the final samples from the flasks.

Samples from fermentations were centrifuged for 10 minutes at 18,000 g to separate the cells from the supernatant. The supernatant was removed and stored under refrigeration until analyzed by gas chromatography and high performance liquid chromatography as described above. All experiments were performed in triplicate.

In the anaerobic fermentations the OD₆₀₀ values of the cultures were similar amongst the three replicates. Notably, after 72 hours in anaerobic fermentations, GEVO2710+pGV2242, GEVO2711+pGV2242 and GEVO2799+pGV2242 (i.e. strains expressing an NADH-dependent KARI) produced isobutanol at an approximately 1.25- to 2-fold higher volumetric productivity, specific productivity, and titer than the same strains containing pGV2227 (i.e. strains expressing an NADPH-dependent KARI). The anaerobic yield increased from about 16-25% of theoretical to 22-35% of theoretical (Table 39).

TABLE 39
72 hour performance parameters from anaerobic fermentations

| | KARI/ADH | Vol. | | Spec. | | | | Specific | |
|---|---|---|---|---|---|---|---|---|---|
| | overexpressed | Productivity | | Productivity | | Yield | | Titer | |
| Sample | from plasmid | [g/L/h] | ± | [g/L/h/OD] | ± | % theor. | ± | [g/L/OD] | ± |
| GEVO2710 + | None/ | 0.000 | 0.000 | 0.0001 | 0.0000 | 1 | 0 | 0.01 | 0.00 |
| pGV2020 | None | | | | | | | | |
| GEVO2710 + | Ec_IlvC^{Q110V}/ | 0.006 | 0.001 | 0.0014 | 0.0001 | 21 | 2 | 0.10 | 0.01 |
| pGV2082 | Dm_Adh | | | | | | | | |
| GEVO2710 + | Ec_IlvC^{Q110V}/ | 0.006 | 0.001 | 0.0017 | 0.0003 | 17 | 9 | 0.12 | 0.02 |
| pGV2227 | Ll_AdhA | | | | | | | | |
| GEVO2710 + | Ec_IlvC^{P2D1}/ | 0.011 | 0.001 | 0.0029 | 0.0003 | 22 | 2 | 0.21 | 0.02 |
| pGV2242 | Ll_AdhA | | | | | | | | |
| GEVO2799 + | None/ | 0.001 | 0.000 | 0.0002 | 0.0000 | 6 | 1 | 0.01 | 0.00 |
| pGV2020 | None | | | | | | | | |
| GEVO2799 + | Ec_IlvC^{Q110V}/ | 0.010 | 0.000 | 0.0019 | 0.0003 | 38 | 2 | 0.14 | 0.02 |
| pGV2082 | Dm_Adh | | | | | | | | |
| GEVO2799 + | Ec_IlvC^{Q110V}/ | 0.009 | 0.001 | 0.0014 | 0.0002 | 20 | 2 | 0.10 | 0.01 |
| pGV2227 | Ll_AdhA | | | | | | | | |
| GEVO2799 + | Ec_IlvC^{P2D1}/ | 0.014 | 0.003 | 0.0026 | 0.0003 | 33 | 10 | 0.19 | 0.03 |
| pGV2242 | Ll_AdhA | | | | | | | | |
| GEVO2711 + | Ec_IlvC^{Q110V}/ | 0.008 | 0.000 | 0.0020 | 0.0000 | 24 | 2 | 0.14 | 0.00 |
| pGV2227 | Ll_AdhA | | | | | | | | |
| GEVO2711 + | Ec_IlvC^{P2D1}/ | 0.014 | 0.004 | 0.0025 | 0.0008 | 37 | 8 | 0.18 | 0.06 |
| pGV2242 | Ll_AdhA | | | | | | | | |

### Example 19

### Overexpression of an NADPH-Dependent GAPDH, GDP1

The purpose of this example is to describe how overexpression of an NADPH-dependent GAPDH can improve isobutanol production under anaerobic conditions.

GDP1 is expressed from plasmid pGV1573 (SEQ ID NO: 106) together with an isobutanol biosynthetic pathway expressed from pGV1485 (SEQ ID NO: 103) and pSA69. As a control the plasmid pGV1573 is replaced by the empty version of this plasmid pGV1572 (SEQ ID NO: 105). These plasmids are transformed into GEVO1859ΔgapA. Overnight cultures of Strain 1: GEVO1859 ΔgapA, pGV1573, pGV1485, pSA69 and Strain 2: GEVO1859ΔgapA, pGV1572, pGV1485, pSA69 are started from individual colonies of previously transformed strains. These cultures are started in 3 mL M9 minimal medium (Miller, J.H. A Short Course in Bacterial Genetics: A laboratory manual and handbook for Escherichia coli and related bacteria. 1992. Cold Spring Harbor Laboratory Press, Cold Spring Harbor, N.Y.), supplemented with 10 g/L yeast extract, 10 μM ferric citrate and trace metals, containing 8.5% glucose and the appropriate antibiotics in snap cap tubes about 14 h prior to the start of the fermentation. Isobutanol fermentations are then carried out in screw cap flasks containing 20 mL of the same medium that was inoculated with 0.2 mL of the overnight culture. The cells are incubated at 37° C./250 rpm until the strains had grown to an OD₆₀₀ of 0.6-0.8 and are then induced with Isopropyl β-D-1-thiogalactopyranoside at 1 mM final concentration.

Three hours after induction the cultures are shifted to anaerobic fermentation conditions by loosening the cap of the flasks and placing the flasks into to a Coy Laboratory Products Type B Vinyl anaerobic chamber (Coy Laboratory Products, Grass Lakes, Mich.) through an airlock in which the flasks are cycled three times with nitrogen and vacuum, and then filled with the a hydrogen gas mix (95% Nitrogen, 5% Hydrogen). Once the flasks are inside the anaerobic chamber, the flasks are closed again and incubated without shaking at 30° C. Inside the chamber, an anaerobic atmosphere (<5 ppm oxygen) was maintained through the hydrogen gas mix (95% Nitrogen, 5% Hydrogen) reacting with a palladium catalyst to remove oxygen. The flasks in the anaerobic chamber are swirled twice a day. Samples (2 mL) are taken at the time of the shift and at 24 h and 48 h after inoculation, spun down at 22,000 g for 1 min to separate the cell pellet from the supernatant and stored frozen at −20° C. until analysis. The samples are analyzed using High performance liquid chromatography (HPLC) and gas chromatography GC. All experiments are performed in duplicate.

### Example 20

### Overexpression of NADPH-Dependent GADPHs GDP1 and gapC

pGV1572 (SEQ ID NO: 105) (PLlacO, p15A, Cm^{R}) was constructed as an empty vector compatible with the plasmids pGV1698 (SEQ ID NO: 112) and pGV1655 (SEQ ID NO: 109) for the expression of the isobutanol pathway. The GAPDHs from Kluyveromyces lactis, and Clostridium acetobutylicum were cloned into pGV1572 to make pGV1573 (SEQ ID NO: 106) (PLlacO1::GDP1, p15A, Cm^{R}), and pGV1573 (SEQ ID NO: 107) (PLlacO1::GapC, p15A, Cm^{R}) respectively. K. lactis GAPDH was subcloned from pGV1323 (SEQ ID NO: 102), which contains the GDP1 gene cloned from genomic DNA of K. lactis. GapC (C. acetobutylicum) was cloned from genomic DNA using primers 1049 and 1050.

E. coli DH5αZ1 (Lutz, R. and Bujard, H, Nucleic Acids Research (1997) 25 1203-1210) was chosen as the host strain. This strain contains the Z1 integration which provides overexpression of lacI from a lacIq expression cassette. DH5aZ1 was transformed with pGV1572, pGV1573, and pGV1575. Transformants were used to inoculate 5 mL cultures, which were incubated at 37° C., 250 rpm overnight. 50 mL cultures were inoculated with 1 mL overnight culture and incubated at 37° C., 250 rpm. The cultures were induced with IPTG when OD₆₀₀ was approximately 0.6 and incubated at 30° C., 250 rpm for 2 hours. The cultures were centrifuged at 2700×g at 4° C. for 10 min and the pellets were frozen at −80° C.

Pellets were resuspended with lysis buffer to 40% (w/v). (lysis buffer was the same as the reaction buffer but without substrate and cofactors). Cells were lysed in a bead mill using 3 times 1 min intervals, placing them on ice for 2 min in between each run. The lysate was centrifuged at 25000×g at 4° C. for 10 min, the supernatant was kept on ice and it was used as whole cell lysate for the enzyme assays.

The total reaction volume was 100 μL consisting of 90 μL of Reaction Buffer: 50 mM glycine buffer pH 9.5, 5 mM EDTA, 40 mM triethanolamine, 3 mM beta-mercaptoethanol, 6 mM NAD+ or NADP+, and 10 pt lysate. 10 pt of lysate were pipette into a UV permeable 96 well plate. 90 pt of reaction buffer was added to the lysate and mixed well by pipetting up and down. The plate was read for 5 min at 340 nm. Results are shown in Table 40.

TABLE 40
Volumetric and specific activity of various GAPDH with NADP⁺

| | NADP⁺ | | | |
|---|---|---|---|---|
| | | Sp. Activity | | |
| | Volumetric | (nmol/min/ | | |
| | Activity | μg total | | |
| Lysate Name | (mU/ml) | cell protein) | pGV# | organism |
| gapC | 10.022 | 0.010 | 1575 | C. acetobutylicum |
| GDP1 | 26.849 | 0.031 | 1573 | K. lactis |
| Control | 3.819 | 0.005 | 1572 | |
| (DH5az1) | | | | |

DH5aZ1 was the host strain for all the plasmids and has its own indigenous GAPDH. The results show that the GAPDH enzymes are expressed and active in E. coli. The strain expressing GDP1 had more than 6 times higher in vitro GAPDH specific activity with the cofactor NADPH than the control strain not overexpressing GAPDH. The strain overexpressing gapC had twice the in vitro GAPDH specific activity with the cofactor NADPH than the control strain not overexpressing GAPDH.

### Example 21

### NADPH-Dependent GAPDH in Yeast

The purpose of this example is to describe how an isobutanol producing yeast which is engineered to express NADPH-dependent GAPDH and produce isobutanol anaerobically.

A yeast strain, GEVO5001, which expresses the isobutanol biosynthetic pathway and is deficient in pyruvate decarboxylase activity, is engineered to overproduce the K. lactis Gdp1. pGV6001 is a yeast integration plasmid carrying a hygromycin resistance marker and the GDP1 gene under the strong constitutive promoter from TDH3. This plasmid is linearized and transformed into GEVO5001 to generate GEVO5003. Expression of GDP1 is confirmed by qRT-PCR. Once confirmed, GEVO5003 and the parent strain GEVO5001 are used in fermentations for the production of isobutanol. Two fermentations are performed with the two strains. Fermentation 1 is an aerobic fermentation and Fermentation 2 is an anaerobic fermentation.

### Example 22

### pyk Bypass 1

This example illustrates that an isobutanol producing microorganism which is engineered to bypass the pyruvate kinase reaction shows increased productivity, titer and yield of isobutanol compared to the control strain without said engineering.

For the pyk bypass experiment, GEVO1385, GEVO1725 (triple deletion strain—tet repressor), and GEVO1751 were transformed with pGV1655 (SEQ ID NO: 109), pGV1698 (SEQ ID NO: 112), and pGV1490 (SEQ ID NO: 104) or pGV1661 (SEQ ID NO: 110). Strains GEVO1725 and GEVO1751 contain the deletions of pyruvate kinase and of the NADH dependent malic enzyme which are part of the pyruvate bypass engineering. All of these transformants were tested in isobutanol fermentations.

The aforementioned strains were grown overnight in two biological replicates for each strain in M9+A5 salts+FeCl3+10 g/L YE media and the appropriate antibiotics in 14 ml snap cap tubes and incubated at 37° C., 250 rpm. Screw cap flasks with 20 ml M9+A5 salts+FeCl3+10 g/L YE media and the appropriate antibiotics were inoculated with overnight culture to an OD₆₀₀ of 0.1. The cells were incubated at 37° C., 250 rpm until they were grown to an OD₆₀₀ of 0.6-0.8 and induced with IPTG [1 mM] and aTc [100 ng/ml]. Afterwards the cultures were incubated at 30° C., 250 rpm. Samples were taken of the medium, at 24 h and 48 h after inoculation. Samples were centrifuged at 15000 g for 1 min to separate the cell pellet from the supernatant and stored in −20° C. until sample submission. The samples were analyzed using High performance liquid chromatography (HPLC) and gas chromatography (GC).

The triple deletion strains GEVO1725 and GEVO1751 have a severe growth defect which is partially rescued by introduction of pGV1661.

The analysis of the fermentation data shows that the partial deletion strain, GEVO1750, with pGV1661 only has negative effects on isobutanol production (Tables 41, 42). However, at the 24 h time point the triple deletion strain with and without the tet repressor (GEVO1725 and GEVO1751 respectively) shows increased yield (Table 41). GEVO1725 shows a 20% increase in yield, with specific productivity similar to the control strain. GEVO1751 shows a 13% increase in yield and specific productivity.

TABLE 41
Analysis of the second pyk bypass fermentation from the 24 hour time point

| | Volumetric | | Specific | | | | | |
|---|---|---|---|---|---|---|---|---|
| | Productivity | | Productivity | | Titer | | Yield | |
| Samples 24 h | [g/L/h] | ± | [g/L/h/OD] | ± | [g/L] | ± | [g/g] | ± |
| GEVO1385 + pGV1655, | 0.205 | 0.008 | 0.031 | 0.001 | 4.93 | 0.18 | 0.277 | 0.002 |
| pGV1698, pGV1490 | | | | | | | | |
| (control) | | | | | | | | |
| GEVO1385 + pGV1655, | 0.197 | 0.003 | 0.028 | 0.002 | 4.65 | 0.01 | 0.285 | 0.035 |
| pGV1698, pGV1661 | | | | | | | | |
| (control) | | | | | | | | |
| GEVO1725 + pGV1655, | 0.125 | 0.009 | 0.034 | 0.005 | 2.83 | 0.19 | 0.331 | 0.029 |
| pGV1698, pGV1490 | | | | | | | | |
| GEVO1725 + pGV1655, | 0.184 | 0.002 | 0.031 | 0.001 | 4.16 | 0.04 | 0.333 | 0.004 |
| pGV1698, pGV1661 | | | | | | | | |
| GEVO1750 + pGV1655, | 0.144 | 0.004 | 0.022 | 0.001 | 3.30 | 0.14 | 0.267 | 0.001 |
| pGV1698, pGV1490 | | | | | | | | |
| GEVO1750 + pGV1655, | 0.080 | 0.005 | 0.013 | 0.001 | 1.84 | 0.09 | 0.305 | |
| pGV1698, pGV1661 | | | | | | | | |
| GEVO1751 + pGV1655, | 0.138 | 0.006 | 0.031 | 0.001 | 3.09 | 0.13 | 0.303 | 0.008 |
| pGV1698, pGV1490 | | | | | | | | |
| GEVO1751 + pGV1655, | 0.204 | 0.004 | 0.035 | 0.001 | 4.55 | 0.08 | 0.318 | 0.006 |
| pGV1698, pGV1661 | | | | | | | | |

TABLE 42
Analysis of the second pyk bypass fermentation from the 48 hour time point

| | Volumetric | | Specific | | | | | |
|---|---|---|---|---|---|---|---|---|
| | Productivity | | Productivity | | Titer | | Yield | |
| samples 48 h | [g/L/h] | ± | [g/L/h/OD] | ± | [g/L] | ± | [g/g] | ± |
| GEVO1385 + pGV1655, | 0.128 | 0.011 | 0.023 | 0.002 | 6.14 | 0.53 | 0.271 | 0.004 |
| pGV1698, pGV1490 | | | | | | | | |
| (control) | | | | | | | | |
| GEVO1385 + pGV1655, | 0.141 | 0.029 | 0.023 | 0.005 | 6.75 | 1.41 | 0.263 | 0.002 |
| pGV1698, pGV1661 | | | | | | | | |
| (control) | | | | | | | | |
| GEVO1725 + pGV1655, | 0.070 | 0.002 | 0.024 | 0.002 | 3.25 | 0.10 | 0.299 | 0.009 |
| pGV1698, pGV1490 | | | | | | | | |
| GEVO1725 + pGV1655, | 0.101 | 0.006 | 0.024 | 0.002 | 4.72 | 0.28 | 0.309 | 0.005 |
| pGV1698, pGV1661 | | | | | | | | |
| GEVO1750 + pGV1655, | 0.102 | 0.013 | 0.018 | 0.002 | 4.77 | 0.54 | 0.277 | 0.013 |
| pGV1698, pGV1490 | | | | | | | | |
| GEVO1750 + pGV1655, | 0.085 | 0.003 | 0.015 | 0.001 | 4.02 | 0.13 | 0.261 | 0.018 |
| pGV1698, pGV1661 | | | | | | | | |
| GEVO1751 + pGV1655, | 0.093 | 0.004 | 0.029 | 0.001 | 4.29 | 0.16 | 0.267 | 0.006 |
| pGV1698, pGV1490 | | | | | | | | |
| GEVO1751 + pGV1655, | 0.123 | 0.002 | 0.041 | 0.001 | 5.68 | 0.06 | 0.302 | 0.009 |
| pGV1698, pGV1661 | | | | | | | | |

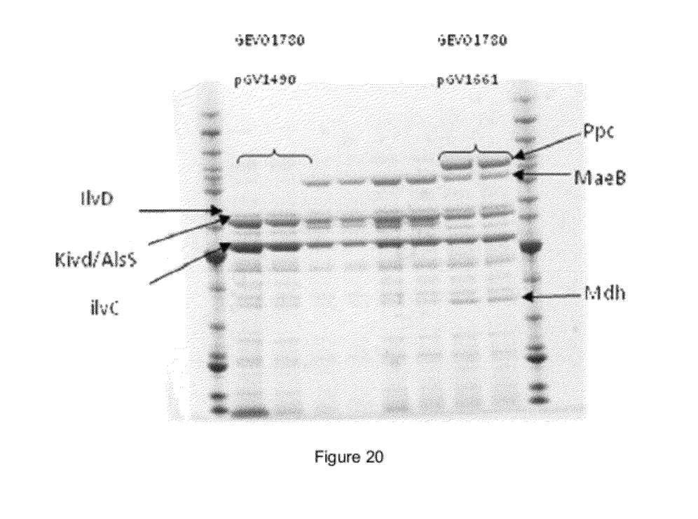

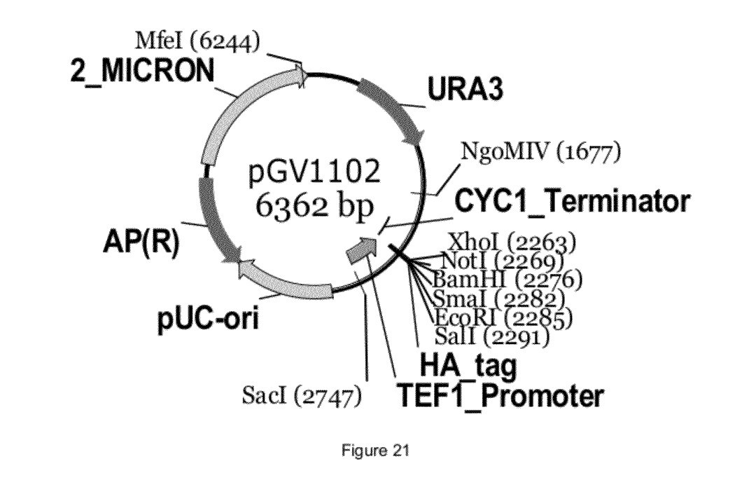

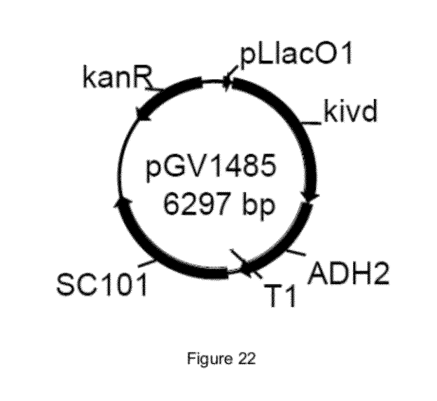

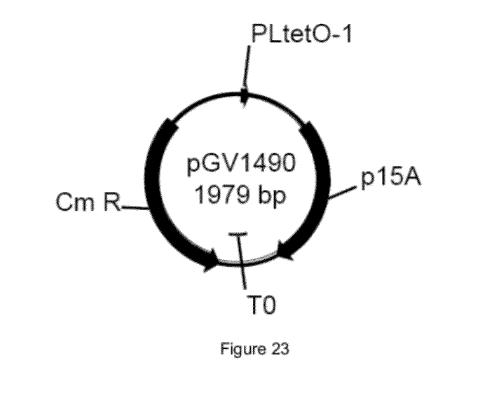

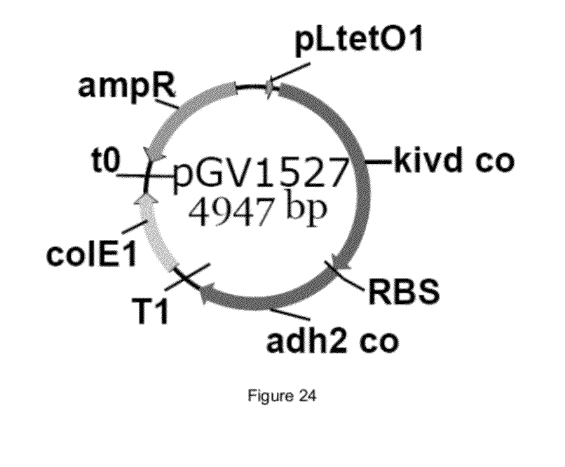

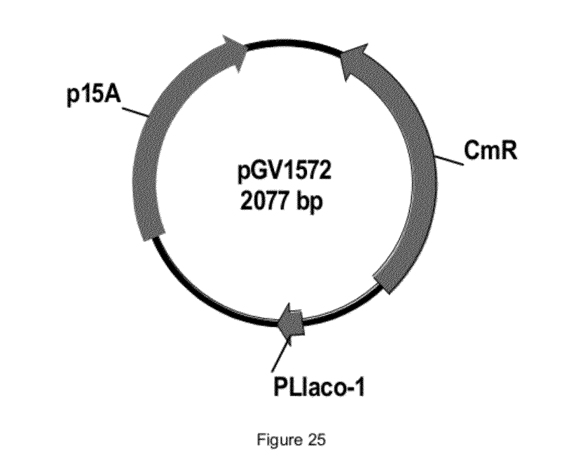

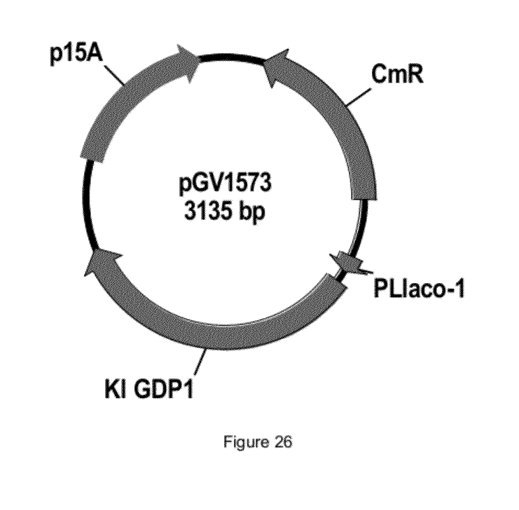

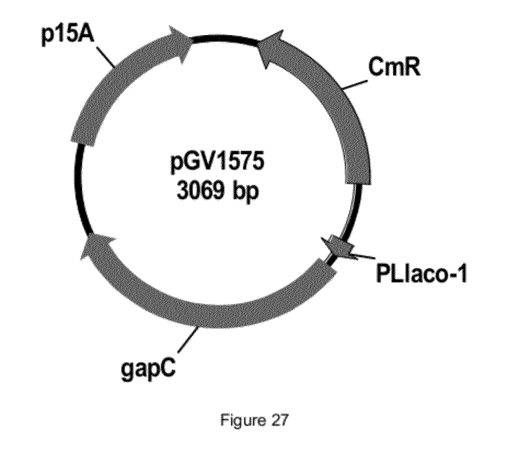

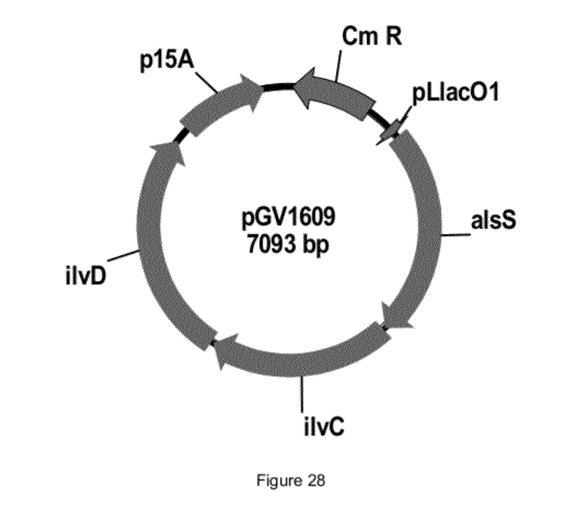

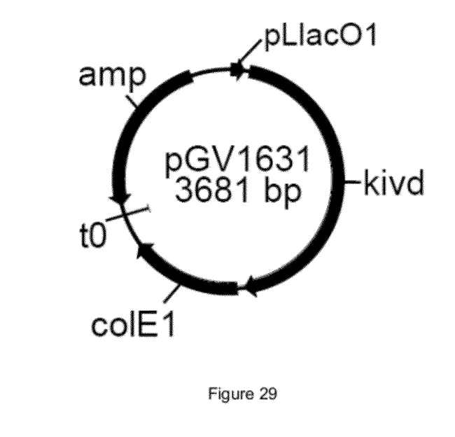

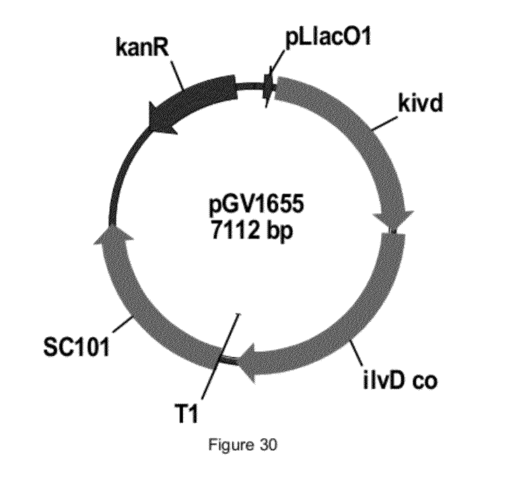

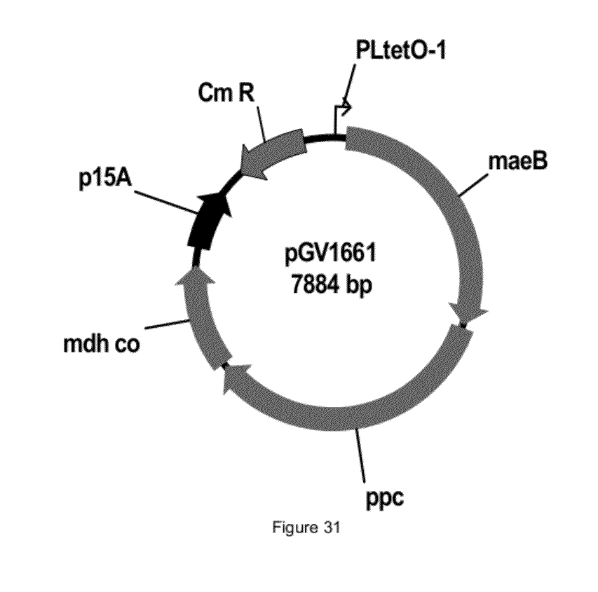

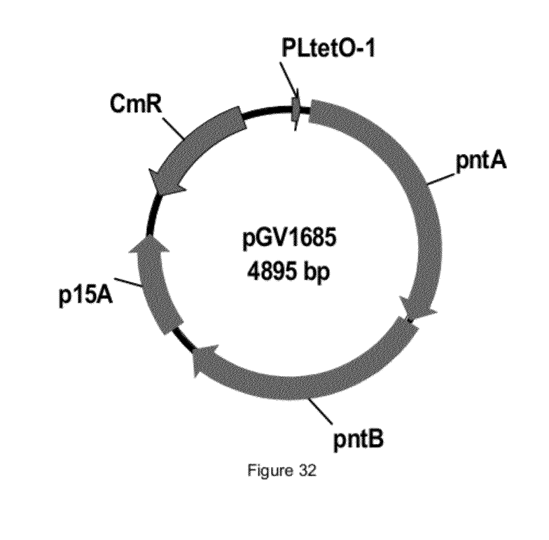

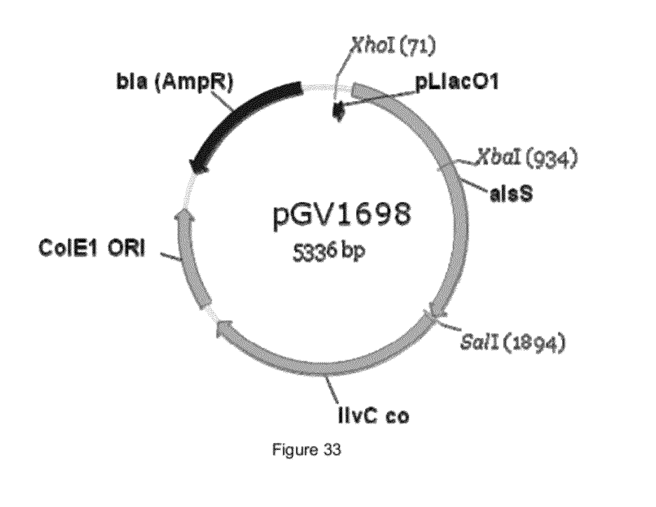

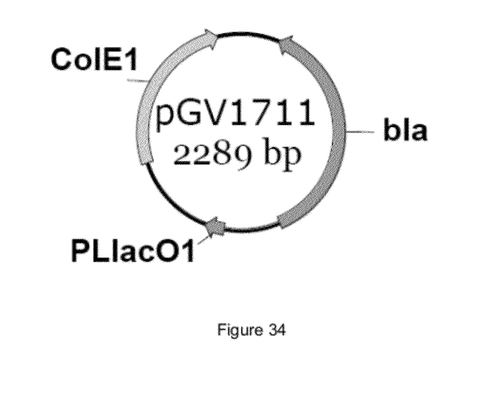

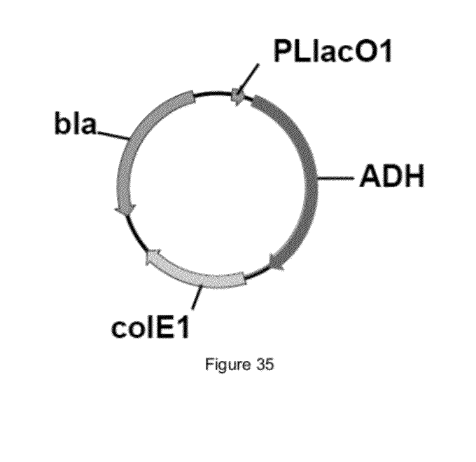

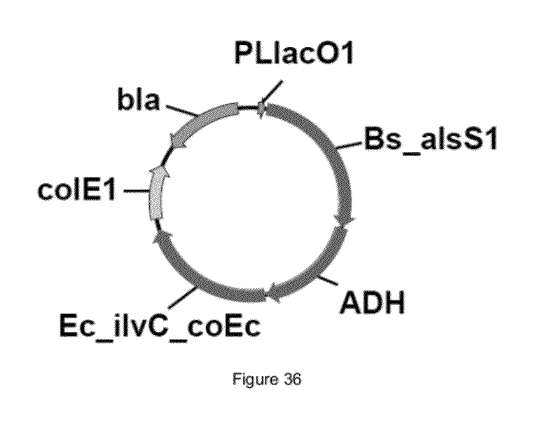

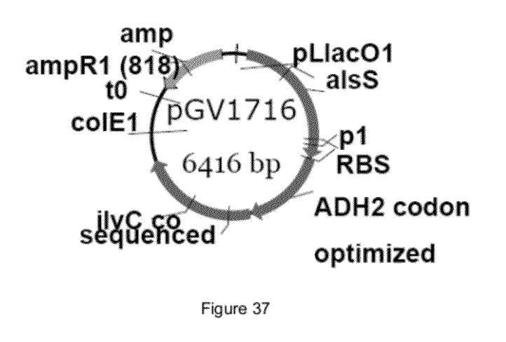

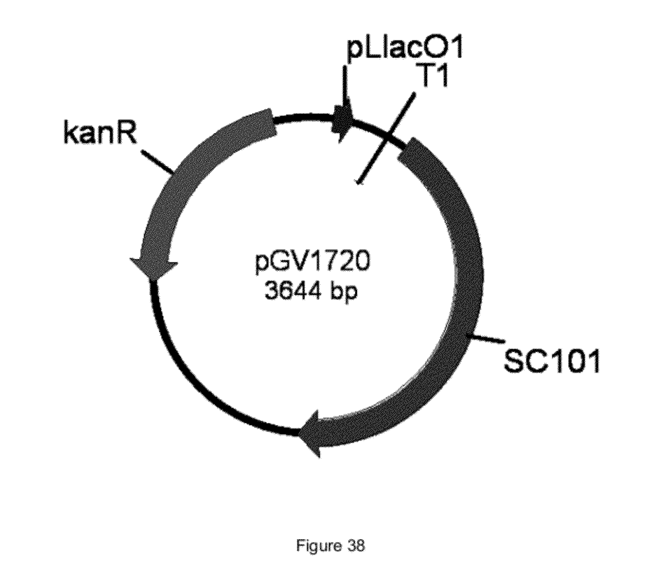

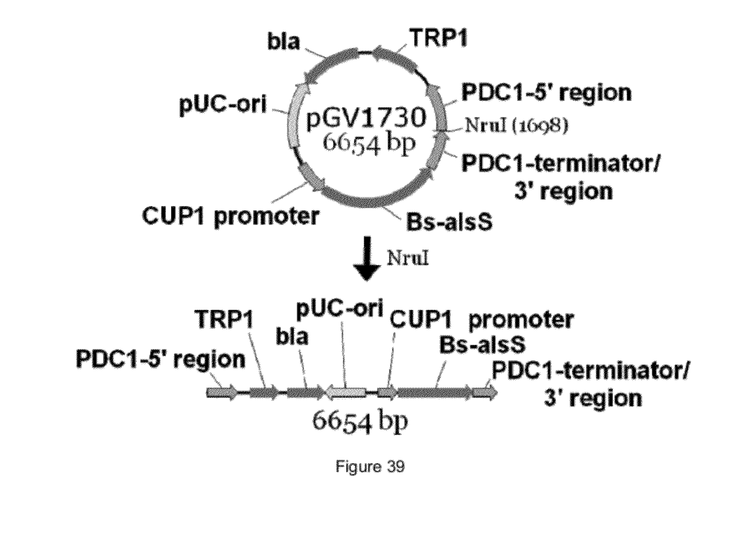

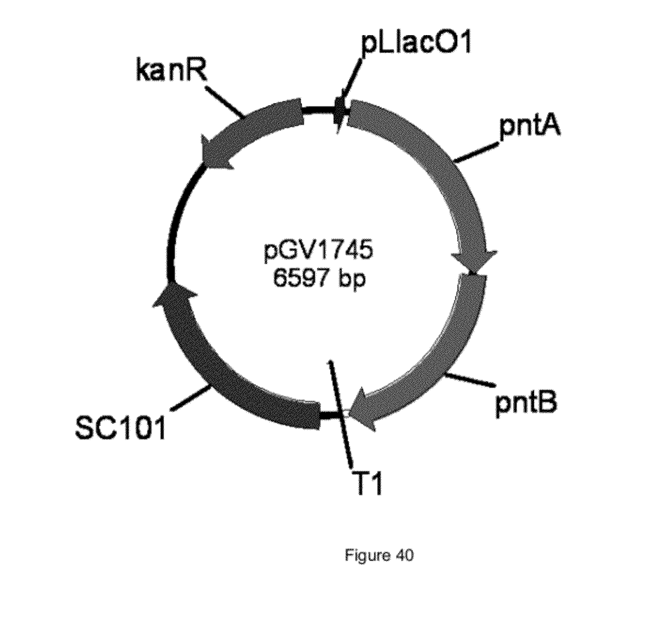

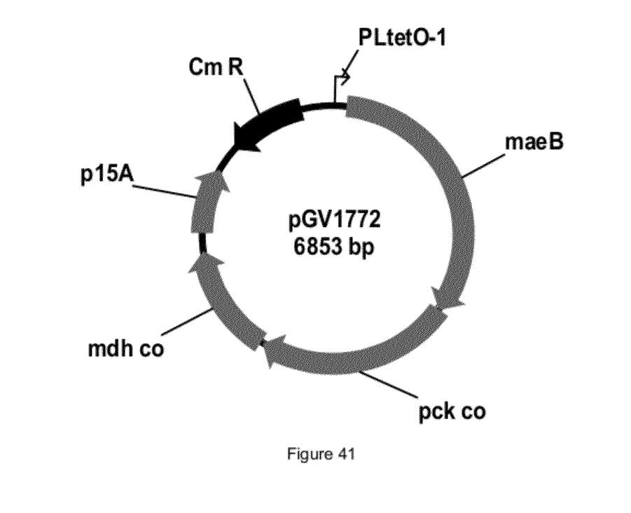

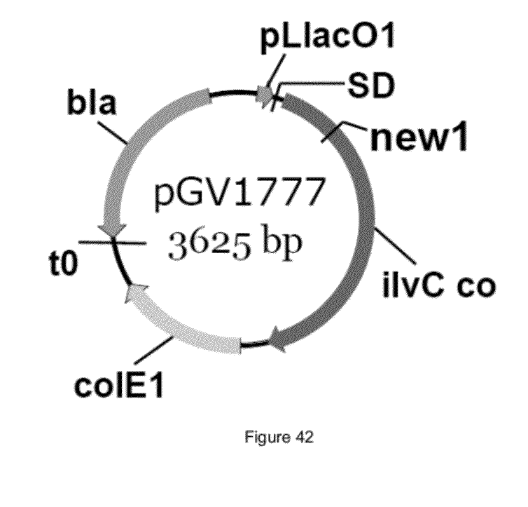

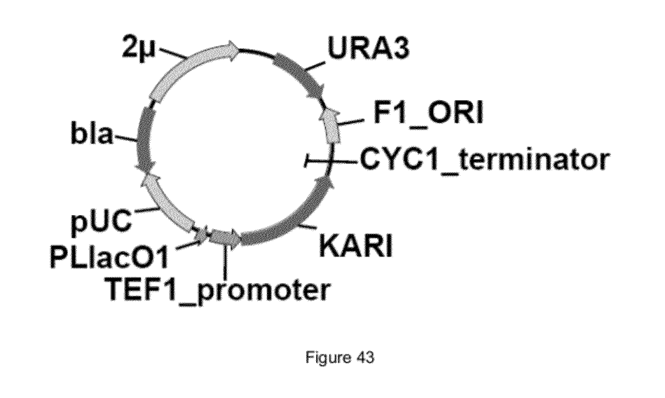

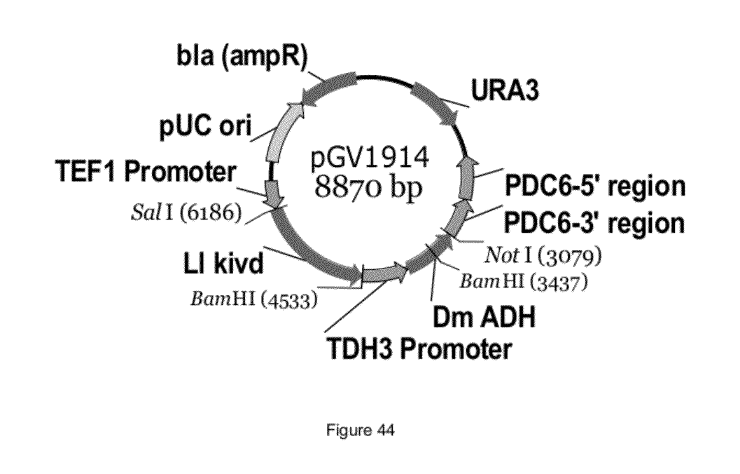

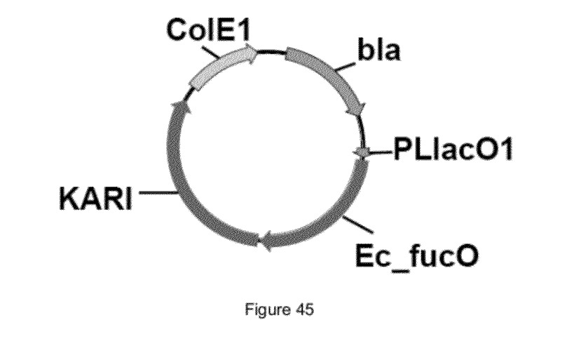

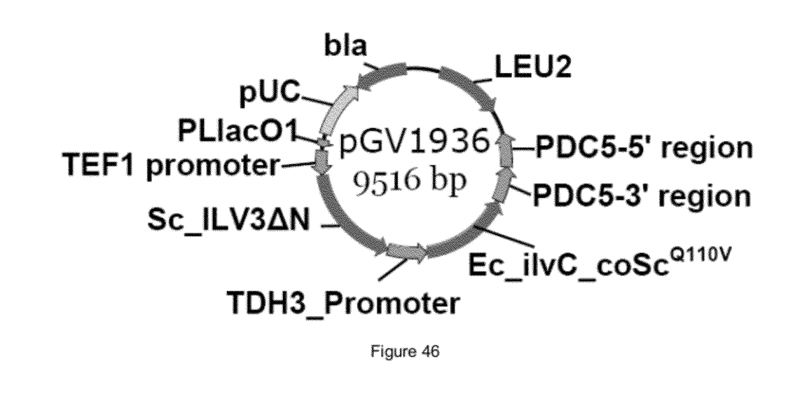

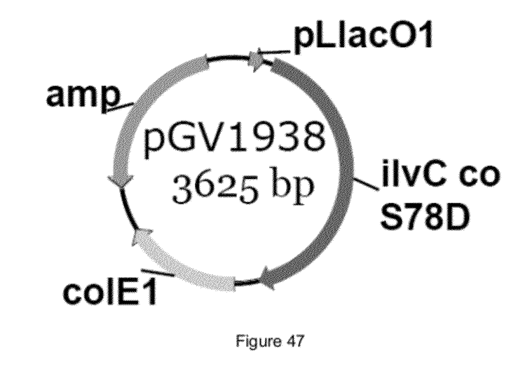

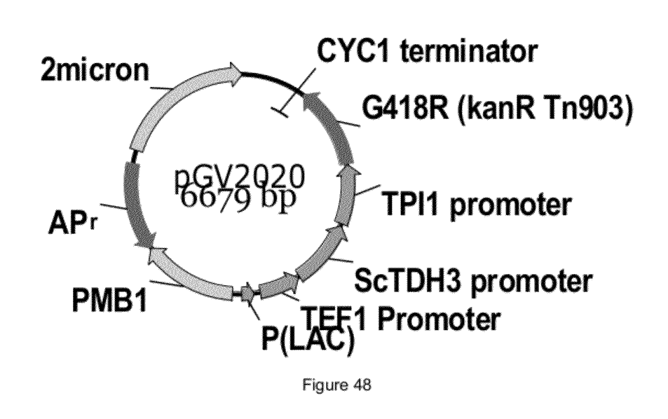

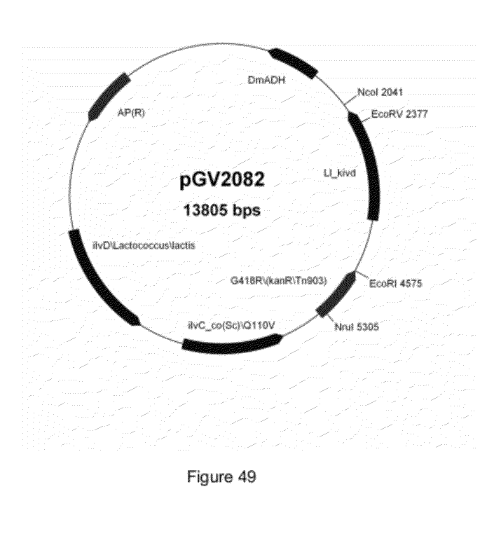

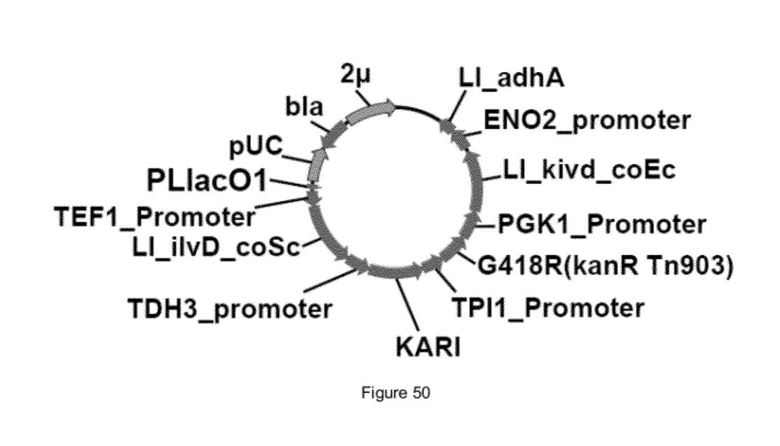

To verify that maeB, ppc, and mdh were expressed, cell lysates were made from GEVO1780 transformed with the above plasmids and run on a protein gel (FIG. 20).

The gel shows that all pathway enzymes are expressed in GEVO1780 with pGV1490 (Ec_IlvD=65.5 kD, LI_Kivd1/Bs_AlsS1=60.9 kD, Ec_IlvC=54.1 kD). The gel also shows that all pathway enzymes and Ppc (99 kD), MaeB (82 kD), and Mdh (32 kD) are expressed in GEVO1780 with pGV1661.

### Example 23

### pyk Bypass 2

This example illustrates that an isobutanol producing microorganism which is engineered to bypass the pyruvate kinase reaction shows increased productivity, titer and yield of isobutanol compared to the control strain without overexpression of ppc or pck.

Both plasmid constructs (pGV1661 (SEQ ID NO: 110) and pGV1772) were sequence verified. GEVO1725, and GEVO1751 were transformed with isobutanol pathway plasmids pGV1655 (SEQ ID NO: 109) and pGV1698 (SEQ ID NO: 112), and pyk bypass plasmids pGV1661 (ppc) or pGV1772 (pck). The controls were the same strains and pathway plasmids, but with the empty vector, pGV1490 (SEQ ID NO: 104), in place of pGV1661 or pGV1772. Strains GEVO1725 and GEVO1751 have deletions of pyruvate kinase (pykAF) and of the NADH dependent malic enzyme, maeA, which are part of the pyruvate kinase bypass engineering. The difference between GEVO1725 and GEVO1751 is that GEVO1725 does not have the tet repressor, and therefore, pGV1490, pGV1661, and pGV1772 are constitutively expressed in this strain.

All of these transformants were tested in isobutanol fermentations.

Overnight cultures were started in duplicate for each transformation in 3 mL M9+A5 salts+FeCl₃+10 g/L YE media and the appropriate antibiotics in 14 mL snap cap tubes and incubated at 37° C., 250 rpm. Screw cap flasks with 20 mL M9+A5 salts+FeCl₃+10 g/L YE media and the appropriate antibiotics were inoculated to a starting OD₆₀₀ of 0.1 with overnight culture. The cells were incubated at 37° C., 250 rpm until they reached an OD₆₀₀ of 0.6-0.8 and were then induced with IPTG [1 mM] and aTc [1 ng/mL]. After induction, the cultures were switched to incubation at 30° C., 250 rpm. Samples were taken of the cultures at 24 and 48 hours after inoculation and OD₆₀₀ and pH were measured. Samples were centrifuged at 22,000×g for 5 min and the supernatant was collected and stored at −20° C. until sample submission. After 48 hour samples were taken, the remainder of the culture was transferred to a 50 ml tube, centrifuged at 4000×g, for 10 min at 4° C. The supernatant was removed, and the cell pellet was stored at −80° C. The samples were analyzed using High performance liquid chromatography (HPLC) and gas chromatography (GC).

The deletion strains with pck (pGV1772) had greater specific productivities than the strains with ppc (pGV1661). When ppc is used in the pyk bypass system in GEVO1725 and GEVO1751, the specific productivity of these strains increased by 3% in GEVO1751 and by 13% in GEVO1725 compared to GEVO1385 with the empty vector. When pck is used instead of ppc, the specific productivity increased by 43% in GEVO1725 and by 50% in GEVO1751. Both of the deletion strains show improved volumetric and specific productivity, titer, and yield when pGV1661 and pGV1772 are expressed compared to the empty vector (Table 43).

TABLE 43
Isobutanol production at 24 hours for pyk bypass system with ppc or pck

| | Volumetric | | Specific | | | | | |
|---|---|---|---|---|---|---|---|---|
| | Productivity | | Productivity | | Titer | | Yield | |
| samples 24 h | [g/L/h] | ± | [g/L/h/OD] | ± | [g/L] | ± | [g/g] | ± |
| GEVO1725 empty | 0.126 | 0.001 | 0.033 | 0.001 | 3.03 | 0.03 | 0.224 | 0.005 |
| vector | | | | | | | | |
| GEVO1725 pGV1661 | 0.266 | 0.003 | 0.045 | 0.001 | 6.38 | 0.07 | 0.304 | 0.022 |
| GEVO1725 pGV1772 | 0.311 | 0.021 | 0.057 | 0.003 | 7.46 | 0.49 | 0.306 | 0.006 |
| GEVO1751 empty | 0.159 | 0.005 | 0.033 | 0.001 | 3.83 | 0.1 | 0.218 | 0.002 |
| vector | | | | | | | | |
| GEVO1751 pGV1661 | 0.262 | 0.054 | 0.041 | 0.005 | 6.29 | 1.29 | 0.236 | 0.035 |
| GEVO1751 pGV1772 | 0.309 | 0.049 | 0.06 | 0.002 | 7.41 | 1.18 | 0.292 | 0.005 |

### Example 24

### NADH Kinase and NADP+ Phosphatase in Yeast

The purpose of this example is to describe how an isobutanol producing yeast which is engineered to express NADPH biosynthesis enzymes to convert NADH into NADPH can produce isobutanol under anaerobic conditions.

A yeast strain GEVO5001 which expresses the isobutanol biosynthetic pathway and is deficient in pyruvate decarboxylase activity is engineered to express NADH kinase and NADP+ phosphatase. pGV6000, which is a yeast integration plasmid carrying an hygromycin resistance marker, NADH kinase and NADP+ phosphatase, is linearized by restriction digestion and transformed into GEVO5001. NADH kinase and NADP+ phosphatase are expressed using the strong constitutive promoters from TEF1 and TDH3, respectively. Clones in which the NADH kinase and NADP+ phosphatase are first identified by resistance to hygromycin. The clones are confirmed to be expressing NADH kinase and NADP+ phosphatase by qRT-PCR. The resulting strain, GEVO5002, along with the parent strain, GEVO5001, is used in fermentations for production of isobutanol.

### Example 25

### Metabolic Transhydrogenation in Yeast

This example describes an isobutanol producing yeast which is engineered to convert NADH into NADPH through the combination of two redox enzymes that are catalyzing a conversion that is part of the same pathway wherein one redox enzyme oxidizes NADH and the other redox enzyme reduces NADP+.

The yeast strain, GEVO5001, is a yeast strain that has been engineered to be deficient in pyruvate decarboxylase activity and also to express the isobutanol pathway. A pyruvate bypass is generated by overexpressing in this yeast the genes for (a) pyruvate carboxylase (PYC1 or PYC2), (b) malate dehydrogenase, MDH2, and (c) malic enzyme (maeB). These genes are cloned to generate the yeast integration plasmid, pGV6004. This plasmid carries the hygromycin resistance marker and expresses PYC1, MDH2 and maeB under the strong promoters from ADH1, TEF1 and TDH3, respectively. pGV6004 is linearized and transformed into GEVO5001 to generate GEVO5006. Over-expressions of PYC1, MDH2 and maeB are confirmed by qRT-PCR.

### Example 26

### Conservation of the Amino Acid Residue Corresponding to Serine 78 of the Wild-Type E. coli KARI

The following example illustrates how additional long-form ketol-acid reductoisomerases (KARIs) are identified.

Long-form ketol-acid reductoisomerases were identified through protein-protein BLAST (blastp) searches of publicly available databases of non-redundant protein sequences (nr database) using the amino acid sequence of E. coli IlvC (SEQ ID NO: 13) with the following search parameters: Expect threshold=10, word size=3, matrix=Blosum62, gap opening=11, gap extension=1.

All sequences of >415 amino acids in length, 347 in total, were selected and are listed in Table 44. The sequences were aligned using a profile alignment in AlignX, a component of Vector NTI Advance 10.3.1 which aligns all selected sequences against a reference sequence, in this case NP_{—}418222 (identical to E. coli IlvC, SEQ ID NO: 13). The term “profile alignment” describes the alignment of two alignments. The method is a simple extension of the profile method of Gribskov, et al. (Gribskov, M., McLachlan, A.D. and Eisenberg, D. (1987) Profile analysis: detection of distantly related proteins. PNAS USA 84, 4355-4358) for aligning a sequence with an alignment. The following parameters were used for the profile alignment: Gap penalty for helix core residue=4; gap penalty for strand core residue=4; gap penalty for structure termini=2; gap penalty for loop regions=1; number of residues inside helix to be treated as terminal=3; number of residues outside a helix to be treated as terminal=0; number of residues inside a strand to be treated as terminal=1; number of residues outside a strand to be treated as terminal=1.

The sequence alignment (FIG. 51) showed complete conservation amongst the long-form ketol-acid reductoisomerases at the amino acid position corresponding to the Serine 78 residue of SEQ ID NO: 13. The only exception is for sequence BAH82904 which has a conservative threonine substitution at this position. Strong conservation was also shown amongst the long-form ketol-acid reductoisomerases at the amino acid positions corresponding to the alanine 71, arginine 76, and glutamine 110 residues of SEQ ID NO: 13. Of the 347 long-form ketol-acid reductoisomerases identified, 326, 339, and 341 harbored alanine, arginine, and glutamine residues corresponding to the alanine 71, arginine 76, and glutamine 110 residues of SEQ ID NO: 13, respectively.

TABLE 44
KARI enzymes of >415 amino acid residues identified by blastp search
with the amino acid sequence of E. coli IlvC:

| GenBank | SEQ ID | | |
|---|---|---|---|
| Accession No. | NO: | Length | Description |
| ZP_08375997 | 331 | 563 | ketol-acid reductoisomerase (Acetohydroxy-acidisomeroreductase) |
| | | | (Alpha-keto-beta-hydroxylacil reductoisomerase) [Escherichia coli |
| | | | TA280]. |
| EGB08261 | 332 | 552 | hypothetical protein AURANDRAFT_53694 [Aureococcus |
| | | | anophagefferens]. |
| EGB85373 | 333 | 541 | ketol-acid reductoisomerase [Escherichia coli MS 117-3]. |
| YP_543282 | 334 | 541 | ketol-acid reductoisomerase [Escherichia coli UTI89]. |
| ZP_04006446 | 335 | 541 | Ketol-acid reductoisomerase [Escherichia coli 83972]. |
| ZP_07098963 | 336 | 541 | ketol-acid reductoisomerase [Escherichia coli MS 107-1]. |
| ZP_07137864 | 337 | 541 | ketol-acid reductoisomerase [Escherichia coli MS 115-1]. |
| ZP_07690695 | 338 | 541 | ketol-acid reductoisomerase [Escherichia coli MS 145-7]. |
| ZP_08350682 | 339 | 541 | ketol-acid reductoisomerase (Acetohydroxy-acidisomeroreductase) |
| | | | (Alpha-keto-beta-hydroxylacil reductoisomerase) [Escherichia coli |
| | | | M605]. |
| ZP_08395131 | 340 | 541 | ketol-acid reductoisomerase [Shigella sp. D9]. |
| ADA76149 | 341 | 536 | Ketol-acid reductoisomerase [Shigella flexneri 2002017]. |
| EGB30597 | 342 | 536 | ketol-acid reductoisomerase [Escherichia coli E1520]. |
| YP_691054 | 343 | 536 | ketol-acid reductoisomerase [Shigella flexneri 5 str. 8401]. |
| ZP_04873037 | 344 | 536 | ketol-acid reductoisomerase [Escherichia sp. 1_1_43]. |
| ZP_08356452 | 345 | 536 | ketol-acid reductoisomerase (Acetohydroxy-acidisomeroreductase) |
| | | | (Alpha-keto-beta-hydroxylacil reductoisomerase) [Escherichia coli |
| | | | M718]. |
| ZP_04630132 | 346 | 532 | Ketol-acid reductoisomerase [Yersinia bercovieri ATCC 43970]. |
| ZP_04642312 | 347 | 532 | Ketol-acid reductoisomerase [Yersinia mollaretii ATCC 43969]. |
| YP_002917111 | 348 | 531 | ketol-acid reductoisomerase [Klebsiella pneumoniae subsp. |
| | | | pneumoniae NTUH-K2044]. |
| YP_001443713 | 349 | 514 | ketol-acid reductoisomerase [Vibrio harveyi ATCC BAA-1116]. |
| ZP_02961747 | 350 | 505 | hypothetical protein PROSTU_03806 [Providencia stuartii ATCC |
| | | | 25827]. |
| ZP_04617113 | 351 | 505 | ketol-acid reductoisomerase [Yersinia ruckeri ATCC 29473]. |
| ZP_04923867 | 352 | 503 | ketol-acid reductoisomerase [Vibrio sp. Ex25]. |
| YP_004578893 | 353 | 500 | ketol-acid reductoisomerase [Lacinutrix sp. 5H-3-7-4]. |
| ZP_02161514 | 354 | 500 | ketol-acid reductoisomerase [Kordia algicida OT-1]. |
| ZP_02181891 | 355 | 500 | ketol-acid reductoisomerase [Flavobacteriales bacterium ALC-1]. |
| ZP_01060688 | 356 | 499 | ketol-acid reductoisomerase [Leeuwenhoekiella blandensis |
| | | | MED217]. |
| YP_004124638 | 357 | 498 | ketol-acid reductoisomerase [Candidatus Blochmannia vafer str. |
| | | | BVAF]. |
| ZP_01050446 | 358 | 498 | ketol-acid reductoisomerase [Dokdonia donghaensis MED134]. |
| ZP_01306608 | 359 | 496 | ketol-acid reductoisomerase [Bermanella marisrubri]. |
| ZP_06156533 | 360 | 495 | ketol-acid reductoisomerase [Photobacterium damselae subsp. |
| | | | damselae CIP 102761]. |
| ABD66583 | 361 | 494 | ketol-acid reductoisomerase [Vibrio cholerae]. |
| AEA77464 | 362 | 494 | Ketol-acid reductoisomerase [Vibrio cholerae LMA3984-4]. |
| EGF42466 | 363 | 494 | ketol-acid reductoisomerase [Vibrio parahaemolyticus 10329]. |
| EGR03889 | 364 | 494 | ketol-acid reductoisomerase [Vibrio cholerae HE39]. |
| EGR10479 | 365 | 494 | ketol-acid reductoisomerase [Vibrio cholerae HE48]. |
| EGU41134 | 366 | 494 | ketol-acid reductoisomerasee [Vibrio splendidus ATCC 33789]. |
| NP_229819 | 367 | 494 | ketol-acid reductoisomerase [Vibrio cholerae O1 biovar El Tor str. |
| | | | N16961]. |
| NP_760028 | 368 | 494 | ketol-acid reductoisomerase [Vibrio vulnificus CMCP6]. |
| NP_796414 | 369 | 494 | ketol-acid reductoisomerase [Vibrio parahaemolyticus RIMD |
| | | | 2210633]. |
| YP_002157314 | 370 | 494 | ketol-acid reductoisomerase [Vibrio fischeri MJ11]. |
| YP_002261639 | 371 | 494 | ketol-acid reductoisomerase [Aliivibrio salmonicida LFI1238]. |
| YP_002415754 | 372 | 494 | ketol-acid reductoisomerase [Vibrio splendidus LGP32]. |
| YP_002891495 | 373 | 494 | ketol-acid reductoisomerase [Tolumonas auensis DSM 9187]. |
| YP_003284611 | 374 | 494 | ketol-acid reductoisomerase [Vibrio sp. Ex25]. |
| YP_004436406 | 375 | 494 | ketol-acid reductoisomerase [Glaciecola sp. 4H-3-7 + YE-5]. |
| YP_004440448 | 376 | 494 | ketol-acid reductoisomerase [Treponema brennaborense DSM |
| | | | 12168]. |
| YP_004564924 | 377 | 494 | Ketol-acid reductoisomerase [Vibrio anguillarum 775]. |
| YP_004843660 | 378 | 494 | Ketol-acid reductoisomerase [Flavobacterium branchiophilum]. |
| YP_004873667 | 379 | 494 | ketol-acid reductoisomerase [Glaciecola nitratireducens FR1064]. |
| YP_128332 | 380 | 494 | ketol-acid reductoisomerase [Photobacterium profundum SS9]. |
| YP_205911 | 381 | 494 | ketol-acid reductoisomerase [Vibrio fischeri ES114]. |
| YP_663808 | 382 | 494 | ketol-acid reductoisomerase [Pseudoalteromonas atlantica T6c]. |
| ZP_00993208 | 383 | 494 | ketol-acid reductoisomerase [Vibrio splendidus 12B01]. |
| ZP_01067040 | 384 | 494 | ketol-acid reductoisomerase [Vibrio sp. MED222]. |
| ZP_01116327 | 385 | 494 | ketol-acid reductoisomerase [Reinekea blandensis MED297]. |
| ZP_01160403 | 386 | 494 | ketol-acid reductoisomerase [Photobacterium sp. SKA34]. |
| ZP_01223049 | 387 | 494 | ketol-acid reductoisomerase [Photobacterium profundum 3TCK]. |
| ZP_01237291 | 388 | 494 | ketol-acid reductoisomerase [Photobacterium angustum S14]. |
| ZP_01262768 | 389 | 494 | ketol-acid reductoisomerase [Vibrio alginolyticus 12G01]. |
| ZP_01816341 | 390 | 494 | ketol-acid reductoisomerase [Vibrionales bacterium SWAT-3]. |
| ZP_01870391 | 391 | 494 | ketol-acid reductoisomerase [Vibrio shilonii AK1]. |
| ZP_01951092 | 392 | 494 | ketol-acid reductoisomerase [Vibrio cholerae 1587]. |
| ZP_02196048 | 393 | 494 | ketol-acid reductoisomerase [Vibrio sp. AND4]. |
| ZP_04414286 | 394 | 494 | ketol-acid reductoisomerase [Vibrio cholerae bv. albensis VL426]. |
| ZP_05056766 | 395 | 494 | ketol-acid reductoisomerase, putative [Verrucomicrobiae bacterium |
| | | | DG1235]. |
| ZP_05120422 | 396 | 494 | ketol-acid reductoisomerase [Vibrio parahaemolyticus 16]. |
| ZP_05717058 | 397 | 494 | ketol-acid reductoisomerase [Vibrio mimicus VM573]. |
| ZP_05720722 | 398 | 494 | ketol-acid reductoisomerase [Vibrio mimicus VM603]. |
| ZP_05879603 | 399 | 494 | ketol-acid reductoisomerase [Vibrio furnissii CIP 102972]. |
| ZP_05883426 | 400 | 494 | ketol-acid reductoisomerase [Vibrio metschnikovii CIP 69.14]. |
| ZP_05883972 | 401 | 494 | ketol-acid reductoisomerase [Vibrio coralliilyticus ATCC BAA-450]. |
| ZP_05911056 | 402 | 494 | ketol-acid reductoisomerase [Vibrio parahaemolyticus AQ4037]. |
| ZP_05927497 | 403 | 494 | ketol-acid reductoisomerase [Vibrio sp. RC341]. |
| ZP_05942658 | 404 | 494 | ketol-acid reductoisomerase [Vibrio orientalis CIP 102891 = ATCC |
| | | | 33934]. |
| ZP_06040407 | 405 | 494 | ketol-acid reductoisomerase [Vibrio mimicus MB451]. |
| ZP_06051452 | 406 | 494 | ketol-acid reductoisomerase [Grimontia hollisae CIP 101886]. |
| ZP_06081449 | 407 | 494 | ketol-acid reductoisomerase [Vibrio sp. RC586]. |
| ZP_06173878 | 408 | 494 | ketol-acid reductoisomerase [Vibrio harveyi 1DA3]. |
| ZP_06943519 | 409 | 494 | ketol-acid reductoisomerase [Vibrio cholerae RC385]. |
| ZP_07743436 | 410 | 494 | ketol-acid reductoisomerase [Vibrio caribbenthicus ATCC BAA- |
| | | | 2122]. |
| ZP_08098316 | 411 | 494 | ketol-acid reductoisomerase [Vibrio brasiliensis LMG 20546]. |
| ZP_08104634 | 412 | 494 | ketol-acid reductoisomerase [Vibrio sinaloensis DSM 21326]. |
| ZP_08310897 | 413 | 494 | ketol-acid reductoisomerase [Photobacterium leiognathi subsp. |
| | | | mandapamensis svers.1.1.]. |
| ZP_08731305 | 414 | 494 | ketol-acid reductoisomerase [Vibrio nigripulchritudo ATCC 27043]. |
| ZP_08744172 | 415 | 494 | ketol-acid reductoisomerase [Vibrio ichthyoenteri ATCC 700023]. |
| ZP_08747945 | 416 | 494 | ketol-acid reductoisomerase [Vibrio scophthalmi LMG 19158]. |
| ZP_08908778 | 417 | 494 | ketol-acid reductoisomerase [Vibrio rotiferianus DAT722]. |
| EGT79222 | 418 | 493 | ketol-acid reductoisomerase [Haemophilus haemolyticus M19107]. |
| YP_001054540 | 419 | 493 | ketol-acid reductoisomerase [Actinobacillus pleuropneumoniae |
| | | | serovar 5b str. L20]. |
| YP_001092419 | 420 | 493 | ketol-acid reductoisomerase [Shewanella loihica PV-4]. |
| YP_001143912 | 421 | 493 | ketol-acid reductoisomerase [Aeromonas salmonicida subsp. |
| | | | salmonicida A449]. |
| YP_001343873 | 422 | 493 | ketol-acid reductoisomerase [Actinobacillus succinogenes 130Z]. |
| YP_001475898 | 423 | 493 | ketol-acid reductoisomerase [Shewanella sediminis HAW-EB3]. |
| YP_001500207 | 424 | 493 | ketol-acid reductoisomerase [Shewanella pealeana ATCC 700345]. |
| YP_001676144 | 425 | 493 | ketol-acid reductoisomerase [Shewanella halifaxensis HAW-EB4]. |
| YP_001758834 | 426 | 493 | ketol-acid reductoisomerase [Shewanella woodyi ATCC 51908]. |
| YP_001785151 | 427 | 493 | ketol-acid reductoisomerase [Haemophilus somnus 2336]. |
| YP_002309779 | 428 | 493 | ketol-acid reductoisomerase [Shewanella piezotolerans WP3]. |
| YP_002475862 | 429 | 493 | ketol-acid reductoisomerase [Haemophilus parasuis SH0165]. |
| YP_003093678 | 430 | 493 | ketol-acid reductoisomerase [Pedobacter heparinus DSM 2366]. |
| YP_003558619 | 431 | 493 | ketol-acid reductoisomerase [Shewanella violacea DSS12]. |
| YP_004274723 | 432 | 493 | ketol-acid reductoisomerase [Pedobacter saltans DSM 12145]. |
| YP_004317509 | 433 | 493 | ketol-acid reductoisomerase [Sphingobacterium sp. 21]. |
| YP_004394576 | 434 | 493 | ketol-acid reductoisomerase [Aeromonas veronii B565]. |
| YP_004822180 | 435 | 493 | ketol-acid reductoisomerase, NAD(P)-binding [Haemophilus |
| | | | parainfluenzae T3T1]. |
| YP_087237 | 436 | 493 | ketol-acid reductoisomerase [Mannheimia succiniciproducens |
| | | | MBEL55E]. |
| YP_271478 | 437 | 493 | ketol-acid reductoisomerase [Colwellia psychrerythraea 34H]. |
| YP_719876 | 438 | 493 | ketol-acid reductoisomerase [Haemophilus somnus 129PT]. |
| YP_749121 | 439 | 493 | ketol-acid reductoisomerase [Shewanella frigidimarina NCIMB 400]. |
| YP_854686 | 440 | 493 | ketol-acid reductoisomerase [Aeromonas hydrophila subsp. |
| | | | hydrophila ATCC 7966]. |
| YP_929154 | 441 | 493 | ketol-acid reductoisomerase [Shewanella amazonensis SB2B]. |
| YP_942294 | 442 | 493 | ketol-acid reductoisomerase [Psychromonas ingrahamii 37]. |
| ZP_00134485 | 443 | 493 | COG0059: Ketol-acid reductoisomerase [Actinobacillus |
| | | | pleuropneumoniae serovar 1 str. 4074]. |
| ZP_01224863 | 444 | 493 | ketol-acid reductoisomerase [gamma proteobacterium HTCC2207]. |
| ZP_01884737 | 445 | 493 | ketol-acid reductoisomerase [Pedobacter sp. BAL39]. |
| ZP_02155927 | 446 | 493 | ketol-acid reductoisomerase [Shewanella benthica KT99]. |
| ZP_02478674 | 447 | 493 | ketol-acid reductoisomerase [Haemophilus parasuis 29755]. |
| ZP_03967662 | 448 | 493 | ketol-acid reductoisomerase [Sphingobacterium spiritivorum ATCC |
| | | | 33300]. |
| ZP_04754564 | 449 | 493 | ketol-acid reductoisomerase [Actinobacillus minor NM305]. |
| ZP_04979281 | 450 | 493 | ketol-acid reductoisomerase [Mannheimia haemolytica PHL213]. |
| ZP_05629499 | 451 | 493 | ketol-acid reductoisomerase [Actinobacillus minor 202]. |
| ZP_07080881 | 452 | 493 | ketol-acid reductoisomerase [Sphingobacterium spiritivorum ATCC |
| | | | 33861]. |
| ZP_07337087 | 453 | 493 | ketol-acid reductoisomerase [Actinobacillus pleuropneumoniae |
| | | | serovar 6 str. Femo]. |
| ZP_07528854 | 454 | 493 | ketol-acid reductoisomerase [Actinobacillus pleuropneumoniae |
| | | | serovar 1 str. 4074]. |
| ZP_07533048 | 455 | 493 | ketol-acid reductoisomerase [Actinobacillus pleuropneumoniae |
| | | | serovar 4 str. M62]. |
| ZP_07539711 | 456 | 493 | ketol-acid reductoisomerase [Actinobacillus pleuropneumoniae |
| | | | serovar 10 str. D13039]. |
| ZP_07546145 | 457 | 493 | ketol-acid reductoisomerase [Actinobacillus pleuropneumoniae |
| | | | serovar 13 str. N273]. |
| ZP_07953211 | 458 | 493 | ketol-acid reductoisomerase [Enterobacteriaceae bacterium |
| | | | 9_2_54FAA]. |
| ZP_08066951 | 459 | 493 | ketol-acid reductoisomerase [Actinobacillus ureae ATCC 25976]. |
| ZP_08077521 | 460 | 493 | ketol-acid reductoisomerase [Succinatimonas hippei YIT 12066]. |
| ZP_08147399 | 461 | 493 | ketol-acid reductoisomerase [Haemophilus parainfluenzae ATCC |
| | | | 33392]. |
| ZP_08522240 | 462 | 493 | ketol-acid reductoisomerase [Aeromonas caviae Ae398]. |
| ZP_08756004 | 463 | 493 | ketol-acid reductoisomerase [Haemophilus pittmaniae HK 85]. |
| ADI19779 | 464 | 492 | hypothetical protein [uncultured gamma proteobacterium |
| | | | EB000_37F04]. |
| ADP11056 | 465 | 492 | ketol-acid reductoisomerase [Erwinia sp. Ejp617]. |
| ADV56009 | 466 | 492 | ketol-acid reductoisomerase [Shewanella putrefaciens 200]. |
| CBY29180 | 467 | 492 | ketol-acid reductoisomerase [Yersinia enterocolitica subsp. |
| | | | palearctica Y11]. |
| EGT77473 | 468 | 492 | ketol-acid reductoisomerase [Haemophilus haemolyticus M19501]. |
| EGT78129 | 469 | 492 | ketol-acid reductoisomerase [Haemophilus haemolyticus M21127]. |
| EGT83109 | 470 | 492 | ketol-acid reductoisomerase [Haemophilus haemolyticus M21639]. |
| EGY30102 | 471 | 492 | ketol-acid reductoisomerase [Aggregatibacter aphrophilus ATCC |
| | | | 33389]. |
| NP_438842 | 472 | 492 | ketol-acid reductoisomerase [Haemophilus influenzae Rd KW20]. |
| NP_667684 | 473 | 492 | ketol-acid reductoisomerase [Yersinia pestis KIM 10]. |
| NP_719873 | 474 | 492 | ketol-acid reductoisomerase [Shewanella oneidensis MR-1]. |
| NP_878859 | 475 | 492 | ketol-acid reductoisomerase [Candidatus Blochmannia floridanus]. |
| NP_931830 | 476 | 492 | ketol-acid reductoisomerase [Photorhabdus luminescens subsp. |
| | | | laumondii TTO1]. |
| YP_001004547 | 477 | 492 | ketol-acid reductoisomerase [Yersinia enterocolitica subsp. |
| | | | enterocolitica 8081]. |
| YP_001052367 | 478 | 492 | ketol-acid reductoisomerase [Shewanella baltica OS155]. |
| YP_001185122 | 479 | 492 | ketol-acid reductoisomerase [Shewanella putrefaciens CN-32]. |
| YP_001292610 | 480 | 492 | ketol-acid reductoisomerase [Haemophilus influenzae PittGG]. |
| YP_001368194 | 481 | 492 | ketol-acid reductoisomerase [Shewanella baltica OS185]. |
| YP_001556549 | 482 | 492 | ketol-acid reductoisomerase [Shewanella baltica OS195]. |
| YP_001906144 | 483 | 492 | ketol-acid reductoisomerase [Erwinia tasmaniensis Et1/99]. |
| YP_002359832 | 484 | 492 | ketol-acid reductoisomerase [Shewanella baltica OS223]. |
| YP_002647213 | 485 | 492 | ketol-acid reductoisomerase [Erwinia pyrifoliae Ep1/96]. |
| YP_002931578 | 486 | 492 | ketol-acid reductoisomerase [Edwardsiella ictaluri 93-146]. |
| YP_002989344 | 487 | 492 | ketol-acid reductoisomerase [Dickeya dadantii Ech703]. |
| YP_003002518 | 488 | 492 | ketol-acid reductoisomerase [Dickeya zeae Ech1591]. |
| YP_003008688 | 489 | 492 | ketol-acid reductoisomerase [Aggregatibacter aphrophilus NJ8700]. |
| YP_003019572 | 490 | 492 | ketol-acid reductoisomerase [Pectobacterium carotovorum subsp. |
| | | | carotovorum PC1]. |
| YP_003042977 | 491 | 492 | ketol-acid reductoisomerase [Photorhabdus asymbiotica subsp. |
| | | | asymbiotica ATCC 43949]. |
| YP_003248407 | 492 | 492 | ketol-acid reductoisomerase [Fibrobacter succinogenes subsp. |
| | | | succinogenes S85]. |
| YP_003261544 | 493 | 492 | ketol-acid reductoisomerase [Pectobacterium wasabiae WPP163]. |
| YP_003294152 | 494 | 492 | ketol-acid reductoisomerase [Edwardsiella tarda EIB202]. |
| YP_003335446 | 495 | 492 | ketol-acid reductoisomerase [Dickeya dadantii Ech586]. |
| YP_003470059 | 496 | 492 | ketol-acid reductoisomerase [Xenorhabdus bovienii SS-2004]. |
| YP_003529522 | 497 | 492 | ketol-acid reductoisomerase [Erwinia amylovora CFBP1430]. |
| YP_003710690 | 498 | 492 | ketol-acid reductoisomerase [Xenorhabdus nematophila ATCC |
| | | | 19061]. |
| YP_003739580 | 499 | 492 | ketol-acid reductoisomerase [Erwinia billingiae Eb661]. |
| YP_003885028 | 500 | 492 | ketol-acid reductoisomerase, NAD(P)-binding protein [Dickeya |
| | | | dadantii 3937]. |
| YP_004041287 | 501 | 492 | ketol-acid reductoisomerase [Paludibacter propionicigenes WB4]. |
| YP_004136070 | 502 | 492 | ketol-acid reductoisomerase, nad(p)-binding [Haemophilus |
| | | | influenzae F3031]. |
| YP_004296355 | 503 | 492 | ketol-acid reductoisomerase [Yersinia enterocolitica subsp. |
| | | | palearctica 105.5R(r)]. |
| YP_004365030 | 504 | 492 | ketol-acid reductoisomerase [Treponema succinifaciens DSM 2489]. |
| YP_004427401 | 505 | 492 | ketol-acid reductoisomerase [Alteromonas macleodii str. ‘Deep |
| | | | ecotype’]. |
| YP_004429149 | 506 | 492 | ketol-acid reductoisomerase [Alteromonas macleodii str. ‘Deep |
| | | | ecotype’]. |
| YP_004469651 | 507 | 492 | ketol-acid reductoisomerase [Alteromonas sp. SN2]. |
| YP_052308 | 508 | 492 | ketol-acid reductoisomerase [Pectobacterium atrosepticum |
| | | | SCRI1043]. |
| YP_068694 | 509 | 492 | ketol-acid reductoisomerase [Yersinia pseudotuberculosis IP |
| | | | 32953]. |
| YP_248373 | 510 | 492 | ketol-acid reductoisomerase [Haemophilus influenzae 86-028NP]. |
| YP_456071 | 511 | 492 | ketol-acid reductoisomerase [Sodalis glossinidius str. ‘morsitans’]. |
| YP_564413 | 512 | 492 | ketol-acid reductoisomerase [Shewanella denitrificans OS217]. |
| YP_677763 | 513 | 492 | ketol-acid reductoisomerase [Cytophaga hutchinsonii ATCC 33406]. |
| YP_732498 | 514 | 492 | ketol-acid reductoisomerase [Shewanella sp. MR-4]. |
| YP_739702 | 515 | 492 | ketol-acid reductoisomerase [Shewanella sp. MR-7]. |
| YP_868004 | 516 | 492 | ketol-acid reductoisomerase [Shewanella sp. ANA-3]. |
| YP_965114 | 517 | 492 | ketol-acid reductoisomerase [Shewanella sp. W3-18-1]. |
| ZP_01789462 | 518 | 492 | ketol-acid reductoisomerase [Haemophilus influenzae 3655]. |
| ZP_01791236 | 519 | 492 | ketol-acid reductoisomerase [Haemophilus influenzae PittAA]. |
| ZP_01797556 | 520 | 492 | ketol-acid reductoisomerase [Haemophilus influenzae R3021]. |
| ZP_03828016 | 521 | 492 | ketol-acid reductoisomerase [Pectobacterium carotovorum subsp. |
| | | | brasiliensis PBR1692]. |
| ZP_04612142 | 522 | 492 | ketol-acid reductoisomerase [Yersinia rohdei ATCC 43380]. |
| ZP_04620984 | 523 | 492 | ketol-acid reductoisomerase [Yersinia aldovae ATCC 35236]. |
| ZP_04626075 | 524 | 492 | ketol-acid reductoisomerase [Yersinia kristensenii ATCC 33638]. |
| ZP_04633131 | 525 | 492 | ketol-acid reductoisomerase [Yersinia frederiksenii ATCC 33641]. |
| ZP_04637919 | 526 | 492 | ketol-acid reductoisomerase [Yersinia intermedia ATCC 29909]. |
| ZP_04957910 | 527 | 492 | ketol-acid reductoisomerase [gamma proteobacterium NOR51-B]. |
| ZP_05850757 | 528 | 492 | ketol-acid reductoisomerase [Haemophilus influenzae NT127]. |
| ZP_06127160 | 529 | 492 | ketol-acid reductoisomerase [Providencia rettgeri DSM 1131]. |
| ZP_06716193 | 530 | 492 | ketol-acid reductoisomerase [Edwardsiella tarda ATCC 23685]. |
| ZP_07393823 | 531 | 492 | ketol-acid reductoisomerase [Shewanella baltica OS183]. |
| ZP_07890225 | 532 | 492 | ketol-acid reductoisomerase [Aggregatibacter segnis ATCC 33393]. |
| ZP_08726579 | 533 | 492 | ketol-acid reductoisomerase [Haemophilus haemolyticus M21621]. |
| AAA24029 | 534 | 491 | acetohydroxy acid isomeroreductase [Escherichia coli]. |
| AAC38126 | 535 | 491 | acetohydroxy acid isomeroreductase [Buchnera aphidicola]. |
| ADI18763 | 536 | 491 | ketol-acid reductoisomerase [uncultured gamma proteobacterium |
| | | | HF4000_36I10]. |
| ADR29158 | 537 | 491 | ketol-acid reductoisomerase [Escherichia coli O83:H1 str. NRG |
| | | | 857C]. |
| AEK00748 | 538 | 491 | ketol-acid reductoisomerase [Klebsiella pneumoniae KCTC 2242]. |
| BAH82904 | 539 | 491 | ketol-acid reductoisomerase [Candidatus Ishikawaella capsulata |
| | | | Mpkobe]. |
| CBA71826 | 540 | 491 | ketol-acid reductoisomerase [Arsenophonus nasoniae]. |
| EFW51086 | 541 | 491 | ketol-acid reductoisomerase [Shigella dysenteriae CDC 74-1112]. |
| EFW53045 | 542 | 491 | ketol-acid reductoisomerase [Shigella boydii ATCC 9905]. |
| EFZ59021 | 543 | 491 | ketol-acid reductoisomerase [Escherichia coli LT-68]. |
| EGB59513 | 544 | 491 | ketol-acid reductoisomerase [Escherichia coli M863]. |
| EGP04347 | 545 | 491 | ketol-acid reductoisomerase [Pasteurella multocida subsp. |
| | | | multocida str. Anand1_goat]. |
| EGW63513 | 546 | 491 | ketol-acid reductoisomerase [Escherichia coli STEC_C165-02]. |
| EGX01490 | 547 | 491 | ketol-acid reductoisomerase [Escherichia coli STEC_MHI813]. |
| NP_246221 | 548 | 491 | ketol-acid reductoisomerase [Pasteurella multocida subsp. |
| | | | multocida str. Pm70]. |
| NP_290405 | 549 | 491 | ketol-acid reductoisomerase [Escherichia coli O157:H7 str. |
| | | | EDL933]. |
| NP_418222 | 13 | 491 | ketol-acid reductoisomerase, NAD(P)-binding [Escherichia coli str. |
| | | | K-12 substr. MG1655]. |
| NP_457839 | 550 | 491 | ketol-acid reductoisomerase [Salmonella enterica subsp. enterica |
| | | | serovar Typhi str. CT18]. |
| NP_462800 | 551 | 491 | ketol-acid reductoisomerase [Salmonella enterica subsp. enterica |
| | | | serovar Typhimurium str. LT2]. |
| NP_660898 | 552 | 491 | ketol-acid reductoisomerase [Buchnera aphidicola str. Sg |
| | | | (Schizaphis graminum)]. |
| NP_778135 | 553 | 491 | ketol-acid reductoisomerase [Buchnera aphidicola str. Bp (Baizongia |
| | | | pistaciae)]. |
| NP_839100 | 554 | 491 | ketol-acid reductoisomerase [Shigella flexneri 2a str. 2457T]. |
| YP_001178715 | 555 | 491 | ketol-acid reductoisomerase [Enterobacter sp. 638]. |
| YP_001337912 | 556 | 491 | ketol-acid reductoisomerase [Klebsiella pneumoniae subsp. |
| | | | pneumoniae MGH 78578]. |
| YP_001439809 | 557 | 491 | ketol-acid reductoisomerase [Cronobacter sakazakii ATCC BAA- |
| | | | 894]. |
| YP_001451723 | 558 | 491 | ketol-acid reductoisomerase [Citrobacter koseri ATCC BAA-895]. |
| YP_001480972 | 559 | 491 | ketol-acid reductoisomerase [Serratia proteamaculans 568]. |
| YP_001572687 | 560 | 491 | ketol-acid reductoisomerase [Salmonella enterica subsp. arizonae |
| | | | serovar 62:z4, z23:—str. RSK2980]. |
| YP_002043148 | 561 | 491 | ketol-acid reductoisomerase [Salmonella enterica subsp. enterica |
| | | | serovar Newport str. SL254]. |
| YP_002148837 | 562 | 491 | ketol-acid reductoisomerase [Salmonella enterica subsp. enterica |
| | | | serovar Agona str. SL483]. |
| YP_002152990 | 563 | 491 | ketol-acid reductoisomerase [Proteus mirabilis HI4320]. |
| YP_002217848 | 564 | 491 | ketol-acid reductoisomerase [Salmonella enterica subsp. enterica |
| | | | serovar Dublin str. CT_02021853]. |
| YP_002241161 | 565 | 491 | ketol-acid reductoisomerase [Klebsiella pneumoniae 342]. |
| YP_002384790 | 566 | 491 | ketol-acid reductoisomerase [Escherichia fergusonii ATCC 35469]. |
| YP_002405159 | 567 | 491 | ketol-acid reductoisomerase [Escherichia coli 55989]. |
| YP_002408941 | 568 | 491 | ketol-acid reductoisomerase [Escherichia coli IAI39]. |
| YP_003195874 | 569 | 491 | ketol-acid reductoisomerase [Robiginitalea biformata HTCC2501]. |
| YP_003208576 | 570 | 491 | ketol-acid reductoisomerase [Cronobacter turicensis z3032]. |
| YP_003367410 | 571 | 491 | ketol-acid reductoisomerase [Citrobacter rodentium ICC168]. |
| YP_003522118 | 572 | 491 | IlvC [Pantoea ananatis LMG 20103]. |
| YP_003615479 | 573 | 491 | ketol-acid reductoisomerase (Acetohydroxy-acidisomeroreductase) |
| | | | [Enterobacter cloacae subsp. cloacae ATCC 13047]. |
| YP_003863317 | 574 | 491 | ketol-acid reductoisomerase [Maribacter sp. HTCC2170]. |
| YP_003932703 | 575 | 491 | ketol-acid reductoisomerase [Pantoea vagans C9-1]. |
| YP_003943772 | 576 | 491 | ketol-acid reductoisomerase [Enterobacter cloacae SCF1]. |
| YP_004117680 | 577 | 491 | ketol-acid reductoisomerase [Pantoea sp. At-9b]. |
| YP_004166417 | 578 | 491 | ketol-acid reductoisomerase [Cellulophaga algicola DSM 14237]. |
| YP_004196697 | 579 | 491 | ketol-acid reductoisomerase [Desulfobulbus propionicus DSM |
| | | | 2032]. |
| YP_004214982 | 580 | 491 | ketol-acid reductoisomerase [Rahnella sp. Y9602]. |
| YP_004263437 | 581 | 491 | ketol-acid reductoisomerase [Cellulophaga lytica DSM 7489]. |
| YP_004419833 | 582 | 491 | ketol-acid reductoisomerase [Gallibacterium anatis UMN179]. |
| YP_004591751 | 583 | 491 | ketol-acid reductoisomerase [Enterobacter aerogenes KCTC 2190]. |
| YP_004732250 | 584 | 491 | ketol-acid reductoisomerase [Salmonella bongori NCTC 12419]. |
| YP_004738981 | 585 | 491 | ketol-acid reductoisomerase [Zobellia galactanivorans]. |
| YP_004789690 | 586 | 491 | ketol-acid reductoisomerase [Muricauda ruestringensis DSM |
| | | | 13258]. |
| YP_004830786 | 587 | 491 | ketol-acid reductoisomerase [Enterobacter asburiae LF7a]. |
| YP_278087 | 588 | 491 | ketol-acid reductoisomerase [Candidatus Blochmannia |
| | | | pennsylvanicus str. BPEN]. |
| YP_312707 | 589 | 491 | ketol-acid reductoisomerase [Shigella sonnei Ss046]. |
| YP_405393 | 590 | 491 | ketol-acid reductoisomerase [Shigella dysenteriae Sd197]. |
| YP_671834 | 591 | 491 | ketol-acid reductoisomerase [Escherichia coli 536]. |
| YP_859360 | 592 | 491 | ketol-acid reductoisomerase [Escherichia coli APEC O1]. |
| YP_862142 | 593 | 491 | ketol-acid reductoisomerase [Gramella forsetii KT0803]. |
| ZP_01076547 | 594 | 491 | ketol-acid reductoisomerase (Acetohydroxy-acid |
| | | | isomeroreductase)(Alpha-keto-beta-hydroxylacil reductoisomerase) |
| | | | [Marinomonas sp. MED121]. |
| ZP_01117623 | 595 | 491 | ketol-acid reductoisomerase [Polaribacter irgensii 23-P]. |
| ZP_01135114 | 596 | 491 | ketol-acid reductoisomerase [Pseudoalteromonas tunicata D2]. |
| ZP_01167291 | 597 | 491 | ketol-acid reductoisomerase [Neptuniibacter caesariensis]. |
| ZP_01216197 | 598 | 491 | ketol-acid reductoisomerase [Psychromonas sp. CNPT3]. |
| ZP_01618023 | 599 | 491 | ketol-acid reductoisomerase [marine gamma proteobacterium |
| | | | HTCC2143]. |
| ZP_01899004 | 600 | 491 | ketol-acid reductoisomerase [Moritella sp. PE36]. |
| ZP_02347072 | 601 | 491 | ketol-acid reductoisomerase [Salmonella enterica subsp. enterica |
| | | | serovar Saintpaul str. SARA29]. |
| ZP_02659286 | 602 | 491 | ketol-acid reductoisomerase [Salmonella enterica subsp. enterica |
| | | | serovar Kentucky str. CDC 191]. |
| ZP_02663422 | 603 | 491 | ketol-acid reductoisomerase (Acetohydroxy-acidisomeroreductase) |
| | | | [Salmonella enterica subsp. enterica serovar Schwarzengrund str. |
| | | | SL480]. |
| ZP_02669892 | 604 | 491 | ketol-acid reductoisomerase [Salmonella enterica subsp. enterica |
| | | | serovar Heidelberg str. SL486]. |
| ZP_02701512 | 605 | 491 | ketol-acid reductoisomerase [Salmonella enterica subsp. enterica |
| | | | serovar Newport str. SL317]. |
| ZP_02904315 | 606 | 491 | ketol-acid reductoisomerase [Escherichia albertii TW07627]. |
| ZP_03001355 | 607 | 491 | ketol-acid reductoisomerase [Escherichia coli 53638]. |
| ZP_03027948 | 608 | 491 | ketol-acid reductoisomerase [Escherichia coli B7A]. |
| ZP_03066877 | 609 | 491 | ketol-acid reductoisomerase [Shigella dysenteriae 1012]. |
| ZP_03214352 | 610 | 491 | ketol-acid reductoisomerase [Salmonella enterica subsp. enterica |
| | | | serovar Virchow str. SL491]. |
| ZP_03318332 | 611 | 491 | hypothetical protein PROVALCAL_01263 [Providencia |
| | | | alcalifaciens DSM 30120]. |
| ZP_03701316 | 612 | 491 | ketol-acid reductoisomerase [Flavobacteria bacterium MS024-3C]. |
| ZP_03802251 | 613 | 491 | hypothetical protein PROPEN_00591 [Proteus penneri ATCC |
| | | | 35198]. |
| ZP_03841558 | 614 | 491 | ketol-acid reductoisomerase [Proteus mirabilis ATCC 29906]. |
| ZP_04558381 | 615 | 491 | ketol-acid reductoisomerase [Citrobacter sp. 30_2]. |
| ZP_04656754 | 616 | 491 | ketol-acid reductoisomerase [Salmonella enterica subsp. enterica |
| | | | serovar Tennessee str. CDC07-0191]. |
| ZP_05919862 | 617 | 491 | ketol-acid reductoisomerase [Pasteurella dagmatis ATCC 43325]. |
| ZP_05970899 | 618 | 491 | ketol-acid reductoisomerase [Enterobacter cancerogenus ATCC |
| | | | 35316]. |
| ZP_05972917 | 619 | 491 | ketol-acid reductoisomerase [Providencia rustigianii DSM 4541]. |
| ZP_06192833 | 620 | 491 | ketol-acid reductoisomerase [Serratia odorifera 4Rx13]. |
| ZP_06354288 | 621 | 491 | ketol-acid reductoisomerase [Citrobacter youngae ATCC 29220]. |
| ZP_06637791 | 622 | 491 | ketol-acid reductoisomerase [Serratia odorifera DSM 4582]. |
| ZP_07167619 | 623 | 491 | ketol-acid reductoisomerase [Escherichia coli MS 175-1]. |
| ZP_07379857 | 624 | 491 | ketol-acid reductoisomerase [Pantoea sp. aB]. |
| ZP_07593268 | 625 | 491 | ketol-acid reductoisomerase [Escherichia coli W]. |
| ZP_07783697 | 626 | 491 | ketol-acid reductoisomerase [Escherichia coli 2362-75]. |
| ZP_08039786 | 627 | 491 | ketol-acid reductoisomerase, NAD(P)-binding [Serratia symbiotica |
| | | | str. Tucson]. |
| ZP_08253005 | 628 | 491 | ketol-acid reductoisomerase [Plautia stali symbiont]. |
| ZP_08371457 | 629 | 491 | ketol-acid reductoisomerase (Acetohydroxy-acidisomeroreductase) |
| | | | (Alpha-keto-beta-hydroxylacil reductoisomerase) [Escherichia coli |
| | | | TA271]. |
| ZP_08495823 | 630 | 491 | ketol-acid reductoisomerase [Enterobacter hormaechei ATCC |
| | | | 49162]. |
| ZP_08571682 | 631 | 491 | ketol-acid reductoisomerase [Rheinheimera sp. A13L]. |
| ADP66401 | 632 | 490 | ketol-acid reductoisomerase [Buchnera aphidicola str. LL01 |
| | | | (Acyrthosiphon pisum)]. |
| ADP67563 | 633 | 490 | ketol-acid reductoisomerase [Buchnera aphidicola str. JF99 |
| | | | (Acyrthosiphon pisum)]. |
| AEO08356 | 634 | 490 | ketol-acid reductoisomerase [Buchnera aphidicola str. Ua |
| | | | (Uroleucon ambrosiae)]. |
| AEO08924 | 635 | 490 | ketol-acid reductoisomerase [Buchnera aphidicola str. Ak |
| | | | (Acyrthosiphon kondoi)]. |
| EHA15408 | 636 | 490 | ketol-acid reductoisomerase [Halomonas sp. HAL1]. |
| ILVC_BUCMH | 637 | 490 | RecName: Full = Ketol-acid reductoisomerase; AltName: |
| | | | Full = Acetohydroxy-acid isomeroreductase; AltName: Full = Alpha- |
| | | | keto-beta-hydroxylacil reductoisomerase [Buchnera aphidicola] |
| NP_240398 | 638 | 490 | ketol-acid reductoisomerase [Buchnera aphidicola str. APS |
| | | | (Acyrthosiphon pisum)]. |
| YP_001342680 | 639 | 490 | ketol-acid reductoisomerase [Marinomonas sp. MWYL1]. |
| YP_002139558 | 640 | 490 | ketol-acid reductoisomerase [Geobacter bemidjiensis Bem]. |
| YP_002603369 | 641 | 490 | ketol-acid reductoisomerase [Desulfobacterium autotrophicum |
| | | | HRM2]. |
| YP_003021297 | 642 | 490 | ketol-acid reductoisomerase [Geobacter sp. M21]. |
| YP_004069265 | 643 | 490 | ketol-acid reductoisomerase [Pseudoalteromonas sp. SM9913]. |
| YP_004199351 | 644 | 490 | ketol-acid reductoisomerase [Geobacter sp. M18]. |
| YP_004313466 | 645 | 490 | ketol-acid reductoisomerase [Marinomonas mediterranea MMB-1]. |
| YP_004429587 | 646 | 490 | ketol-acid reductoisomerase [Krokinobacter sp. 4H-3-7-5]. |
| YP_004482911 | 647 | 490 | ketol-acid reductoisomerase [Marinomonas posidonica IVIA-Po- |
| | | | 181]. |
| YP_339373 | 648 | 490 | ketol-acid reductoisomerase [Pseudoalteromonas haloplanktis |
| | | | TAC125]. |
| YP_357926 | 649 | 490 | ketol-acid reductoisomerase [Pelobacter carbinolicus DSM 2380]. |
| YP_802928 | 650 | 490 | ketol-acid reductoisomerase [Buchnera aphidicola str. Cc (Cinara |
| | | | cedri)]. |
| ZP_01053665 | 651 | 490 | ketol-acid reductoisomerase [Polaribacter sp. MED152]. |
| ZP_01104734 | 652 | 490 | ketol-acid reductoisomerase [Congregibacter litoralis KT71]. |
| ZP_01313517 | 653 | 490 | ketol-acid reductoisomerase [Desulfuromonas acetoxidans DSM |
| | | | 684]. |
| ZP_01613990 | 654 | 490 | ketol-acid reductoisomerase [Alteromonadales bacterium TW-7]. |
| ZP_03702229 | 655 | 490 | ketol-acid reductoisomerase [Flavobacteria bacterium MS024-2A]. |
| ZP_05096116 | 656 | 490 | ketol-acid reductoisomerase, putative [marine gamma |
| | | | proteobacterium HTCC2148]. |
| ZP_05129242 | 657 | 490 | ketol-acid reductoisomerase [gamma proteobacterium NOR5-3]. |
| ZP_08410735 | 658 | 490 | ketol-acid reductoisomerase [Pseudoalteromonas haloplanktis |
| | | | ANT/505]. |
| ZP_08568206 | 659 | 490 | ketol-acid reductoisomerase [Shewanella sp. HN-41]. |
| ADI23720 | 660 | 489 | ketol-acid reductoisomerase [uncultured Oceanospirillales bacterium |
| | | | HF4000_21D01]. |
| ILVC_BUCDN | 661 | 489 | RecName: Full = Ketol-acid reductoisomerase; AltName: |
| | | | Full = Acetohydroxy-acid isomeroreductase; AltName: Full = Alpha- |
| | | | keto-beta-hydroxylacil reductoisomerase [Buchnera aphidicola]. |
| ILVC_BUCSC | 662 | 489 | RecName: Full = Ketol-acid reductoisomerase; AltName: |
| | | | Full = Acetohydroxy-acid isomeroreductase; AltName: Full = Alpha- |
| | | | keto-beta-hydroxylacil reductoisomerase [Buchnera aphidicola]. |
| YP_001195200 | 663 | 489 | ketol-acid reductoisomerase [Flavobacterium johnsoniae UW101]. |
| YP_004739827 | 664 | 489 | alpha-keto-beta-hydroxylacil reductoisomerase [Capnocytophaga |
| | | | canimorsus Cc5]. |
| ZP_01627670 | 665 | 489 | ketol-acid reductoisomerase [marine gamma proteobacterium |
| | | | HTCC2080]. |
| ZP_01873277 | 666 | 489 | ketol-acid reductoisomerase [Lentisphaera araneosa HTCC2155]. |
| ZP_01890129 | 667 | 489 | ketol-acid reductoisomerase [unidentified eubacterium SCB49]. |
| YP_004590210 | 668 | 488 | ketol-acid reductoisomerase [Buchnera aphidicola (Cinara |
| | | | tujafilina)]. |
| ZP_01253273 | 669 | 476 | ketol-acid reductoisomerase [Psychroflexus torquis ATCC 700755]. |
| EGX01646 | 670 | 469 | ketol-acid reductoisomerase [Escherichia coli G58-1]. |
| YP_003585316 | 671 | 469 | ketol-acid reductoisomerase [Zunongwangia profunda SM-A87]. |
| ZP_08269825 | 672 | 467 | ketol-acid reductoisomerase [gamma proteobacterium IMCC3088]. |
| ZP_03342290 | 673 | 463 | ketol-acid reductoisomerase, partial [Salmonella enterica subsp. |
| | | | enterica serovar Typhi str. 404ty]. |
| ZP_03378451 | 674 | 452 | ketol-acid reductoisomerase [Salmonella enterica subsp. enterica |
| | | | serovar Typhi str. J185]. |
| ZP_01785421 | 675 | 450 | ketol-acid reductoisomerase [Haemophilus influenzae 22.1-21]. |
| EGP05382 | 676 | 442 | ketol-acid reductoisomerase [Pasteurella multocida subsp. gallicida |
| | | | str. Anand1_poultry]. |

### Example 27

### NADH-Dependent KARI Derived from Shewanella and Salmonella

The following example illustrates exemplary long-form KARI enzymes from Shewanella sp. and Salmonella enterica and corresponding NADH-dependent ketol-acid reductoisomerases (NKR) derived therefrom.

Plasmids and primers disclosed in this example are shown in Tables 45-46 below.

TABLE 45
Plasmids Disclosed in Example 27.

| Plasmids | Genotype |
|---|---|
| pET22b(+) | PT7, bla, ori pBR322, lacI, C-term 6xHis |
| pGV3195 | PT7::Se1_KARI^{his6}, bla, oripBR322, lacI |
| pGV3627 | PT7::Sh_sp_KARI_coSc^{his6}, bla, oripBR322, lacI |
| pGV3628 | PT7::Sh_sp_NKR_coSc^{DDhis6}, bla, oripBR322, |
| | lacI |
| pGVSh_sp_S78D | PT7::Sh_sp_KARI_coSc^{S78Dhis6}, bla, oripBR322, |
| | lacI |
| pGV3629 | PT7::Sh_sp_NKR_coSc^{6E6his6}, bla, oripBR322, |
| | lacI |
| pGV3630 | PT7::Se2_KARI_coSc^{his6}, bla, oripBR322, lacI |
| pGVSe2_S78D | PT7::Se2_KARI_coSc^{S78Dhis6}, bla, oripBR322, |
| | lacI |
| pGV3631 | PT7::Se2_NKR_coSc^{DDhis6}, bla, oripBR322, lacI |
| pGV3632 | PT7::Se2_NKR_coSc^{6E6his6}, bla, oripBR322, lacI |

TABLE 46
Oligonucleotide Primers Disclosed in Example 27.

| Primer | |
|---|---|
| name | Sequence |
| Sh_S78D_for | GCACAAAAGAGAGCCGATTGGCAAAAAGCGAC |
| | (SEQ ID NO: 677) |
| Sh_S78D_rev | GTCGCTTTTTGCCAATCGGCTCTCTTTTGTGC |
| | (SEQ ID NO: 678) |
| Se2_S78D_for | GCAGAAAAGAGAGCCGATTGGCGTAAAGCGACGGA |
| | (SEQ ID NO: 679) |
| Se2_S78D_rev | TCCGTCGCTTTACGCCAATCGGCTCTCTTTTCTGC |
| | (SEQ ID NO: 680) |

Mutations relative to wild-type Salmonella enterica KARI (Se2_KARI) and Shewanella sp. KARI (Sh_sp_KARI) are listed in Table 47 below.

TABLE 47
Mutations Relative to Se2_KARI and Sh_KARI.

| Source | Variant (enzyme) | Mutations |
|---|---|---|
| Salmenella enterica | Se2_KARI | n/a |
| | Se2_KARI^{S78D} | S78D |
| | Se2_NKR^{DD} | R76D, S78D |
| | Se2_NKR^{6E6} | A71S, R76D, S78D, Q110V |
| Shewanella sp. | Sh_sp_KARI | n/a |
| | Sh_sp_NKR^{S78D} | S78D |
| | Sh_sp_NKR^{DD} | R76D, S78D |
| | Sh_sp_NKR^{6E6} | A71S, R76D, S78D, Q110V |

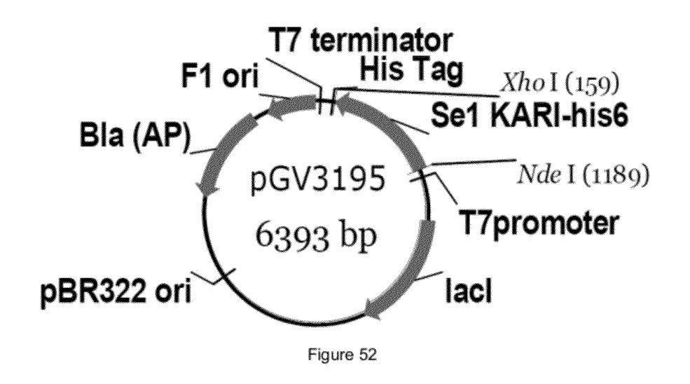

Genes encoding Se2_KARI, Se2_NKR^{DD}, Se2_NKR^{6E6}, Sh_sp_KARI, Sh_sp_NKR^{DD}, and Sh_sp_NKR^{6E6} were synthesized by GenScript USA Inc. (Piscataway, N.J. 08854 USA) with flanking NdeI and XhoI sites. The genes were isolated by restriction enzyme digestion with NdeI and XhoI for 1 hour at 37° C. The expression vector, pGV3195 (FIG. 52), was also digested with NdeI and XhoI for 1 hour at 37° C. The fragments were ligated using T4 DNA ligase from New England Biolabs (Ipswich, Mass. USA). The ligated DNAs were transformed into chemically competent E. coli DH5α cells, incubated for 1 h at 37° C. in SOC medium, and plated to LBₐₘₚ agar plates (Luria Bertani Broth, Research Products International Corp, supplemented with 100 μg/mL ampicillin) to yield single colonies. After confirming the correct sequence, E. coli BL21(DE3) (Table 3) cells were transformed with the correct plasmids for expression

Genes encoding Se2_KARI^{S78D} and Sh_sp_NKR^{S78D}: Single aspartic acid substitutions were introduced using the QuikChange site-directed mutagenesis kit according to manufacturer's protocol (Stratagene). Plasmids pGV3627 and pGV3630 encoding Sh_sp_KARI and Se2_KARI, respectively, were used as templates. The respective primer pairs were primers Sh_S78D_for and Sh_S78D_rev and primers Se2_S78D_for and Se2_S78D_rev. Pfu turbo polymerase (Stratagene) was used as the polymerase in the following PCR program: 95° C. for 2 min; 95° C. for 30 s, 55° C. for 30 s, 72° C. for 8 min (repeat 15 times); 72° C. for 10 min. After the PCR program was completed, the reaction mixtures were digested with DpnI for 1 h at 37° C. Then, chemically competent E. coli XL1-Gold cells were transformed with 3 μL of the un-cleaned PCR mixtures and the cells were allowed to recover in SOC medium at 37° C. with shaking at 250 rpm for 1 h. The recovery allowed the cells to close the nick-containing DNA produced during the PCR and thus to generate circularized plasmids. We then plated varying volumes on LBₐₘₚ agar plates (Luria Bertani Broth, Research Products International Corp, supplemented with 100 μg/mL ampicillin) to yield single colonies. After confirming the correct sequence, E. coli BL21 (DE3) cells were transformed with the correct plasmids for expression.

Heterologous expression of Se2_KARI and Sh_sp_KARI variants in E. coli: The expression of Se2_KARI, Sh_sp_KARI, and their corresponding NKR variants (Table 47) was conducted in 0.5 L Erlenmeyer flasks filled with 0.2 L LBₐₘₚ (Luria Bertani Broth, Research Products International Corp, supplemented with 100 μg/mL ampicillin) inoculated with overnight culture to an initial OD₆₀₀ of 0.1. After growing the expression cultures at 37° C. with shaking at 250 rpm for 4 h, the cultivation temperature was dropped to 25° C., and KARI expression was induced with IPTG to a final concentration of 0.5 mM. After 24 h at 25° C. and shaking at 250 rpm, the cells were pelleted at 5,300 g for 10 min and then frozen at −20° C. until further use.

Histrap purification of KARI^{hiS6} variants: Cell pellets used for purification were resuspended in purification buffer A (20 mM Tris, 20 mM imidazole, 100 mM NaCl, 10 mM MgCl2, pH 7.4). KARI enzymes and their corresponding NKR variants were purified by IMAC (Immobilized metal affinity chromatography) over a 1-ml Histrap High Performance (histrap HP) column pre-charged with Nickel (GE Healthcare) using an AKTApurifier™ FPLC system (GE Healthcare). The column was equilibrated with four column volumes (cv) of buffer A. After injecting the crude extract, the column was washed with buffer A for 2 cv, followed by a wash step with a mixture of 10% elution buffer B (20 mM Tris, 300 mM imidazole, 100 mM NaCl, 10 mM MgCl2, pH 7.4) for 5 cv. KARI enzymes and their corresponding NKR variants were eluted at 40% buffer B and stored at 4° C.

Preparation of enantiopure (S)-2-acetolactate: Enzymatic synthesis of (S)-2-acetolactate was performed in an anaerobic flask. The reaction was carried out in a total volume of 55 mL containing 20 mM potassium phosphate buffer, pH 7.0, 1 mM MgCl₂, 0.05 mM thiamine pyrophosphate (TPP), and 200 mM sodium pyruvate. The synthesis was initiated by the addition of 65 units of purified B. subtilis acetolactate synthase (Bs_AlsS), and the reaction was incubated at 30° C. (in a static incubator) for 7.5 hours. A buffer exchange was performed on the purified Bs_AlsS before the synthesis to remove as much glycerol as possible. This was done using a microcon filter with a 50 kDa nominal molecular weight cutoff membrane to filter 0.5 mL of the purified enzyme until only 50 μL were left on top of the membrane. 450 μL of 20 mM KPO₄ pH 7.0, 1 mM MgCl₂, and 0.05 mM TPP were then added to the membrane and filtered again, this process was repeated three times. The final acetolactate concentration was determined by liquid chromatography and was 218 mM in this batch used here.

KARI assay in 1-mL scale with the purpose to measure the NADPH and NADH K_{M} values: Activities KARI enzymes and their corresponding NKR variants were assayed kinetically by monitoring the decrease in NADPH or NADH concentration by measuring the change in absorbance at 340 nm. An assay buffer was prepared containing 100 mM potassium phosphate pH 7.0, 1 mM DTT, 10 mM (S)-2-acetolactate, and 10 mM MgCl₂ (final concentrations in the 1-mL assay, accounting for dilution with enzyme and cofactor). Fifty μL purified enzyme and 930 μL of the assay buffer were placed into a 1-mL cuvette. The reaction was initiated by addition of 20 μL NADPH or NADH (200 μM final concentration) for a general activity assay. Michaelis-Menten constants of the cofactors were determined with varying concentrations of NADPH or NADH (6-200 μM final concentrations).

Protein quantification: For determination of the specific activity values, we quantified the protein concentration of the purified enzymes using the BioRad Bradford Protein Assay Reagent Kit (Cat#500-0006, BioRad Laboratories) using BSA for the standard curve.

Sh_sp_KARI and its NKR variants were expressed in 200-mL cultures and purified over a 1-mL histrap HP column. The K_{M}, k_{cat}, and specific acitivity values were measured as described above, and the results are summarized in Table 48. In terms of the ratio of catalytic efficiency with NADH over NADPH, variants Sh_sp_NKR^{S78D}, Sh_sp_NKR^{R76DS78D}, and Sh_sp_NKR^{6E6} can be defined as being NADH-dependent KARIs (NKR) in terms of their catalytic efficiencies.

Se2_KARI and its NKR variants were expressed in 200-mL cultures and purified over a 1-mL histrap HP column. The K_{M}, k_{cat}, and specific acitivity values were measured as described above, and the results are summarized in Table 49. In terms of the ratio of catalytic efficiency with NADH over NADPH, variants Se2_NKR^{DD} and Se2_NKR^{6E6} can be defined as being NADH-dependent KARIs (NKR).

TABLE 48
Comparison of properties of wild-type Shewanella sp. KARI (Sh_sp_KARI), the single and double
aspartic acid variants, and the “6E6” variant. Data is based on measurements using purified proteins.

| | Sp. Activity [U/mg] | | | | | | Kₘ [μM] for cofactor | | | |
|---|---|---|---|---|---|---|---|---|---|---|
| | NADH | ± | NADPH | ± | ratio | ± | NADH | ± | NADPH | ± |
| Sh_sp_KARI | 0.30 | 0.012 | 1.2 | 0.013 | 0.2 | 0.0 | 415 | 44 | ≦1 | |
| Sh_sp_NKR^{S78D} | 0.36 | 0.024 | 0.5 | 0.042 | 0.7 | 0.1 | 130 | 15 | 267 | 26 |
| Sh_sp_NKR^{DD} | 0.34 | 0.001 | 0.03 | 0.004 | 10.7 | 1.5 | 90 | 24 | >1000 | |
| Sh_sp_NKR^{6E6} | 0.66 | 0.00 | 0.1 | 0.013 | 9.1 | 1.7 | 75 | 10 | 600 | 130 |

| | k_{cat} [^{s-1}] | | | | k_{cat}/Kₘ [M⁻¹ * s⁻¹] | | | | | |
|---|---|---|---|---|---|---|---|---|---|---|
| | NADH | ± | NADPH | ± | NADH | ± | NADPH | ± | ratio | ± |
| Sh_sp_KARI | 1.1 | 0.05 | 4.5 | 0.05 | 2,649 | 301 | 4,479,890 | | 0.0006 | |
| Sh_sp_NKR^{S78D} | 1.3 | 0.09 | 2.0 | 0.16 | 10,269 | 1,360 | 7,373 | 924 | 1.4 | 0.3 |
| Sh_sp_NKR^{DD} | 1.3 | 0.01 | 0.1 | 0.02 | 14,022 | 3,740 | <117 | | 119 | |
| Sh_sp_NKR^{6E6} | 2.4 | 0.00 | 0.3 | 0.05 | 32,410 | 4,322 | 446 | 127 | 73 | 22.9 |

TABLE 49
Comparison of properties of wild-type Salmonella enterica KARI (Se2_KARI), the single and double
aspartic acid variants, and the “6E6” variant. Data is based on measurements using purified proteins.

| | Sp. Activity [U/mg] | | | | | | Kₘ [μM] for cofactor | | | |
|---|---|---|---|---|---|---|---|---|---|---|
| | NADH | ± | NADPH | ± | ratio | ± | NADH | ± | NADPH | ± |
| Se2_KARI | 0.14 | 0.009 | 1.1 | 0.062 | 0.1 | 0.0 | 157 | 4 | 8 | 2 |
| Se2_NKR^{S78D} | 0.17 | 0.005 | 0.4 | 0.015 | 0.4 | 0.0 | 233 | 43 | 272 | 23 |
| Se2_NKR^{DD} | 0.37 | 0.002 | 0.03 | 0.005 | 12.4 | 1.9 | 121 | 20 | >1000 | |
| Se2_NKR^{6E6} | 0.64 | 0.10 | 0.1 | 0.045 | 6.6 | 3.2 | 24 | 4 | 630 | 291 |

| | k_{cat} [^{s-1}] | | | | k_{cat}/Kₘ [M⁻¹ * s⁻¹] | | | | | |
|---|---|---|---|---|---|---|---|---|---|---|
| | NADH | ± | NADPH | ± | NADH | ± | NADPH | ± | ratio | ± |
| Se2_KARI | 0.51 | 0.03 | 3.96 | 0.23 | 3,229 | 222 | 495,409 | 127,118 | 0.01 | 0.0 |
| Se2_NKR^{S78D} | 0.63 | 0.02 | 1.47 | 0.05 | 2,696 | 503 | 5,402 | 498 | 0.5 | 0.1 |
| Se2_NKR^{DD} | 1.36 | 0.01 | 0.11 | 0.02 | 11,222 | 1,856 | <110 | | >102 | |
| Se2_NKR^{6E6} | 2.33 | 0.35 | 0.36 | 0.01 | 97,284 | 21,857 | 565 | 261 | 172 | 88.5 |

The foregoing detailed description has been given for clearness of understanding only and no unnecessary limitations should be understood there from as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

What is claimed is:

1. A recombinant yeast microorganism comprising an engineered metabolic pathway for producing isobutanol, wherein said engineered metabolic pathway comprises the following pathway steps:
(a) pyruvate to acetolactate;
(b) acetolactate to 2,3-dihydroxyisovalerate;
(c) 2,3-dihydroxyisovalerate to α-ketoisovalerate;
(d) α-ketoisovalerate to isobutyraldehyde; and
(e) isobutyraldehyde to isobutanol,
wherein the recombinant yeast microorganism expresses:
(i) a modified class II ketol-acid reductoisomerase (KARI), wherein said modified class II KARI is derived from an unmodified class II KARI that has been engineered to comprise an amino acid substitution corresponding to amino acid residue Ser78 of the E. coli KARI (SEQ ID NO: 13), and wherein said modified class II KARI exhibits an increased ability to use NADH as a cofactor to catalyze the conversion of acetolactate to 2,3-dihydroxyisovalerate as compared to a corresponding unmodified class II KAR1; and
(ii) an exogenously encoded alcohol dehydrogenase that is naturally NADH-dependent, wherein the alcohol dehydrogenase catalyzes the conversion of isobutyraldehyde to isobutanol.

2. The recombinant yeast microorganism of claim 1, wherein the amino acid residue corresponding to Ser78 of the E. coli ketol-acid reductoisomerase (SEQ ID NO: 13) is replaced with an amino acid residue selected from the group consisting of aspartic acid and glutamic acid.

3. The recombinant yeast microorganism of claim 1, wherein said unmodified class II KARI is derived from a genus selected from the group consisting of Escherichia, Shigella, Yersinia, Klebsiella, Vibrio, Providencia, Haemophilus, Shewanella, and Salmonella.

4. The recombinant yeast microorganism of claim 1, wherein said unmodified class II KARI comprises SEQ ID NO: 13.

5. The recombinant yeast microorganism of claim 1, wherein said unmodified class II KARI comprises an amino acid sequence selected from the group consisting of SEQ ID NOs: 331-676.

6. The recombinant yeast microorganism of claim 1, wherein the enzyme that catalyzes the conversion of pyruvate to acetolactate is an acetolactate synthase.

7. The recombinant yeast microorganism of claim 6, wherein said acetolactate synthase is derived from Bacillus subtilis.

8. The recombinant yeast microorganism of claim 1, wherein the enzyme that catalyzes the conversion of 2,3-dihydroxyisovalerate to α-ketoisovalerate is a dihydroxy acid dehydratase.

9. The recombinant yeast microorganism of claim 1, wherein the enzyme that catalyzes the conversion of α-ketoisovalerate to isobutyraldehyde is a 2-keto acid decarboxylase.

10. The recombinant yeast microorganism of claim 9, wherein said 2-keto acid decarboxylase is derived from Lactococcus lactis.

11. The recombinant yeast microorganism of claim 1, wherein said alcohol dehydrogenase is encoded by a gene selected from the group consisting of the Lactococcus lactis adhA, the Drosophila melanogaster ADH, the Klebsiella pneumoniae dhaT, and the Escherichia coli fucO.

12. The recombinant yeast microorganism of claim 1, wherein said recombinant yeast microorganism has been engineered to reduce or eliminate pyruvate decarboxylase (PDC) activity.

13. The recombinant yeast microorganism of claim 1, wherein said recombinant yeast microorganism has been engineered to reduce or eliminate glycerol-3-phosphate dehydrogenase (GPD) activity.

14. The recombinant yeast microorganism of claim 1, wherein said modified class II, KARI is encoded by a nucleic acid molecule which has been codon-optimized for expression in yeast.

15. A method of producing isobutanol, said method comprising:
(a) providing a recombinant yeast microorganism according to claim 1;
(b) cultivating the recombinant yeast microorganism in a culture medium containing a feedstock providing a carbon source until the isobutanol is produced.

16. The method of claim 15, wherein said recombinant yeast microorganism is cultivated under anaerobic conditions.
